SEQUENCE LISTING (140 sequences)

SEQ ID NO: 1 - DNA, 3 bp, Artificial Sequence
  Synthetic
  misc_feature (1)..(1): n is a, c, g, or t
  r (2)..(2): r is a, or g
nrg

SEQ ID NO: 2 - DNA, 3 bp, Artificial Sequence
  Synthetic
  misc_feature (1)..(1): n is a, c, g, or t
ngg

SEQ ID NO: 3 - DNA, 3 bp, Artificial Sequence
  Synthetic
  misc_feature (1)..(1): n is a, c, g, or t
nag

SEQ ID NO: 4 - DNA, 5 bp, Artificial Sequence
  Synthetic
  misc_feature (1)..(1): n is a, c, g, or t
  misc_feature (4)..(4): n is a, c, g, or t
nggng

SEQ ID NO: 5 - DNA, 7 bp, Artificial Sequence
  Synthetic
  misc_feature (1)..(2): n is a, c, g, or t
  w (7)..(7): w is a, or t
nnagaaw

SEQ ID NO: 6 - DNA, 4 bp, Artificial Sequence
  Synthetic
  misc_feature (1)..(1): n is a, c, g, or t
  r (4)..(4): r is a, or g
naar

SEQ ID NO: 7 - DNA, 5 bp, Artificial Sequence
  Synthetic
  misc_feature (1)..(2): n is a, c, g, or t
  r (4)..(5): r is a, or g
nngrr

SEQ ID NO: 8 - DNA, 6 bp, Artificial Sequence
  Synthetic
  misc_feature (1)..(2): n is a, c, g, or t
  r (4)..(5): r is a, or g
  misc_feature (6)..(6): n is a, c, g, or t
nngrrn

SEQ ID NO: 9 - DNA, 6 bp, Artificial Sequence
  Synthetic
  misc_feature (1)..(2): n is a, c, g, or t
  r (4)..(5): r is a, or g
nngrrt

SEQ ID NO: 10 - DNA, 6 bp, Artificial Sequence
  Synthetic
  misc_feature (1)..(2): n is a, c, g, or t
  r (4)..(5): r is a, or g
  v (6)..(6): v is a, c, or g
nngrrv

SEQ ID NO: 11 - DNA, 8 bp, Artificial Sequence
  Synthetic
  misc_feature (1)..(4): n is a, c, g, or t
nnnngatt

SEQ ID NO: 12 - DNA, 8 bp, Artificial Sequence
  Synthetic
  misc_feature (1)..(4): n is a, c, g, or t
  misc_feature (6)..(8): n is a, c, g, or t
nnnngnnn

SEQ ID NO: 13 - DNA, 3 bp, Artificial Sequence
  Synthetic
  misc_feature (1)..(1): n is a, c, g, or t
nga

SEQ ID NO: 14 - DNA, 6 bp, Artificial Sequence
  Synthetic
  misc_feature (1)..(3): n is a, c, g, or t
  r (4)..(5): r is a, or g
nnnrrt

SEQ ID NO: 15 - DNA, 6 bp, Artificial Sequence
  Synthetic
attcct

SEQ ID NO: 16 - DNA, 4 bp, Artificial Sequence
  Synthetic
  misc_feature (1)..(1): n is a, c, g, or t
  misc_feature (4)..(4): n is a, c, g, or t
ngan

SEQ ID NO: 17 - DNA, 4 bp, Artificial Sequence
  Synthetic
  misc_feature (1)..(1): n is a, c, g, or t
  misc_feature (3)..(3): n is a, c, g, or t
ngng

SEQ ID NO: 18 - DNA, 86 bp, Artificial Sequence
  Synthetic
gtttaagagctatgctggaaacagcatagcaagtttaaataaggctagtccgttatcaacttgaaaaagtggcaccgagtcggtgc

SEQ ID NO: 19 - RNA, 86 nt, Artificial Sequence
  Synthetic
guuuaagagcuaugcuggaaacagcauagcaaguuuaaauaaggcuaguccguuaucaacuugaaaaaguggcaccgagucggugc

SEQ ID NO: 20 - protein, 1368 aa, Streptococcus pyogenes [sequence omitted]

SEQ ID NO: 21 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 22 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 23 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 24 - DNA, 3159 bp, Artificial Sequence
  Synthetic
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 25 - DNA, 3159 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 26 - DNA, 4107 bp, Artificial Sequence
  Synthetic
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 27 - DNA, 3158 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 28 - DNA, 3159 bp, Artificial Sequence
  Synthetic
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 29 - DNA, 3159 bp, Artificial Sequence
  Synthetic
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 30 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 31 - DNA, 3242 bp, Artificial Sequence
  Synthetic
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaattc

SEQ ID NO: 32 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 33 - DNA, 3156 bp, Artificial Sequence
  Synthetic
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 34 - DNA, 7009 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 35 - protein, 2414 aa, Artificial Sequence
  Synthetic
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 36 - protein, 617 aa, Artificial Sequence
  Synthetic
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 37 - protein, 1497 aa, Artificial Sequence
  Synthetic
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 38 - DNA, 4491 bp, Artificial Sequence
  Synthetic
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 39 - DNA, 4560 bp, Artificial Sequence
  Synthetic
atggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttga

SEQ ID NO: 40 - protein, 1519 aa, Artificial Sequence
  Synthetic
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 41 - DNA, 3570 bp, Artificial Sequence
  Synthetic
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 42 - protein, 1190 aa, Artificial Sequence
  Synthetic
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 43 - DNA, 2154 bp, Artificial Sequence
  Synthetic
ctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

SEQ ID NO: 44 - protein, 718 aa, Artificial Sequence
  Synthetic
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 45 - protein, 378 aa, Artificial Sequence
  Synthetic
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 46 - DNA, 1134 bp, Artificial Sequence
  Synthetic
gatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 47 - protein, 522 aa, Artificial Sequence
  Synthetic
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 48 - DNA, 1566 bp, Artificial Sequence
  Synthetic
gatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtcccaaaaagaagaggaaagtgggatcccagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 49 - protein, 7 aa, Artificial Sequence
  Synthetic
PKKKRKV

SEQ ID NO: 50 - protein, 5 aa, Artificial Sequence
  Synthetic
  REPEAT (1)..(5): repeat 0 to 10 times
GGGGS

SEQ ID NO: 51 - protein, 5 aa, Artificial Sequence
  Synthetic
GGGGG

SEQ ID NO: 52 - protein, 5 aa, Artificial Sequence
  Synthetic
GGAGG

SEQ ID NO: 53 - protein, 7 aa, Artificial Sequence
  Synthetic
GGGGSSS

SEQ ID NO: 54 - protein, 7 aa, Artificial Sequence
  Synthetic
GGGGAAA

SEQ ID NO: 55 - DNA, 22 bp, Artificial Sequence
  Synthetic
acatttcctctctatacaaatg

SEQ ID NO: 56 - DNA, 24 bp, Artificial Sequence
  Synthetic
atatagtaatgaaattattggcac

SEQ ID NO: 57 - RNA, 22 nt, Artificial Sequence
  Synthetic
acauuuccucucuauacaaaug

SEQ ID NO: 58 - RNA, 24 nt, Artificial Sequence
  Synthetic
auauaguaaugaaauuauuggcac

SEQ ID NO: 59 - DNA, 130 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 60 - DNA, 157 bp, Artificial Sequence
  Synthetic
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggg

SEQ ID NO: 61 - DNA, 144 bp, Artificial Sequence
  Synthetic
gggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 62 - DNA, 119 bp, Artificial Sequence
  Synthetic
ctagtccactccctctctgcgcgctcgctcgctcactgaggccgggcgaccaaaggtcgcccgacgcccgggctttgcccgggcggcctcagtgagcgagcgagcgcgcagagagggac

SEQ ID NO: 63 - DNA, 249 bp, Artificial Sequence
  Synthetic
ggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctc

SEQ ID NO: 64 - DNA, 215 bp, Artificial Sequence
  Synthetic
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 65 - DNA, 252 bp, Artificial Sequence
  Synthetic
tcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgg

SEQ ID NO: 66 - DNA, 449 bp, Artificial Sequence
  Synthetic
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccag

SEQ ID NO: 67 - DNA, 387 bp, Artificial Sequence
  Synthetic
gagctccaccgcggtggcggccgtccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatat

SEQ ID NO: 68 - DNA, 810 bp, Artificial Sequence
  Synthetic
gtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcggcgcgccc

SEQ ID NO: 69 - DNA, 3156 bp, Artificial Sequence; identical to SEQ ID NO: 33
  Synthetic

SEQ ID NO: 70 - DNA, 60 bp, Artificial Sequence
  Synthetic
tagcaataaaggatcgtttattttcattggaagcgtgtgttggttttttgatcaggcgcg

SEQ ID NO: 71 - DNA, 232 bp, Artificial Sequence
  Synthetic
ctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 72 - DNA, 164 bp, Artificial Sequence
  Synthetic
tctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttac

SEQ ID NO: 73 - DNA, 7236 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcatttgtatagagaggaaatgtgttttagtactctggaaacagaatctactaaaacaaggcaaaatgccgtgtttatctcgtcaacttgttggcgagatttttctcgagtcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcggccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattctagcaataaaggatcgtttattttcattggaagcgtgtgttggttttttgatcaggcgcgggtaccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacatatagtaatgaaattattggcacgttttagtactctggaaacagaatctactaaaacaaggcaaaatgccgtgtttatctcgtcaacttgttggcgagatttttggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 74 - DNA, 7444 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcatttgtatagagaggaaatgtgttttagtactctggaaacagaatctactaaaacaaggcaaaatgccgtgtttatctcgtcaacttgttggcgagatttttctcgagctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattctagcaataaaggatcgtttattttcattggaagcgtgtgttggttttttgatcaggcgcgggtaccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacatatagtaatgaaattattggcacgttttagtactctggaaacagaatctactaaaacaaggcaaaatgccgtgtttatctcgtcaacttgttggcgagatttttggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 75 - DNA, 7236 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgagtcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcggccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattctagcaataaaggatcgtttattttcattggaagcgtgtgttggttttttgatcaggcgcgggtaccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacatatagtaatgaaattattggcacgttttagtactctggaaacagaatctactaaaacaaggcaaaatgccgtgtttatctcgtcaacttgttggcgagatttttggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 76 - DNA, 7444 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgagctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattctagcaataaaggatcgtttattttcattggaagcgtgtgttggttttttgatcaggcgcgggtaccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacatatagtaatgaaattattggcacgttttagtactctggaaacagaatctactaaaacaaggcaaaatgccgtgtttatctcgtcaacttgttggcgagatttttggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 77 - DNA, 7381 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgaggagctccaccgcggtggcggccgtccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatataccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattctagcaataaaggatcgtttattttcattggaagcgtgtgttggttttttgatcaggcgcgggtaccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacatatagtaatgaaattattggcacgttttagtactctggaaacagaatctactaaaacaaggcaaaatgccgtgtttatctcgtcaacttgttggcgagatttttggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 78 - DNA, 7804 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgaggtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcggcgcgcccaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattctagcaataaaggatcgtttattttcattggaagcgtgtgttggttttttgatcaggcgcgggtaccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacatatagtaatgaaattattggcacgttttagtactctggaaacagaatctactaaaacaaggcaaaatgccgtgtttatctcgtcaacttgttggcgagatttttggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 79 - DNA, 7408 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgagtcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcggccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacatatagtaatgaaattattggcacgttttagtactctggaaacagaatctactaaaacaaggcaaaatgccgtgtttatctcgtcaacttgttggcgagatttttggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 80 - DNA, 7616 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgagctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacatatagtaatgaaattattggcacgttttagtactctggaaacagaatctactaaaacaaggcaaaatgccgtgtttatctcgtcaacttgttggcgagatttttggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 81 - DNA, 7553 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgaggagctccaccgcggtggcggccgtccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatataccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacatatagtaatgaaattattggcacgttttagtactctggaaacagaatctactaaaacaaggcaaaatgccgtgtttatctcgtcaacttgttggcgagatttttggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 82 - DNA, 7976 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgaggtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcggcgcgcccaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacatatagtaatgaaattattggcacgttttagtactctggaaacagaatctactaaaacaaggcaaaatgccgtgtttatctcgtcaacttgttggcgagatttttggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 83 - DNA, 7373 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagtcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcggccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgactagtagaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 84 - DNA, 7570 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgactagtagaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 85 - DNA, 7508 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggagctccaccgcggtggcggccgtccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatatccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgactagtagaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 86 - DNA, 7913 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgactagtagaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 87 - DNA, 7009 bp, Artificial Sequence; identical to SEQ ID NO: 34
  Synthetic

SEQ ID NO: 88 - protein, 1052 aa, Artificial Sequence
  Synthetic
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 89 - DNA, 76 bp, Artificial Sequence
  Synthetic
tctcgccaacaagttgacgagataaacacggcattttgccttgttttagtagattctgtttccagagtactaaaac

SEQ ID NO: 90 - RNA, 76 nt, Artificial Sequence
  Synthetic
ucucgccaacaaguugacgagauaaacacggcauuuugccuuguuuuaguagauucuguuuccagaguacuaaaac

SEQ ID NO: 91 - DNA, 6396 bp, Artificial Sequence
  Synthetic
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatatagtaatgaaattattggcacgttttagtactctggaaacagaatctactaaaacaaggcaaaatgccgtgtttatctcgtcaacttgttggcgagatttttttgctcctagggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcatttgtatagagaggaaatgtgttttagtactctggaaacagaatctactaaaacaaggcaaaatgccgtgtttatctcgtcaacttgttggcgagattttttctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgacctcgagcagtgtggttttgcaagaggaagcaaaaagcctctccacccaggcctggaatgtttccacccaagtcgaaggcagtgtggttttgcaagaggaagcaaaaagcctctccacccaggcctggaatgtttccacccaatgtcgagcaaccccgcccagcgtcttgtcattggcgaattcgaacacgcagatgcagtcggggcggcgcggtcccaggtccacttcgcatattaaggtgacgcgtgtggcctcgaacaccgagcgaccctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggctgca

SEQ ID NO: 92 - DNA, 3993 bp, Artificial Sequence
  Synthetic
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcatatgccaagtacgccccctattgacgtcaatgacggtaaatggcccgcctggcattatgcccagtacatgaccttatgggactttcctacttggcagtacatctacgtattagtcatcgctattaccatggtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatatagtaatgaaattattggcacgttttagtactctggaaacagaatctactaaaacaaggcaaaatgccgtgtttatctcgtcaacttgttggcgagattttttgaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcatttgtatagagaggaaatgtgttttagtactctggaaacagaatctactaaaacaaggcaaaatgccgtgtttatctcgtcaacttgttggcgagatttttagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 93 - DNA, 23 bp, Artificial Sequence
  Synthetic
ctctgataacccagctgtgtgtt

SEQ ID NO: 94 - DNA, 23 bp, Artificial Sequence
  Synthetic
ctagaccatttcccaccagttct

SEQ ID NO: 95 - DNA, 21 bp, Artificial Sequence
  Synthetic
tgcctttcaatcattgtttcg

SEQ ID NO: 96 - DNA, 20 bp, Artificial Sequence
  Synthetic
aaggccccaaaatgtgaaat

SEQ ID NO: 97 - DNA, 20 bp, Artificial Sequence
  Synthetic
cgggcttggacagaacttac

SEQ ID NO: 98 - DNA, 20 bp, Artificial Sequence
  Synthetic
ctgcgtagtgccaaaacaaa

SEQ ID NO: 99 - DNA, 23 bp, Artificial Sequence
  Synthetic
gtttccagagctttacctgagaa

SEQ ID NO: 100 - DNA, 22 bp, Artificial Sequence
  Synthetic
cttttatgaatgcttctccaag

SEQ ID NO: 101 - DNA, 20 bp, Artificial Sequence
  Synthetic
aacaaatatcccttagtatc

SEQ ID NO: 102 - DNA, 20 bp, Artificial Sequence
  Synthetic
aatgtatttcttctattcaa

SEQ ID NO: 103 - DNA, 20 bp, Artificial Sequence
  Synthetic
ctccggaatgtctccatttg

SEQ ID NO: 104 - DNA, 20 bp, Artificial Sequence
  Synthetic
atgagggagagactggcatc

SEQ ID NO: 105 - DNA, 21 bp, Artificial Sequence
  Synthetic
tgcctttcaatcattgtttcg

SEQ ID NO: 106 - DNA, 20 bp, Artificial Sequence
  Synthetic
agaaggcaaattggcacaga

SEQ ID NO: 107 - DNA, 22 bp, Artificial Sequence
  Synthetic
tggttgtccagtgtgagtctcc

SEQ ID NO: 108 - DNA, 20 bp, Artificial Sequence
  Synthetic
gggctgcgtagtgccaaaac

SEQ ID NO: 109 - DNA, 21 bp, Artificial Sequence
  Synthetic
cctgctctggcagatttcaac

SEQ ID NO: 110 - DNA, 19 bp, Artificial Sequence
  Synthetic
acccaccatcaccctctgt

SEQ ID NO: 111 - DNA, 23 bp, Artificial Sequence
  Synthetic
aaagccagtcggtaagttctgtc

SEQ ID NO: 112 - DNA, 20 bp, Artificial Sequence
  Synthetic
gccgagtttatcgcctcctt

SEQ ID NO: 113 - DNA, 20 bp, Artificial Sequence
  Synthetic
ggtactcgcggtaggtgatg

SEQ ID NO: 114 - DNA, 21 bp, Artificial Sequence
  Synthetic
tctgagtggaaggcggtaaac

SEQ ID NO: 115 - DNA, 20 bp, Artificial Sequence
  Synthetic
cctccggttctgaaggtgtt

SEQ ID NO: 116 - DNA, 30 bp, Artificial Sequence
  Synthetic
tctttcaaaggctccaatagtggtcagtcc

SEQ ID NO: 117 - DNA, 53 bp, Artificial Sequence
  Synthetic
tcgtcggcagcgtcagatgtgtataagagacagttgccaccaatcttctggtt

SEQ ID NO: 118 - DNA, 54 bp, Artificial Sequence
  Synthetic
gtctcgtgggctcggagatgtgtataagagacagtgaagagggctgaacttgct

SEQ ID NO: 119 - DNA, 53 bp, Artificial Sequence
  Synthetic
tcgtcggcagcgtcagatgtgtataagagacagggtgggtgaccttgaggata

SEQ ID NO: 120 - DNA, 58 bp, Artificial Sequence
  Synthetic
gtctcgtgggctcggagatgtgtataagagacagtgagagtaatgtgtttgctgagag

SEQ ID NO: 121 - DNA, 30 bp, Artificial Sequence
  Synthetic
acaaatggtatcttaaggacctccaaggtg

SEQ ID NO: 122 - DNA, 19 bp, Artificial Sequence
  Synthetic
ctgagaattgggaacatgc

SEQ ID NO: 123 - DNA, 18 bp, Artificial Sequence
  Synthetic
catcggaaccttccaggg

SEQ ID NO: 124 - DNA, 21 bp, Artificial Sequence
  Synthetic
ggtgcttccagagtgctgagg

SEQ ID NO: 125 - DNA, 20 bp, Artificial Sequence
  Synthetic
aatggcggcgttttcattat

SEQ ID NO: 126 - DNA, 22 bp, Artificial Sequence
  Synthetic
ggtgtcctttgaatatgcaggt

SEQ ID NO: 127 - DNA, 20 bp, Artificial Sequence
  Synthetic
ttcttggcgtattgccttct

SEQ ID NO: 128 - DNA, 899 bp, Artificial Sequence
  Synthetic
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatatagtaatgaaattattggcacgttttagtactctggaaacagaatctactaaaacaaggcaaaatgccgtgtttatctcgtcaacttgttggcgagatttttttgctcctagggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcatttgtatagagaggaaatgtgttttagtactctggaaacagaatctactaaaacaaggcaaaatgccgtgtttatctcgtcaacttgttggcgagattttt

SEQ ID NO: 129 - DNA, 1298 bp, Artificial Sequence
  Synthetic
gggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcatatgccaagtacgccccctattgacgtcaatgacggtaaatggcccgcctggcattatgcccagtacatgaccttatgggactttcctacttggcagtacatctacgtattagtcatcgctattaccatggtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatatagtaatgaaattattggcacgttttagtactctggaaacagaatctactaaaacaaggcaaaatgccgtgtttatctcgtcaacttgttggcgagattttttgaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcatttgtatagagaggaaatgtgttttagtactctggaaacagaatctactaaaacaaggcaaaatgccgtgtttatctcgtcaacttgttggcgagatttttagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagtccactccctctctgcgcgctcgctcgctcactgaggccgggcgaccaaaggtcgcccgacgcccgggctttgcccgggcggcctcagtgagcgagcgagcgcgcagagagggac

SEQ ID NO: 130 - DNA, 6396 bp, Artificial Sequence; identical to SEQ ID NO: 91
  Synthetic

SEQ ID NO: 131 - DNA, 3993 bp, Artificial Sequence; identical to SEQ ID NO: 92
  Synthetic

SEQ ID NO: 132 - DNA, 157 bp, Artificial Sequence; identical to SEQ ID NO: 60
  Synthetic

SEQ ID NO: 133 - DNA, 249 bp, Artificial Sequence
  Synthetic
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 134 - DNA, 24 bp, Artificial Sequence
  Synthetic
atatagtaatgaaattattggcac

SEQ ID NO: 135 - DNA, 22 bp, Artificial Sequence
  Synthetic
catttgtatagagaggaaatgt

SEQ ID NO: 136 - RNA, 24 nt, Artificial Sequence
  Synthetic
auauaguaaugaaauuauuggcac

SEQ ID NO: 137 - RNA, 22 nt, Artificial Sequence
  Synthetic
cauuuguauagagaggaaaugu

SEQ ID NO: 138 - DNA, 81 bp, Artificial Sequence
  Synthetic
gttttagtactctggaaacagaatctactaaaacaaggcaaaatgccgtgtttatctcgtcaacttgttggcgagattttt

SEQ ID NO: 139 - RNA, 81 nt, Artificial Sequence
  Synthetic
guuuuaguacucuggaaacagaaucuacuaaaacaaggcaaaaugccguguuuaucucgucaacuuguuggcgagauuuuu

SEQ ID NO: 140 - DNA, 119 bp, Artificial Sequence; identical to SEQ ID NO: 62
  Synthetic

(19) United States
(12) Patent Application Publication
(10) Pub. No.: US 2023/0392132 A1
(43) Pub. Date: Dec. 7, 2023

(54) DUAL AAV VECTOR-MEDIATED DELETION OF LARGE MUTATIONAL HOTSPOT FOR TREATMENT OF DUCHENNE MUSCULAR DYSTROPHY

(71) Applicant: Duke University, Durham, NC (US)

(72) Inventors: Charles A. Gersbach, Chapel Hill, NC (US); Karen Bulaklak, Durham, NC (US)

(21) Appl. No.: 18/033,049

(22) PCT Filed: Oct. 21, 2021
(86) PCT No.: PCT/US2021/056122

§ 371 Date: Apr. 20, 2023

Related U.S. Application Data
(60) Provisional application No. 63/094,742, filed on Oct. 21, 2020.

(51) Int. Cl. C12N 9/22; C12N 15/11; C12N 15/86; C12N 15/90; A61K 48/00

(52) U.S. Cl. CPC C12N 9/22; C12N 15/11; C12N 15/86; C12N 15/907; A61K 48/0058; A61K 48/0066; C12N 2310/20; C12N 2750/14143; C12N 2750/14171; C12N 2800/40; C12N 2800/80

(57) ABSTRACT

Disclosed herein are therapeutic targets for the correction of the human dystrophin gene by gene editing and methods of use. The compositions and methods may include gRNAs targeting exons 44 and 55. The compositions and methods may also Include a mutant inverted terminal repeat (ITR) to generate a self-complementary vector genome.

### CROSS-REFERENCE TO RELATED APPLICATIONS

This application claims priority to U.S. Provisional Patent Application No. 63/094,742, filed Oct. 21, 2020, which is incorporated herein by reference in its entirety.

### STATEMENT REGARDING FEDERALLY SPONSORED RESEARCH

This invention was made with government support under grant R01AR069085, grant DP2-OD008586, grant T32GM008555 awarded by the National Institutes of Health, and grant 17PRE33350013 awarded by the National Science Foundation. The government has certain rights in the invention.

### FIELD

The present disclosure relates to the field of gene expression alteration, genome engineering, and genomic alteration of genes using Clustered Regularly Interspaced Short Palindromic Repeats (CRISPR)/CRISPR-associated (Cas) 9-based systems and viral delivery systems. The present disclosure also relates to the field of genome engineering and genomic alteration of genes in muscle, such as skeletal muscle and cardiac muscle.

### INTRODUCTION

Duchenne muscular dystrophy (DMD) is a degenerative muscle wasting disease that leads to loss of ambulation and premature death due to cardiac and respiratory complications. DMD is the most prevalent childhood genetic disease, affecting approximately 1 in every 3500 to 5000 male births. The most common mutations in the DMD gene are deletions of one or more exons that disrupt the reading frame and lead to a lack of dystrophin protein. In contrast, Becker muscular dystrophy (BMD) results from intragenic DMD deletions that maintain the correct translational reading frame, leading to a shortened, but still largely functional dystrophin protein. Because BMD typically presents with a later onset of symptoms and slower disease progression, strategies to address the genetic cause of DMD are often designed to restore the reading frame of the endogenous DMD gene and shift the DMD genotype towards a BMD genotype.

Although DMD is a candidate for gene therapy treatment, the large size of the wild type dystrophin cDNA (˜11.5 kb coding sequence) is not compatible with size-restricted gene delivery vehicles that efficiently transduce skeletal and cardiac muscle, such as adeno-associated virus (AAV). Therefore, some therapeutic strategies aim to restore an internally truncated, but functional dystrophin protein as found in BMD patients. These approaches include delivery of minidystrophin or microdystrophin6, oligonucleotide-mediated exon skipping, and use of genome editing nucleases. A common strategy is skipping or excising exon 51, which would lead to dystrophin restoration in approximately 13% of the patient population, the largest population of DMD patients treatable by removal of a single exon.

Genome editing involves targeted alteration of genome sequences by harnessing DNA repair pathways after the cleavage of genomic DNA at a target site by an engineered, programmable nuclease. The most commonly used genome editing technologies include zinc finger nucleases (ZFNs), meganucleases, transcription activator-like effector nucleases (TALENs), and the CRISPR/Cas9 system. Genome editing of the mutated dystrophin gene has the advantage of permanently modifying the genome in the target cell and all daughter cells after a single treatment. Additionally, editing the endogenous gene retains a large portion of the normal DMD structure and function and leaves the multiple isoforms of the gene under control of their natural promoters. Most examples of genome editing for DMD have focused on removing exons from the gene or introducing small insertions or deletions (indels) into splice sites to restore the correct reading frame of the dystrophin gene. Both approaches use the non-homologous end joining (NHEJ) DNA repair mechanism. Alternatively, the homology-directed repair (HDR) pathway can be used to introduce or exchange DNA sequences. However, this mechanism is significantly downregulated in post-mitotic cells, including skeletal and cardiac muscle. Additionally, gene correction by HDR would require patient-specific genome editing tools, creating additional challenges to reaching the largest possible patient population.

Early efforts to apply genome editing to DMD have employed meganucleases, ZFNs, and TALENs. More recently, CRISPR/Cas9 has been utilized for preclinical proof-of-concept of DMD treatment in patient cells and animal models. In patient-derived cells, genome editing with Cas9 and a single gRNA targeted within exon 51 generated indels that restored the DMD reading frame and dystrophin protein expression, similar to an approach previously reported with TALENs. A similar strategy was recently used in vivo at the orthologous regions of the mouse and dog gene. The reading frame of the DMD gene has also been restored by editing with Cas9 and two gRNAs in vitro to completely excise exon 51 and also larger regions such as the mutational hotspot spanning exons 45-55, which could address ˜40-62% of the patient population. Furthermore, AAV-mediated genome editing with CRISPR/Cas9 to excise single or multiple exons has been used to restore dystrophin expression in vivo in mouse models. Importantly, these examples utilize the mdx or mdx4cv mouse models or mice harboring specific deletions in the mouse Dmd gene and facilitate excision of one or more exons to restore expression of a partially functional mouse dystrophin protein. These mutations in the mouse gene may not accurately replicate patient mutations, and the sequence diversity between the human and mouse dystrophin genes may prevent testing of most human-targeted gRNAs in these models. In addition, mice are often treated with higher doses than are commonly used in the clinic and at a younger age before significant pathology has developed. There is a need for efficient CRISPR/Cas9 approaches targeting the human dystrophin gene in, for example, adult subjects, and for more clinically-relevant gene editing-based DMD therapeutics.

### SUMMARY

In an aspect, the disclosure relates to a CRISPR-Cas vector system comprising one or more vectors. At least one of the one or more vectors comprises a sequence encoding: (a) first guide RNA (gRNA) targeting an intron or an exon of dystrophin and a second gRNA targeting an intron or an exon of dystrophin; and (b) a Cas9 protein. In some embodiments, the system comprises a first vector and a second vector, the first vector encoding the first gRNA and the second gRNA and the second vector encoding the Cas9 protein.

In a further aspect, the disclosure relates to a CRISPR-Cas dual vector system comprising: (a) a first vector encoding a first guide RNA (gRNA) targeting an intron or an exon of dystrophin and a second gRNA targeting an intron or an exon of dystrophin; and (b) a second vector encoding a Cas9 protein.

In some embodiments, the first vector comprises a first ITR and a second ITR. In some embodiments, first ITR is operably linked to and upstream of the polynucleotide sequences encoding the first gRNA and the second gRNA, and wherein the second ITR is operably linked to and downstream of the polynucleotide sequence encoding the first gRNA and the second gRNA. In some embodiments, the first ITR or second ITR is a wild-type ITR, and the other of the first ITR and second ITR is a mutant ITR, and wherein the mutant ITR directs vector genome replication to generate a self-complementary transcript that forms a double-stranded polynucleotide. In some embodiments, the wild-type ITR comprises a polynucleotide having a sequence selected from SEQ ID NOs: 59-61 or 132. In some embodiments, the mutant ITR comprises a polynucleotide having the sequence of SEQ ID NO: 62 or 140. In some embodiments, the first vector comprises a first promoter operably linked to the polynucleotide sequence encoding the first gRNA molecule, and a second promoter operably linked to the polynucleotide sequence encoding the second gRNA molecule. In some embodiments, the first vector comprises an expression cassette comprising 5′-[wild-type ITR]-[promoter]-[first gRNA]-[promoter]-[second gRNA]-[mutant ITR]-3′, wherein “-” is an optional linker independently comprising a polynucleotide of 0-60 nucleotides. In some embodiments, the vector genome replicated from the first vector is self-complementary and comprises 5′-[wild-type ITR]-[promoter]-[first gRNA]-[promoter]-[second gRNA]-[mutant ITR]-[second gRNA]-[promoter]-[first gRNA]-[promoter]-[wild-type ITR]-3′ and forms a double-stranded RNA hairpin. In some embodiments, the first promoter and the second promoter comprise the same or different polynucleotide sequence. In some embodiments, the first promoter and the second promoter are each independently selected from a ubiquitous promoter or a tissue-specific promoter. In some embodiments, the first promoter and the second promoter are each independently selected from a human U6 promoter and a H1 promoter. In some embodiments, the second vector comprises a third promoter driving expression of the Cas9 protein, and wherein the third promoter comprises a ubiquitous promoter or a tissue-specific promoter. In some embodiments, the ubiquitous promoter comprises a CMV promoter. In some embodiments, the tissue-specific promoter is a muscle-specific promoter comprising a MHCK7 promoter, a CK8 promoter, or a Spc512 promoter. In some embodiments, the first vector further encodes at least one Cas9 gRNA scaffold. In some embodiments, the first gRNA and the second gRNA each comprise a Cas9 gRNA scaffold. In some embodiments, the Cas9 gRNA scaffold comprises the polynucleotide sequence of SEQ ID NO: 89 or 18 or 138. In some embodiments, the first or second gRNA targets intron 44 of dystrophin. In some embodiments, the first or second gRNA targets intron 55 of dystrophin. In some embodiments, the first gRNA targets intron 44 of dystrophin and the second gRNA targets intron 55 of dystrophin, or wherein the first gRNA targets intron 55 of dystrophin and the second gRNA targets intron 44 of dystrophin. In some embodiments, the first or second gRNA targeting intron 44 of dystrophin targets a polynucleotide comprising the sequence of SEQ ID NO: 55 or 135 or a 5′ truncation thereof. In some embodiments, the first gRNA or the second gRNA targets intron 44 of dystrophin and comprises the polynucleotide sequence of SEQ ID NO: 57 or 137 or a 5′ truncation thereof. In some embodiments, the first or second gRNA targeting intron 55 of dystrophin targets a polynucleotide comprising the sequence of SEQ ID NO: 56 or 134 or a 5′ truncation thereof. In some embodiments, the first gRNA or the second gRNA targets intron 55 of dystrophin and comprises the polynucleotide sequence of SEQ ID NO: 58 or 136 or a 5′ truncation thereof. In some embodiments, the Cas9 protein comprises SpCas9, SaCas9, or St1Cas9 protein. In some embodiments, the Cas9 protein comprises a SaCas9 protein comprising the amino acid sequence of SEQ ID NO: 88 or is encoded by a polynucleotide comprising the sequence of SEQ ID NO: 69. In some embodiments, the first vector comprises a polynucleotide having the sequence selected from SEQ ID NOs: 91, 92, 128, 129, 130, or 131. In some embodiments, the first vector and/or the second vector is a viral vector. In some embodiments, the viral vector is an Adeno-associated virus (AAV) vector. In some embodiments, the AAV vector is AAV1, AAV2, AAV3, AAV4, AAV5, AAV6, AAV7, AAV8, AAV9, AAV-10, AAV-11, AAV-12, AAV-13, or AAVrh.74. In some embodiments, the first vector is present in a concentration at least 2-fold, at least 3-fold, at least 4-fold, at least 5-fold, at least 6-fold, at least 7-fold, at least 8-fold, at least 9-fold, or at least 10-fold greater than the concentration of the second vector. In some embodiments, the system comprises one or more vectors, and at least one vector of the one or more vectors comprises a sequence encoding, from the 5′ to 3′ direction: (a) a first ITR; (b) a first promoter; (c) a first gRNA targeting an intron or exon of the dystrophin gene; (d) a Cas9 gRNA scaffold; (e) a second promoter; (f) a second gRNA targeting an intron or exon of dystrophin the gene; (g) a Cas9 gRNA scaffold; and (h) a second ITR. In some embodiments, vector genome replication from the at least one vector results in a genome comprising, from the 5′ to 3′ direction: (a) a complementary sequence of the second ITR; (b) a complementary sequence of the second gRNA; (c) a complementary sequence of the second promoter; (d) a complementary sequence of the Cas9 gRNA scaffold; (e) a complementary sequence of the first gRNA; (f) a complementary sequence of the first promoter; (h) the first ITR; (i) the first promoter; (g) the first gRNA; (k) the Cas9 gRNA scaffold; (l) the second promoter; (m) the second gRNA; and (n) the second ITR.

Another aspect of the disclosure provides a cell comprising a system as detailed herein.

Another aspect of the disclosure provides a kit comprising a system as detailed herein.

Another aspect of the disclosure provides a method of correcting a mutant dystrophin gene in a cell. The method may include administering to a cell a system as detailed herein.

Another aspect of the disclosure provides a method of genome editing a mutant dystrophin gene in a subject. The method may include administering to the subject a system as detailed herein or a cell of as detailed herein.

Another aspect of the disclosure provides a method of treating a subject having a mutant dystrophin gene. The method may include administering to the subject a system as detailed herein or a cell as detailed herein.

In some embodiments, the subject is an adult, an adolescent, or a pre-adolescent. In some embodiments, the subject is an adult. In some embodiments, the system as detailed herein or the cell as detailed herein is administered to the subject intravenously. In some embodiments, the system as detailed herein or the cell as detailed herein is administered to the subject systemically.

Another aspect of the disclosure provides a CRISPR-Cas dual vector system comprising one or more vectors, wherein the one or more vectors comprises a vector that comprises an expression cassette. The expression cassette may include, from the 5′ to 3′ direction: (a) a first AAV ITR sequence; (b) a first promoter sequence: (c) a guide sequence targeting a first intron of dystrophin gene; (d) a Cas9 scaffold sequence; (e) a second promoter sequence; (f) a guide sequence targeting a second intron of dystrophin gene; and (g) a second AAV ITR sequence. n some embodiments, the expression cassette is a single stranded (“ss”) expression cassette or a self-complementary (“sc”) expression cassette. In some embodiments, the self-complementary (“sc”) expression cassette, from the 5′ to 3′ direction, comprises: (a) a complementary sequence of the second AAV ITR sequence; (b) a complementary sequence of the guide sequence targeting the second intron of dystrophin gene: (c) a complementary sequence of the second promoter sequence; (d) a complementary sequence of the Cas9 scaffold sequence; (e) a complementary sequence of the guide sequence targeting a first intron of dystrophin gene; (f) a complementary sequence of the first promoter sequence; (h) a first AAV ITR sequence; (i) a first promoter sequence; (g) a guide sequence targeting a first intron of dystrophin gene; (k) a Cas9 scaffold sequence; (l) a second promoter sequence: (m) a guide sequence targeting a second intron of dystrophin gene; and (n) a second AAV ITR sequence. In some embodiments, the first intron is intron 44 and the second intron is intron 55 of the dystrophin gene, or wherein the first intron is intron 55 and the second intron is intron of 44 of the dystrophin gene. In some embodiments, the dystrophin gene comprises a mutation compared to a wild-type dystrophin gene. In some embodiments, the guide sequence targeting a first intron of dystrophin gene comprises a nucleotide sequence of SEQ ID NO: 134 or 56 and the guide sequence targeting a second intron of dystrophin gene comprises a nucleotide sequence of SEQ ID NO: 135 or 55, or wherein the guide sequence targeting a first intron of dystrophin gene comprises a nucleotide sequence of SEQ ID NO: 135 or 55 and the guide sequence targeting a second intron of dystrophin gene comprises a nucleotide sequence of SEQ ID NO: 134 or 56. In some embodiments, the promoter is a constitutive promoter or a tissue-specific promoter. In some embodiments, the promoter is a muscle-specific promoter. In some embodiments, the muscle-specific promoter comprises a human skeletal actin gene element, cardiac actin gene element, myocyte-specific enhancer binding factor mef, muscle creatine kinase (MCK), truncated MCK (tMCK), myosin heavy chain (MHC), MHCK7, C5-12, murine creatine kinase enhancer element, skeletal fast-twitch troponin c gene element, slow-twitch cardiac troponin c gene element, the slow-twitch troponin i gene element, hypoxia-inducible nuclear factor binding element, steroid-inducible element, or glucocorticoid response element (gre). In some embodiments, the constitutive promoter comprises CMV, human U6 promoter, or H1 promoter. In some embodiments, the constitutive promoter comprises a sequence of SEQ ID NO: 133 or 63. In some embodiments, the first AAV ITR sequence comprises a sequence of SEQ ID NO: 132 or 59. In some embodiments, the second AAV ITR sequence comprises a sequence of SEQ ID NO: 140 or 62. In some embodiments, the expression cassette comprises a sequence of SEQ ID NO: 128. In some embodiments, the expression cassette comprises a sequence of SEQ ID NO: 129. In some embodiments, the Cas9 scaffold sequence is a spCas9 scaffold sequence or SaCas9 scaffold sequence. In some embodiments, the Cas9 scaffold sequence is a SaCas9 scaffold sequence. In some embodiments, the Cas9 scaffold sequence comprises a sequence of SEQ ID NO: 138 or 139 or 89 or 90. In some embodiments, the one or more vectors encodes a Cas9 protein. In some embodiments, the Cas9 protein is a SaCas9 or a spCas9 protein. In some embodiments, the SaCas9 protein comprises an amino acid sequence of SEQ ID NO: 21 or 88 or is encoded by a polynucleotide comprising the sequence of SEQ ID NO: 69. In some embodiments, the one or more vectors are viral vectors. In some embodiments, the viral vector is an Adeno-associated virus (AAV) vector. In some embodiments, the AAV vector is AAV1, AAV2, AAV3, AAV4, AAV5, AAV6, AAV7, AAV8, AAV9, AAV-10, AAV-11, AAV-12, AAV-13, orAAVrh.74. In some embodiments, the vector that comprises an expression cassette is present in a concentration at least 2-fold, at least 3-fold, at least 4-fold, at least 5-fold, at least 6-fold, at least 7-fold, or at least 8-fold greater than the concentration of the vector encoding the Cas9 protein.

Another aspect of the disclosure provides a cell comprising a system as detailed herein.

Another aspect of the disclosure provides a kit comprising a system as detailed herein.

Another aspect of the disclosure provides a method of correcting a mutant dystrophin gene in a cell, the method comprising administering to a cell a system as detailed herein.

Another aspect of the disclosure provides a method of genome editing a mutant dystrophin gene in a subject, the method comprising administering to the subject a system as detailed herein.

Another aspect of the disclosure provides a method of treating a subject having a mutant dystrophin gene, the method comprising administering to the subject a system as detailed herein or a cell as detailed herein.

In some embodiments, the subject is a human. In some embodiments, the system as detailed herein or the cell as detailed herein is administered to the subject intravenously. In some embodiments, the system as detailed herein or the cell as detailed herein is administered to the subject systemically.

Another aspect of the disclosure provides a plasmid expressing an expression cassette as detailed herein, wherein the plasmid comprises a sequence selected from SEQ ID NOs: 87, 91, 92, 128, 129, 130, or 131.

The disclosure provides for other aspects and embodiments that will be apparent in light of the following detailed description and accompanying figures.

### BRIEF DESCRIPTION OF THE DRAWINGS

FIG. 1 shows the two deletion prone hotspots in dystrophin. The dystrophin gene (which may be referred to as DMD) is the largest known gene in humans (2.3 Mbp). Approximately, 68% of mutations are large exon deletions that lead to frameshift errors.

FIG. 2 shows details relating to the exon 45 through exon 55 mutational hotspot. Approximately 45% of all DMD mutations, and many commonly deleted single exons, are located in this region. Patients with exon 45 to 55 in-frame deletion display milder dystrophic phenotype. AONs (antisense oligonucleotides) have been used to induce exon skipping in this region.

FIG. 3 shows excision of exons 45 through 55 of dystrophin. This system is being tested in a humanized mouse carrying the human gene with a deletion of exon 52.

FIG. 4 shows injection of a system to excise exons 45 through 55 of dystrophin in neonatal mice. Neonatal mice were systemically injected at 2 days postnatal (P2). Muscles were harvested 8 weeks post-treatment. PCR bands show the intended deletion.

FIG. 5 shows dystrophin expression in systemically treated mice. 10× magnification, dual vector P2 injected, 8 weeks post-treatment.

FIG. 6 shows the traditional two vector system as compared to the one vector system. Advantages to the one vector system may include: having all necessary editing components on a single vector, ability to increase effective dose, streamlining of other vector production (single therapeutic agent), use/incorporation of muscle-specific promoters (CK8, Spc512, MHCK7), and ability to target combinations of exons and large deletions (by changing guide sequences).

FIG. 7 shows a vector design comparison. The all-in-one vector components (total packaged DNA<4.8 kb include: SaCas9 (˜3.2 kb); mini polyadenylation signal (60 bp) or bGH polyadenylation signal (232 bp); constitutive EFS promoter (252 bp) or muscle specific promoter).

FIG. 8 shows the all-in-one vector for deletion of exons 45-55 and in vitro analyses in HEK293s.

FIG. 9A is a schematic diagram of the dystrophin gene from immortalized myoblasts isolated from a DMD patient, showing the deletion of exons 48-50. FIG. 9B shows results from deletion PCR of genomic DNA and cDNA from treated DMD patients, indicating that exon 45-55 was effectively deleted with vectors as detailed herein. FIG. 90C is a Western blot of cell lysates, showing that untreated myoblasts produced no dystrophin protein, while transfected myoblasts expressed a smaller dystrophin protein compared to the positive control, consistent with hotspot deletion.

FIG. 10 are images of cardiac muscle cells from neonatal hDMDΔ52/mdx mice injected with either AAV-CRISPR targeting a control locus (top panel) or targeting exon 45-55 (bottom panel). Cells were harvested 8 weeks post injection. Cells were stained with DAPI or for dystrophin. 10× magnification, scale bar=200 μm.

FIG. 11 is a schematic diagram of the versions of all-in-one vector 5.

FIG. 12 are images of TA muscle cells 8 weeks after injection with the vectors as indicated, at 10× magnification.

FIG. 13 are graphs showing SaCas9 and gRNA in vivo expression resulting from treatment with the indicated all-in-one vectors, as determined by qRT-PCR using TA samples 8 weeks post-injection. N=3-4.

FIG. 14 are graphs showing the stability of all-in-one (AIO) vectors in vivo. The left graph are results from qPCR using TA samples 8 weeks post-injection. The right graphs are results from IFN-gamma ELISpot assay against SaCas9. N=3-4 for both.

FIG. 15A-FIG. 15C. Comparison of dual vector strategies. (FIG. 15A) Approach #1 was validated in del52/mdx mice with Exon 51 deletion via systemic and local injection. (FIG. 15B) Approach #2 was Validated in mdx with Exon 23 deletion via systemic and local injection. The gRNA to Cas9 ratio may be increased. (FIG. 15C) Approach #3 showed immediate expression, increased persistence in tissue, and greater editing. The gRNA to Cas9 ratio may be increased.

FIG. 16. In vitro validation. HEK293 cells were transduced with AAV2 crude lysate at MOI 2e5. gDNA was extracted 3 days post-transduction.

FIG. 17. Local injection of dual vector approaches in hDMDΔ52/mdx mice induces hotspot deletion. Transcript deletion was measured by ddPCR. The Cas9:guide ratio shown in parenthesis. One-way ANOVA with Tukey multiple comparison test were performed. *P<0.05, **P<0.01.

FIG. 18. Local injection of dual vector approaches in hDMDΔ52/mdx mice restores dystrophin expression. Shown are images of dystrophin IF staining, 8 weeks post-injection. Vectors were administered at a ratio of 1:1.

FIG. 19A-FIG. 19B. Self-complementary AAV increases guide RNA expression. (FIG. 19A-FIG. 19B) Shown are graphs of Cas9 expression and gRNA expression. Two-way ANOVA with Tukey multiple comparison test was performed. The (*) compares with Approach #2, and (#) compares with Approach #2 (1:3), P<0.05.

FIG. 20A-FIG. 20E. CRISPR enables single exon deletion in humanized mouse models of Duchenne muscular dystrophy. (FIG. 20A) A humanized mouse model of Duchenne muscular dystrophy. Founder mice containing the full-length human dystrophin gene with a deletion of exon 52 were crossed onto the mdx background to generate the hDMDdel52/mdx line. A separate utrophin-deficient line, or double KO (dKO), was generated by crossing the hDMDdel52/mdx with the Utrn^{tm1kEd}Dmd^{mdx}/J mouse, which harbors a neomycin cassette in exon 7 of the mouse utrophin gene, preventing Utrn expression. (FIG. 20B) A schematic representing the exon 51 deletion strategy, creating an in-frame mutation in order to restore dystrophin expression. Guide RNAs were designed to target intronic regions flanking exon 51 to recruit the SaCas9 nuclease to create double-stranded breaks, which are repaired via NHEJ. Two single-stranded AAV vectors were generated encoding SaCas9 and a single guide: SaCas9-guide RNA 1+SaCas9-guide RNA 2. These constructs were packaged into AAV9 and used to treat adult hDMDdel52/mdx and hDMDdel52/mdx/Utrn KO mice via tail vein injection at a dose of 4E12 vg. (FIG. 20C) Endpoint PCR of dystrophin transcripts from untreated (NT) and CRISPR-treated mice. Primers were designed to amplify the intended region across exon 51. The untreated control contains a wildtype band (black triangle), while CRISPR-treated mice display both the wildtype band and a shortened transcript (white triangle). (FIG. 20D) Dystrophin restoration in CRISPR-treated mice. Histological sections from hDMD/mdx, untreated hDMDdel52/mdx, and treated hDMDdel52/mdx and hDMDdel52/mdx/Utrn KO (dKO) mice were stained for dystrophin. Dystrophin expression (shown in red) was observed at the sarcolemma of cardiac and skeletal muscles of CRISPR-treated mice. Scale bars=100 μm. (FIG. 20E) Quantification of exon 51 deletion in DMD transcripts from the heart (top), tibialis anterior (TA, middle), and diaphragm (bottom) of untreated and treated hDMDdel52/mdx and dKO mice via ddPCR. Data represent mean±SEM and symbols represent individual values. Lines represent statistical significance between two groups. Statistics were performed using one-way ANOVA with Tukey's multiple comparisons test. *P<0.05, **P<0.01, ****P<0.0001.

FIG. 21A-FIG. 21C. Dystrophic pathology is improved after CRISPR-mediated exon 51 deletion in a severe mouse model of DMD. (FIG. 21A) Representative histological sections from the heart (top), TA (middle), and diaphragm (bottom) muscles from hDMD/mdx and hDMDdel52/mdx/Utrn KO mice were stained to examine muscle pathology (H&E, left columns) and fibrosis (Masson trichrome, right columns). Mice treated with a non-targeting control vector display a pronounced dystrophic phenotype that is similar to untreated utrophin-deficient humanized mice (not shown), which is characterized by muscle degeneration (centronucleation, apoptotic cells, and infiltrating immune cells) and fibrotic deposition (blue staining, right panel). CRISPR-mediated exon 51 deletion reduced muscle degeneration in the skeletal muscle. Fibrosis was also reduced in the heart and skeletal muscle. Scale bars=100 μm. (FIG. 21B) Fibrotic area quantification in heart and skeletal muscles of dKO mice. At least 3 images were quantified per mouse. Data represent mean±SEM and symbols represent individual values. Lines represent statistical significance between two groups. Statistics were performed using two-way ANOVA with Tukey's multiple comparisons test. N=3 to 6 per group. *P<0.05, **P<0.01, ***P<0.001, ****P<0.0001. (FIG. 21C) Kaplan-Meier survival curve of untreated and treated hDMDdel52/mdx mice. N=3 to 6 per group. Statistical differences between survival curves were compared using log-rank test. Results from statistical analysis are shown in the table.

FIG. 22A-FIG. 22G. Mutational hotspot deletion following local administration of AAV9-CRISPR vectors. (FIG. 22A) A schematic representing the mutational hotspot deletion strategy to create an in-frame mutation. Guide RNAs were designed to target intronic regions flanking exons 45 and 55 to recruit the SaCas9 nuclease to create double-stranded breaks, which are repaired via NHEJ. (FIG. 22B) Three different dual vector strategies were designed and packaged into AAV9. These approaches were administered at equal dose of 2E11 vg at a 1:1, 1:3, or 1:5 Cas9 to guide ratio via intramuscular injection in adult hDMDdel52/mdx mice. (FIG. 22C) Endpoint PCR illustrating deletion of exons 45-55 in DMD transcripts of treated mice. (FIG. 22D) Quantification of SaCas9 expression and (FIG. 22E) guide RNA expression following local injection. Cas9 expression was normalized to Approach #1. Data represent mean±SEM. (FIG. 22F) Quantification of exon 45-55 deleted transcripts via ddPCR. Data represent mean±SEM and symbols represent individual values. Lines represent statistical significance between two groups. Statistics were performed using one-way ANOVA with Tukey's multiple comparisons test. N=6 mice per group. *P<0.05, **P<0.01. (FIG. 22G) Representative histological images of mice treated locally with the different dual vector strategies. Approach #3 administered at a 1:5 Cas9 to guide ratio resulted in greater dystrophin-positive fibers (shown in red) compared to the other strategies.

FIG. 23A-FIG. 23F. Mutational hotspot deletion following systemic administration of AAV9-CRISPR vectors restores dystrophin expression. (FIG. 23A) Adult hDMDdel52/mdx mice were treated with a control vector, a single-stranded guide approach (Approach #1 in FIG. 3), or a self-complementary guide strategy (Approach #3, 1:5 ratio in FIG. 22A-FIG. 22G) at a dose of 4E12 vg. (FIG. 23B) Endpoint PCR showing exon 45-55 deletion in DMD transcripts from heart and skeletal muscle (white triangle). (FIG. 23C) Representative histological images showing dystrophin expression (red) in muscles from untreated and treated hDMDdel52/mdx treated with either a ssAAV-guides or scAAV-guides dual vector approach. (FIG. 23D) Quantification of exon 45-55 deleted transcripts was performed via ddPCR. Grip force (FIG. 23E) and specific force (FIG. 23F) were measured at 8 weeks post-treatment. Grip force was normalized to body weight. Data represent mean±SEM and symbols represent individual values. Lines represent statistical significance between two groups. Statistics were performed using one-way ANOVA with Tukey's multiple comparisons test. N=5 to 8 per group. *P<0.05, **P<0.01, ***P<0.001, ****P<0.0001.

FIG. 24A-FIG. 24D. (FIG. 24A) Chromatogram from Sanger sequencing showing the loss of exon 52 and (FIG. 24B) resulting insertion in the gDNA of hDMDΔ52/mdx mice. (FIG. 24C and FIG. 24D) Characterization of the hDMDΔ52/mdx mouse by Western blot shows absence of dystrophin expression in protein extracted from the TA.

FIG. 25A-FIG. 25C. (FIG. 25A) Shown is deletion of exon 51 in the genomic DNA of HEK293T cells and immortalized DMD patient myoblasts transfected with plasmids encoding SaCas9 and two gRNAs by lipofection and electroporation, respectively. Droplet digital PCR (ddPCR) shows deletions in 16% and 12% of the alleles in the HEK293T cells and DMD patient myoblasts, respectively. (FIG. 25B) The gel shows Exon 51 was absent in a fraction of cDNA from differentiated patient myoblasts treated with SaCas9 and gRNAs. ddPCR showed lack of exon 51 in 14% of the cDNA. (FIG. 25C) Shown is dystrophin protein restoration by Western blot in DMD patient myoblasts treated with SaCas9 and both gRNAs, indicative that exon 51 was deleted and the reading frame was restored.

FIG. 26A-FIG. 26D. Local injection of right tibialis anterior (TA) muscles with AAV encoding the exon 51-targeted CRISPR/SaCas9 system. (FIG. 26A) Schematic of the AAV vector design. (FIG. 26B) Deletion of exon 51 in the genomic DNA of the treated right (R) TA muscles compared to no deletions in the contralateral PBS-injected left (L) TA muscles across three mice. (FIG. 26C) Positive dystrophin immunofluorescence staining in a treated right TA muscle. (FIG. 26D) Variable levels of dystrophin restoration in the treated right (R) TA muscles by western blot. WT was protein from an hDMD/mdx mouse,

FIG. 27A-FIG. 27E. End-point PCR analysis on genomic DNA and chromatogram from sequencing. (FIG. 27A-FIG. 27D) End-point nested PCR amplification of exon 51 deletion in gDNA from (FIG. 27A) heart, (FIG. 27B) diaphragm, (FIG. 27C) gastrocnemius, and (FIG. 27D) tibialis anterior for mice treated as adults and neonates. (−) is a negative control untreated mouse and (+) is a treated mouse. Although detection of deletions was stochastic by end-point PCR, the more sensitive deep sequencing methods detected consistent editing (FIG. 21A-FIG. 21C). (FIG. 27E) Sequencing of the deletion band in the gDNA, which showed the junction of the predicted gRNA cut sites 3 bp upstream from the PAM as expected.

FIG. 28A-FIG. 28E. Quantitative analysis by ddPCR of AAV vector DNA encoding SaCas9 present in mice treated as adults (triangles) and neonates (squares), including in the (FIG. 28A) heart, (FIG. 28B) TA, (FIG. 28C) diaphragm, (FIG. 28D) gastrocnemius, and (FIG. 28E) liver. The two high data points in the mice treated as adults (purple) in TA and diaphragm gDNA were different mice.

### DETAILED DESCRIPTION

As described herein, certain methods and engineered gRNAs have been discovered to be useful with CRISPR/CRISPR-associated (Cas) 9-based gene editing systems for altering the expression, genome engineering, and correcting or reducing the effects of mutations in the dystrophin gene involved in genetic diseases, such as DMD. The disclosed gRNAs were generated to target sites that are more amenable to clinical translation. For example, the gene encoding S. pyogenes Cas9 (SpCas9) is too large to be delivered by adeno-associated virus (AAV), a vector used for the systemic gene delivery to muscle when all other necessary regulatory sequences are included. Instead, the disclosed gRNAs were selected and screened for use with S. aureus Cas9 (SaCas9), which is about 1 kb smaller than SpCas9. The disclosed gRNAs, which target human dystrophin gene sequences, can be used with the CRISPR/Cas9-based system to target exons 45 to 55 of the human dystrophin gene, causing genomic deletions of this region in order to restore expression of functional dystrophin in cells from DMD patients. Further detailed herein is a dual vector system, which may include a self-complementary vector. The self-complementary vector includes a mutant ITR that directs vector genome replication to generate a self-complementary transcript that forms a double-stranded polynucleotide. The dual vector system with a self-complementary vector may improve expression of the CRISPR/Cas-based system components.

Also described herein are genetic constructs, compositions, and methods for delivering CRISPR/Cas9-based gene editing system and multiple gRNAs to target the dystrophin gene. The presently disclosed subject matter also provides for methods for delivering the genetic constructs (for example, vectors) or compositions comprising thereof to skeletal muscle and cardiac muscle. The vector can be an AAV, including modified AAV vectors. The presently disclosed subject matter describes a way to deliver active forms of this class of therapeutics to skeletal muscle or cardiac muscle that is effective, efficient, and facilitates successful genome modification, as well as provide a means to rewrite the human genome for therapeutic applications and target model species for basic science applications. The methods may relate to the use of a single AAV vector for the delivery of all of the editing components necessary for the excision of exons 45 through 55 of dystrophin.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, particularly with respect to biological systems or processes, the term can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Binding region” as used herein refers to the region within a nuclease target region that is recognized and bound by the nuclease.

“Cardiac muscle” or “heart muscle” as used interchangeably herein means a type of involuntary striated muscle found in the walls and histological foundation of the heart, the myocardium. Cardiac muscle is made of cardiomyocytes or myocardiocytes. Myocardiocytes show striations similar to those on skeletal muscle cells but contain only one, unique nucleus, unlike the multinucleated skeletal cells. In certain embodiments, “cardiac muscle condition” refers to a condition related to the cardiac muscle, such as cardiomyopathy, heart failure, arrhythmia, and inflammatory heart disease.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

“Correcting”, “genome editing,” and “restoring” as used herein refers to changing a mutant gene that encodes a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site, or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

The term “directional promoter” refers to two or more promoters that are capable of driving transcription of two separate sequences in both directions. In one embodiment, one promoter drives transcription from 5′ to 3′ and the other promoter drives transcription from 3′ to 5. In one embodiment, bidirectional promoters are double-strand transcription control elements that can drive expression of at least two separate sequences, for example, coding or non-coding sequences, in opposite directions. Such promoter sequences may be composed of two individual promoter sequences acting in opposite directions, such as one nucleotide sequence linked to the other (complementary) nucleotide sequence, including packaging constructs comprising the two promoters in opposite directions, for example, by hybrid, chimeric or fused sequences comprising the two individual promoter sequences, or at least core sequences thereof, or else by only one transcription regulating sequence that can initiate the transcription in both directions. The two individual promoter sequences, in some embodiments, may be juxtaposed or a linker sequence can be located between the first and second sequences. A promoter sequence may be reversed to be combined with another promoter sequence in the opposite orientation. Genes located on both sides of a bidirectional promoter can be operably linked to a single transcription control sequence or region that drives the transcription in both directions. In other embodiments, the bidirectional promoters are not juxtaposed. For example, one promoter may drive transcription on the 5′ end of a nucleotide fragment, and another promoter may drive transcription from the 3′ end of the same fragment. In another embodiment, a first gene can be operably linked to the bidirectional promoter with or without further regulatory elements, such as a reporter or terminator elements, and a second gene can be operably linked to the bidirectional promoter in the opposite direction and by the complementary promoter sequence, again with or without further regulatory elements.

“Donor DNA”, “donor template,” and “repair template” as used interchangeably herein refers to a double-stranded DNA fragment or molecule that includes at least a portion of the gene of interest. The donor DNA may encode a full-functional protein or a partially-functional protein.

“Duchenne Muscular Dystrophy” or “DMD” as used interchangeably herein refers to a recessive, fatal, X-linked disorder that results in muscle degeneration and eventual death. DMD is a common hereditary monogenic disease and occurs in 1 in 3500 males. DMD is the result of inherited or spontaneous mutations that cause nonsense or frame shift mutations in the dystrophin gene. The majority of dystrophin mutations that cause DMD are deletions of exons that disrupt the reading frame and cause premature translation termination in the dystrophin gene. DMD patients typically lose the ability to physically support themselves during childhood, become progressively weaker during the teenage years, and die in their twenties.

“Dystrophin” as used herein refers to a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb. 79 exons code for the protein which is over 3500 amino acids.

“Exons 45 through 55” of dystrophin as used herein refers to an area where roughly 45% of all dystrophin mutations are located. Exon 45-55 deletions are associated with very mild Becker phenotypes and have even been found in asymptomatic individuals. Exon 45-55 multiexon skipping would be beneficial for roughly 50% of all DMD patients.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operably linked to a coding sequence that encodes a protein such that when present in the cell of the individual, the coding sequence will be expressed.

“Genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, Becker Muscular Dystrophy (BMD), hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Genome editing” as used herein refers to changing a gene. Genome editing may include correcting or restoring a mutant gene. Genome editing may include knocking out a gene, such as a mutant gene or a normal gene. Genome editing may be used to treat disease or enhance muscle repair by changing the gene of interest.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number, of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nuclease mediated NHEJ” as used herein refers to NHEJ that is initiated after a nuclease, such as a Cas9 molecule, cuts double stranded DNA.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Partially-functional” as used herein describes a protein that is encoded by a mutant gene and has less biological activity than a functional protein but more than a non-functional protein.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively (constitutive promoter), or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, H1 promoter, EFS promoter, human U6 (hU6) promoter, and CMV IE promoter. Examples of muscle-specific promoters may include, for example, a human skeletal actin gene element, cardiac actin gene element, myocyte-specific enhancer binding factor mef, muscle creatine kinase (MCK), truncated MCK (tMCK), myosin heavy chain (MHC), MHCK7, C5-12, murine creatine kinase enhancer element, skeletal fast-twitch troponin c gene element, slow-twitch cardiac troponin c gene element, the slow-twitch troponin i gene element, hypoxia-inducible nuclear factor binding element, steroid-inducible element or glucocorticoid response element (gre). Examples of muscle-specific promoters may include a MHCK7 promoter, a CK8 promoter, and a Spc512 promoter.

“Skeletal muscle” as used herein refers to a type of striated muscle, which is under the control of the somatic nervous system and attached to bones by bundles of collagen fibers known as tendons. Skeletal muscle is made up of individual components known as myocytes, or “muscle cells” sometimes colloquially called “muscle fibers.” Myocytes are formed from the fusion of developmental myoblasts (a type of embryonic progenitor cell that gives rise to a muscle cell) in a process known as myogenesis. These long, cylindrical, multinucleated cells are also called myofibers.

“Skeletal muscle condition” as used herein refers to a condition related to the skeletal muscle, such as muscular dystrophies, aging, muscle degeneration, wound healing, and muscle weakness or atrophy.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

“Target gene” as used herein refers to any nucleotide sequence encoding a known or putative gene product. The target gene may be a mutated gene involved in a genetic disease. In certain embodiments, the target gene is a human dystrophin gene. In certain embodiments, the target gene is a mutant human dystrophin gene.

“Target region” as used herein refers to the region of the target gene to which the CRISPR/Cas9-based gene editing system is designed to bind and cleave.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity, A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide, Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Dystrophin

Dystrophin is a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane. The dystrophin gene is 2.2 megabases at locus Xp21. The primary transcription measures about 2,400 kb with the mature mRNA being about 14 kb. 79 exons include approximately 2.2 million nucleotides and code for the protein which is over 3500 amino acids. Normal skeleton muscle tissue contains only small amounts of dystrophin, but its absence of abnormal expression leads to the development of severe and incurable symptoms. Some mutations in the dystrophin gene lead to the production of defective dystrophin and severe dystrophic phenotype in affected patients. Some mutations in the dystrophin gene lead to partially-functional dystrophin protein and a much milder dystrophic phenotype in affected patients.

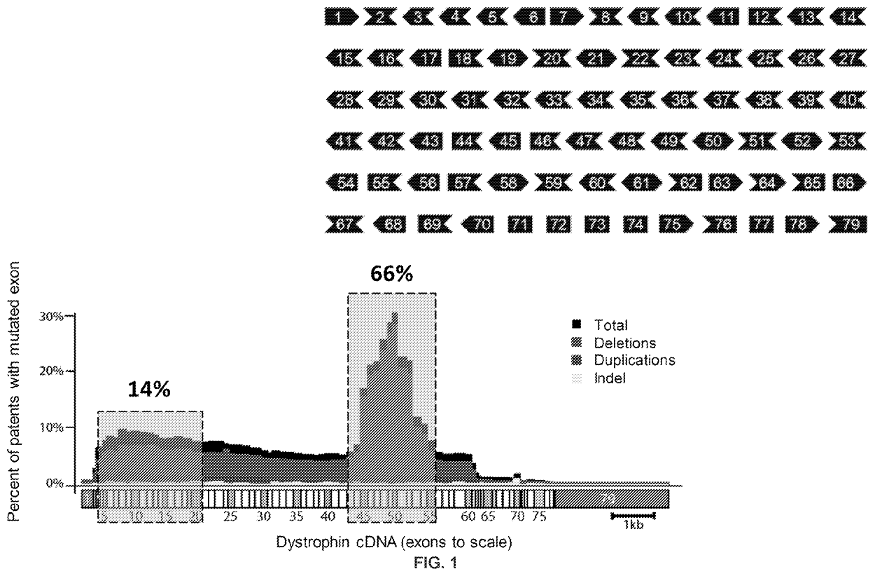

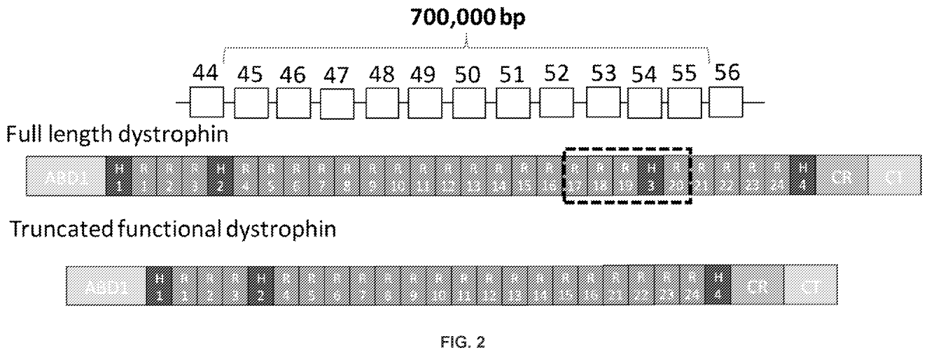

DMD is the result of inherited or X-linked recessive spontaneous mutation(s) that cause nonsense or frame shift mutations in the dystrophin gene. DMD is a severe, highly debilitating and incurable muscle disease and is the most prevalent lethal heritable childhood disease and affects approximately one in 5,000 newborn males. DMD is characterized by muscle deterioration, progressive muscle weakness, often leading to mortality in subjects at age mid-twenties and premature death, due to the lack of a functional dystrophin gene. Most mutations are deletions in the dystrophin gene that disrupt the reading frame. Naturally occurring mutations and their consequences are relatively well understood for DMD. In-frame deletions that occur in the exon 45-55 regions (FIG. 1, FIG. 2) contained within the rod domain can produce highly functional dystrophin proteins, and many carriers are asymptomatic or display mild symptoms. Exons 45-55 of dystrophin are a mutational hotspot. Furthermore, more than 60% of patients may theoretically be treated by targeting this region as a whole (exons 45 through 55) or specific exons in this region of the dystrophin gene (for example, targeting exon 51 only). Efforts have been made to restore the disrupted dystrophin reading frame in DMD patients by skipping non-essential exon(s) (for example, exon 51 skipping) during mRNA splicing to produce internally deleted but functional dystrophin proteins. The deletion of internal dystrophin exon(s) (for example, deletion of exon 51) retains the proper reading frame but cause the less severe Becker muscular dystrophy (BMD). The BMD genotype is similar to DMD in that deletions are present in the dystrophin gene. However, the deletions in BMD leave the reading frame intact. Thus, an internally truncated but partially functional dystrophin protein is created. BMD has a wide array of phenotypes, but often if deletions are between exons 45-55 of dystrophin, the phenotype is much milder compared to DMD. Thus, changing a DMD genotype to a BMD genotype is a common strategy to correct dystrophin. There are many strategies to correct dystrophin, many of which rely on restoring the reading frame of the endogenous dystrophin. This shifts the disease genotype from DMD to Becker muscular dystrophy. Many BMD patients have intragenic deletions that maintain the translational reading frame, leading to a shorter but largely functional dystrophin protein.

A dystrophin gene may be a mutant dystrophin gene. A dystrophin gene may be a wild-type dystrophin gene. A dystrophin gene may be a human dystrophin gene. A dystrophin gene may be a rhesus monkey dystrophin gene. A dystrophin gene may have a sequence that is functionally identical to a wild-type dystrophin gene, for example, the sequence may be codon-optimized but still encode for the same protein as the wild-type dystrophin. A mutant dystrophin gene may include one or more mutations relative to the wild-type dystrophin gene. Mutations may include, for example, nucleotide deletions, substitutions, additions, transversions, or combinations thereof, A mutation in the dystrophin gene may be a functional deletion of the dystrophin gene. In some embodiments, the mutation in the dystrophin gene comprises an insertion or deletion in the dystrophin gene that prevents protein expression from the dystrophin gene. Mutations may be in one or more exons and/or introns. Mutations may include deletions of all or parts of at least one intron and/or exon. An exon of a mutant dystrophin gene may be mutated or at least partially deleted from the dystrophin gene. An exon of a mutant dystrophin gene may be fully deleted. A mutant dystrophin gene may have a portion or fragment thereof that corresponds to the corresponding sequence in the wild-type dystrophin gene. In some embodiments, a disrupted dystrophin gene caused by a deleted or mutated exon can be restored in DMD patients by adding back the corresponding wild-type exon.

In certain embodiments, modification of exons 45-55 (such as deletion or excision of exons 45 through 55 by, for example, NHEJ) to restore reading frame ameliorates the phenotype DMD in subjects, including DMD subjects with deletion mutations. Exons 45 through 55 of a dystrophin gene refers to the 45th exon, 46th exon, 47th exon, 48th exon, 49th exon, 50th exon, 51st exon, 52nd exon, 53rd exon, 54th exon, and the 55th exon of the dystrophin gene. Mutations in the 45th through 55th exon region are ideally suited for permanent correction by NHEJ-based genome editing.

The presently disclosed genetic constructs can generate deletions in the dystrophin gene. The dystrophin gene may be a human dystrophin gene. In certain embodiments, the vector is configured to form two double stand breaks (a first double strand break and a second double strand break) in two introns (a first intron and a second intron) flanking a target position of the dystrophin gene, thereby deleting a segment of the dystrophin gene comprising the dystrophin target position. A “dystrophin target position” can be a dystrophin exonic target position or a dystrophin intra-exonic target position, as described herein. Deletion of the dystrophin exonic target position can optimize the dystrophin sequence of a subject suffering from Duchenne muscular dystrophy. For example, it can increase the function or activity of the encoded dystrophin protein, and/or result in an improvement in the disease state of the subject. In certain embodiments, excision of the dystrophin exonic target position restores reading frame. The dystrophin exonic target position can comprise one or more exons of the dystrophin gene. In certain embodiments, the dystrophin target position comprises exon 51 of the dystrophin gene (e.g., human dystrophin gene).

A presently disclosed genetic construct can mediate highly efficient gene editing at the exon 45 through exon 55 region of a dystrophin gene. A presently disclosed genetic construct can restore dystrophin protein expression in cells from DMD patients.

Elimination of exons 45 through 55 from the dystrophin transcript by exon skipping can be used to treat approximately 50% of all DMD patients. This class of dystrophin mutations is suited for permanent correction by NHEJ-based genome editing and HDR. The genetic constructs described herein have been developed for targeted modification of exon 45 through exon 55 in the human dystrophin gene. A presently disclosed genetic construct may be transfected into human DMD cells and mediate efficient gene modification and conversion to the correct reading frame. Protein restoration may be concomitant with frame restoration and detected in a bulk population of CRISPR/Cas9-based gene editing system-treated cells.

### 3. Systems for Genome Editing of Dystrophin Gene

Provided herein are systems and genetic constructs for genome editing, genomic alteration, and/or altering gene expression of a dystrophin gene. The disclosed gRNAs can be included in a CRISPR/Cas9-based gene editing system, including systems that use SaCas9, to target, for example, exons 45 through 55 of the human dystrophin gene. The disclosed gRNAs, which may be included in a CRISPR/Cas9-based gene editing system, can cause genomic deletions of the region of exons 45 through 55 of the human dystrophin gene in order to restore expression of functional dystrophin in cells from DMD patients.

a. CRISPR/Cas9-Based System

A presently disclosed system or genetic construct may encode a CRISPR/Cas9-based gene editing system that is specific for a dystrophin gene. “Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a ‘memory’ of past exposures. Cas proteins include, for example, Cas12a, Cas9, and Cascade proteins. Cas12a may also be referred to as “Cpf1.” Cas12a causes a staggered cut in double stranded DNA, while Cas9 produces a blunt cut. In some embodiments, the Cas protein comprises Cas12a. In some embodiments, the Cas protein comprises Cas9. Cas9 forms a complex with the 3′ end of the sgRNA (also referred interchangeably herein as “gRNA”), and the protein-RNA pair recognizes its genomic target by complementary base pairing between the 5′ end of the gRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, 11, and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex. Cas12a systems include crRNA for successful targeting, whereas Cas9 systems include both crRNA and tracrRNA.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Cas and Cas Type II systems have differing PAM requirements. For example, Cas12a may function with PAM sequences rich in thymine “T.”

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, or wound healing. The CRISPR/Cas9-based gene editing system can include a Cas9 protein or a Cas9 fusion protein.

i) Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pieuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Canpylobacler lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 20. In certain embodiments, the Cas9 molecule is a Staphylococcus aureus Cas9 molecule (also referred herein as “SaCas9”). SaCas9 may comprise an amino acid sequence of SEQ ID NO: 21.

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi:10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi:10.1038/nmeth.2681). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R=A or G) (SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G) (SEQ ID NO: 7), NNGRRN (R=A or G) (SEQ ID NO: 8), NNGRRT (R=A or G) (SEQ ID NO: 9), or NNGRRV (R=A or G; V=A or C or G) (SEQ ID NO: 10). In the aforementioned embodiments, N can be any nucleotide residue, for example, any of A, G, C, or T.

Additionally or alternatively, a nucleic acid encoding a Cas9 molecule or Cas9 polypeptide may comprise a nuclear localization sequence (NLS). Nuclear localization sequences are known in the art, for example, SV40 NLS (Pro-Lys-Lys-Lys-Arg-Lys-Val; SEQ ID NO: 49.

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A. A S. pyogenes Cas9 protein with the D10A mutation may comprise an amino acid sequence of SEQ ID NO: 22. A S. pyogenes Cas9 protein with DIOA and H849A mutations may comprise an amino acid sequence of SEQ ID NO: 23. Exemplary mutations with reference to the S. aureus Cas9 sequence to inactivate the nuclease activity include D10A and N580A. In certain embodiments, the mutant S. aureus Cas9 molecule comprises a D10A mutation. The nucleotide sequence encoding this mutant S. aureus Cas9 is set forth in SEQ ID NO: 24. In certain embodiments, the mutant S. aureus Cas9 molecule comprises a N580A mutation. The nucleotide sequence encoding this mutant S. aureus Cas9 molecule is set forth in SEQ ID NO: 25.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 26. Exemplary codon optimized nucleic acid sequences encoding a Cas9 molecule of S. aureus, and optionally containing nuclear localization sequences (NLSs), are set forth in SEQ ID NOs: 27-33. Another exemplary codon optimized nucleic acid sequence encoding a Cas9 molecule of S. aureus comprises the nucleotides 1293-4451 of SEQ ID NO: 34.

ii) Cas Fusion Protein

Alternatively or additionally, the CRISPR/Cas-based gene editing system can include a fusion protein. The fusion protein can comprise two heterologous polypeptide domains. The first polypeptide domain comprises a Cas protein or a mutated Cas protein. The first polypeptide domain is fused to at least one second polypeptide domain. The second polypeptide domain has a different activity that what is endogenous to Cas protein. For example, the second polypeptide domain may have an activity such as transcription activation activity, transcription repression activity, transcription release factor activity, histone modification activity, nuclease activity, nucleic acid association activity, methylase activity, demethylase activity, acetylation activity, and/or deacetylation activity. The activity of the second polypeptide domain may be direct or indirect. The second polypeptide domain may have this activity itself (direct), or it may recruit and/or interact with a polypeptide domain that has this activity (indirect). In some embodiments, the second polypeptide domain has transcription activation activity. In some embodiments, the second polypeptide domain has transcription repression activity. In some embodiments, the second polypeptide domain comprises a synthetic transcription factor. The second polypeptide domain may be at the C-terminal end of the first polypeptide domain, or at the N-terminal end of the first polypeptide domain, or a combination thereof. The fusion protein may include one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

The linkage from the first polypeptide domain to the second polypeptide domain can be through reversible or irreversible covalent linkage or through a non-covalent linkage, as long as the linker does not interfere with the function of the second polypeptide domain. For example, a Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

In some embodiments, the fusion protein includes at least one linker. A linker may be included anywhere in the polypeptide sequence of the fusion protein, for example, between the first and second polypeptide domains. A linker may be of any length and design to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may include, for example, a GS linker (Gly-Gly-Gly-Gly-Ser)ₙ, wherein n is an integer between 0 and 10 (SEQ ID NO: 50). In a GS linker, n can be adjusted to optimize the linker length and achieve appropriate separation of the functional domains. Other examples of linkers may include, for example, Gly-Gly-Gly-Gly-Gly (SEQ ID NO: 51), Gly-Gly-Ala-Gly-Gly (SEQ ID NO: 52), Gly/Ser rich linkers such as Gly-Gly-Gly-Gly-Ser-Ser-Ser (SEQ ID NO: 53), or Gly/Ala rich linkers such as Gly-Gly-Gly-Gly-Ala-Ala-Ala (SEQ ID NO: 54).

(1) Transcription Activation Activity

The second polypeptide domain can have transcription activation activity, for example, a transactivation domain. For example, gene expression of endogenous mammalian genes, such as human genes, can be achieved by targeting a fusion protein of a first polypeptide domain, such as dCas9, and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, TET1, VPR, VPH, Rta, and/or p300. For example, the fusion protein may comprise dCas9-p300. In some embodiments, p300 comprises a polypeptide having the amino acid sequence of SEQ ID NO: 35 or SEQ ID NO: 36, In other embodiments, the fusion protein comprises dCas9-VP64. In other embodiments, the fusion protein comprises VP64-dCas9-VP64. VP64-dCas9-VP64 may comprise a polypeptide having the amino acid sequence of SEQ ID NO: 37, encoded by the polynucleotide of SEQ ID NO: 38. VPH may comprise a polypeptide having the amino acid sequence of SEQ ID NO: 45, encoded by the polynucleotide of SEQ ID NO: 46. VPR may comprise a polypeptide having the amino acid sequence of SEQ ID NO: 47, encoded by the polynucleotide of SEQ ID NO: 48.

(2) Transcription Repression Activity

The second polypeptide domain can have transcription repression activity. Non-limiting examples of repressors include Kruppel associated box activity such as a KRAB domain or KRAB, MECP2, EED, ERF repressor domain (ERD), Mad mSIN3 interaction domain (SID) or Mad-SID repressor domain, SID4X repressor domain, Mxil repressor domain, SUV39H1, SUV39H2, G9A, ESET/SETBD1, Cir4, Su(var)3-9, Pr-SET7%8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jrnj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof. In some embodiments, the second polypeptide domain has a KRAB domain activity, ERF repressor domain activity, Mxil repressor domain activity, SID4X repressor domain activity, Mad-SID repressor domain activity, DNMT3A or DNMT3L or fusion thereof activity, LSD1 histone demethylase activity, or TATA box binding protein activity. In some embodiments, the polypeptide domain comprises KRAB. For example, the fusion protein may be S. pyogenes dCas9-KRAB (polynucleotide sequence SEQ ID NO: 39; protein sequence SEQ ID NO: 40). The fusion protein may be S. aureus dCas9-KRAB (polynucleotide sequence SEQ ID NO: 41; protein sequence SEQ ID NO: 42).

(3) Transcription Release Factor Activity

The second polypeptide domain can have transcription release factor activity. The second polypeptide domain can have eukaryotic release factor 1 (ERF1) activity or eukaryotic release factor 3 (ERF3) activity.
(4) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 comprises a polypeptide of SEQ ID NO: 35 or SEQ ID NO: 36.

(5) Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease.

(6) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, and TAL effector DNA-binding domain.

(7) Methylase Activity

The second polypeptide domain can have methylase activity, which involves transferring a methyl group to DNA, RNA, protein, small molecule, cytosine, or adenine. In some embodiments, the second polypeptide domain includes a DNA methyltransferase.

(8) Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Tet1, also known as Tet1CD (Ten-eleven translocation methylcytosine dioxygenase 1; polynucleotide sequence SEQ ID NO: 43; amino acid sequence SEQ ID NO: 44). In some embodiments, the second polypeptide domain has histone demethylase activity. In some embodiments, the second polypeptide domain has DNA demethylase activity.

iii) Guide RNA (gRNA)

The CRISPR/Cas9-based gene editing system includes at least one gRNA molecule, for example, two gRNA molecules. The at least one gRNA molecule can bind and recognize a target region. The gRNA is the part of the CRISPR-Cas system that provides DNA targeting specificity to the CRISPR/Cas-based gene editing system. The gRNA is a fusion of two noncoding RNAs: a crRNA and a tracrRNA. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. The “target region” or “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds: “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region or scaffold of the gRNA may include the sequence of SEQ ID NO: 19 or 90 or 139 (RNA), which is encoded by a sequence comprising SEQ ID NO: 18 or 89 or 138 (DNA), respectively. The CRISPR/Cas9-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome. Different Type II systems have differing PAM requirements, as detailed above.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The CRISPR/Cas9-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules encoded by a presently disclosed genetic construct can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules encoded by a presently disclosed genetic construct can be less than 50 gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, or less than 3 different gRNAs. The number of gRNAs encoded by a presently disclosed genetic construct can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

The gRNA may target a region of the dystrophin gene (DMD). The at least one gRNA molecule can bind and recognize a target region, and in some embodiments, the target region is chosen immediately upstream of possible out-of-frame stop codons such that insertions or deletions during the repair process restore the dystrophin reading frame by frame conversion. Target regions can also be splice acceptor sites or splice donor sites, such that insertions or deletions during the repair process disrupt splicing and restore the dystrophin reading frame by splice site disruption and exon exclusion. Target regions can also be aberrant stop codons such that insertions or deletions during the repair process restore the dystrophin reading frame by eliminating or disrupting the stop codon. In certain embodiments, the gRNA can target at least one of exons, introns, the promoter region, the enhancer region, the transcribed region of the dystrophin gene. In certain embodiments, the gRNA molecule targets intron 44 of the human dystrophin gene. In certain embodiments, the gRNA molecule targets intron 55 of the human dystrophin gene. In some embodiments, a first gRNA and a second gRNA each target an intron of a human dystrophin gene such that exons 45 through 55 are deleted. A gRNA may bind and target a polynucleotide sequence corresponding to SEQ ID NO: 55 or 135 or a fragment thereof or a complement thereof. A gRNA may be encoded by a polynucleotide sequence comprising SEQ ID NO: 55 or 135 or a fragment thereof or a complement thereof. The targeting sequence of the gRNA may comprise the polynucleotide of SEQ ID NO: 55 or 135 or 57 or 137 or a fragment thereof, such as a 5′ truncation thereof, or a complement thereof, Truncations may be, for example, 1, 2, 3, 4, 5, 6, 7, 8, 9, 10, 11, 12, 13, 14, or 15 nucleotides shorter than SEQ ID NO: 55 or 135 or 57 or 137. In some embodiments, the gRNA may bind and target the polynucleotide of SEQ ID NO: 55 or 135. In some embodiments, the gRNA may bind and target a 5′ truncation of the polynucleotide of SEQ ID NO: 55 or 135. A gRNA may bind and target a polynucleotide sequence corresponding to SEQ ID NO: 56 or 134 or a fragment thereof or a complement thereof. A gRNA may be encoded by a polynucleotide sequence comprising SEQ ID NO: 56 or 134 or a fragment thereof or a complement thereof. The targeting sequence of the gRNA may comprise the polynucleotide of SEQ ID NO: 56 or 134 or 58 or 136 or a fragment thereof, such as a 5′ truncation thereof, or a complement thereof. Truncations may be, for example, 1, 2, 3, 4, 5, 6, 7, 8, 9, 10, 11, 12, 13, 14, or 15 nucleotides shorter than SEQ ID NO: 56 or 134 or 58 or 136. In some embodiments, the gRNA may bind and target the polynucleotide of SEQ ID NO: 56 or 134. In some embodiments, the gRNA may bind and target a 5′ truncation of the polynucleotide of SEQ ID NO: 56 or 134. In some embodiments, a gRNA that binds and targets or is encoded by a polynucleotide sequence comprising or corresponding to SEQ ID NO: 55 or 135 or truncation thereof is paired with a gRNA that binds and targets or is encoded by a polynucleotide sequence comprising or corresponding to SEQ ID NO: 56 or 134 or truncation thereof. In certain embodiments, the presently disclosed systems include a first gRNA and a second gRNA. The first gRNA molecule and the second gRNA molecule may bind or target a polynucleotide of SEQ ID NO: 55 or 135 and SEQ ID NO: 56 or 134, respectively, or a truncation or a complement thereof. The first gRNA molecule and the second gRNA molecule may comprise a polynucleotide corresponding to SEQ ID NO: 55 or 135 and SEQ ID NO: 56 or 134, respectively, or a truncation or a complement thereof. The first gRNA molecule and the second gRNA molecule may comprise a polynucleotide corresponding to SEQ ID NO: 57 or 137 and SEQ ID NO: 58 or 136, respectively, or a truncation or a complement thereof.

Single or multiplexed gRNAs can be designed to restore the dystrophin reading frame by targeting the mutational hotspot in exons 45-55 of dystrophin. Following treatment with a presently disclosed vector, dystrophin expression can be restored in Duchenne patient muscle cells in vitro. Human dystrophin was detected in vivo following transplantation of genetically corrected patient cells into immunodeficient mice. Significantly, the unique multiplex gene editing capabilities of the CRISPR/Cas9-based gene editing system enable efficiently generating large deletions of this mutational hotspot region that can correct up to 62% of patient mutations by universal or patient-specific gene editing approaches. In some embodiments, candidate gRNAs are evaluated and chosen based on off-target activity, on-target activity as measured by surveyor, and distance from the exon.

The deletion efficiency of the presently disclosed vectors can be related to the deletion size, i.e., the size of the segment deleted by the vectors. In certain embodiments, the length or size of specific deletions is determined by the distance between the PAM sequences in the gene being targeted. In certain embodiments, a specific deletion of a segment of the dystrophin gene, which is defined in terms of its length and a sequence it comprises (e.g., exon 51), is the result of breaks made adjacent to specific PAM sequences within the target gene (e.g., a dystrophin gene).

In certain embodiments, the deletion size is about 50 to about 2,000 base pairs (bp), e.g., about 50 to about 1999 bp, about 50 to about 1900 bp, about 50 to about 1800 bp, about 50 to about 1700 bp, about 50 to about 1650 bp, about 50 to about 1600 bp, about 50 to about 1500 bp, about 50 to about 1400 bp, about 50 to about 1300 bp, about 50 to about 1200 bp, about 50 to about 1150 bp, about 50 to about 1100 bp, about 50 to about 1000 bp, about 50 to about 900 bp, about 50 to about 850 bp, about 50 to about 800 bp, about 50 to about 750 bp, about 50 to about 700 bp, about 50 to about 600 bp, about 50 to about 500 bp, about 50 to about 400 bp, about 50 to about 350 bp, about 50 to about 300 bp, about 50 to about 250 bp, about 50 to about 200 bp, about 50 to about 150 bp, about 50 to about 100 bp, about 100 to about 1999 bp, about 100 to about 1900 bp, about 100 to about 1800 bp, about 100 to about 1700 bp, about 100 to about 1650 bp, about 100 to about 1600 bp, about 100 to about 1500 bp, about 100 to about 1400 bp, about 100 to about 1300 bp, about 100 to about 1200 bp, about 100 to about 1150 bp, about 100 to about 1100 bp, about 100 to about 1000 bp, about 100 to about 900 bp, about 100 to about 850 bp, about 100 to about 800 bp, about 100 to about 750 bp, about 100 to about 700 bp, about 100 to about 600 bp, about 100 to about 1000 bp, about 100 to about 400 bp, about 100 to about 350 bp, about 100 to about 300 bp, about 100 to about 250 bp, about 100 to about 200 bp, about 100 to about 150 bp, about 200 to about 1999 bp, about 200 to about 1900 bp, about 200 to about 1800 bp, about 200 to about 1700 bp, about 200 to about 1650 bp, about 200 to about 1600 bp, about 200 to about 1500 bp, about 200 to about 1400 bp, about 200 to about 1300 bp, about 200 to about 1200 bp, about 200 to about 1150 bp, about 200 to about 1100 bp, about 200 to about 1000 bp, about 200 to about 900 bp, about 200 to about 850 bp, about 200 to about 800 bp, about 200 to about 750 bp, about 200 to about 700 bp, about 200 to about 600 bp, about 200 to about 2000 bp, about 200 to about 400 bp, about 200 to about 350 bp, about 200 to about 300 bp, about 200 to about 250 bp, about 300 to about 1999 bp, about 300 to about 1900 bp, about 300 to about 1800 bp, about 300 to about 1700 bp, about 300 to about 1650 bp, about 300 to about 1600 bp, about 300 to about 1500 bp, about 300 to about 1400 bp, about 300 to about 1300 bp, about 300 to about 1200 bp, about 300 to about 1150 bp, about 300 to about 1100 bp, about 300 to about 1000 bp, about 300 to about 900 bp, about 300 to about 850 bp, about 300 to about 800 bp, about 300 to about 750 bp, about 300 to about 700 bp, about 300 to about 600 bp, about 300 to about 3000 bp, about 300 to about 400 bp, or about 300 to about 350 bp. In certain embodiments, the deletion size can be about 118 base pairs, about 233 base pairs, about 326 base pairs, about 766 base pairs, about 805 base pairs, or about 1611 base pairs.

iv) Repair Pathways

The CRISPR/Cas9-based gene editing system may be used to introduce site-specific double strand breaks at targeted genomic loci in the dystrophin gene. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

(1) Homology-Directed Repair (HDR)

Restoration of protein expression from a gene may involve homology-directed repair (HDR). A donor template may be administered to a cell. The donor template may include a nucleotide sequence encoding a full-functional protein or a partially functional protein. In such embodiments, the donor template may include fully functional gene construct for restoring a mutant gene, or a fragment of the gene that after homology-directed repair, leads to restoration of the mutant gene. In other embodiments, the donor template may include a nucleotide sequence encoding a mutated version of an inhibitory regulatory element of a gene. Mutations may include, for example, nucleotide substitutions, insertions, deletions, or a combination thereof. In such embodiments, introduced mutation(s) into the inhibitory regulatory element of the gene may reduce the transcription of or binding to the inhibitory regulatory element.

(2) Non-Homologous End Joining (NHEJ)

Restoration of protein expression from gene may be through template-free NHEJ-mediated DNA repair. In certain embodiments, NHEJ is a nuclease mediated NHEJ, which in certain embodiments, refers to NHEJ that is initiated a Cas9 molecule that cuts double stranded DNA. The method comprises administering a presently disclosed CRISPR/Cas9-based gene editing system or a composition comprising thereof to a subject for gene editing.

Nuclease mediated NHEJ may correct a mutated target gene and offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment.

### 4. Genetic Constructs for Genome Editing

Disclosed herein is a genetic construct or a composition thereof for genome editing a target gene in a subject, such as, for example, a target gene in skeletal muscle and/or cardiac muscle of a subject. The genetic construct may be a vector. The vector may be a modified AAV vector. The composition may include a polynucleotide sequence encoding a CRISPR/Cas9-based gene editing system. The composition may deliver active forms of CRISPR/Cas9-based gene editing systems to skeletal muscle or cardiac muscle. The presently disclosed genetic constructs can be used in correcting or reducing the effects of mutations in the dystrophin gene involved in genetic diseases and/or other skeletal or cardiac muscle conditions, such as, for example, DMD. The composition may further comprise a donor DNA or a transgene. These compositions may be used in genome editing, genome engineering, and correcting or reducing the effects of mutations in genes involved in genetic diseases and/or other skeletal and/or cardiac muscle conditions.

The CRISPR/Cas9-based gene editing system may be encoded by or comprised within one or more genetic constructs. The CRISPR/Cas9-based gene editing system may comprise one or more genetic constructs. The genetic construct, such as a plasmid or expression vector, may comprise a nucleic acid that encodes the CRISPR/Cas9-based gene editing system and/or at least one of the gRNAs. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein. In some embodiments, a first genetic construct encodes one gRNA molecule and one donor sequence, and a second genetic construct encodes a Cas9 molecule or fusion protein. In some embodiments, a first genetic construct encodes one gRNA molecule and a Cas9 molecule or fusion protein, and a second genetic construct encodes one donor sequence. In certain embodiments, the genetic construct (for example, an AAV vector) encodes one gRNA molecule, i.e., a first gRNA molecule, and optionally a Cas9 molecule. In certain embodiments, a first genetic construct (for example, a first AAV vector) encodes one gRNA molecule, i.e., a first gRNA molecule, and optionally a Cas9 molecule, and a second genetic construct (for example, a second AAV vector) encodes one gRNA molecule, i.e., a second gRNA molecule, and optionally a Cas9 molecule. In certain embodiments, a first genetic construct (for example, a first AAV vector) encodes two gRNA molecules, i.e., a first gRNA molecule and a second gRNA molecule, and a second genetic construct (for example, a second AAV vector) encodes a Cas9 molecule.

In some embodiments, the vector comprises at least one polynucleotide sequence selected from SEQ ID NOs: 55, 56, 89, 59-72, 132-135, 138, 140. In some embodiments, the vector comprises the polynucleotide sequence selected from SEQ ID NOs: 91, 92, 128, 129, 130, and 131.

In embodiments include more than one vector, the vectors may be present in the same or different concentrations. The first vector and second vector may be administered or comprised within a composition in various ratios. For example, the first vector may be present in a concentration of at least 2-fold, at least 3-fold, at least 4-fold, at least 5-fold, at least 6-fold, at least 7-fold, at least 8-fold, at least 9-fold, or at least 10-fold greater than the concentration of the second vector. The first vector may be present in a concentration of at least 2-fold, at least 3-fold, at least 4-fold, at least 5-fold, at least 6-fold, at least 7-fold, at least 8-fold, at least 9-fold, or at least 10-fold less than the concentration of the second vector. The first vector may be present in a concentration of less than 10-fold, less than 9-fold, less than 8-fold, less than 7-fold, less than 6-fold, or less than 5-fold greater than the concentration of the second vector. The first vector may be present in a concentration of less than 10-fold, less than 9-fold, less than 8-fold, less than 7-fold, less than 6-fold, or less than 5-fold less than the concentration of the second vector. The first vector may be present in a concentration that is about 2-fold to about 10-fold, about 3-fold to about 9-fold, about 2-fold to about 8-fold, about 4-fold to about 6-fold, or about 3-fold to about 7-fold greater than the concentration of the second vector. The first vector may be present in a concentration that is about 2-fold to about 10-fold, about 3-fold to about 9-fold, about 2-fold to about 8-fold, about 4-fold to about 6-fold, or about 3-fold to about 7-fold less than the concentration of the second vector. The first vector and the second vector may be present or administered in a ratio of about 1:1, 1:2, 1:3, 1:4, 1:5, 1:6, 1:7, 1:8, 1:9, 1:1010:1, 9:1, 8:1, 7:1, 6:1, 5:1, 4:1, 3:1, or 2:1.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The genetic construct may include more than one stop codon, which may be downstream of the CRISPR/Cas-based gene editing system coding sequence. In some embodiments, the genetic construct includes 1, 2, 3, 4, or 5 stop codons. In some embodiments, the genetic construct includes 1, 2, 3, 4, or 5 stop codons downstream of the sequence encoding the donor sequence. A stop codon may be in-frame with a coding sequence in the CRISPR/Cas-based gene editing system. For example, one or more stop codons may be in-frame with the donor sequence. The genetic construct may include one or more stop codons that are out of frame of a coding sequence in the CRISPR/Cas-based gene editing system. For example, one stop codon may be in-frame with the donor sequence, and two other stop codons may be included that are in the other two possible reading frames. A genetic construct may include a stop codon for all three potential reading frames. The initiation and termination codon may be in frame with the CRISPR/Cas-based gene editing system coding sequence.

The vector may also comprise a promoter that is operably linked to the CRISPR/Cas-based gene editing system coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may be a human U6 promoter. The promoter may be a H1 promoter. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a MHCK7 promoter, for example. Promoters may comprise a polynucleotide sequence selected from, for example, SEQ ID NOs: 63-68 and 133.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human p-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

The genetic construct may be useful for transfecting cells with nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. Further provided are stem cell-derived neurons, such as neurons derived from iPSCs transformed or transduced with a DNA targeting system or component thereof as detailed herein.

a. Self-Complementary Vector

In some embodiments, the CRISPR/Cas-based systems detailed herein include a self-complementary vector. A vector may include two inverted terminal repeats (ITRs), one ITR on either end of the coding sequence for gRNA(s) and/or Cas protein. A self-complementary vector includes a mutant ITR. The mutant ITR directs vector genome replication to generate a self-complementary vector genome. The self-complementary genome may form a double-stranded polynucleotide. The self-complementary genome may be about the same length of a (non-self-complementary) genome comprising an open reading frame flanked on both ends by a wild-type ITR. When formed as a double-stranded polynucleotide, the self-complementary genome may be about the same length as a (non-self-complementary) genome comprising an open reading frame flanked on both ends by a wild-type ITR. When present as a single-stranded polynucleotide, the self-complementary genome may be about twice the length as a (non-self-complementary) genome comprising an open reading frame flanked on both ends by a wild-type ITR. The self-complementary vector may also include a wild-type ITR. In some embodiments, the self-complementary vector includes a polynucleotide comprising an open reading frame with a wild-type ITR at one end and a mutant ITR at the other end. In some embodiments the wild-type ITR comprises a polynucleotide sequence selected from SEQ ID NOs: 59-61 and 132. In some embodiments the mutant ITR comprises a polynucleotide sequence of SEQ ID NO: 62 or 140.

In some embodiments, the CRISPR/Cas-based systems detailed herein include a dual vector system. The CRISPR/Cas-based system may include a first vector and a second vector. The first vector may include a first and a second gRNA, and the second vector may encode a Cas protein or a Cas fusion protein. The first vector and/or the second vector may be a self-complementary vector. In some embodiments, the first vector is a self-complementary vector and encodes a first and a second gRNA.

b. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver CRISPR/Cas9-based gene editing systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

### 5. Pharmaceutical Compositions

Further provided herein are pharmaceutical compositions comprising the above-described genetic constructs or gene editing systems. In some embodiments, the pharmaceutical composition may comprise about 1 ng to about 10 mg of DNA encoding the CRISPR/Cas-based gene editing system. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 6. Administration

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject, Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. In certain embodiments, the system, genetic construct, or composition comprising the same, is administered to a subject systemically. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibialis anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration. In some embodiments, the presently disclosed genetic construct (e.g., a vector) or a composition thereof is administered by 1) tail vein injections (systemic) into adult mice; 2) intramuscular injections, for example, local injection into a muscle such as the TA or gastrocnemius in adult mice; 3) intraperitoneal injections into P2 mice; or 4) facial vein injection (systemic) into P2 mice.

Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein.

a. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a CRISPR/Cas9 system as detailed herein. Suitable cell types are detailed herein. In some embodiments, the cell is an immune cell. Immune cells may include, for example, lymphocytes such as T cells and B cells and natural killer (NK) cells. In some embodiments, the cell is a T cell. T cells may be divided into cytotoxic T cells and helper T cells, which are in turn categorized as TH1 or TH2 helper T cells. Immune cells may further include innate immune cells, adaptive immune cells, tumor-primed T cells, NKT cells, IFN-γ producing killer dendritic cells (IKDC), memory T cells (TCMs), and effector T cells (TEs). The cell may be a stem cell such as a human stem cell. In some embodiments, the cell is an embryonic stem cell or a hematopoietic stem cell. The stem cell may be a human induced pluripotent stem cell (iPSCs). Further provided are stem cell-derived neurons, such as neurons derived from iPSCs transformed or transduced with a DNA targeting system or component thereof as detailed herein. The cell may be a muscle cell. Cells may further include, but are not limited to, immortalized myoblast cells, such as wild-type and DMD patient derived lines, for example Δ48-50 DMD, DMD 6594 (del48-50), DMD 8036 (del48-50), C25C14 and DMD-7796 cell lines, primal DMD dermal fibroblasts, dermal fibroblasts, bone marrow-derived progenitors, skeletal muscle progenitors, human skeletal myoblasts from DMD patients, human skeletal myoblasts, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells.

Immortalization of human myogenic cells can be used for clonal derivation of genetically corrected myogenic cells. Cells can be modified ex vivo to isolate and expand clonal populations of immortalized DMD myoblasts that include a genetically corrected dystrophin gene and are free of other nuclease-introduced mutations in protein coding regions of the genome. Alternatively, transient in vivo delivery of CRISPR/Cas9-based systems by non-viral or non-integrating viral gene transfer, or by direct delivery of purified proteins and gRNAs containing cell-penetrating motifs may enable highly specific correction in situ with minimal or no risk of exogenous DNA integration.

### 7. Kits

Provided herein is a kit, which may be used for editing a dystrophin gene. The kit comprises genetic constructs or a composition comprising the same, and instructions for using said composition. In some embodiments, the kit comprises at least one gRNA comprising or encoded by a polynucleotide sequence of SEQ ID NO: 55 or 57 or 135 or 137, a complement thereof, a variant thereof, or fragment thereof, or gRNA targeting a polynucleotide sequence of SEQ ID NO: 56 or 58 or 134 or 136, a complement thereof, a variant thereof, or fragment thereof. The kit may further include a mutant ITR. The kit may further include at least one self-complementary vector. The kit may further include instructions for using the CRISPR/Cas-based gene editing system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions. The genetic constructs or a composition comprising thereof for modifying a dystrophin gene may include a modified AAV vector that includes a gRNA molecule(s) and a Cas9 protein or fusion protein, as described above, that specifically binds and cleaves a region of the dystrophin gene. The CRISPR/Cas-based gene editing system, as described above, may be included in the kit to specifically bind and target a particular region in the gene.

### 8. Methods

a. Methods of Genome Editing in Muscle

Disclosed herein are methods of genome editing in subject. The method may include administering to the subject a CRISPR/Cas9-based system as detailed herein or a cell comprising a CRISPR/Cas9-based system as detailed herein. The genome editing may be in a skeletal muscle and/or cardiac muscle of a subject. The method may comprise administering to the skeletal muscle and/or cardiac muscle of the subject the system or composition for genome editing, as described above. The genome editing may include correcting a mutant gene or inserting a transgene. Correcting the mutant gene may include deleting, rearranging, or replacing the mutant gene. Correcting the mutant gene may include nuclease-mediated NHEJ or HDR.

In some embodiments, the subject is an adult, an adolescent, or a pre-adolescent. In some embodiments, the system or the cell is administered to the subject intravenously. In some embodiments, the system or the cell is administered to the subject systemically.

b. Methods of Correcting a Mutant Gene and Treating a Subject

Disclosed herein are methods of correcting a mutant dystrophin gene in a cell or a subject. The method may include administering to the cell or subject a CRISPR/Cas9-based system as detailed herein, or administering to the a cell comprising a CRISPR/Cas9-based system as detailed herein. Further disclosed herein are methods of correcting a mutant gene (such as a mutant dystrophin gene, such as a mutant human dystrophin gene) in a cell and treating a subject suffering from a genetic disease, such as DMD. The method can include administering to a cell or a subject a presently disclosed system or genetic construct (e.g., a vector) or a composition comprising thereof as described above. The method can comprise administering to the skeletal muscle and/or cardiac muscle of the subject the presently disclosed system or genetic construct (e.g., a vector) or a composition comprising the same for genome editing in skeletal muscle and/or cardiac muscle, as described above. Use of the presently disclosed system or genetic construct (e.g., a vector) or a composition comprising the same to deliver the CRISPR/Cas9-based gene editing system to the skeletal muscle or cardiac muscle may restore the expression of a fully-functional or partially-functional protein with a repair template or donor DNA, which can replace the entire gene or the region containing the mutation. The CRISPR/Cas9-based gene editing system may be used to introduce site-specific double strand breaks at targeted genomic loci. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

Provided herein is genome editing with a CRISPR/Cas9-based gene editing system without a repair template, which can efficiently correct the reading frame and restore the expression of a functional protein involved in a genetic disease. The disclosed CRISPR/Cas9-based gene editing systems may involve using homology-directed repair or nuclease-mediated non-homologous end joining (NHEJ)-based correction approaches, which enable efficient correction in proliferation-limited primary cell lines that may not be amenable to homologous recombination or selection-based gene correction. This strategy integrates the rapid and robust assembly of active CRISPR/Cas9-based gene editing systems with an efficient gene editing method for the treatment of genetic diseases caused by mutations in nonessential coding regions that cause frameshifts, premature stop codons, aberrant splice donor sites or aberrant splice acceptor sites.

The present disclosure is directed to genome editing with CRISPR/Cas9-based gene editing system without a repair template, which can efficiently correct the reading frame and restore the expression of a functional protein involved in a genetic disease. The disclosed CRISPR/Cas9-based gene editing system and methods may involve using homology-directed repair or nuclease-mediated non-homologous end joining (NHEJ)-based correction approaches, which enable efficient correction in proliferation-limited primary cell lines that may not be amenable to homologous recombination or selection-based gene correction. This strategy integrates the rapid and robust assembly of active CRISPR/Cas9-based gene editing system with an efficient gene editing method for the treatment of genetic diseases caused by mutations in nonessential coding regions that cause frameshifts, premature stop codons, aberrant splice donor sites or aberrant splice acceptor sites.

The present disclosure provides methods of correcting a mutant gene in a cell and treating a subject suffering from a genetic disease, such as DMD. The method may include administering to a cell or subject a CRISPR/Cas9-based gene editing system, a polynucleotide or vector encoding said CRISPR/Cas9-based gene editing system, or composition of said CRISPR/Cas9-based gene editing system as described above. The method may include administering a CRISPR/Cas9-based gene editing system, such as administering a Cas9 protein or Cas9 fusion protein containing a second domain having nuclease activity, a nucleotide sequence encoding said Cas9 protein or Cas9 fusion protein, and/or at least one gRNA, wherein the gRNAs target different DNA sequences. The target DNA sequences may be overlapping. The number of gRNA administered to the cell may be at least 1 gRNA, at least 2 different gRNA, at least 3 different gRNA at least 4 different gRNA, at least 5 different gRNA, at least 6 different gRNA, at least 7 different gRNA, at least 8 different gRNA, at least 9 different gRNA, at least 10 different gRNA, at least 15 different gRNA, at least 20 different gRNA, at least 30 different gRNA, or at least 50 different gRNA, as described above. The method may involve homology-directed repair or non-homologous end joining.

In some embodiments, the subject is an adult, an adolescent, or a pre-adolescent. In some embodiments, the system or the cell is administered to the subject intravenously. In some embodiments, the system or the cell is administered to the subject systemically.

c. Methods of Treating Disease

Provided herein is a method of treating subject having a mutant dystrophin gene. The method may include administering to the subject a CRISPR/Cas9-based system as detailed herein or a cell comprising a CRISPR/Cas9-based system as detailed herein. The present disclosure is also directed to a method of treating a subject in need thereof. The method comprises administering to a tissue of a subject the presently disclosed system or genetic construct (e.g., a vector) or a composition comprising thereof, as described above. In certain embodiments, the method may comprise administering to the skeletal muscle or cardiac muscle of the subject the presently disclosed system or genetic construct (e.g., a vector) or composition comprising thereof, as described above. In certain embodiments, the method may comprise administering to a vein of the subject the presently disclosed system or genetic construct (e.g., a vector) or composition comprising thereof, as described above. In certain embodiments, the subject is suffering from a skeletal muscle or cardiac muscle condition causing degeneration or weakness or a genetic disease. For example, the subject may be suffering from Duchenne muscular dystrophy, as described above.

In some embodiments, the subject is an adult, an adolescent, or a pre-adolescent. In some embodiments, the system or the cell is administered to the subject intravenously. In some embodiments, the system or the cell is administered to the subject systemically.

The method, as described above, may be used for correcting the dystrophin gene and recovering full-functional or partially-functional protein expression of said mutated dystrophin gene. In some aspects and embodiments the disclosure provides a method for reducing the effects (e.g., clinical symptoms/indications) of DMD in a patient. In some aspects and embodiments the disclosure provides a method for treating DMD in a patient. In some aspects and embodiments the disclosure provides a method for preventing DMD in a patient. In some aspects and embodiments the disclosure provides a method for preventing further progression of DMD in a patient.

### 9. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure details multiple embodiments and aspects, illustrated by the following non-limiting examples.

### Example 1

### Dual Vector System

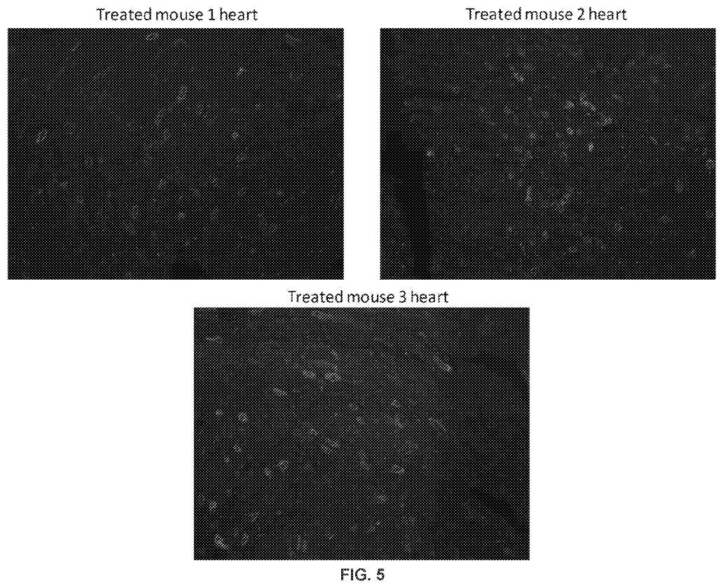

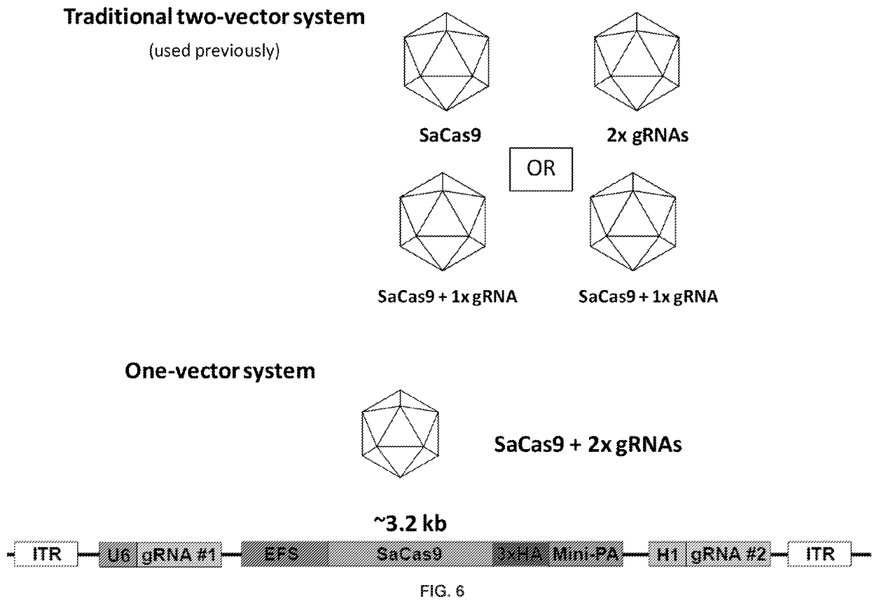

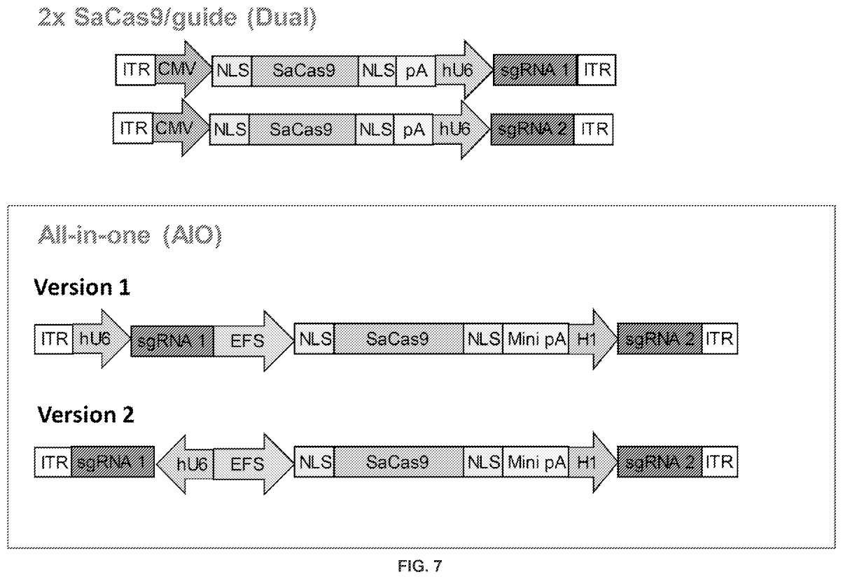

Conventional CRISPR/Cas9 systems for the treatment of DMD typically include more than one vector (FIG. 6, FIG. 7). For example, one vector may encode a Cas9 protein, and a second vector may encode two gRNAs. As another example, one vector may encode a Cas9 protein and a first gRNA, and a second vector may encode a Cas9 protein and a second gRNA.

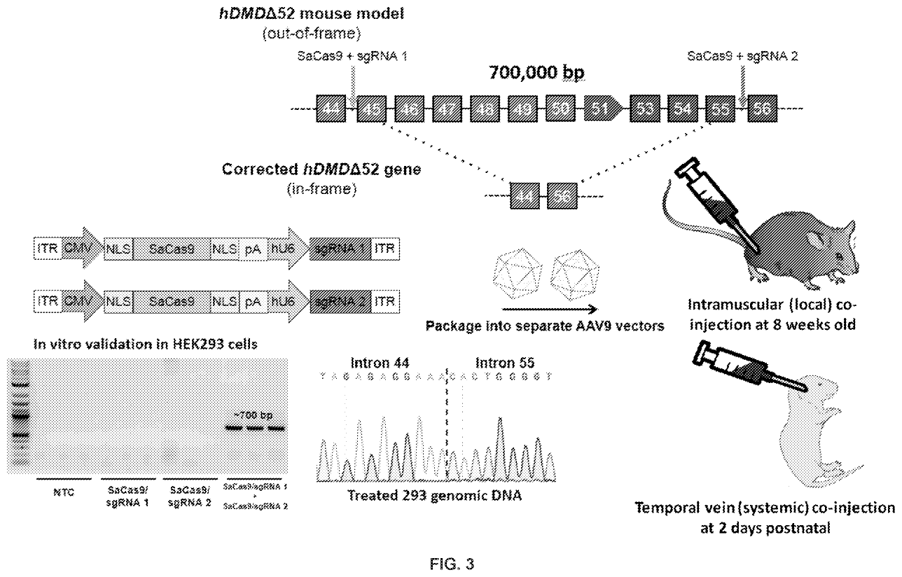

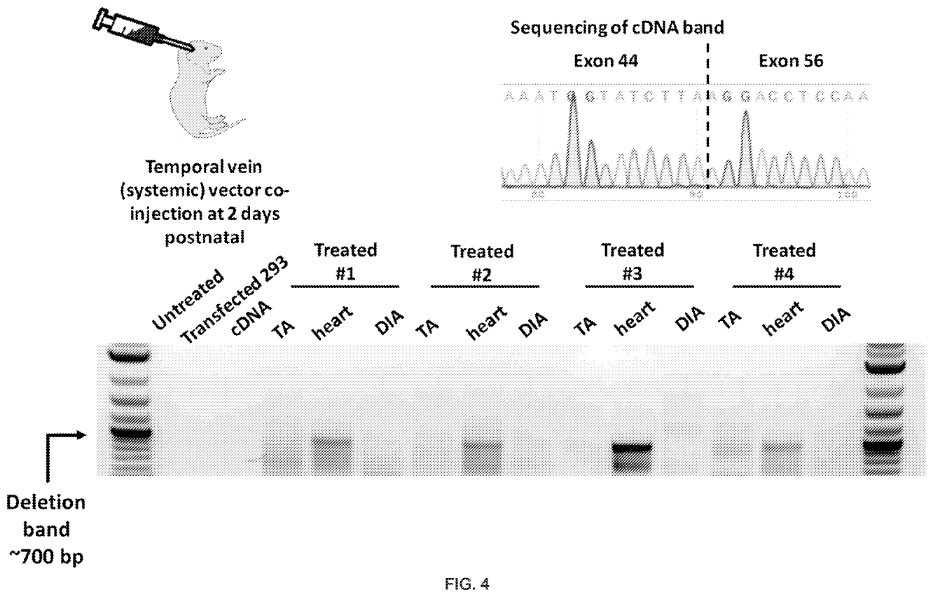

A schematic of an experiment that uses multiple vectors to excise exons 45-55 of dystrophin in mice is shown in FIG. 3 with results shown in FIG. 4, FIG. 5, and FIG. 10. Neonatal mice were treated with the dual vector system via systemic/temporal vein injection. At 8 weeks post-treatment, tissue was harvested. As shown in FIG. 4, PCR and sequencing confirmed the deletion of the mutational hotspot exon 45-55. Additional results are shown in FIG. 10 with either AAV-CRISPR targeting a control locus (FIG. 10, top panel) or targeting exon 45-55 (FIG. 10, bottom panel), showing that widespread dystrophin expression was observed in cardiac muscle after deletion of exon 45-55, but not in sham vector-treated mice.

### Example 2

### Validation of Therapeutic Approach for Dual Vector System

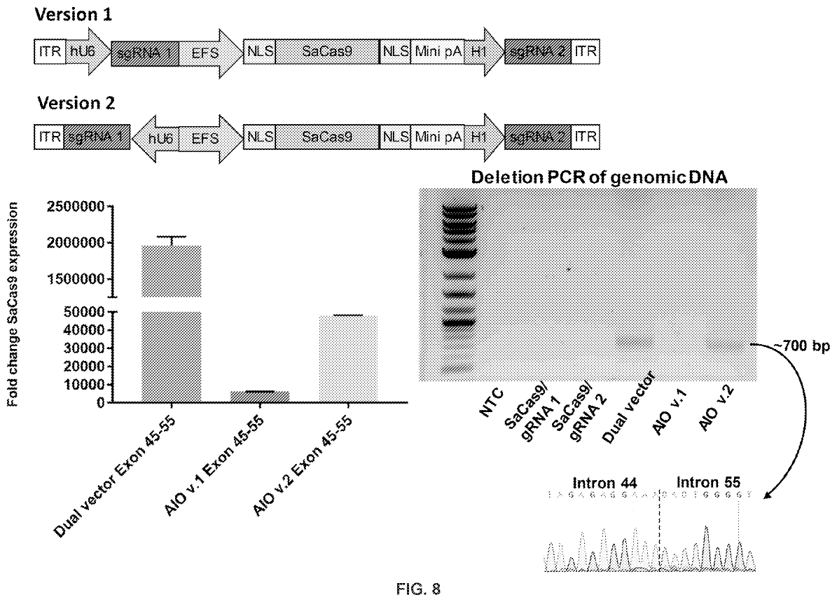

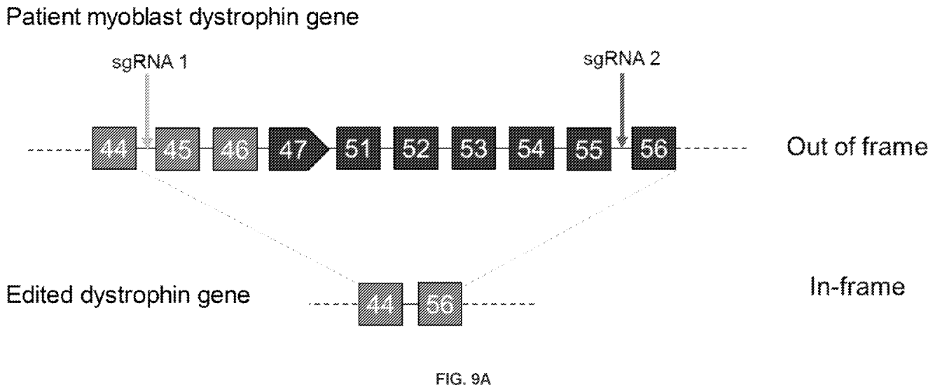

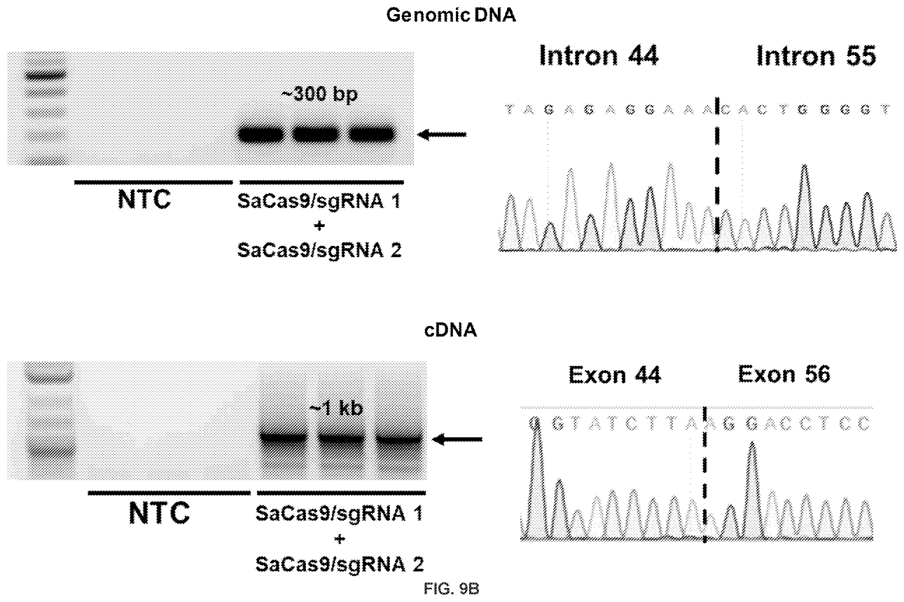

Additional validation of the CRISPR-based approach to restore functional dystrophin gene with the dual vectors of Example 1 was performed using immortalized myoblasts isolated from a DMD patient. The immortalized myoblasts contained a deletion of exons 48-50, creating an out-of-frame mutation (FIG. 9A). Patient myoblasts were transfected with the same AAV plasmids used in the HEK293 in vitro experiment in Example 1.

Deletion PCR of genomic DNA and cDNA revealed that exon 45-55 was effectively deleted, which was confirmed by Sanger sequencing (FIG. 9B). Western blot of cell lysates showed that untreated myoblasts produced no dystrophin protein, while transfected myoblasts expressed a smaller dystrophin protein compared to the positive control, consistent with hotspot deletion (FIG. 9C). These results additionally provided in vitro validation that the dual vector constructs can be used to edit a human mutation and restore dystrophin expression.

### Example 3

### Components for All-In-One Vectors

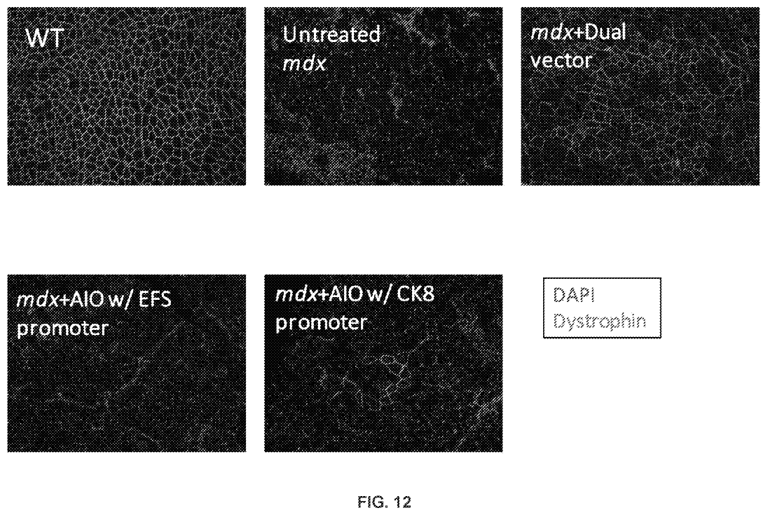

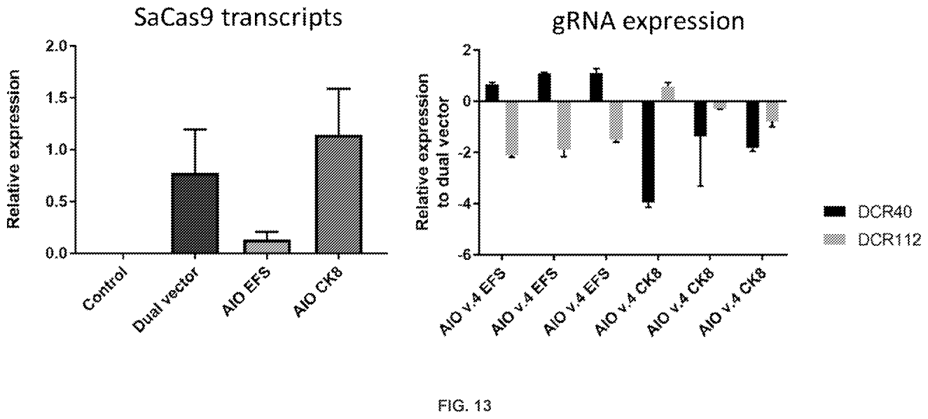

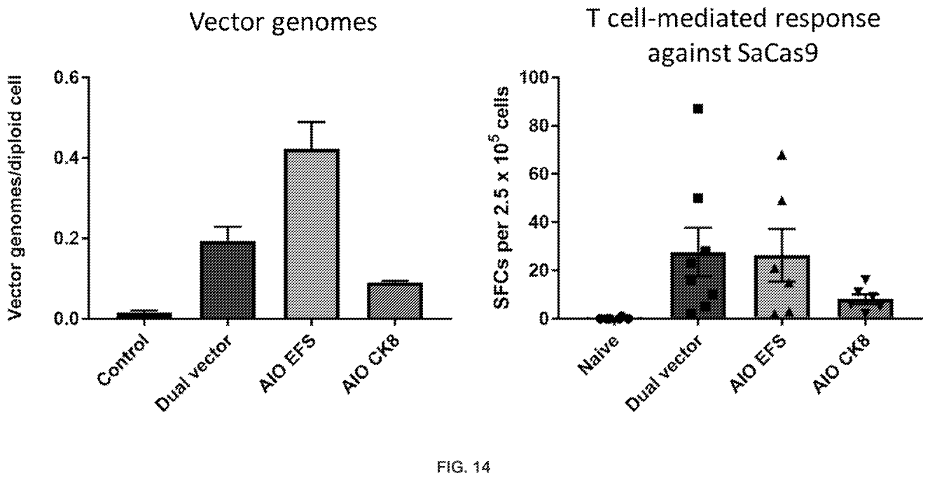

A one-vector CRISPR/Cas9 system was developed for the treatment of DMD (FIG. 6, FIG. 7). Advantages to a one vector system may include having all necessary editing components on a single vector, ability to increase effective dose, streamlining of other vector production (single therapeutic agent), use/incorporation of muscle-specific promoters (for example, CK8, Spc512, MHCK7), and ability to target combinations of exons and large deletions (for example, by changing guide sequences). A schematic diagram of the all-in-one vectors developed is shown in FIG. 8. Sequences included in some or all of the herein described all-in-one vectors are shown in TABLE 1. FIG. 12, FIG. 13, and FIG. 14 show results from testing these constructs in the mdx mouse. The all-in-one vectors are further detailed in Examples 4-7.

TABLE 1

| Component | Sequence |
|---|---|
| AAV ITR (wild- | CCTGCAGGCAGCTGCGCGCTCGCTCGCTCACTGAGGCCGCCC |
| type ITR-1) | GGGCGTCGGGCGACCTTTGGTCGCCCGGCCTCAGTGAGCGAG |
| | CGAGCGCGCAGAGAGGGAGTGGCCAACTCCATCACTAGGGGT |
| | TCCT (SEQ ID NO: 59) |
| AAV ITR (wild- | GGGGGGGGGGGGGGGGGGTTGGCCACTCCCTCTCTGCGCGC |
| type ITR-2) | TCGCTCGCTCACTGAGGCCGGGCGACCAAAGGTCGCCCGACG |
| | CCCGGGCTTTGCCCGGGCGGCCTCAGTGAGCGAGCGAGCGC |
| | GCAGAGAGGGAGTGGCCAACTCCATCACTAGGG |
| | (SEQ ID NO: 60) |
| AAV ITR (wild- | GGGCCACTCCCTCTCTGCGCGCTCGCTCGCTCACTGAGGCCG |
| type ITR-3) | GGCGACCAAAGGTCGCCCGACGCCCGGGCTTTGCCCGGGCG |
| | GCCTCAGTGAGCGAGCGAGCGCGCAGAGAGGGAGTGGCCAAC |
| | TCCATCACTAGGGGTTCCT (SEQ ID NO: 61) |
| Mutant ITR | CTAGTCCACTCCCTCTCTGCGCGCTCGCTCGCTCACTGAGGCC |
| | GGGCGACCAAAGGTCGCCCGACGCCCGGGCTTTGCCCGGGC |
| | GGCCTCAGTGAGCGAGCGAGCGCGCAGAGAGGGAC |
| | (SEQ ID NO: 62) |
| JCR143: | ACATTTCCTCTCTATACAAATG (SEQ ID NO: 55) |
| guide sequence | |
| RNA targeting | |
| human dystrophin | |
| intron 44 region | |
| JCR120: | ATATAGTAATGAAATTATTGGCAC (SEQ ID NO: 56) |
| guide sequence | |
| RNA targeting | |
| human dystrophin | |
| intron 55 region | |
| SaCas9 guide | TCTCGCCAACAAGTTGACGAGATAAACACGGCATTTTGCCTTGT |
| RNA scaffold | TTTAGTAGATTCTGTTTCCAGAGTACTAAAAC |
| | (SEQ ID NO: 89) |
| U6 promoter | GGTGTTTCGTCCTTTCCACAAGATATATAAAGCCAAGAAATCGA |
| | AATACTTTCAAGTTACGGTAAGCATATGATAGTCCATTTTAAAAC |
| | ATAATTTTAAAACTGCAAACTACCCAAGAAATTATTACTTTCTAC |
| | GTCACGTATTTTGTACTAATATCTTTGTGTTTACAGTCAAATTAA |
| | TTCCAATTATCTCTCTAACAGCCTTGTATCGTATATGCAAATATG |
| | AAGGAATCATGGGAAATAGGCCCTC (SEQ ID NO: 63) |
| H1 promoter | GAACGCTGACGTCATCAACCCGCTCCAAGGAATCGCGGGCCC |
| | AGTGTCACTAGGCGGGAACACCCAGCGCGCGTGCGCCCTGGC |
| | AGGAAGATGGCTGTGAGGGACAGGGGAGTGGCGCCCTGCAAT |
| | ATTTGCATGTCGCTATGTGTTCTGGGAAATCACCATAAACGTGA |
| | AATGTCTTTGGATTTGGGAATCTTATAAGTTCTGTATGAGACCA |
| | C (SEQ ID NO: 64) |
| EFS promoter | TCGAGTGGCTCCGGTGCCCGTCAGTGGGCAGAGCGCACATCG |
| | CCCACAGTCCCCGAGAAGTTGGGGGGAGGGGTCGGCAATTGA |
| | ACCGGTGCCTAGAGAAGGTGGCGCGGGGTAAACTGGGAAAGT |
| | GATGTCGTGTACTGGCTCCGCCTTTTTCCCGAGGGTGGGGGAG |
| | AACCGTATATAAGTGCAGTAGTCGCCGTGAACGTTCTTTTTCGC |
| | AACGGGTTTGCCGCCAGAACACAGGTGTCGTGACCGCGG |
| | (SEQ ID NO: 65) |
| CK8 promoter | CTAGACTAGCATGCTGCCCATGTAAGGAGGCAAGGCCTGGGG |
| | ACACCCGAGATGCCTGGTTATAATTAACCCAGACATGTGGCTG |
| | CCCCCCCCCCCCCAACACCTGCTGCCTCTAAAAATAACCCTGC |
| | ATGCCATGTTCCCGGCGAAGGGCCAGCTGTCCCCCGCCAGCT |
| | AGACTCAGCACTTAGTTTAGGAACCAGTGAGCAAGTCAGCCCT |
| | TGGGGCAGCCCATACAAGGCCATGGGGCTGGGCAAGCTGCAC |
| | GCCTGGGTCCGGGGTGGGCACGGTGCCCGGGCAACGAGCTG |
| | AAAGCTCATCTGCTCTCAGGGGCCCCTCCCTGGGGACAGCCC |
| | CTCCTGGCTAGTCACACCCTGTAGGCTCCTCTATATAACCCAG |
| | GGGCACAGGGGCTGCCCTCATTCTACCACCACCTCCACAGCAC |
| | AGACAGACACTCAGGAGCCAGCCAG (SEQ ID NO: 66) |
| Spc512 promoter | GAGCTCCACCGCGGTGGCGGCCGTCCGCCTTCGGCACCATCC |
| | TCACGACACCCAAATATGGCGACGGGTGAGGAATGGTGGGGÅ |
| | GTTATTTTTAGAGCGGTGAGGAAGGTGGGCAGGCAGCAGGTGT |
| | TGGCGCTCTAAAAATAACTCCCGGGAGTTATTTTTAGAGCGGAG |
| | GAATGGTGGACACCCAAATATGGCGACGGTTCCTCACCCGTCG |
| | CCATATTTGGGTGTCCGCCCTCGGCCGGGGCCGCATTCCTGG |
| | GGGCCGGGCGGTGCTCCCGCCCGCCTCGATAAAAGGCTCCGG |
| | GGCCGGCGGCGGCCCACGAGCTACCCGGAGGAGCGGGAGGC |
| | GCCAAGCTCTAGAACTAGTGGATCCCCCGGGCTGCAGGAATTC |
| | GATAT (SEQ ID NO: 67) |
| MHCK7 promoter | GTTTAAACAAGCTTGCATGTCTAAGCTAGACCCTTCAGATTAAA |
| | AATAACTGAGGTAAGGGCCTGGGTAGGGGAGGTGGTGTGAGA |
| | CGCTCCTGTCTCTCCTCTATCTGCCCATCGGCCCTTTGGGGAG |
| | GAGGAATGTGCCCAAGGACTAAAAAAAGGCCATGGAGCCAGAG |
| | GGGCGAGGGCAACAGACCTTTCATGGGCAAACCTTGGGGCCC |
| | TGCTGTCTAGCATGCCCCACTACGGGTCTAGGCTGCCCATGTA |
| | AGGAGGCAAGGCCTGGGGACACCCGAGATGCCTGGTTATAATT |
| | AACCCAGACATGTGGCTGCCCCCCCCCCCCCAACACCTGCTGC |
| | CTCTAAAAATAACCCTGTCCCTGGTGGATCCCCTGCATGCGAA |
| | GATCTTCGAACAAGGCTGTGGGGGACTGAGGGCAGGCTGTAA |
| | CAGGCTTGGGGGCCAGGGCTTATACGTGCCTGGGACTCCCAA |
| | AGTATTACTGTTCCATGTTCCCGGCGAAGGGCCAGCTGTCCCC |
| | CGCCAGCTAGACTCAGCACTTAGTTTAGGAACCAGTGAGCAAG |
| | TCAGCCCTTGGGGCAGCCCATACAAGGCCATGGGGCTGGGCA |
| | AGCTGCACGCCTGGGTCCGGGGTGGGCACGGTGCCCGGGCA |
| | ACGAGCTGAAAGCTCATCTGCTCTCAGGGGCCCCTCCCTGGG |
| | GACAGCCCCTCCTGGCTAGTCACACCCTGTAGGCTCCTCTATA |
| | TAACCCAGGGGCACAGGGGCTGCCCTCATTCTACCACCACCTC |
| | CACAGCACAGACAGACACTCAGGAGCCAGCCAGCGGCGCGCC |
| | C (SEQ ID NO: 68) |
| SaCas9 | AAGCGGAACTACATCCTGGGCCTGGACATCGGCATCACCAGCG |
| | TGGGCTACGGCATCATCGACTACGAGACACGGGACGTGATCGA |
| | TGCCGGCGTGCGGCTGTTCAAAGAGGCCAACGTGGAAAACAA |
| | CGAGGGCAGGCGGAGCAAGAGAGGCGCCAGAAGGCTGAAGC |
| | GGCGGAGGCGGCATAGAATCCAGAGAGTGAAGAAGCTGCTGT |
| | TCGACTACAACCTGCTGACCGACCACAGCGAGCTGAGCGGCAT |
| | CAACCCCTACGAGGCCAGAGTGAAGGGCCTGAGCCAGAAGCT |
| | GAGCGAGGAAGAGTTCTCTGCCGCCCTGCTGCACCTGGCCAA |
| | GAGAAGAGGCGTGCACAACGTGAACGAGGTGGAAGAGGACAC |
| | CGGCAACGAGCTGTCCACCAAAGAGCAGATCAGCCGGAACAG |
| | CAAGGCCCTGGAAGAGAAATACGTGGCCGAACTGCAGCTGGA |
| | ACGGCTGAAGAAAGACGGCGAAGTGCGGGGCAGCATCAACAG |
| | ATTCAAGACCAGCGACTACGTGAAAGAAGCCAAACAGCTGCTG |
| | AAGGTGCAGAAGGCCTACCACCAGCTGGACCAGAGCTTCATCG |
| | ACACCTACATCGACCTGCTGGAAACCCGGCGGACCTACTATGA |
| | GGGACCTGGCGAGGGCAGCCCCTTCGGCTGGAAGGACATCAA |
| | AGAATGGTACGAGATGCTGATGGGCCACTGCACCTACTTCCCC |
| | GAGGAACTGCGGAGCGTGAAGTACGCCTACAACGCCGACCTG |
| | TACAACGCCCTGAACGACCTGAACAATCTCGTGATCACCAGGG |
| | ACGAGAACGAGAAGCTGGAATATTACGAGAAGTTCCAGATCAT |
| | CGAGAACGTGTTCAAGCAGAAGAAGAAGCCCACCCTGAAGCAG |
| | ATCGCCAAAGAAATCCTCGTGAACGAAGAGGATATTAAGGGCT |
| | ACAGAGTGACCAGCACCGGCAAGCCCGAGTTCACCAACCTGAA |
| | GGTGTACCACGACATCAAGGACATTACCGCCCGGAAAGAGATT |
| | ATTGAGAACGCCGAGCTGCTGGATCAGATTGCCAAGATCCTGA |
| | CCATCTACCAGAGCAGCGAGGACATCCAGGAAGAACTGACCAA |
| | TCTGAACTCCGAGCTGACCCAGGAAGAGATCGAGCAGATCTCT |
| | AATCTGAAGGGCTATACCGGCACCCACAACCTGAGCCTGAAGG |
| | CCATCAACCTGATCCTGGACGAGCTGTGGCACACCAACGACAA |
| | CCAGATCGCTATCTTCAACCGGCTGAAGCTGGTGCCCAAGAAG |
| | GTGGACCTGTCCCAGCAGAAAGAGATCCCCACCACCCTGGTG |
| | GACGACTTCATCCTGAGCCCCGTCGTGAAGAGAAGCTTCATCC |
| | AGAGCATCAAAGTGATCAACGCCATCATCAAGAAGTACGGCCT |
| | GCCCAACGACATCATTATCGAGCTGGCCCGCGAGAAGAACTCC |
| | AAGGACGCCCAGAAAATGATCAACGAGATGCAGAAGCGGAACC |
| | GGCAGACCAACGAGCGGATCGAGGAAATCATCCGGACCACCG |
| | GCAAAGAGAACGCCAAGTACCTGATCGAGAAGATCAAGCTGCA |
| | CGACATGCAGGAAGGCAAGTGCCTGTACAGCCTGGAAGCCATC |
| | CCTCTGGAAGATCTGCTGAACAACCCCTTCAACTATGAGGTGG |
| | ACCACATCATCCCCAGAAGCGTGTCCTTCGACAACAGCTTCAA |
| | CAACAAGGTGCTCGTGAAGCAGGAAGAAAACAGCAAGAAGGG |
| | CAACCGGACCCCATTCCAGTACCTGAGCAGCAGCGACAGCAAG |
| | ATCAGCTACGAAACCTTCAAGAAGCACATCCTGAATCTGGCCAA |
| | GGGCAAGGGCAGAATCAGCAAGACCAAGAAAGAGTATCTGCTG |
| | GAAGAACGGGACATCAACAGGTTCTCCGTGCAGAAAGACTTCA |
| | TCAACCGGAACCTGGTGGATACCAGATACGCCACCAGAGGCCT |
| | GATGAACCTGCTGCGGAGCTACTTCAGAGTGAACAACCTGGAC |
| | GTGAAAGTGAAGTCCATCAATGGCGGCTTCACCAGCTTTCTGC |
| | GGCGGAAGTGGAAGTTTAAGAAAGAGCGGAACAAGGGGTACA |
| | AGCACCACGCCGAGGACGCCCTGATCATTGCCAACGCCGATTT |
| | CATCTTCAAAGAGTGGAAGAAACTGGACAAGGCCAAAAAAGTG |
| | ATGGAAAACCAGATGTTCGAGGAAAAGCAGGCCGAGAGCATGC |
| | CCGAGATCGAAACCGAGCAGGAGTACAAAGAGATCTTCATCAC |
| | CCCCCACCAGATCAAGCACATTAAGGACTTCAAGGACTACAAG |
| | TACAGCCACCGGGTGGACAAGAAGCCTAATAGAGAGCTGATTA |
| | ACGACACCCTGTACTCCACCCGGAAGGACGACAAGGGCAACA |
| | CCCTGATCGTGAACAATCTGAACGGCCTGTACGACAAGGACAA |
| | TGACAAGCTGAAAAAGCTGATCAACAAGAGCCCCGAAAAGCTG |
| | CTGATGTACCACCACGACCCCCAGACCTACCAGAAACTGAAGC |
| | TGATTATGGAACAGTACGGCGACGAGAAGAATCCCCTGTACAA |
| | GTACTACGAGGAAACCGGGAACTACCTGACCAAGTACTCCAAA |
| | AAGGACAACGGCCCCGTGATCAAGAAGATTAAGTATTACGGCA |
| | ACAAACTGAACGCCCATCTGGACATCACCGACGACTACCCCAA |
| | CAGCAGAAACAAGGTCGTGAAGCTGTCCCTGAAGCCCTACAGA |
| | TTCGACGTGTACCTGGACAATGGCGTGTACAAGTTCGTGACCG |
| | TGAAGAATCTGGATGTGATCAAAAAAGAAAACTACTACGAAGTG |
| | AATAGCAAGTGCTATGAGGAAGCTAAGAAGCTGAAGAAGATCA |
| | GCAACCAGGCCGAGTTTATCGCCTCCTTCTACAACAACGATCT |
| | GATCAAGATCAACGGCGAGCTGTATAGAGTGATCGGCGTGAAC |
| | AACGACCTGCTGAACCGGATCGAAGTGAACATGATCGACATCA |
| | CCTACCGCGAGTACCTGGAAAACATGAACGACAAGAGGCCCCC |
| | CAGGATCATTAAGACAATCGCCTCCAAGACCCAGAGCATTAAG |
| | AAGTACAGCACAGACATTCTGGGCAACCTGTATGAAGTGAAATC |
| | TAAGAAGCACCCTCAGATCATCAAAAAGGGC |
| | (SEQ ID NO: 69) |
| Mini | TAGCAATAAAGGATCGTTTATTTTCATTGGAAGCGTGTGTTGGT |
| polyadenylation | TTTTTGATCAGGCGCG (SEQ ID NO: 70) |
| signal | |
| bGH | CTAGAGCTCGCTGATCAGCCTCGACTGTGCCTTCTAGTTGCCA |
| polyadenylation | GCCATCTGTTGTTTGCCCCTCCCCCGTGCCTTCCTTGÅCCCTG |
| signal | GAAGGTGCCACTCCCACTGTCCTTTCCTAATAAAATGAGGAAAT |
| | TGCATCGCATTGTCTGAGTAGGTGTCATTCTATTCTGGGGGGT |
| | GGGGTGGGGCAGGACAGCAAGGGGGAGGATTGGGAAGAGAA |
| | TAGCAGGCATGCTGGGGA (SEQ ID NO: 71) |
| SV40 intron | TCTAGAGGATCCGGTACTCGAGGAACTGAAAAACCAGAAAGTT |
| | AACTGGTAAGTTTAGTCTTTTTGTCTTTTATTTCAGGTCCCGGAT |
| | CCGGTGGTGGTGCAAATCAAAGAACTGCTCCTCAGTGGATGTT |
| | GCCTTTACTTCTAGGCCTGTACGGAAGTGTTAC |
| | (SEQ ID NO: 72) |

### Example 4

### All-In-One Vector 1 (Versions 1 and 2)

Two versions of vector 1 were generated. Vector I contained exon 45-55 targeted gRNAs with all promoters (U6, H1, and SaCas9-driving) in forward direction and mini polyadenylation signal for SaCas9.

Version 1 of vector 1 contained an EFS constitutive promoter. The sequence for version 1 of vector 1 is in SEQ ID NO: 73.

Version 2 of vector 1 contained a CK8 constitutive promoter. The sequence for version 2 of vector 1 is in SEQ ID NO: 74.

### Example 5

### All-In-One Vector 2 (Versions 1-4)

Four versions of vector 2 were generated. Vector 2 contained exon 45-55 targeted gRNAs with U6 promoter in reverse direction facing away from SaCas9-driving promoter and mini polyadenylation signal for SaCas9.

Version 1 of vector 2 contained an EFS constitutive promoter. The sequence for version 1 of vector 2 is in SEQ ID NO: 75.

Version 2 of vector 2 contained a CK8 constitutive promoter. The sequence for version 2 of vector 2 is as in SEQ ID NO: 76,

Version 3 of vector 2 contained a Spc512 promoter. The sequence for version 3 of vector 2 is as in SEQ ID NO: 77

Version 4 of vector 2 contained a MHCK7 promoter. The sequence for version 4 of vector 2 is as in SEQ ID NO: 78.

### Example 6

### All-In-One Vector 3 (Versions 1-4)

Four versions of vector 3 were generated. Vector 3 contained exon 45-55 targeted gRNAs with U6 promoter in reverse direction facing away from SaCas9-driving promoter and mini polyadenylation signal for SaCas9.

Version 1 of vector 3 contained an EFS constitutive promoter. The sequence for version 1 of vector 3 is as in SEQ ID NO: 79.

Version 2 of vector 3 contained a CK8 promoter. The sequence for version 2 of vector 3 is as in SEQ ID NO: 80.

Version 3 of vector 3 contained a Spc512 promoter. The sequence for version 3 of vector 3 is as in SEQ ID NO: 81.

Version 4 of vector 3 contained a MHCK7 promoter. The sequence for version 4 of vector 3 is as in SEQ ID NO: 82.

### Example 7

### All-In-One Vector 5 (Versions 1-4)

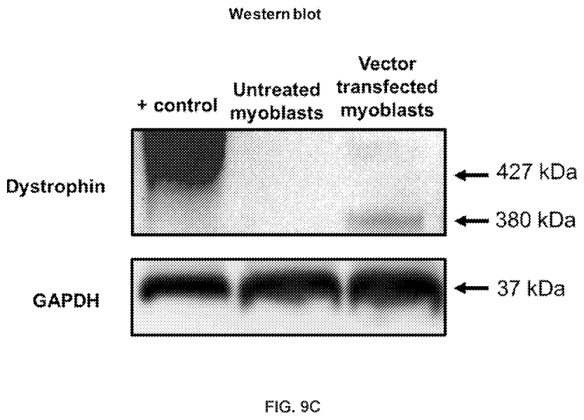

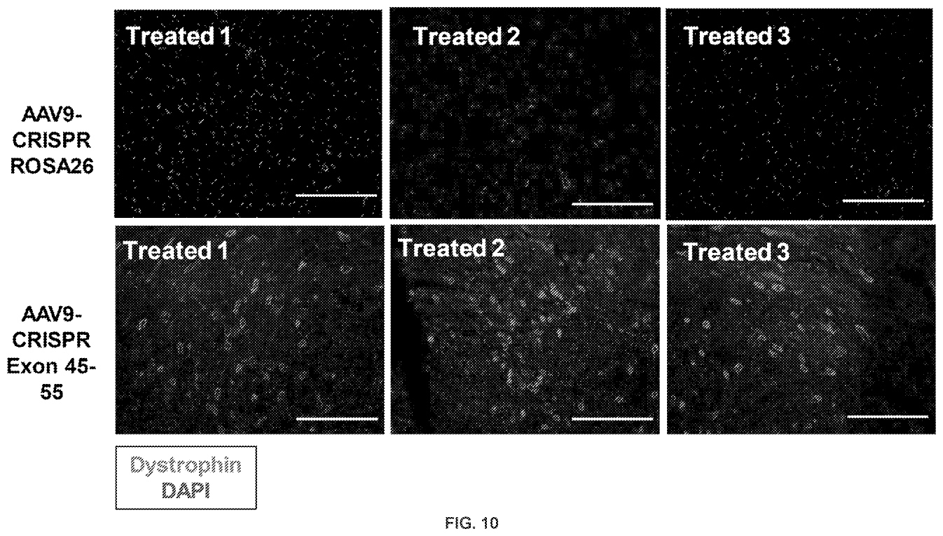

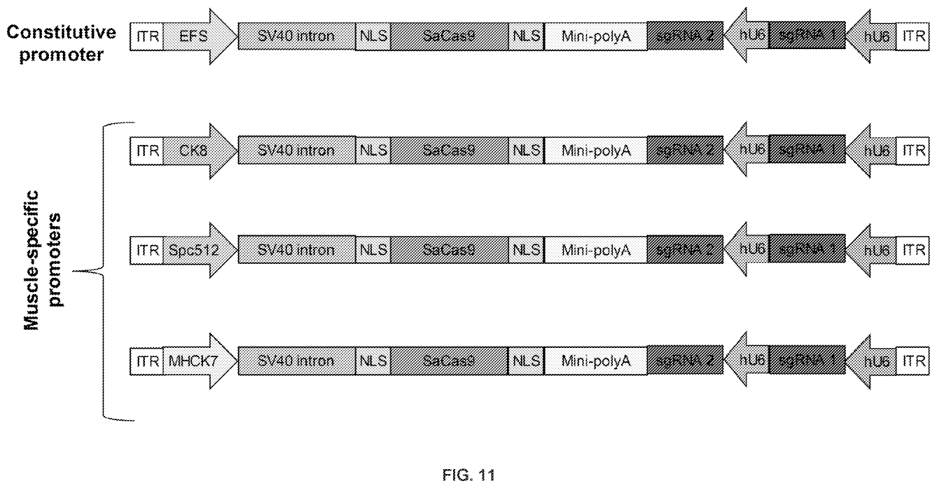

After screening a panel of all-in-one vector designs to determine the effect of guide placement, regulatory elements, and Pol-III promoters, a new set of all-in-one vectors was created with constitutive and muscle-specific promoters (FIG. 11). Versions of vector 5 of the all-in-one vector included an SV40 intron (see SEQ ID NO: 72) and placement of different elements.

Version 1 of vector 5 included a constitutive promoter. The sequence for version 1 of vector 5 is as in SEQ ID NO: 83.

Version 2 of vector 5 included a CK8 promoter. The sequence for version 2 of vector 5 is as in SEQ ID NO: 84.

Version 3 of vector 5 included a Spc-512 promoter. The sequence for version 3 of vector 5 is as in SEQ ID NO: 85.

Version 4 of vector 5 included a MHCK7 promoter. The sequence for version 4 of vector 5 is as in SEQ ID NO: 86.

### Example 8

### Additional Materials & Methods

Generation of the hDMDΔ52/mdx mouse. All animal studies herein were conducted with adherence to the guidelines for the care and use of laboratory animals of the National Institutes of Health (NIH). All the experiments involving animals were approved by the Institutional Animal Care and Use Committee at Duke University. The hDMD/mdx mouse (t Hoen, et al. J. Biol. Chem. 2008, 283, 5899-5907) was provided under Materials Transfer Agreement by Leiden University Medical Center. The expression cassettes for the S. pyogenes gRNA (Plasmid #47108) and human codon optimized SpCas9 nuclease (Plasmid #41815) were obtained from Addgene and used as previously described (Ousterout, et al. Nat. Commun. 2015, 5, 6244). gRNAs targeting the intronic region around exon 52 were selected based on maximal editing activity in HEK293T cells, including indel formation by individual gRNAs as measured by Surveyor assay and deletion of exon 52 by pairs of gRNAs as measured by end-point PCR (see sequences in TABLE 2). The generation of the hDMDΔ52/mdx mouse was completed by the Duke Transgenic Mouse Facility. Briefly, B6SJLF1/J donor females were superovulated by IP injection of 5 IU PMSG on day one and 5 IU HCG on day three, followed by mating with fertile hDMD/mdx males. On day 4, embryos were harvested and injected with mRNA encoding the gRNAs and SpCas9. Injected embryos were then implanted into pseudo-pregnant CD1 female mice. Genomic DNA was extracted from ear punches of chimeric pups using the DNEasy Blood and Tissue Kit (Qiagen) and screened for presence or deletion of exon 52. Mice with loss of exon 52 were bred with C57BL/10ScSn-Dmdmdx/J (mdx) mice. The resulting male hDMDΔ52/mdx (het;hemi) mice were used for experiments.

TABLE 2

| Description | Sequence |
|---|---|
| SaCas9 | CTCTGATAACCCAGCTGTGTGTT |
| proto- | (SEQ ID NO: 93) |
| spacers | CTAGACCATTTCCCACCAGTTCT |
| | (SEQ ID NO: 94) |
| Upstream | TGCCTTTCAATCATTGTTTCG |
| cell gDNA | (SEQ ID NO: 95) |
| primers | AAGGCCCCAAAATGTGAAAT |
| | (SEQ ID NO: 96) |
| Downstream | CGGGCTTGGACAGAACTTAC |
| cell gDNA | (SEQ ID NO: 97) |
| primers | CTGCGTAGTGCCAAAACAAA |
| | (SEQ ID NO: 98) |
| cDNA | GTTTCCAGAGCTTTACCTGAGAA |
| primers | (SEQ ID NO: 99) |
| | CTTTTATGAATGCTTCTCCAAG |
| | (SEQ ID NO: 100) |
| SpCas9 | AACAAATATCCCTTAGTATC |
| proto- | (SEQ ID NO: 101) |
| spacers | AATGTATTTCTTCTATTCAA |
| | (SEQ ID NO: 102) |
| Δ52 gDNA | CTCCGGAATGTCTCCATTTG |
| detection | (SEQ ID NO: 103) |
| primers | ATGAGGGAGAGACTGGCATC |
| | (SEQ ID NO: 104) |
| Tissue | TGCCTTTCAATCATTGTTTCG |
| gDNA Δ51 | (SEQ ID NO: 105) |
| primer | AGAAGGCAAATTGGCACAGA |
| set 1 | (SEQ ID NO: 106) |
| Tissue | TGGTTGTCCAGTGTGAGTCTCC |
| gDNA Δ51 | (SEQ ID NO: 107) |
| primer | GGGCTGCGTAGTGCCAAAAC |
| set 2 | (SEQ ID NO: 108) |
| Cell | ThermoFisher Hs02563140_s1 |
| gDNA | with VIC-MGB dye, Catalog |
| ddPCR | #: 4351372 |
| primers | DMDEx51_CCCSVXJ with FAM- |
| and | MGB dye, custom array: |
| probe | CCTGCTCTGGCAGATTTCAAC |
| | (SEQ ID NO: 109) |
| | ACCCACCATCACCCTCTGT |
| | (SEQ ID NO: 110) |
| | AAAGCCAGTCGGTAAGTTCTGTC |
| | (SEQ ID NO: 111) |
| SaCas9 | GCCGAGTTTATCGCCTCCTT |
| ddPCR | (SEQ ID NO: 112) |
| primers | GGTACTCGCGGTAGGTGATG |
| | (SEQ ID NO: 113) |
| Mouse | ThermoFisher Hs02563140_s1 |
| cDNA | with VIC-MGB dye, Catalog |
| ddPCR | #: 4351372 |
| primers | VGH50_53_APCE4DK with FAM- |
| and | MGB dye, custom array: |
| probe | TCTGAGTGGAAGGCGGTAAAC |
| | (SEQ ID NO: 114) |
| | CCTCCGGTTCTGAAGGTGTT |
| | (SEQ ID NO: 115) |
| | TCTTTCAAAGGCTCCAATAGTGGTCAGTCC |
| | (SEQ ID NO: 116) |
| Upstream | tcgtcggcagcgtcagatgtgtataag |
| deep | agacagttgccaccaatcttctggtt |
| sequencing | (SEQ ID NO: 117) |
| indels | gtctcgtgggctcggagatgtgtataa |
| primers | gagacagtgaagagggctgaacttgct |
| | (SEQ ID NO: 118) |
| Downstream | tcgtcggcagcgtcagatgtgtataagag |
| deep | acagggtgggtgaccttgaggata |
| sequencing | (SEQ ID NO: 119) |
| indels | gtctcgtgggctcggagatgtgtataaga |
| primers | gacagtgagagtaatgtgtttgctgagag |
| | (SEQ ID NO: 120) |
| Exon 45-55 | Probe: |
| ddPCR | ACAAATGGTATCTTAAGGACCTCCAAGGTG |
| deletion | (SEQ ID NO: 121) |
| probe | FW: CTGAGAATTGGGAACATGC |
| and | (SEQ ID NO: 122) |
| primers | RV: CATCGGAACCTTCCAGGG |
| | (SEQ ID NO: 123) |
| Exon 45-55 | GGTGCTTCCAGAGTGCTGAGG |
| cDNA | (SEQ ID NO: 124) |
| deletion | AATGGCGGCGTTTTCATTAT |
| PCR primers | (SEQ ID NO: 125) |
| Exon 45-55 | GGTGTCCTTTGAATATGCAGGT |
| gDNA | (SEQ ID NO: 126) |
| deletion | TTCTTGGCGTATTGCCTTCT |
| PCR primers | (SEQ ID NO: 127) |

AAV preparation. For exon 51 deletion experiments, an AAV cis plasmid containing a Staphylococcus aureus Cas9 expression cassette and hU6 polIII-driven gRNA cassette was obtained from Addgene (Watertown, MA; gRNAs were cloned in via BsaI or BbsI restriction sites. For hotspot deletion, two gRNA cassettes each driven by an hU6 promoter were cloned into either a single-stranded (Nelson, et al. Science 2016, 351, 403-407) or self-complementary (Plasmid #32396) AAV backbone. Intact ITRs were verified by Smal digestion and plasmids were Sanger sequenced prior to AAV production. AAV9 was generated by the Asokan laboratory and University of Massachusetts Viral Vector Core.

In vivo AAV-CRISPR administration. For intramuscular injections, male 6- to 8-week-old hDMDΔ52/mdx mice were anesthetized and injected with 2E11 vg/mouse into the left TA. For systemic injections, 7-8 week old male hDMDΔ52/mdx mice were tail vein injected at a dose of 4E12 vg/mouse. A t 8 weeks post-injection, mice were euthanized via CO₂ inhalation and tissues were collected for DNA, RNA, or protein extraction and histological analysis.

Genomic DNA analysis and transposon-mediated next-generation sequencing. Genomic DNA was extracted using the DNEasy kit (Qiagen, Hilden, Germany) according to the manufacturer's protocol. For endpoint PCR, primers flanking the SaCas9/gRNA cut sites in the intronic regions were designed and AccuPrime High Fidelity Taq Polymerase (Invitrogen, Waltham, MA) was used to amplify the area of intended deletion. PCR products were visualized via gel electrophoresis. The deletion product was extracted using the QIAQuick Gel Extraction kit (Qiagen, Hilden, Germany) and Sanger sequenced. Gene editing was detected by Tn5-mediated next generation sequencing. Tn5 was generated and preloaded with custom oligos to enable enrichment based on previously described methods (Picelli, et al. Genome Research 2014, 24, 2033-2040; Giannoukos, et al. BMC Genomics 2018, 19, 1-10; Nelson, et al. Nature Medicine 2019, 25, 427-432). 200 μg genomic DNA was tagmented and target enrichment was performed via PCR using AccuPrime High Fidelity Taq Polymerase and primer sets flanking the intended target site. A second PCR was used to add Illumina flowcell-binding sequences and barcodes (TABLE 2). The resulting PCR products were sequenced with 150-bp paired-end reads on a Miseq instrument (Illumina).

Deep sequencing for off-target activity. Potential off-target sites were identified by Cas-OFFinder (Bae, et al. Bioinformatics 2014, 30, 1473-1475) for both gRNAs with up to 9 mismatches with 0 bulge, and with up to 4 mismatches with up to 2 bulge in DNA and RNA. Results were compiled and scored by applying higher weight to genome mismatches distal to the PAM. Bulge location was not taken into account in the weighting. The sequence that had the least amount of mismatches in the genome and no bulge was selected for evaluation regardless of score. The sequences with highest the 4 highest scores for sequences with no bulge, not including this initial lowest mismatch, were also selected. Sequences with the 5 highest scores including bulges were selected for analysis, but sequences that were effectively repeats of each other with the exception of different bulge location were not included. Thus, each gRNA was analyzed for off-targets at 5 locations with no bulge and 5 locations with 1-2 bulges in the DNA or RNA. Primers were designed to amplify the on-target and off-target regions in amplicons of less than 200 base pairs (TABLE 2).

Off-target analysis was completed as previously described (Nelson, et al. Science 2016, 351, 403-407). Briefly, 100 ng gDNA from HEK293T cells that were transfected with SaCas9 and only one of the two gRNAs was PCR amplified with primers flanking the on-target or one of the 20 off target regions of interest (TABLE 2) for 30 cycles using the AccuPrime High Fidelity PCR kit (Invitrogen, Waltham, MA) and cleaned with Agencourt AMPure XP Beads (Beckman Coulter). A second round of ten cycles of PCR amplification was used to add Illumina flowcell binding sequences and experiment-specific barcodes on the 5′ end of the primer sequence. The PCR products were pooled and sequenced with 150 base pair paired-end reads on an Illumina MiSeq instrument. Samples were demultiplexed according to assigned barcode sequences and the added Illumina sequences were trimmed from reads. Because the amplicons are less than 200 base pairs, there was overlap in the paired-end reads. This overlap was used to create a consensus PCR amplicon for each pair-end read using single ungapped alignment. Indel analysis was performed using default CRISPResso settings and a 20 bp window (Pinello, et al. Nature Biotechnology 2016, 34, 695-697).

Deep sequencing for on-target activity in samples from mice. Deep sequencing for detection of indels created by genome editing was performed on genomic DNA samples from heart, diaphragm, TA, and gastrocnemius for all mice treated as adults (n=10 except for gastrocnemius n=5) and mice treated as neonates (n=4). PCR of the genomic DNA was completed using two primer pairs designed to flank the two cut sites. A second round of PCR was used to add Illumina flowcell binding sequencing and experiment-specific barcodes on the 5′ end of the primer sequencing. The PCR products were pooled and sequenced with 150 bp paired-end reads on an Illumina MiSeq instrument. Indel analysis was performed using CRISPResso (Pinello, et al. Nature Biotechnology 2016, 34, 695-697) with a window of 5 and default parameters. Deep sequencing to detect deletions of exon 51 was adapted from a previously published method for linear amplification-mediated high-throughput genome-wide translocation sequencing (Hu, et al. Nat. Protoc. 2016, 11, 853-871); n=4 for heart samples and n=7 for TA samples.

RNA extraction and vector expression analysis. RNA was extracted from tissues using a TissueLyser LT (Qiagen, Hilden, Germany) and the RNEasy Plus Universal Kit (Qiagen, Hilden, Germany). cDNA was synthesized using 500 ng of RNA and the SuperScript VILO cDNA Synthesis Kit and Master Mix (Life Technologies, Carlsbad, CA). To quantify AAV vector expression, primers and probes were designed for SaCas9 and gRNAs (TABLE 2). qRT-PCR was performed using Perfect Fastmix II (Quantabio, Beverly, MA) and Perfecta SYBR Green Fastmix (Quantabio, Beverly, MA) on a Bio-rad CFX96 Real-time PCR instrument (BioRad, Hercules, CA).

DIMD transcript analysis. Endpoint PCR of extracted cDNA was performed using AccuPrime polymerase and primers flanking the intended target site. Amplicons were visualized via gel electrophoresis. Deletion bands were purified using the QIAQuick Gel Extraction kit (Qiagen, Hilden, Germany) and Sanger sequenced. For quantifying exon deletion, digital droplet PCR (ddPCR) was performed using a QX200 Droplet Digital PCR System. Probe-based assays were designed against the edited and unedited sequences for exon 51 or hotspot deletion (TABLE 2). Reactions were prepared using ddPCR Supermix for Probes, no dUTP (BioRad, Hercules, CA). The fractional abundance of edited to unedited transcripts was calculated and expressed as deletion percentage.

Protein analysis and Western blot Muscle tissues were homogenized in RIPA buffer (Sigma, St. Louis, MO) with a protease inhibitor cocktail (Roche, Basel, Switzerland) and incubated for 30 minutes on ice with intermittent vortexing. Samples were centrifuged at 16,000×g for 30 minutes at 4° C. and the supernatant was isolated. Total protein amount was quantified using BCA assay according to the manufacturer's instructions (Pierce, Waltham, MA), Protein isolate was mixed with NuPAGE loading buffer (Invitrogen, Waltham, MA) and 5% β-mercaptoethanol and boiled at 100° C. for 10 minutes. 25 μg total protein per lane was loaded into 4-12% NuPAGE Bis-Tris gels (Invitrogen, Waltham, MA) and electrophoresed for 30 minutes at 200V. Protein was transferred to nitrocellulose membranes for 1 hour in 1×Tris-glycine transfer buffer containing 10% methanol and 0.01% SDS at 4° C. at 400 mA. The blot was blocked overnight at 4° C. in 5% milk-TBST. Blots were probed with Mandys106 antibody (1:50, Millipore MABT827, Burlington, MA) or rabbit anti-GAPDH (1:5000, Cell Signaling 2118S, Danvers, MA) for 1 hour in 5% milk-TBST at room temperature or overnight at 4° C. Blots were then incubated with mouse or rabbit horseradish peroxidase-conjugated secondary antibodies (Santa CruZ Biotechnology, Dallas, TX) for 30 minutes in 5% milk-TBST. Blots were visualized using ECL substrate (BioRad, Hercules, CA) on a ChemiDoc chemiluminescent system (BioRad, Hercules, CA).

Histological staining. Frozen muscles were mounted using OCT and 10 μm cryosections were prepared. For immunofluorescence staining, dystrophin was detected using Mandys106 primary antibody (1:200, Millipore MABT827, Burlington, MA) and goat anti-mouse IgG2a secondary antibody conjugated to Alexa Fluor 594 (1:500, ThermoFisher, A-21135, Waltham, MA). Nuclei were stained using DAPI (1:5000). H&E staining was performed using Harris-modified hematoxylin and eosin Y solution following using established protocols (Cardiff, et al. Cold Spring Harbor Protocols 2014, pdb. prot073411). Fibrosis staining was performed using a Masson trichrome kit (Sigma, St. Louis, MO).

Motor function analysis. Motor function analyses were performed at the Duke University Mouse Behavioral and Neuroendocrine Core. Forelimb grip strength was measured using conventional methods (De Luca, SOP DMD_M 2, 2008) on a San Diego Instruments Animal Grip Strength system (San Diego Instruments). Mice were allowed to grasp the bar with their front paws and were held in a horizontal position. Mice were gently pulled back until their grasp was broken for a total of 5 times. The highest three recorded values were averaged and normalized to body weight.

Statistics. Statistical analysis was performed using GraphPad Prism software (v.8). One-way ANOVA with Tukey's multiple comparisons test was used to compare groups for ddPCR quantification, vector expression, motor function testing, and in situ force analysis. Two-way ANOVA with post-hoc Tukey test was used to evaluate fibrosis staining and vector expression. All plotted dots are independent biological replicates (individual mice). Statistical differences between survival curves were compared using log-rank test. P values are reported in each figure.

Molecular cloning and AAV production. A Staphylococcus aureus Cas9 expression plasmid containing and hU6-driving gRNA cassette was obtained from AddGene (Watertown, MA; Plasmid #61591, Zhang lab). The CMV-SaCas9-polyA, without a gRNA cassette, was transferred to a new plasmid (pSaCas9) without ITRs for stability in cell culture experiments. A separate plasmid with the hU6-driven gRNA cassette and EBsI cloning sites for the gRNA was also created for cell culture experiments. gRNAs were cloned into the ITR or non-ITR containing plasmid via BsaI or BbsI cloning sites. After cloning and sequence verification of ITR-containing plasmids, ITRs were verified by Smal digestion before AAV production. AAV8 was generated by the Nationwide Children's Hospital Viral Vector Core. AAV9 was generated by the Asokan laboratory at the University of North Carolina Chapel Hill.

Cell culture and gRNA screening. gRNAs were designed to target sites in intron 50 and intron 51 of human DMD that were also conserved in the rhesus macaque and cynomolgus monkey genome. gRNAs were chosen based on off-target assessment by CasOFFinder, allowing up to 2 bp bulge and up to 4 mismatches. gRNAs chosen had no off-targets with 1, 2, or 3 mismatches with 0 bulge (see TABLE 2 for sequences). HEK293T cells were cultured in DMEM, 10% fetal bovine calf serum, and 1% penicillin/streptomnycin and maintained at 37° C. at 5% CO₂. HEK293T cells were transfected with Lipofectamine 2000 and 800 ng of plasmid DNA total in a 24-well plate. Cells were incubated for 48-72 hours and genomic DNA was isolated with a DNEasy kit (Qiagen, Hilden, Germany). Immortalized DMD patient myoblasts were maintained in skeletal muscle media (PromoCell) supplemented with 20% bovine calf serum (Sigma, St. Louis, MO), 50 μg/mL fetuin (Sigma, St. Louis, MO), 10 ng/mL human epidermal growth factor (Sigma, St. Louis, MO), 1 ng/mL human basic fibroblast growth factor (Sigma, St. Louis, MO), 10 μg/mL human insulin (Sigma, St. Louis, MO), 1% GlutaMAX (Invitrogen, Waltham, MA), and 1% penicillin/streptomycin (Invitrogen, Waltham, MA) at 37° C. at 5% CO₂. Immortalized DMD patient myoblasts were electroporated using the Gene PulserXCell (BioRad, Hercules, CA) with phosphate-buffered saline as an electroporation buffer using conditions previously optimized (Nelson, et al. Science 2016, 351, 403-407). Indels were identified by PCR of the region of interest performed using Invitrogen AccuPrime High Fidelity PCR kit, and 8 μL of the PCR product was incubated with the Surveyor Nuclease and Enhancer per kit directions. DNA was denatured in SDS and electrophoresed on TBE gels (Life Technologies, Carlsbad, CA) for 30 minutes at 200V. Gels were stained with ethidium bromide and imaged on a ChemiDoc™ chemiluminescence system (BioRad, Hercules, CA).

Creating the hDMDΔ52/mdx mouse. The hDMD/mdx mouse (lyombe-Engembe, et al. Molecular Therapy Nucleic Acids 2016, 5, e283) was provided under Materials Transfer Agreement by Leiden University Medical Center. The expression cassettes for the S. pyogenes gRNA (Plasmid #47108) and human codon optimized SpCas9 nuclease (Plasmid #41815) were obtained from Addgene and used as previously described (Long, et al. Science 2016, 351, 400-403). gRNAs targeting the intronic region around exon 52 were selected based on maximal editing activity in HEK293T cells, including indel formation by individual gRNAs as measured by Surveyor assay and deletion of exon 52 by pairs of gRNAs as measured by end-point PCR (see TABLE 2). The generation of the hDMDΔ52/mdx mouse was completed by the Duke Transgenic Mouse Facility. Briefly, B6SJLF1/J donor females were superovulated by IP injection of 5 IU PMSG on day one and 5 IU HCG on day three, followed by mating with fertile hDMD/mdx males. On day 4 embryos were harvested and injected with mRNA encoding the gRNAs and SpCas9. Injected embryos were then implanted into pseudo-pregnant CD1 female mice. gDNA was extracted from ear punches of chimeric pups using the DNEasy Blood and Tissue Kit (Qiagen, Hilden, Germany) and screened for presence or deletion of exon 52. Mice with loss of exon 52 were bred with mdx mice. The resulting male hDMDΔ52/mdx (het;hemi) mice were used for experiments.

Intramuscular injections of AA V. 7-8 week old male hDMDΔ52/mdx mice were anesthetized and placed on a warming pad. The tibialis anterior (TA) muscle was prepared for injection of 30 μL of AAV8 solution (˜5E11 vg/mouse) or saline into the right or left TA, respectively. After 8 weeks mice were euthanized via CO₂ inhalation and tissues were collected into RNALater (Life Technologies, Carlsbad, CA) for DNA, RNA, or protein analysis.

Systemic injection of AAV into adult and neonatal mice. P2 neonatal hDMDΔ52/mdx male mice were anesthetized by hypothermia and then injected with 40 μL AAV9 solution (˜1.5E12 vg/mouse) into the temporal vein. 7-8 week old adult male hDMDΔ52/mdx mice were injected via the tail vein with 200 μL of AAV9 solution (˜4E12-7.5E12 vg/mouse). At 16 weeks of age mice were euthanized by CO₂ inhalation and tissues were collected into RNALater (Life Technologies, Carlsbad, CA) for DNA, RNA, or protein analysis, or embedded in OCT for frozen tissue sections.

Genomic DNA analysis. Mouse tissues were digested in Buffer ALT and proteinase K at 56° C. in a shaking heat block. Cells were digested in Buffer AL and proteinase K at 56° C. for 10 minutes. DNEasy kit (Qiagen, Hilden, Germany) was used to collect genomic DNA. Nested endpoint PCR was performed with primers flanking the SaCas9/gRNA cut sites in the intronic regions using AccuPrime High Fidelity PCR kit. PCR products were electrophoresed in a 1% agarose gel and viewed on a BioRad (Hercules, CA) GelDoc imager to observe the parent band and deletion product. The deletion product was sequenced by first purification of the sample using the QIAQuick Gel Extraction kit (Qiagen, Hilden, Germany) then Sanger sequencing (Eton Bioscience).

Droplet digital PCR. Quantitative ddPCR was performed on cell gDNA and cDNA samples using a QX200 Droplet Digital PCR System. Exon 51 deletions from cells were detected using the QX200 ddPCR Supermix for Probes (BioRad, Hercules, CA) and Taqman assays with probe designed to bind to exon 51 and exon 59 (Thermo Fischer Scientific, Waltham, MA). The AAV vector genome was detected with primers targeting the SaCas9 coding sequence in gDNA extracted from animal tissues with QX200 ddPCR EvaGreen Supermix (BioRad, Hercules, CA). Exon 51 deletion in cDNA extracted from animal tissues were detected using the QX200 ddPCR Supermix for Probes (BioRad, Hercules, CA) and Taqman assays with probes designed to bind to the junction of human dystrophin exon 50 and exon 53, as well as a probe for exon 59. ddPCR for deletion of exon 51 in cDNA from animal tissues analysis was conducted by using the same threshold across all wells.

Deep sequencing. Deep sequencing for detection of indels created by genome editing was performed on genomic DNA samples from heart, diaphragm, TA, and gastrocnemius for all mice treated as adults (n=10 except for gastrocnemius n=5) and mice treated as neonates (n=4). PCR of the genomic DNA was completed using two primer pairs designed to flank the two cut sites. A second round of PCR was used to add Illumina flowcell binding sequencing and experiment-specific barcodes on the 5′ end of the primer sequencing. The PCR products were pooled and sequenced with 150 bp paired-end reads on an Illumina MiSeq instrument. Indel analysis was performed using CRISPResso (Zincarelli, et al. Molecular therapy: The Journal of the American Society of Gene Therapy 2008, 16, 1073-1080) with a window of 5 and default parameters. Deep sequencing to detect deletions of exon 51 was adapted from a previously published method for linear amplification-mediated high-throughput genome-wide translocation sequencing (Aartsma-Rus, A. et al. Neuromuscular Disorders 2002, 12, S71-S77; n=4 for heart samples and n=7 for TA samples).

RNA analysis. Immortalized DMD patient myoblasts were differentiated into myofibers by replacing the growth medium with DMEM supplemented with 1% insulin-transferrin-selenium (Invitrogen, Waltham, MA) and 1% antibiotic/antimycotic for 6-7 days. RNA was extracted from cells using the RNeasy Mini Kit and Qiashredder (Qiagen, Hilden, Germany). RNA was extracted from tissues that had been stabilized in RNALater (Invitrogen, Waltham, MA) using a TissueLyser LT (Qiagen, Hilden, Germany) and the RNEasy Plus Universal Kit (Qiagen, Hilden, Germany). cDNA was synthesized using up to 500 ng of RNA and the SuperScript VILO cDNA Synthesis Kit and Master Mix (Life Technologies, Carlsbad, CA). Endpoint PCR was performed using AccuPrime polymerase and electrophoresed on 1% agarose gels.

Protein analysis and western blot Muscle biopsies were disrupted with a probe sonicator (Fisher Scientific FB50) or a BioMasherII homogenizer in RIPA buffer (Sigma, St. Louis, MO) with a protease inhibitor cocktail (Roche) and incubated for 30 minutes on ice with intermittent vortexing. Samples were centrifuged at 16000×g for 30 minutes at 4° C., and the supernatant was isolated. Differentiated immortalized DMD patient myoblasts were collected and lysed in RIPA buffer (Sigma, St. Louis, MO) and supplemented with a protease inhibitor cocktail (Roche). Total protein amount was quantified using the bicinchronic acid assay according to the manufacturer's instructions (Pierce, Waltham, MA). Protein isolate was mixed with NuPAGE loading buffer (Invitrogen, Waltham, MA) and 5% β-mercaptoethanol and boiled at 100° C. for 10 minutes. 25 μg total protein per lane was loaded into 4-12% NuPAGE Bis-Tris gels (Invitrogen, Waltham, MA) with MOPS buffer (Invitrogen, Waltham, MA) and electrophoresed for 30 minutes at 200V. hDMD/mdx, labeled as +C, was loaded at 20% other samples. Protein was transferred to nitrocellulose membranes for 1 hour in 1×tris-glycine transfer buffer containing 10% methanol and 0.01% SDS at 4° C. at 400 mA. The blot was blocked overnight at 4° C. in 5% milk-TBST. Blots were probed with MANDYS8 (1:200, Sigma D8168, St. Louis, MO) for cells, MANDYS106 (1:50, Millipore MABT827) for animal tissues, HA (1:1000, Biolegend 901502) for SaCas9, or rabbit anti-GAPDH (1:5000, Cell Signaling 2118S) for 1 hour in 5% milk-TBST at room temperature or overnight at 4° C. Blots were then incubated with mouse or rabbit horseradish peroxidase-conjugated secondary antibodies (Santa Cruz) for 30 minutes in 5% milk-TBST. Blots were visualized using Western-C ECL substrate (BioRad, Hercules, CA) on a ChemiDoc chemiluminescent system (BioRad, Hercules, CA).

Histological stains. Muscles were dissected and embedded in OCT using liquid nitrogen-cooled isopentane. 10 μm sections were cut onto pre-treated histological slides (Fisher Scientific 12-550-15). Dystrophin was detected with the MANDYS8 (1:200, Sigma D8168, St. Louis, MO) antibody.

Motor function analysis. Motor function analyses were performed at the Duke University Mouse Behavioral and Neuroendocrine Core. Mice were allowed free exploration of an open field arena (20×20×30 cm) for 30 minutes (Omnitech Versamax Legacy, Columbus, OH). Automated monitoring of activity and location of animals was conducted with infrared diodes (x, y, and z axis) and interfaced to a computer running Fusion Activity software (version 5.3 Omnitech, Columbus, OH). Activity was reported in 6 samples of 5 minutes over the full 30-minute time. Grip strength was assessed by giving mice 3-5 trials each using the San Diego Instruments Animal Grip Strength system (San Diego Instruments), the average was reported. The bars on the grip strength system were adjusted to the angle which best suited the mouse's ability to grip and pull, as determined by the Mouse Behavioral and Neuroendocrine Core Facility at Duke University.

### Example 9

### Generation of a Humanized Mouse Model of DMD

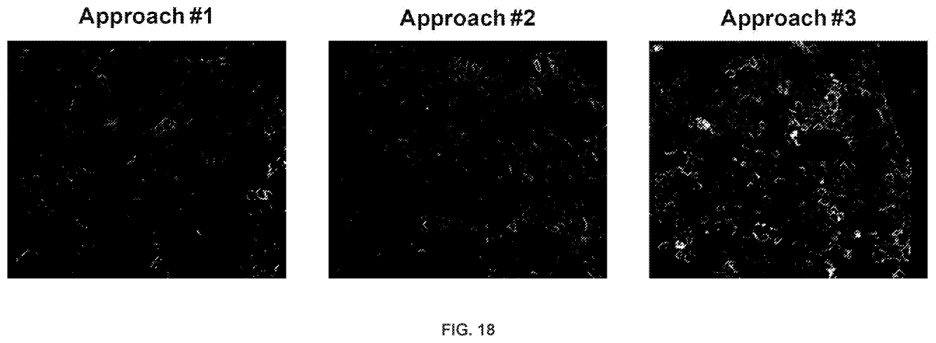

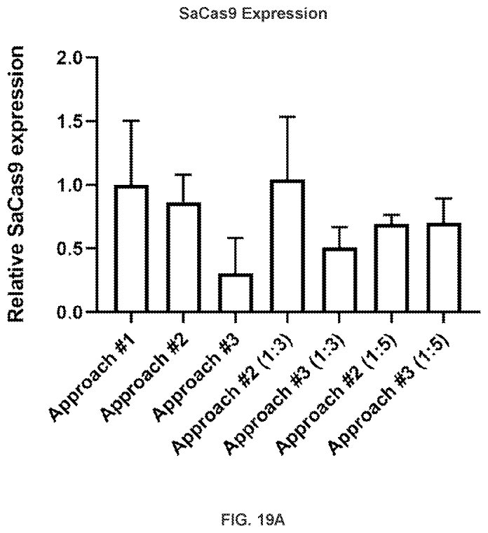

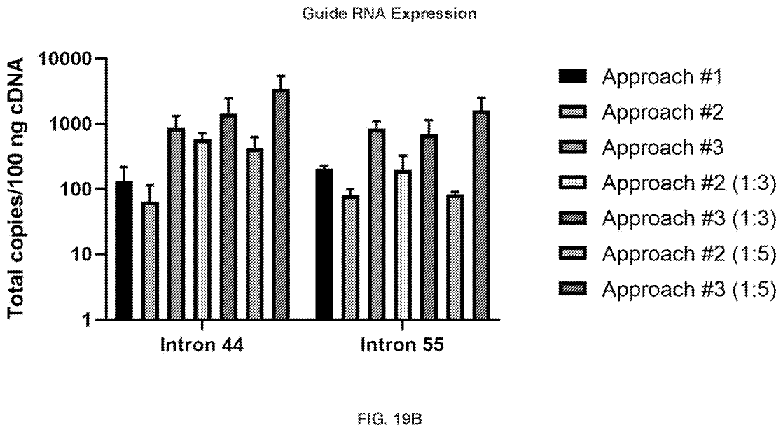

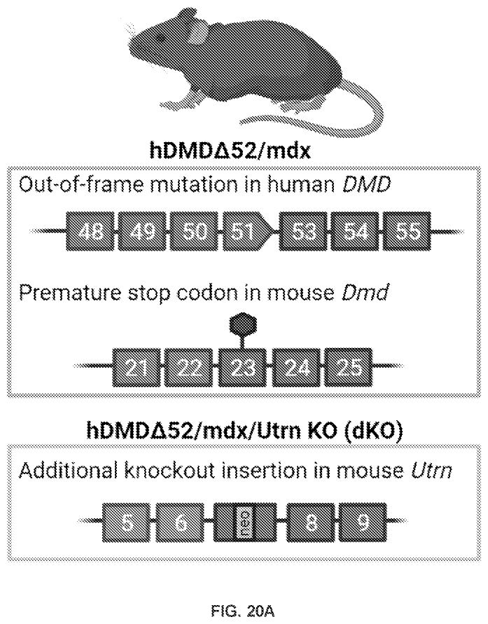

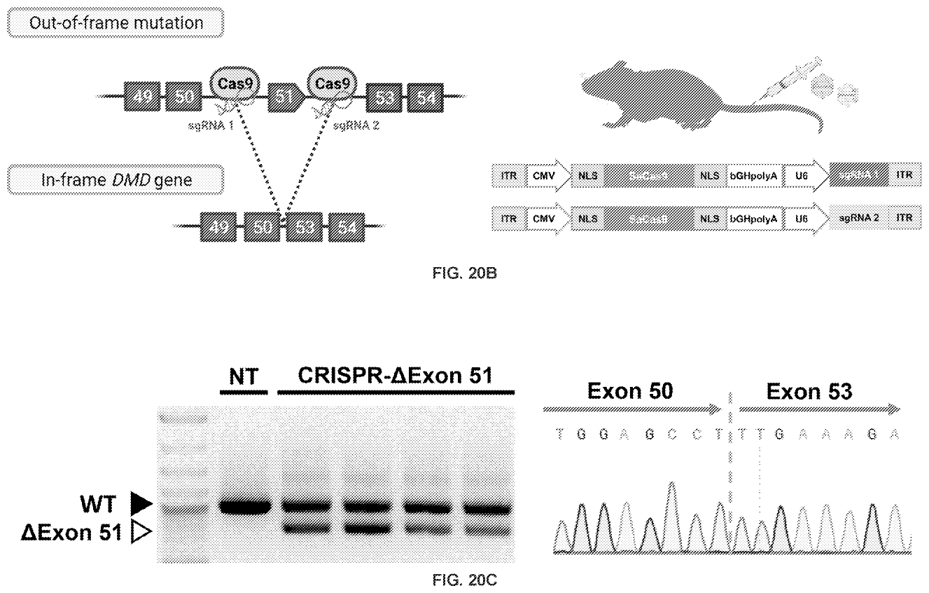

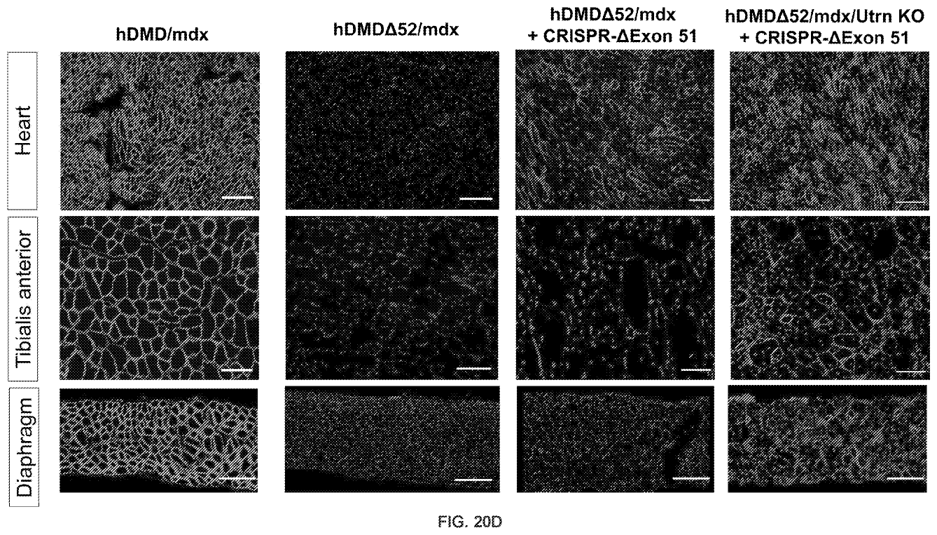

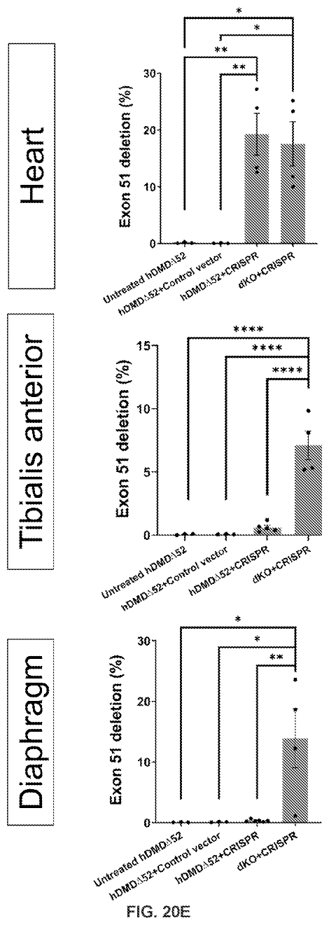

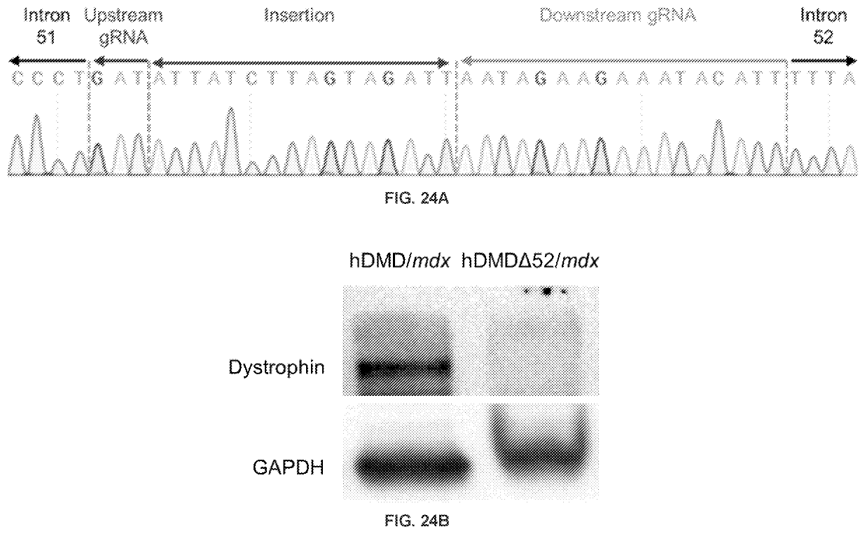

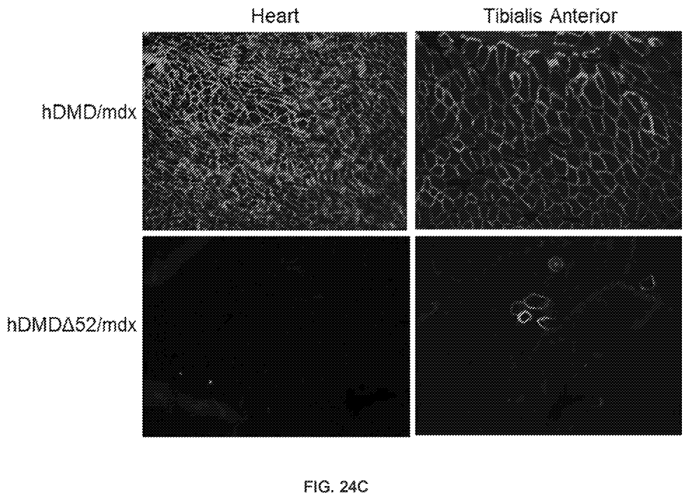

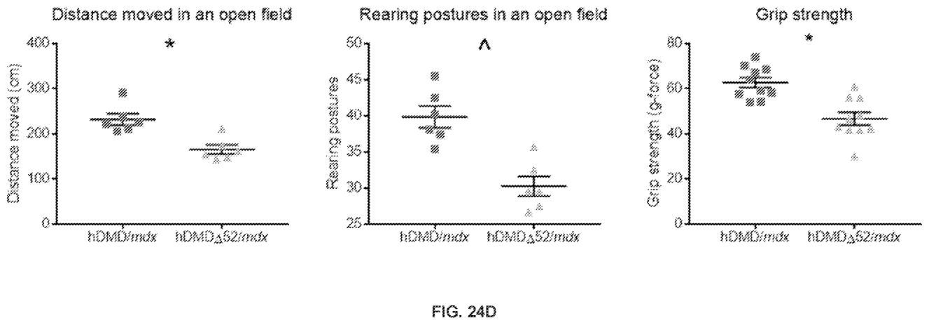

The hDMD/mdx mouse contains the full-length wild-type human DMD gene, complete with promoters and introns, on mouse chromosome 5 (t Hoen, et al. J. Bo. Chem. 2008, 283, 5899-5907). A dystrophic model was created in order to recapitulate a patient mutation by deleting exon 52 of the DMD gene. The resulting A52 mutation would disrupt the reading frame of the human DMD gene, but is correctable by removal of exon 51, exon 53, or exons 45-55 (Aartsma-Rus, et al. Neuromuscular Disorders 2002, 12, S71-S77). S. pyogenes Cas9 and gRNAs targeting intronic regions flanking exon 52 of the human DMD gene were delivered to hDMD/mdx zygotes to generate the hDMDΔ52/mdx mouse model (FIG. 20A). Deletion was confirmed by PCR across the gRNA target sites and subsequent Sanger sequencing of the PCR product (FIG. 24A). We confirmed that the hDMDΔ52/mdx does not produce mouse or human dystrophin protein via Western blot and histological staining (FIG. 24B and FIG. 24C). Additionally, the hDMDΔ52/mdx mouse exhibits a dystrophic phenotype as measured by overall activity in an open field and grip strength at 16 weeks of age (FIG. 24D), similar to the mdx mouse. These results are in line with an independently generated hDMDΔ52/mdx mouse. To assess our CRISPR-based treatment strategies in a severe dystrophic mouse model, we crossed our hDMDΔ52/mdx mouse with the Utrn^{tm1Ked} Dmd^{mdx/J} line to generate a double knockout mouse that is both dystrophin and utrophin-deficient, which we called hDMDΔ52/mdx/Utrn KO or “dKO” (FIG. 20A).

### Example 10

### Validation of a CRISPR/SaCas9 Approach for Excision of Exon 51

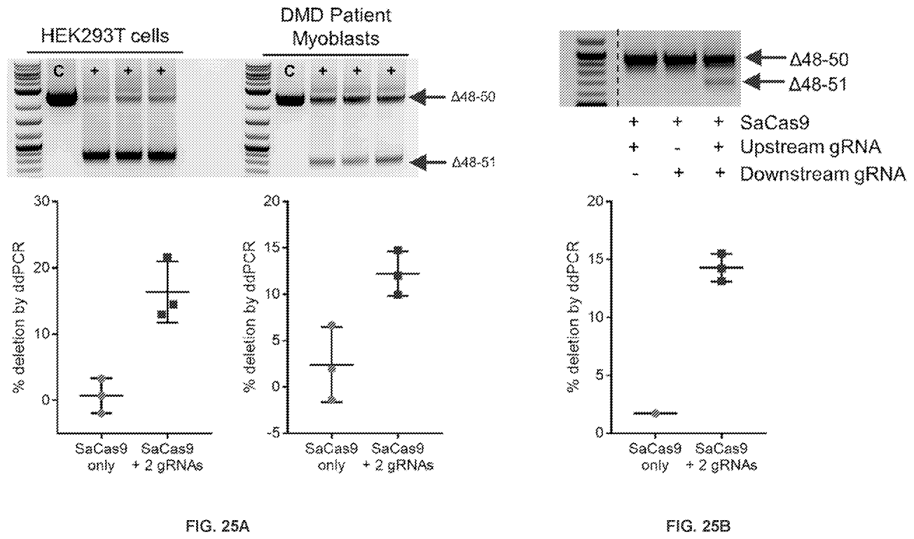

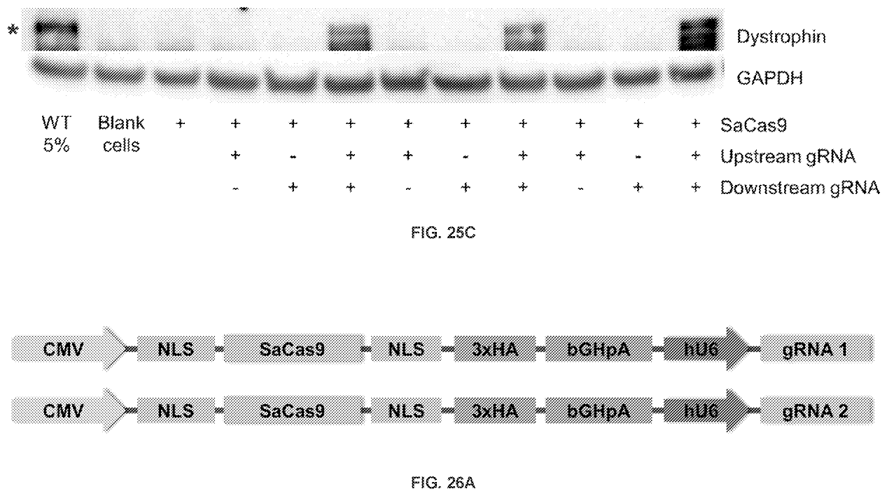

To evaluate single exon deletion as a therapeutic approach for DMD, we designed a strategy using Staphylococcus aureus Cas9 (SaCas9) and gRNAs targeting the surrounding intronic regions to remove exon 51 (FIG. 20B) (Ran, et al. Nature 2015, 520, 186-191). SaCas9 was used due to its smaller coding sequence (3.2 kb), which is compatible with size-restricted AAV vectors. We screened gRNAs corresponding to SaCas9 targeting sites in the intronic regions around DMD exon 51 that are conserved across human, rhesus macaque, and cynomolgus monkey genomes to facilitate future studies in large animal models. These gRNAs were validated in human HEK293T cells and immortalized DMD patient myoblasts that lack exons 48-50 and are thus amenable to exon 51 skipping. CRISPR treatment resulted in exon 51 deletion in the dystrophin gene and transcripts, leading to protein restoration (FIG. 25A-FIG. 25C), confirming activity and specificity of our CRISPR/SaCas9 system.

To evaluate this approach in vivo, we packaged the CRISPR/SaCas9 system into AAV9, which has a high tropism for cardiac and skeletal muscle. We utilized a dual vector system in which each vector contains SaCas9 and one gRNA (FIG. 20B). Adult 8 week-old hDMDΔ52/mdx and dKO mice were treated intravenously by co-injecting equal concentrations of vector for a total dose of 4×10¹² vector genomes per mouse. Animals were harvested at 8 weeks post-treatment for further analysis. Endpoint PCR was performed to assess exon 51 removal in dystrophin cDNA after AAV treatment. In mice that received CRISPR treatment, a smaller product was observed in addition to the parental band, indicative of exon 51 excision (FIG. 20C). Sanger sequencing of this PCR product confirmed correct splicing of exon 50 to exon 53. To examine dystrophin expression, histological staining was performed on heart, tibialis anterior (TA), and diaphragm muscles. We observed membrane-localized dystrophin staining in both treated hDMDΔ52/mdx and dKO muscle compared to untreated and control vector treated mice, with highest expression in the hearts (FIG. 20D). Lower levels of dystrophin were found in the TA and diaphragm, which may be due to increased cell turnover in these muscles. Digital droplet PCR quantification of exon 51 deletion corroborated these results and showed 19% and 18% deletion in heart transcripts, 0.6% and 7% in TAs, and 0.3% and 14% in diaphragms from hDMDΔ52/mdx and dKO mice, respectively (FIG. 20E). Interestingly, we observed significantly greater dystrophin restoration in dKO TA and diaphragm muscles compared to hDMDΔ52/mdx muscles. Since the dose was the same between the two models, this could be the result of body mass differences, increasing the effective dose in dKOs. Another possible explanation could be the advanced pathology in dKO mice at the time of treatment. While mdx mice show acute degeneration at 3 to 4 weeks of age, muscle degeneration is largely steady within the first year of life. On the other hand, mdx/utrophin˜ mice decline rapidly and quickly deplete their muscle progenitor pool. In our dKO mice, muscle regeneration may also be impaired, with only stabilized dystrophin-expressing fibers remaining. In contrast, progenitor cells in hDMDΔ52/rdx continue to replenish the muscle, potentially out-competing CRISPR-edited fibers. Similarly, lower levels of dystrophin in the skeletal muscle of mdxcv mice compared to the heart have been observed after CRISPR editing. Since neither of our humanized models display overt cardiac pathology, our results could also be attributed to higher turnover in skeletal muscle.

### Example 11

### Exon 51 Deletion Improves Pathology in Severely Dystrophic Mice

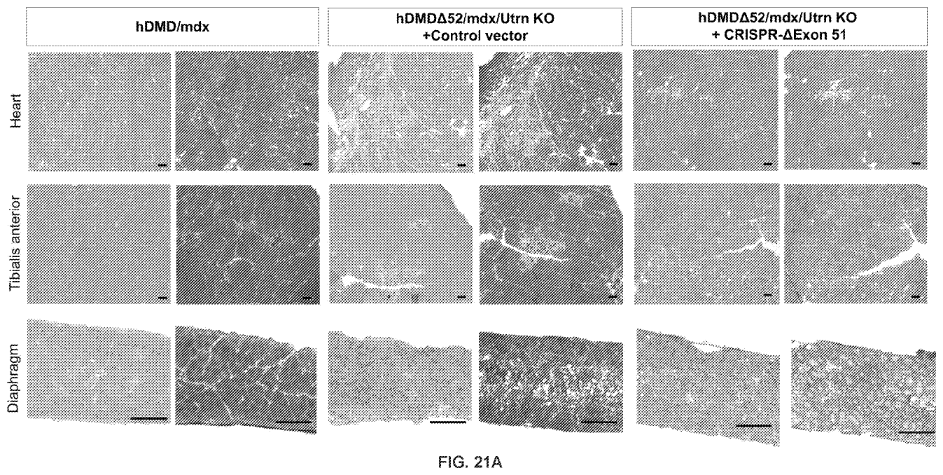

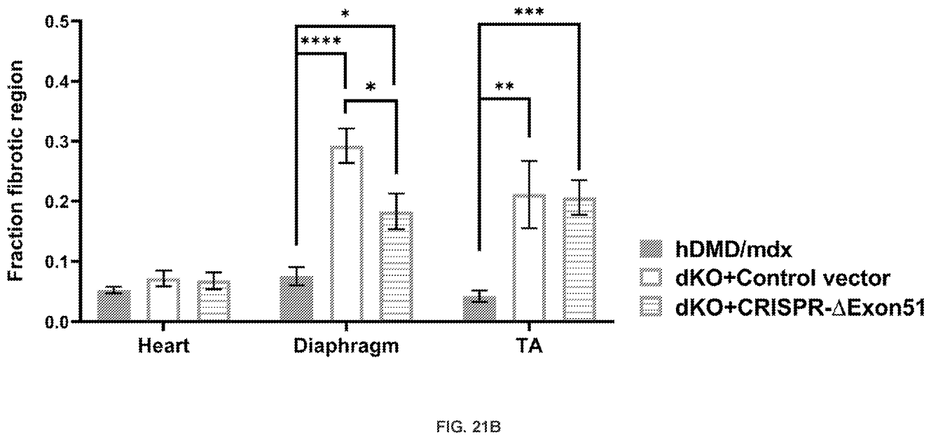

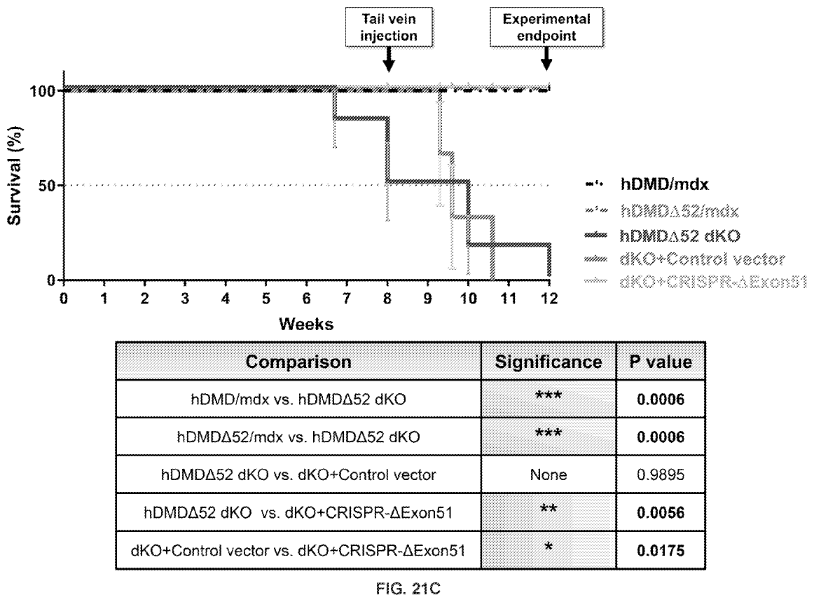

To assess phenotypic improvements after exon 51 deletion, we focused on the dKO mouse model, which recapitulates the severe muscle pathology and shortened lifespan of DMD patients. Heart, TA, and diaphragm muscles from dKO mice displayed hallmarks of dystrophic muscle, including actively degenerating and regenerating fibers, centronucleation, increased immune cell infiltration, and fibrotic deposition, as shown by H&E and Masson trichrome staining (FIG. 21A). The diaphragm was particularly affected in dKO mice, which is a well-known feature of mdx mice and most resembles DMD patient pathology. We found that CRISPR treatment significantly reduced fibrotic area in the diaphragm compared to treatment with a control vector (FIG. 21B). While fibrosis was also apparent in the heart and TA, it appeared to be more localized and less uniform between mice in comparison to the diaphragm, which could explain the similarity in calculated fibrotic areas between untreated and treated mice. Survival analysis was conducted to examine the effect of treatment in our dystrophic mouse models (FIG. 21C). We observed that hDMD/mdx, hDMDΔ52/mdx and CRISPR treated dKO mice survived for the entirety of the study, while untreated and control treated dKO mice had a median life expectancy of 9 and 9.6 weeks, respectively. Thus, treatment significantly improved dKO survival within the experimental period, which mirrored the WT and mildly affected hDMDΔ52/mdx model.

### Example 12

### AAV Vector Optimization for Deletion of a Large Mutational Hotspot

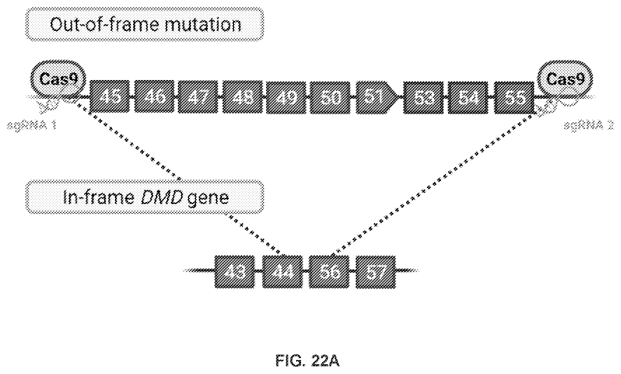

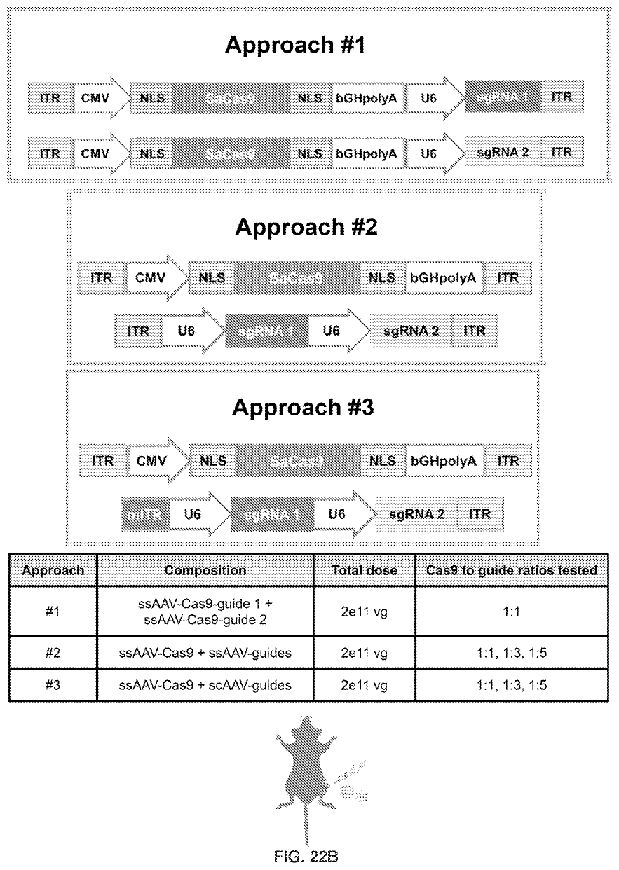

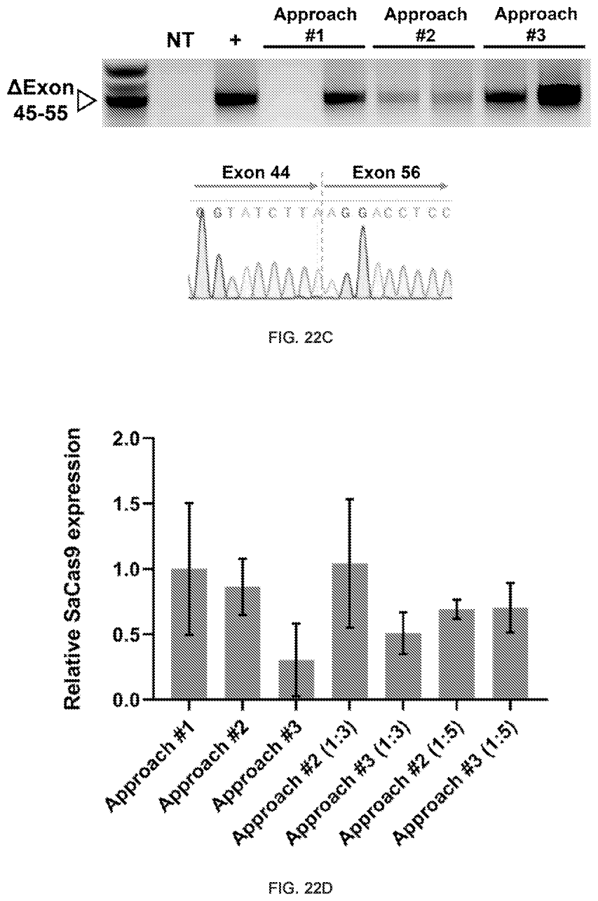

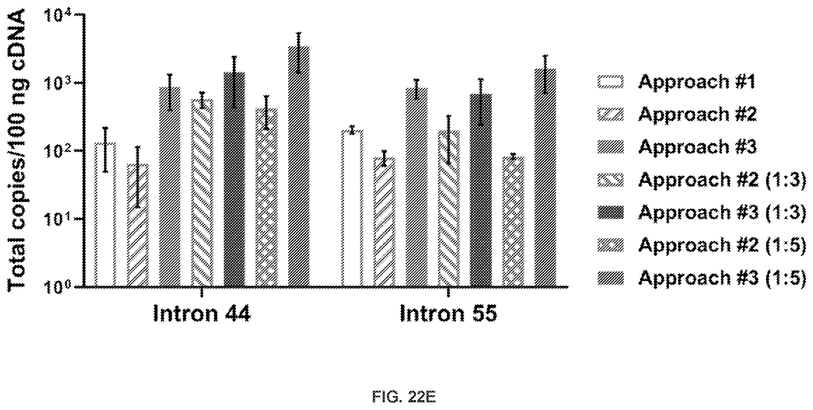

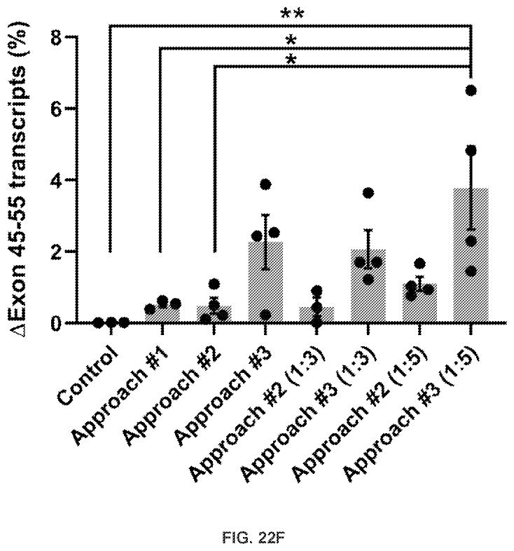

After successful single exon deletion in our mouse models, we next focused on excision of the mutational hotspot spanning dystrophin exons 45 through 55. Similar to our exon 51 deletion strategy, we utilized SaCas9 and identified gRNAs flanking exon 45-55 to induce deletion (FIG. 22A).

Following in vitro validation in HEK293 cells and patient myoblasts, we conducted a preliminary experiment in neonate-treated mice, which revealed that our existing dual AAV vector strategy could not sufficiently restore dystrophin expression in skeletal muscle (data not shown). Due to the large size of the deletion, we anticipated that removal of the mutational hotspot would be much less efficient. Thus, we compared multiple dual vector designs in vivo using hDMDΔ52/mdx mice to find the most efficacious strategy. Compared to the dKO, hDMDΔ52/mdx are easier to breed and maintain a normal lifespan, which is better suited for early proof-of-concept experiments. Other groups have shown that increased gRNA expression can improve editing, with a recent paper examining whether gRNA expression from a self-complementary AAV (scAAV) can further enhance this effect (Min, et al. Science Advances 2019, 5, eaav4324; Hakim, et al. JCI Insight 2018, 3, e124297; Zhang, et al. Science Advances 2020, 6, eaay6812). We generated three different approaches (FIG. 22B), which consist of (1) two single-stranded AAVs encoding SaCas9 and one of each gRNA, identical to our exon 51 approach, (2) a single stranded AAV encoding SaCas9 and a single stranded AAV containing the two gRNAs, and (3) a single stranded AAV encoding SaCas9 and a self-complementary AAV vector containing the two gRNAs.

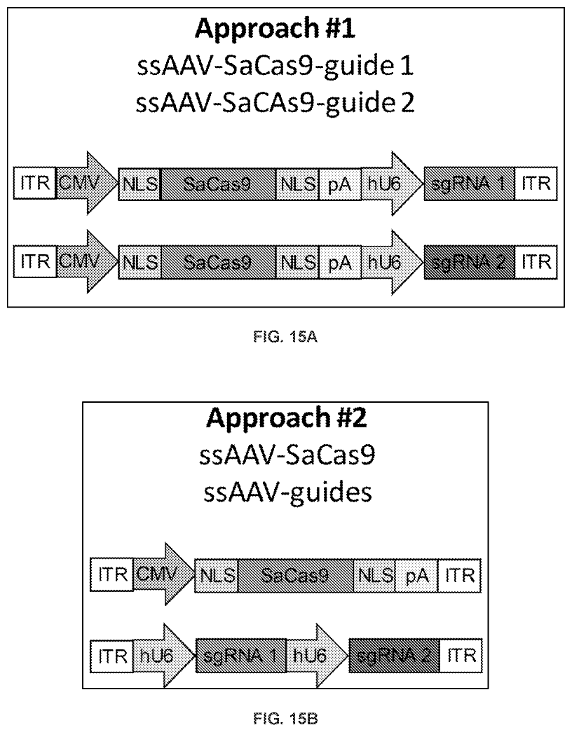

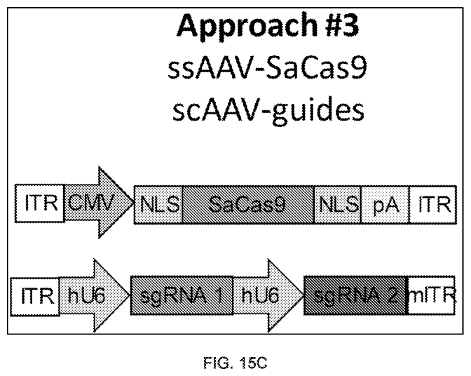

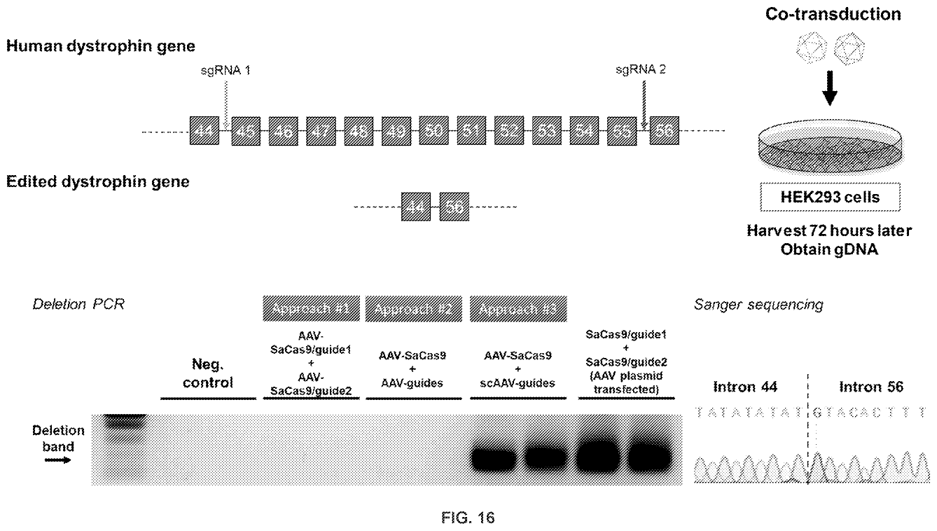

To determine feasibility of hotspot deletion with this new approach, the self-complementary construct and a SaCas9 encoding construct (Approach #3) were packaged into an AAV2 capsid. HEK293 cells were transduced with the AAV2 vectors. Results were compared to previously used dual vector strategies (FIG. 15A-FIG. 15C and FIG. 16), After 72 hours, genomic DNA was extracted. PCR using primers flanking the guide RNA target sites was performed, which showed that deletion of exons 45 through 55 was achieved using this new approach (FIG. 16).

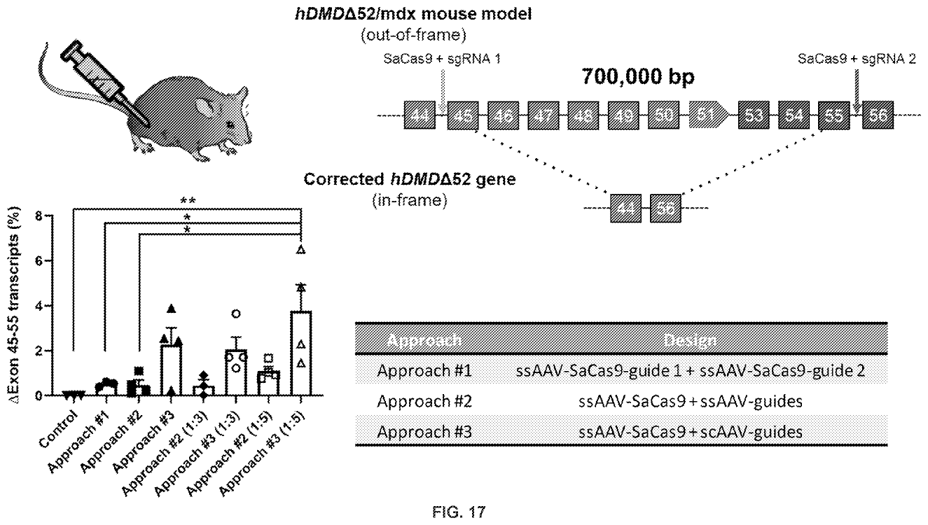

The dual vectors (Approach #3) were packaged into an AAV9 capsid and used for intramuscular injection in the tibialis anterior (TA) muscle of adult hDMDΔ52/mdx mice at varying ratios and average total dose of 1e11 vg (FIG. 17). After 8 weeks, mRNA was extracted from the TA muscle, and deletion of the exon 45-55 region was measured using digital droplet PCR, The approach using SaCas9 and guides vectors in a 1:5 ratio induced the highest deletion of the mutational hotspot. Dystrophin expression in the muscle also appeared to increase after treatment with Approach #3 compared to the other dual vector strategies (FIG. 18). Finally, SaCas9 and guide RNA expression were measured by qPCR, which showed that placing guide RNAs in the self-complementary configuration significantly increased levels compared to the other approaches (FIG. 19A-FIG. 19B).

This analysis was repeated. The three approaches were packaged into AAV9 and injected intramuscularly using different ratios at a total dose of 2×10¹¹ vector genomes into the TA of 8 week-old hDMDΔ52/mdx mice (FIG. 22B). Tibialis anterior (TA) muscles were harvested 8 weeks post-injection for further analysis. Endpoint PCR of dystrophin transcripts was performed, which showed prominent deletion bands for each dual vector strategy (FIG. 22C). No product is indicative of absence of deletion as the unedited transcript is too large (>1 kb), which was seen in the untreated control and one Approach #1 mouse. Sanger sequencing of the PCR product confirmed correct splicing of exon 44 to exon 56. SaCas9 and gRNA expression were also measured via qRT-PCR (FIG. 22D and FIG. 22E), which showed relatively similar Cas9 expression across conditions and increased gRNA levels with increasing guide to Cas9 ratios and treatment with scAAV-gRNA (Approach #3). Deletion of exon 45-55 in dystrophin transcripts was quantified via ddPCR, which showed significant transcript editing (4%) after treatment with ssAAV-SaCas9 and scAAV-guides at the 1:5 Cas9 to gRNA ratio (FIG. 22F). This strategy also restored noticeably higher levels of dystrophin in the TA compared to the other approaches (FIG. 22G),

### Example 13

### Hotspot Deletion Restores Dystrophin Expression and Improves Muscle Function Following Systemic Injection

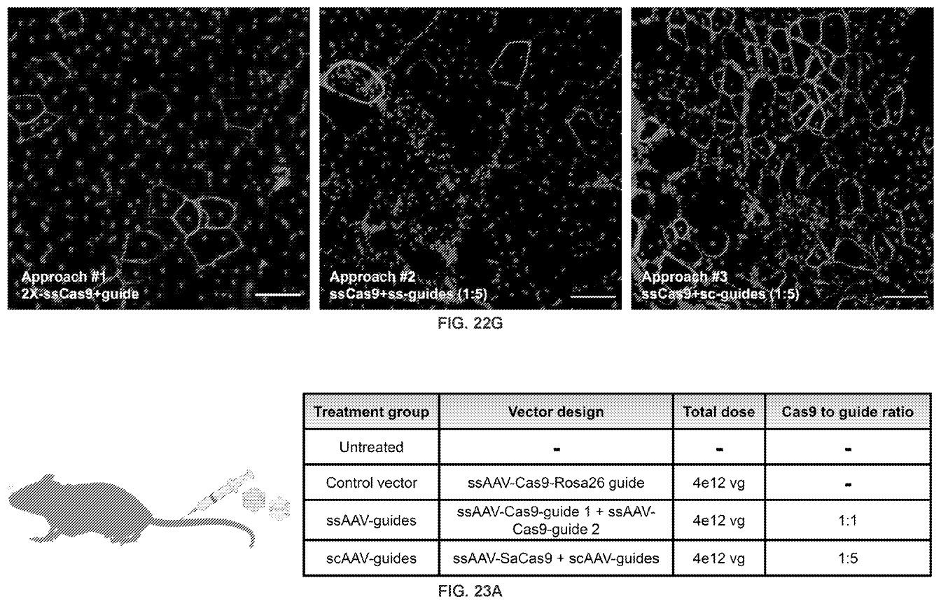

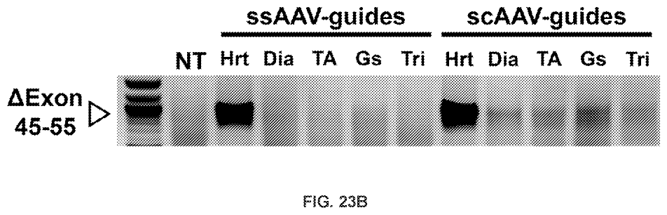

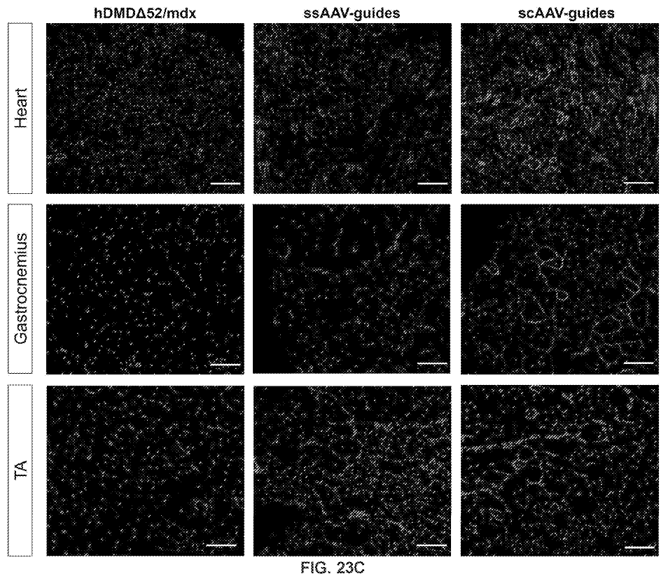

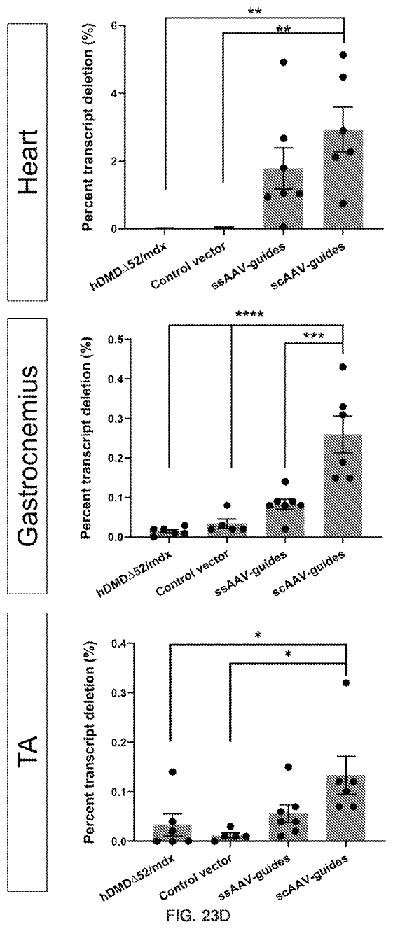

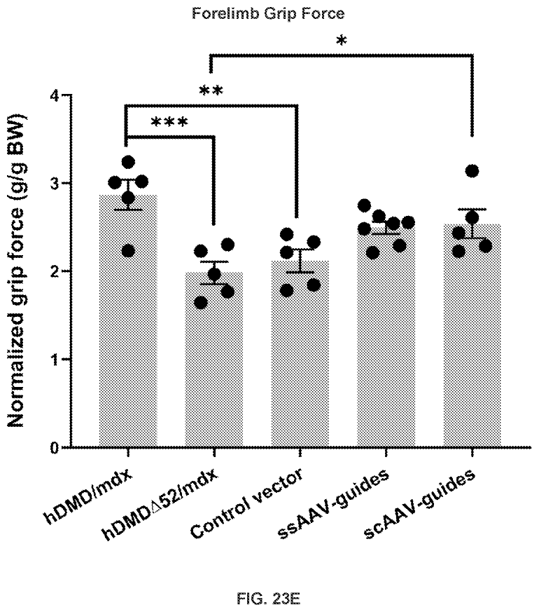

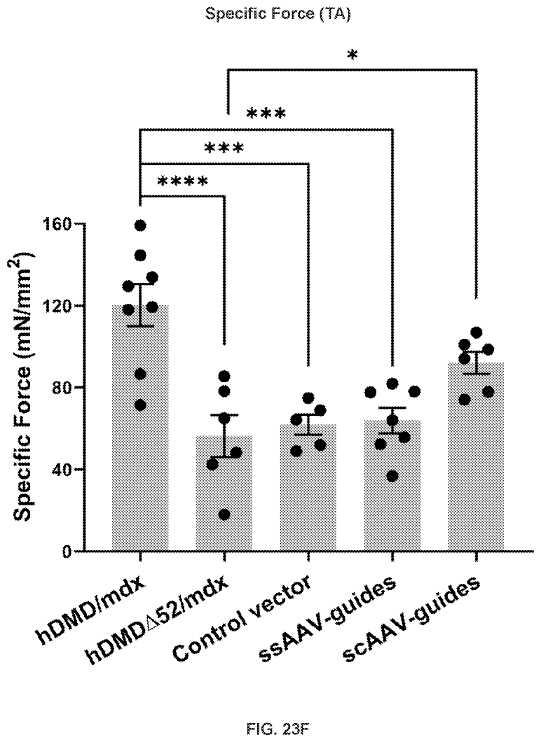

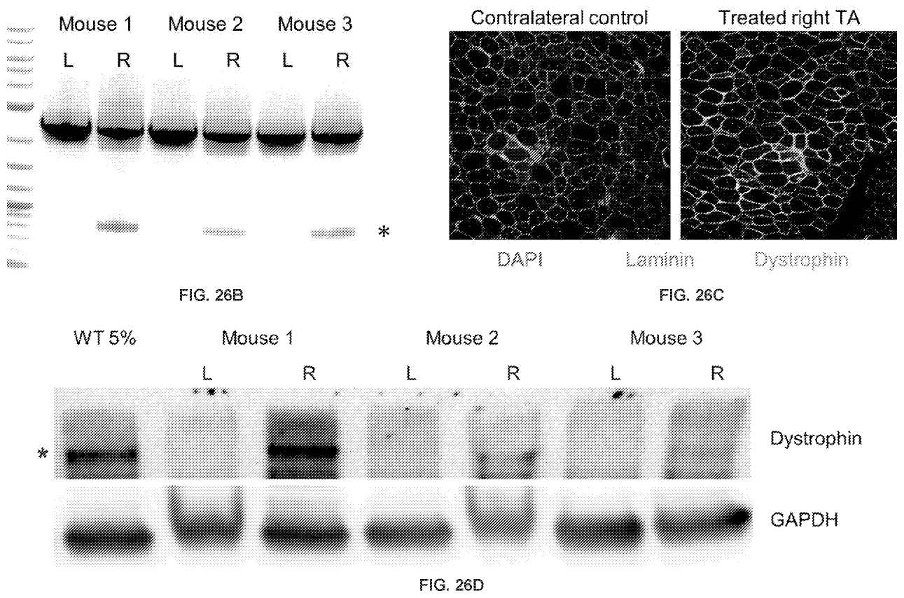

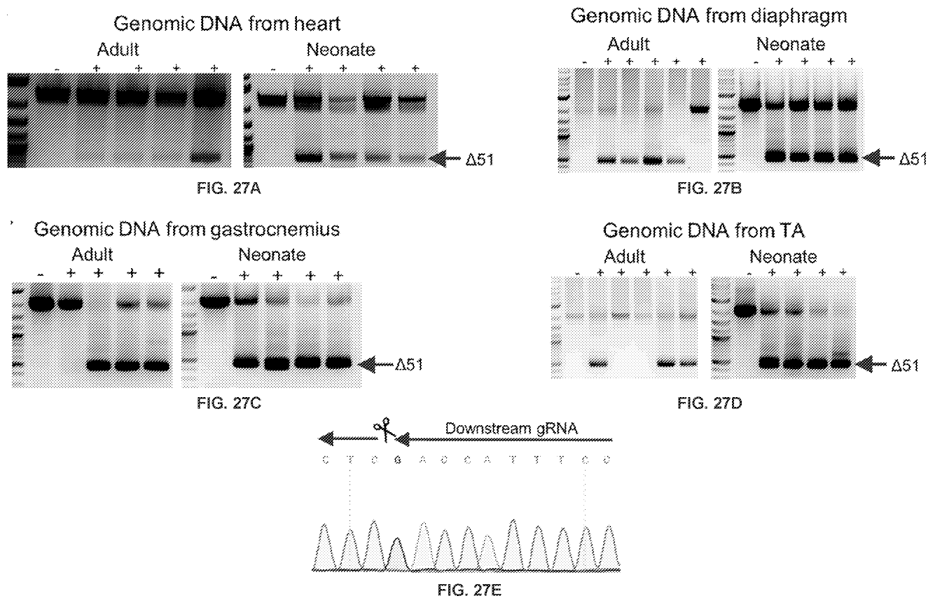

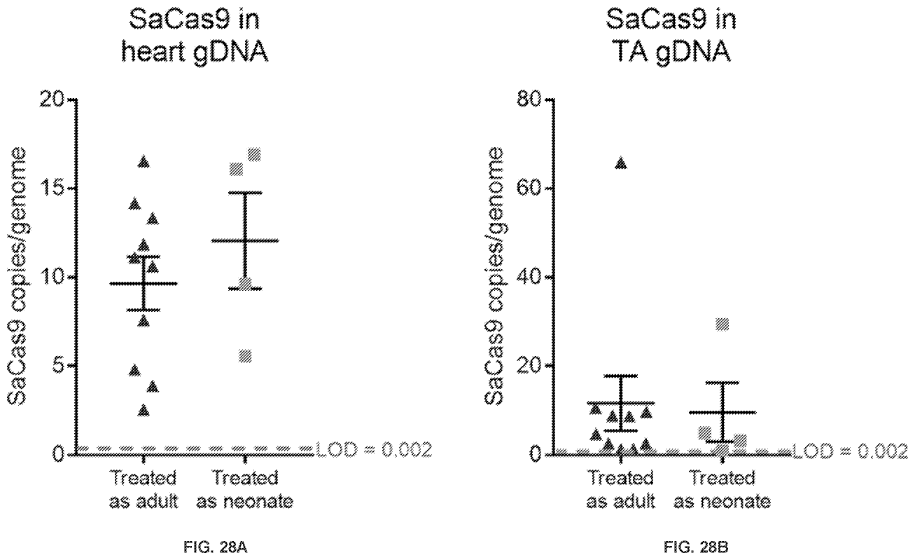

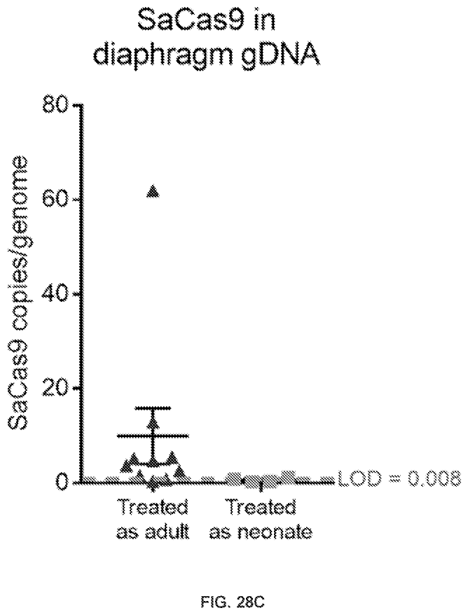

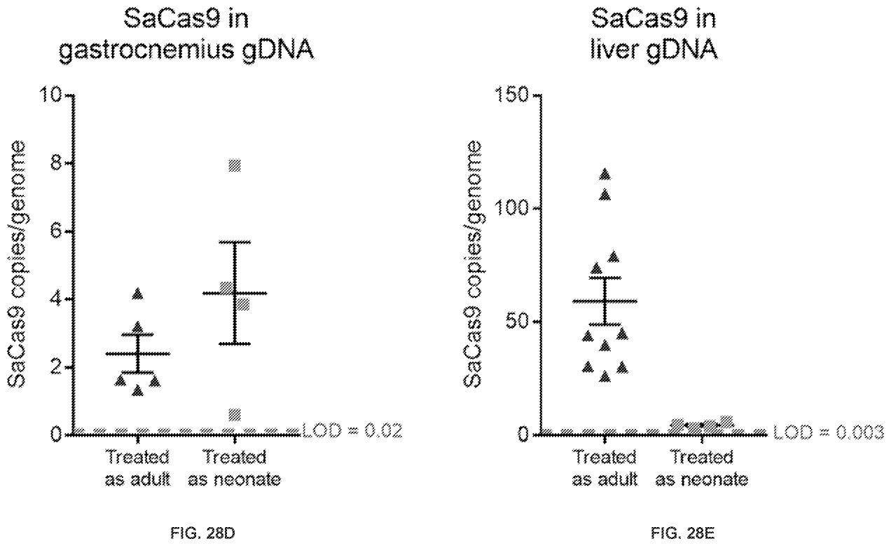

Next, we explored the therapeutic efficacy of CRISPR-mediated hotspot deletion following systemic injection. Control AAV, ssAAV-guides and scAAV-guides dual vector strategies were intravenously injected at a dose of 4×10¹² vector genomes in 8 week-old hDMDΔ52/mdx (FIG. 23A). Following harvest at 8 weeks post-injection, endpoint PCR of dystrophin transcripts was performed as described previously. An apparent deletion product was observed in the hearts of both ssAAV-guides and scAAV-guides treated mice (FIG. 23B). In addition, faint deletion bands were also observed in the skeletal muscle treated with scAAV-guides. Histological staining revealed high levels of membrane-localized dystrophin expression in the heart and lower levels in representative skeletal muscles after treatment with scAAV-guides compared to ssAAV-guides (FIG. 23C). In contrast to the heart, many low-expressing and partial dystrophin-positive fibers were observed in the skeletal muscles, suggestive of low correction efficiency. Still, these positive fibers were more apparent in scAAV-guide-treated mice. Quantification of exon 45-55 excision via ddPCR showed the highest percentage of deletion in scAAV-guides-treated mice compared to the other groups, with a mean of 3%, 0.1%, and 0.3% in the heart, TA, and gastrocnemius, respectively (FIG. 23D). Furthermore, mice treated with scAAV-guides exhibited significantly improved forelimb grip strength and TA specific force compared to the untreated group (FIG. 23E and FIG. 23F). These results suggest that hotspot deletion can effectively ameliorate functional deficits associated with the dystrophic phenotype. Additional results are shown in FIG. 26A-FIG. 26D, FIG. 27A-FIG. 27E, and FIG. 28A-FIG. 28E.

### Example 14

### Discussion

This study shows the potential of using a CRISPR/SaCas9 system to target human DMD and induce exon deletion to restore dystrophin expression in humanized mouse models. Because our hDMDΔ52/mdx displays a mild phenotype, similar to the mdx mouse, we generated a utrophin-deficient line to more closely recapitulate acute muscle degeneration seen in humans. Genome editing offers the potential advantage of only requiring one administration to correct the gene, as opposed to multiple, and possibly lifelong, doses of antisense oligonucleotides for exon skipping or episomal AAV vectors encoding mini- and micro-dystrophin that may be lost over time. We explored single exon excision in both humanized models and are the first to demonstrate improvements in dystrophic pathology in a severely affected mouse model. We expanded our approach to remove exons 45-55 in the hDMDΔ52/mdx mouse—a region of particular interest due to its designation as a mutational “hotspot.” BMD patients with this deletion often retain ambulation until their late 40s or older with very few cases of dilated cardiomyopathy. This presented a greater challenge as deletion is typically reported to decrease with increasing size of the deletion. As detailed herein, we desired to use a method suitable for systemic delivery in the clinic and thus worked to optimize an AAV strategy. Due to the limited packaging capacity of AAV vectors, we optimized a dual vector approach utilizing the smaller SaCas9 and self-complementary vectors. We demonstrated that this approach can be applied to multi-exon deletion in vivo and improved functional outcomes in dystrophic mice.

With the enormous size of the deletion (˜700 kb), systemic vector administration, and injection into adult mice with obvious pathology, our results show that this approach, while relatively less efficient, is feasible under clinically relevant circumstances. Different Cas9 orthologs may be used in the future. Restriction of Cas9 expression to the target tissues, the immune response to SaCas9 and AAV, and any off-target activity may be assessed. Genome editing over time may be assessed in longitudinal studies. Collectively, this work advances the field of gene editing for neuromuscular disease and demonstrates a pathway for preclinical development of gene editing therapeutics for DMD in small animal models.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the disclosure are set out in the following numbered clauses:

Clause 1. A CRISPR-Cas vector system comprising one or more vectors, wherein at least one of the one or more vectors comprises a sequence encoding: (a) first guide RNA (gRNA) targeting an intron or an exon of dystrophin and a second gRNA targeting an intron or an exon of dystrophin; and (b) a Cas9 protein.

Clause 2. The CRISPR-Cas vector system of clause 1, wherein the system comprises a first vector and a second vector, the first vector encoding the first gRNA and the second gRNA and the second vector encoding the Cas9 protein.

Clause 3. A CRISPR-Cas dual vector system comprising: (a) a first vector encoding a first guide RNA (gRNA) targeting an intron or an exon of dystrophin and a second gRNA targeting an intron or an exon of dystrophin; and (b) a second vector encoding a Cas9 protein.

Clause 4. The system of clause 2 or 3, wherein the first vector comprises a first ITR and a second ITR.

Clause 5. The system of clause 4, wherein the first ITR is operably linked to and upstream of the polynucleotide sequences encoding the first gRNA and the second gRNA, and wherein the second ITR is operably linked to and downstream of the polynucleotide sequence encoding the first gRNA and the second gRNA.

Clause 6. The system of any one of clauses 4-5, wherein the first ITR or second ITR is a wild-type ITR, and the other of the first ITR and second ITR is a mutant ITR, and wherein the mutant ITR directs vector genome replication to generate a self-complementary transcript that forms a double-stranded polynucleotide.

Clause 7. The system of clause 6, wherein the wild-type ITR comprises a polynucleotide having a sequence selected from SEQ ID NOs: 59-61 or 132.

Clause 8. The system of clause 4 or 5, wherein the mutant ITR comprises a polynucleotide having the sequence of SEQ ID NO: 62 or 140.

Clause 9. The system of any one of clauses 1-8, wherein the first vector comprises a first promoter operably linked to the polynucleotide sequence encoding the first gRNA molecule, and a second promoter operably linked to the polynucleotide sequence encoding the second gRNA molecule.

Clause 10. The system of clause 9, wherein the first vector comprises an expression cassette comprising 5′-[wild-type ITR]-[promoter]-[first gRNA]-[promoter]-[second gRNA]-[mutant ITR]-3, wherein “-” is an optional linker independently comprising a polynucleotide of 0-60 nucleotides.

Clause 11. The system of clause 10, wherein the vector genome replicated from the first vector is self-complementary and comprises 5′-[wild-type ITR]-[promoter]-[first gRNA]-[promoter]-[second gRNA]-[mutant ITR]-[second gRNA]-[promoter]-[first gRNA]-[promoter]-[wild-type ITR]-3′ and forms a double-stranded RNA hairpin.

Clause 12. The system of any one of clauses 9-11, wherein the first promoter and the second promoter comprise the same or different polynucleotide sequence.

Clause 13. The system of any one of clauses 9-12, wherein the first promoter and the second promoter are each independently selected from a ubiquitous promoter or a tissue-specific promoter.

Clause 14. The system of any one of clauses 9-13, wherein the first promoter and the second promoter are each independently selected from a human U6 promoter and a H1 promoter.

Clause 15. The system of any one of clauses 2-14, wherein the second vector comprises a third promoter driving expression of the Cas9 protein, and wherein the third promoter comprises a ubiquitous promoter or a tissue-specific promoter.

Clause 16. The system of clause 13, where the ubiquitous promoter comprises a CMV promoter.

Clause 17. The system of clause 13 or 15, where the tissue-specific promoter is a muscle-specific promoter comprising a MHCK7 promoter, a CK8 promoter, or a Spc512 promoter.

Clause 18. The system of any one of clauses 2-17, wherein the first vector further encodes at least one Cas9 gRNA scaffold.

Clause 19. The system of any one of clauses 1-18, wherein the first gRNA and the second gRNA each comprise a Cas9 gRNA scaffold.

Clause 20. The system of clause 18 or 19, wherein the Cas9 gRNA scaffold comprises the polynucleotide sequence of SEQ ID NO: 89 or 18 or 19 or 138 or 90 or 139.

Clause 21. The system of any one of clauses 1-20, wherein the first or second gRNA targets intron 44 of dystrophin.

Clause 22. The system of any one of clauses 1-21, wherein the first or second gRNA targets intron 55 of dystrophin.

Clause 23. The system of any one of clauses 1-22, wherein the first gRNA targets intron 44 of dystrophin and the second gRNA targets intron 55 of dystrophin, or wherein the first gRNA targets intron 55 of dystrophin and the second gRNA targets intron 44 of dystrophin.

Clause 24. The system of clause 21 or 23, wherein the first or second gRNA targeting intron 44 of dystrophin targets a polynucleotide comprising the sequence of SEQ ID NO: 55 or 135 or a 5′ truncation thereof.

Clause 25. The system of any one of clauses 1-22, wherein the first gRNA or the second gRNA targets intron 44 of dystrophin and comprises the polynucleotide sequence of SEQ ID NO: 57 or 137 or a 5′ truncation thereof.

Clause 26. The system of clause 22 or 23, wherein the first or second gRNA targeting intron 55 of dystrophin targets a polynucleotide comprising the sequence of SEQ ID NO: 56 or 134 or a 5′ truncation thereof.

Clause 27. The system of any one of clauses 1-26, wherein the first gRNA or the second gRNA targets intron 55 of dystrophin and comprises the polynucleotide sequence of SEQ ID NO: 58 or 136 or a 5′ truncation thereof.

Clause 28. The system of any one of clauses 1-27, wherein the Cas9 protein comprises SpCas9, SaCas9, or St1 Cas9 protein.

Clause 29. The system of any one of clauses 1-28, wherein the Cas9 protein comprises a SaCas9 protein comprising the amino acid sequence of SEQ ID NO: 88 or is encoded by a polynucleotide comprising the sequence of SEQ ID NO: 69.

Clause 30. The system of any one of clauses 2-29, wherein the first vector comprises a polynucleotide having the sequence selected from SEQ ID NOs: 91, 92, 128, 129, 130, or 131.

Clause 31. The system of any one of clauses 2-30, wherein the first vector and/or the second vector is a viral vector.

Clause 32. The system of clause 31, wherein the viral vector is an Adeno-associated virus (AAV) vector.

Clause 33. The system of clause 32, wherein the AAV vector is AAV1, AAV2, AAV3, AAV4, AAV5, AAV6, AAV7, AAV8, AAV9, AAV-10, AAV-11, AAV-12, AAV-13, or AAVrh.74.

Clause 34. The system of any one of clauses 2-33, wherein the first vector is present in a concentration at least 2-fold, at least 3-fold, at least 4-fold, at least 5-fold, at least 6-fold, at least 7-fold, at least 8-fold, at least 9-fold, or at least 10-fold greater than the concentration of the second vector.

Clause 35. The CRISPR-Cas vector system of any one of clauses 1-34, the system comprising one or more vectors, wherein at least one vector of the one or more vectors comprises a sequence encoding, from the 5′ to 3′ direction: (a) a first ITR; (b) a first promoter; (c) a first gRNA targeting an intron or exon of the dystrophin gene; (d) a Cas9 gRNA scaffold; (e) a second promoter; (f) a second gRNA targeting an intron or exon of dystrophin the gene: (g) a Cas9 gRNA scaffold; and (h) a second ITR.

Clause 36. The system of clause 35, wherein vector genome replication from the at least one vector results in a genome comprising, from the 5′ to 3′ direction: (a) a complementary sequence of the second ITR; (b) a complementary sequence of the second gRNA; (c) a complementary sequence of the second promoter; (d) a complementary sequence of the Cas9 gRNA scaffold; (e) a complementary sequence of the first gRNA; (f) a complementary sequence of the first promoter; (h) the first ITR; (i) the first promoter; (g) the first gRNA; (k) the Cas9 gRNA scaffold; (l) the second promoter; (m) the second gRNA; and (n) the second ITR.

Clause 37. A cell comprising the system of any one of clauses 1-36.

Clause 38. A kit comprising the system of any one of clauses 1-36.

Clause 39. A method of correcting a mutant dystrophin gene in a cell, the method comprising administering to a cell the system of any one of clauses 1-36.

Clause 40. A method of genome editing a mutant dystrophin gene in a subject, the method comprising administering to the subject the system of any one of clauses 1-36 or the cell of clause 37.

Clause 41. A method of treating a subject having a mutant dystrophin gene, the method comprising administering to the subject the system of any one of clauses 1-36 or the cell of clause 37.

Clause 42. The method of any one of clauses 40-41, wherein the subject is an adult, an adolescent, or a pre-adolescent.

Clause 43. The method of clause 42, wherein the subject is an adult.

Clause 44. The method of any one of clauses 40-43, wherein the system of any one of clauses 1-36 or the cell of clause 37 is administered to the subject intravenously.

Clause 45. The method of any one of clauses 40-44, wherein the system of any one of clauses 1-36 or the cell of clause 37 is administered to the subject systemically.

Clause 46. A CRISPR-Cas dual vector system comprising one or more vectors, wherein the one or more vectors comprises a vector that comprises an expression cassette, from the 5′ to 3′ direction, comprising: (a) a first AAV ITR sequence; (b) a first promoter sequence; (c) a guide sequence targeting a first intron of dystrophin gene; (d) a Cas9 scaffold sequence; (e) a second promoter sequence; (f) a guide sequence targeting a second intron of dystrophin gene; and (g) a second AAV ITR sequence.

Clause 47. The system of clause 46, wherein the expression cassette is a single stranded (“ss”) expression cassette or a self-complementary (“sc”) expression cassette.

Clause 48. The system of 47, wherein the self-complementary (“sc”) expression cassette, from the 5′ to 3′ direction, comprises: (a) a complementary sequence of the second AAV ITR sequence; (b) a complementary sequence of the guide sequence targeting the second intron of dystrophin gene: (c) a complementary sequence of the second promoter sequence; (d) a complementary sequence of the Cas9 scaffold sequence; (e) a complementary sequence of the guide sequence targeting a first intron of dystrophin gene; (f) a complementary sequence of the first promoter sequence; (h) a first AAV ITR sequence; (i) a first promoter sequence; (g) a guide sequence targeting a first intron of dystrophin gene; (k) a Cas9 scaffold sequence; (l) a second promoter sequence; (m) a guide sequence targeting a second intron of dystrophin gene; and (n) a second AAV ITR sequence.

Clause 49. The system of any one of clauses 46-48, wherein the first intron is intron 44 and the second intron is intron 55 of the dystrophin gene, or wherein the first intron is intron 55 and the second intron is intron of 44 of the dystrophin gene.

Clause 50. The system of any one of clauses 46-49, wherein the dystrophin gene comprises a mutation compared to a wild-type dystrophin gene.

Clause 51. The system of clause 46, wherein the guide sequence targeting a first intron of dystrophin gene comprises a nucleotide sequence of SEQ ID NO: 134 or 56 and the guide sequence targeting a second intron of dystrophin gene comprises a nucleotide sequence of SEQ ID NO: 135 or 55, or wherein the guide sequence targeting a first intron of dystrophin gene comprises a nucleotide sequence of SEQ ID NO: 135 or 55 and the guide sequence targeting a second intron of dystrophin gene comprises a nucleotide sequence of SEQ ID NO: 134 or 56.

Clause 52. The system of any one of clauses 46-51, wherein the promoter is a constitutive promoter or a tissue-specific promoter.

Clause 53. The system of any one of clauses 46-52, wherein the promoter is a muscle-specific promoter.

Clause 54. The system of clause 53, wherein the muscle-specific promoter comprises a human skeletal actin gene element, cardiac actin gene element, myocyte-specific enhancer binding factor mef, muscle creatine kinase (MCK), truncated MCK (tMCK), myosin heavy chain (MHC), MHCK7, C5-12, murine creatine kinase enhancer element, skeletal fast-twitch troponin c gene element, slow-twitch cardiac troponin c gene element, the slow-twitch troponin i gene element, hypoxia-inducible nuclear factor binding element, steroid-inducible element, or glucocorticoid response element (gre).

Clause 55. The system of clause 52, wherein the constitutive promoter comprises CMV, human U6 promoter, or H1 promoter.

Clause 56. The system of clause 52, wherein the constitutive promoter comprises a sequence of SEQ ID NO: 133 or 63.

Clause 57. The system of clause 46, wherein the first AAV ITR sequence comprises a sequence of SEQ ID NO: 132 or 59.

Clause 58. The system of clause 46, wherein the second AAV ITR sequence comprises a sequence of SEQ ID NO: 140 or 62.

Clause 59. The system of any one of clauses 46-58, wherein the expression cassette comprises a sequence of SEQ ID NO: 128.

Clause 60. The system of any one of clauses 46-59, wherein the expression cassette comprises a sequence of SEQ ID NO: 129.

Clause 61. The system of clause 46, wherein the Cas9 scaffold sequence is a spCas9 scaffold sequence or SaCas9 scaffold sequence.

Clause 62. The system of clause 61, wherein the Cas9 scaffold sequence is a SaCas9 scaffold sequence.

Clause 63. The system of clause 62, wherein the Cas9 scaffold sequence comprises a sequence of SEQ ID NO: 138 or 139 or 89 or 90 or 18.

Clause 64. The system of clause 46, wherein the one or more vectors encodes a Cas9 protein.

Clause 65. The system of clause 64, wherein the Cas9 protein is a SaCas9 or a spCas9 protein.

Clause 66. The system of clause 65, wherein the SaCas9 protein comprises an amino acid sequence of SEQ ID NO: 21 or 88 or is encoded by a polynucleotide comprising the sequence of SEQ ID NO: 69.

Clause 67. The system of any one of clauses 46-66, wherein the one or more vectors are viral vectors.

Clause 68. The system of clause 67, wherein the viral vector is an Adeno-associated virus (AAV) vector.

Clause 69. The system of clause 68, wherein the AAV vector is AAV1, AAV2, AAV3, AAV4, AAV5, AAV6, AAV7, AAV8, AAV9, AAV-10, AAV-11, AAV-12, AAV-13, or AAVrh.74.

Clause 70. The system of any one of clauses 46-69, wherein the vector that comprises an expression cassette is present in a concentration at least 2-fold, at least 3-fold, at least 4-fold, at least 5-fold, at least 6-fold, at least 7-fold, or at least 8-fold greater than the concentration of the vector encoding the Cas9 protein.

Clause 71. A cell comprising the system of any one of clauses 46-70.

Clause 72. A kit comprising the system of any one of clauses 46-70.

Clause 73. A method of correcting a mutant dystrophin gene in a cell, the method comprising administering to a cell the system of any one of clauses 46-70.

Clause 74. A method of genome editing a mutant dystrophin gene in a subject, the method comprising administering to the subject the system of any one of clauses 46-70 or the cell of clause 71.

Clause 75. A method of treating a subject having a mutant dystrophin gene, the method comprising administering to the subject the system of any one of clauses 46-70 or the cell of clause 71.

Clause 76. The method of any one of clauses 73-75, wherein the subject is a human.

Clause 77. The method of any one of clauses 73-76, wherein the system of any one of clauses 46-70 or the cell of clause 71 is administered to the subject intravenously.

Clause 78. The method of any one of clauses 73-78, wherein the system of any one of clauses 46-70 or the cell of clause 71 is administered to the subject systemically.

Clause 79. A plasmid expressing the expression cassette of clause 46, wherein the plasmid comprises a sequence selected from SEQ ID NOs: 87, 91, 92, 128, 129, 130, or 131.

SEQUENCES
SEQ ID NO: 1
NRG (R = A or G; N can be any nucleotide residue, e.g., any of A, G, C, or T)
SEQ ID NO: 2
NGG (N can be any nucleotide residue, e.g., any of A, G, C, or T)
SEQ ID NO: 3
NAG (N can be any nucleotide residue, e.g., any of A, G, C, or T)
SEQ ID NO: 4
NGGNG (N can be any nucleotide residue, e.g., any of A, G, C, or T)
SEQ ID NO: 5
NNAGAAW (W = A or T; N can be any nucleotide residue,
e.g., any of A, G, C, or T)
SEQ ID NO: 6
NAAR (R = A or G; N can be any nucleotide residue, e.g., any of A, G, C, or T)
SEQ ID NO: 7
NNGRR (R = A or G; N can be any nucleotide residue, e.g., any of A, G, C, or T)
SEQ ID NO: 8
NNGRRN (R = A or G; N can be any nucleotide residue, e.g., any of A, G, C, or T)
SEQ ID NO: 9
NNGRRT (R = A or G; N can be any nucleotide residue, e.g., any of A, G, C, or T)
SEQ ID NO: 10
NNGRRV (R = A or G; N can be any nucleotide residue, e.g.,
any of A, G, C, or T; V = A or C or G)
SEQ ID NO: 11
NNNNGATT (N can be any nucleotide residue, e.g., any of A, G, C, or T)
SEQ ID NO: 12
NNNNGNNN (N can be any nucleotide residue, e.g., any of A, G, C, or T)
SEQ ID NO: 13
NGA (N can be any nucleotide residue, e.g., any of A, G, C, or T)
SEQ ID NO: 14
NNNRRT (R = A or G; N can be any nucleotide residue, e.g., any of A, G, C, or T)
SEQ ID NO: 15
ATTCCT
SEQ ID NO: 16
NGAN (N can be any nucleotide residue, e.g., any of A, G, C, or T)
SEQ ID NO: 17
NGNG (N can be any nucleotide residue, e.g., any of A, G, C, or T)
DNA sequence of the gRNA constant region
SEQ ID NO: 18
gtttaagagctatgctggaaacagcatagcaagtttaaataaggctagtccgttatcaacttgaaaaa
gtggcaccgagtcggtgc
RNA sequence of the gRNA constant region
SEQ ID NO: 19
guuuaagagcuaugcuggaaacagcauagcaaguuuaaauaaggcuaguccguuaucaacuugaaaaa
guggcaccgagueggugc
Streptococcus pyogenes Cas9
SEQ ID NO: 20
MDKKYSIGLDIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKERGHFLIEGDLNPDNSDVDKLFIQLVQTYNQLFEENPINAS
GVDAKAILSARLSKSRRLENLIAQLPGEKKNGLFGNLIALSLGLTPNFKSNEDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
Staphylococcus aureus Cas9
SEQ ID NO: 21
MKRNYILGLDIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVQKAYHQLDQSFIDTYIDL
LETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKE
IIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDVKVKSINGGF
TSFLRRKWKFKKERNKGYKHHAEDALIIANADFIFKEWKKLDKAKKVMENQMFEEKQAESMPEIETEQ
EYKEIFITPHQIKHIKDFKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
Streptococcus pyogenes Cas9 (with D10A)
SEQ ID NO: 22
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
Streptococcus pyogenes Cas9 (with D10A, H849A)
SEQ ID NO: 23
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
Polynucleotide sequence of D10A mutant of S. aureus Cas9
SEQ ID NO: 24
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
Polynucleotide sequence of N580A mutant of S. aureus Cas9
SEQ ID NO: 25
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
codon optimized polynucleotide encoding S. pyogenes Cas9
SEQ ID NO: 26
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tcttgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagccaga aaatattgtg
atcgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tgggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga aacacggatc
gacctctctc aactgggcgg cgactag
codon optimized nucleic acid sequences encoding S. aureus Cas9
SEQ ID NO: 27
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
codon optimized nucleic acid sequences encoding S. aureus Cas9
SEQ ID NO: 28
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccgc ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tcttcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgatc
aacaagagcc ccgaaaagct gctgatgtac caccacgacc cccagaccta ccagaaactg
aagctgatta tggaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
codon optimized nucleic acid sequence encoding S. aureus Cas9
SEQ ID NO: 29
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctcca tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tcttcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tgtgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcatc aagaaggga
codon optimized nucleic acid sequence encoding S. aureus Cas9
SEQ ID NO: 30
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
codon optimized nucleic acid sequence encoding S. aureus Cas9
SEQ ID NO: 31
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac cgggaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgcctc taagactcag agtatcaaaa agtactcaac cgacattctg
ggaaacctgt atgaggtgaa gagcaaaaag caccctcaga ttatcaaaaa gggctaagaa
ttc
codon optimized nucleic acid sequences encoding S. aureus Cas9
SEQ ID NO: 32
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
codon optimized nucleic acid sequences encoding S. aureus Cas9
SEQ ID NO: 33
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
Vector (pDO242) encoding codon optimized nucleic acid sequence
encoding S. aureus Cas9
SEQ ID NO: 34
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac
Human p300 (with L553M mutation) protein
SEQ ID NO: 35
MAENVVEPGPPSAKRPKLSSPALSASASDGTDFGSLFDLEHDLPDELINSTELGLTNGGDINQLQTSL
GMVQDAASKHKQLSELLRSGSSPNLNMGVGGPGQVMASQAQQSSPGLGLINSMVKSPMTQAGLTSPNM
GMGTSGPNQGPTQSTGMMNSPVNQPAMGMNTGMNAGMNPGMLAAGNGQGIMPNQVMNGSIGAGRGRQN
MQYPNPGMGSAGNLLTEPLQQGSPQMGGQTGLRGPQPLKMGMMNNPNPYGSPYTQNPGQQIGASGLGL
QIQTKTVLSNNLSPFAMDKKAVPGGGMPNMGQQPAPQVQQPGLVTPVAQGMGSGAHTADPEKRKLIQQ
QLVLLLHAHKCQRREQANGEVRQCNLPHCRTMKNVLNHMTHCQSGKSCQVAHCASSRQIISHWKNCTR
HDCPVCLPLKNAGDKRNQQPILTGAPVGLGNPSSLGVGQQSAPNLSTVSQIDPSSIERAYAALGLPYQ
VNQMPTQPQVQAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMENLVAYA
RKVEGDMYESANNRAEYYHLLAEKIYKIQKELEEKRRTRLQKQNMLPNAAGMVPVSMNPGPNMGQPQP
GMTSNGPLPDPSMIRGSVPNQMMPRITPQSGLNQFGQMSMAQPPIVPRQTPPLQHHGQLAQPGALNPP
MGYGPRMQQPSNQGQFLPQTQFPSQGMNVTNIPLAPSSGQAPVSQAQMSSSSCPVNSPIMPPGSQGSH
IHCPQLPQPALHQNSPSPVPSRTPTPHHTPPSIGAQQPPATTIPAPVPTPPAMPPGPQSQALHPPPRQ
TPTPPTTQLPQQVQPSLPAAPSADQPQQQPRSQQSTAASVPTPTAPLLPPQPATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPESLPFRQPVDPQLLGIPD
YFDIVKSPMDLSTIKRKLDTGQYQEPWQYVDDIWLMFNNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDDPSQPQT
TINKEQFSKRKNDTLDPELFVECTECGRKMHQICVLHHEIIWPAGFVCDGCLKKSARTRKENKFSAKR
LPSTRLGTFLENRVNDFLRRQNHPESGEVTVRVVHASDKTVEVKPGMKARFVDSGEMAESFPYRTKAL
FAFEEIDGVDLCFFGMHVQEYGSDCPPPNQRRVYISYLDSVHFFRPKCLRTAVYHEILIGYLEYVKKL
GYTTGHIWACPPSEGDDYIFHCHPPDQKIPKPKRLQEWYKKMLDKAVSERIVHDYKDIFKQATEDRLT
SAKELPYFEGDFWPNVLEESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTMCMLVELHTQSQDRFVYTCNECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
KGCKRKTNGGCPICKQLIALCCYHAKHCQENKCPVPFCLNIKQKLRQQQLQHRLQQAQMLRRRMASMQ
RTGVVGQQQGLPSPTPATPTTPTGQQPTTPQTPQPTSQPQPTPPNSMPPYLPRTQAAGPVSQGKAAGQ
VTPPTPPQTAQPPLPGPPPAAVEMAMQIQRAAETQRQMAHVQIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVLSILHANPQLLAAFIKQRAAKYANSNPQPIPGQPGMPQ
GQPGLQPPTMPGQQGVHSNPAMQNMNPMQAGVQRAGLPQQQPQQQLQPPMGGMSPQAQQMNMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPNPMSPQQHMLPNQAQSPHLQGQQIPNSLSNQVRSPQP
VPSPRPQSQPPHSSPSPRMQPQPSPHHVSPQTSSPHPGLVAAQANPMEQGHFASPDQNSMLSQLASNP
GMANLHGASATDLGLSTDNSDLNSNLSQSTLDIH
Human p300 Core Effector protein (aa 1048-1664 of SEQ ID NO: 35)
SEQ ID NO: 36
IFKPEELRQALMPTLEALYRQDPESLPFRQPVDPQLLGIPDYFDIVKSPMDLSTIKRKLDTGQYQEPW
QYVDDIWLMFNNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDDPSQPQTTINKEQFSKRKNDTLDPELFVECTECG
RKMHQICVLHHEIIWPAGFVCDGCLKKSARTRKENKFSAKRLPSTRLGTFLENRVNDFLRRQNHPESG
EVTVRVVHASDKTVEVKPGMKARFVDSGEMAESFPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDFWPNVLEESIKELEQE
EEERKREENTSNESTDVTKGDSKNAKKKNNKKTSKNKSSLSRGNKKKPGMPNVSNDLSQKLYATMEKH
KEVFFVIRLIAGPAANSLPPIVDPDPLIPCDLMDGRDAFLTLARDKHLEFSSLRRAQWSTMCMLVELH
TQSQD
VP64-dCas9-VP64 protein
SEQ ID NO: 37
RADALDDFDLDMLGSDALDDFDLDMLGSDALDDFDLDMLGSDALDDFDLDMVNPKKKRKVGRGMDKKY
SIGLAIGTNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRIDLSQL
GGDSRADPKKKRKVASRADALDDFDLDMLGSDALDDFDLDMLGSDALDDFDLDMLGSDALDDFDLDML
I
VP64-dCas9-VP64 DNA
SEQ ID NO: 38
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatggttaaccccaagaagaagaggaaggtgggccgcggaatggacaagaagtac
tccattgggctcgccatcggcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctgctacctgcaggagatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagggggacctgaacccagacaacagcgatgtcgacaaactctttatcc
aactggttcagacttacaatcagcttttcgaagagaacccgatcaacgcatccggagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatggctacgccggatacattgacggcggagcaag
ccaggaggaattttacaaatttattaagcccatcttggaaaaaatggacggcaccgaggagctgctgg
taaagcttaacagagaagatctgttgcgcaaacagcgcactttcgacaatggaagcatcccccaccag
attcacctgggcgaactgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgagtacttcacagtttataacgagctcaccaagg
tcaaatacgtcacagaagggatgagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcacccttacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggattttcttaagtcc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtgtggatgaactcgtcaaagtaatgggaagg
cataagcccgagaatatcgttatcgagatggcccgagagaaccaaactacccagaagggacagaagaa
cagtagggaaaggatgaagaggattgaagagggtataaaagaactggggtcccaaatccttaaggaac
acccagttgaaaacacccagcttcagaatgagaagctctacctgtactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggggcagctgctgaacgcc
aaactgatcacacaacggaagttcgataatctgactaaggctgaacgaggtggcctgtctgagttgga
taaagccggcttcatcaaaaggcagcttgttgagacacgccagatcaccaagcacgtggcccaaattc
tcgattcacgcatgaacaccaagtacgatgaaaatgacaaactgattcgagaggtgaaagttattact
ctgaagtctaagctggtctcagatttcagaaaggactttcagttttataaggtgagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagtct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cgacaagctgatcgcacgcaaaaaagattgggaccccaagaaatacggcggattcgattctcctacag
tcgcttacagtgtactggttgtggccaaagtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctagtgcgggcgagctgcagaaaggtaacgagctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
tttcgatctcgacatgctgggttctgatgccctcgatgactttgacctggatatgttgggaagcgacg
cattggatgactttgatctggacatgctcggctccgatgctctggacgatttcgatctcgatatgtta
atc
Polynucleotide sequence encoding Streptococcus pyogenes dCas9-KRAB
SEQ ID NO: 39
atggactacaaagaccatgacggtgattataaagatcatgacatcgattacaaggatgacgatgacaa
gatggcccccaagaagaagaggaaggtgggccgcggaatggacaagaagtactccattgggctcgcca
tcggcacaaacagcgtcggctgggccgtcattacggacgagtacaaggtgccgagcaaaaaattcaaa
gttctgggcaataccgatcgccacagcataaagaagaacctcattggcgccctcctgttcgactccgg
ggaaaccgccgaagccacgcggctcaaaagaacagcacggcgcagatatacccgcagaaagaatcgga
tctgctacctgcaggagatctttagtaatgagatggctaaggtggatgactctttcttccataggctg
gaggagtcctttttggtggaggaggataaaaagcacgagcgccacccaatctttggcaatatcgtgga
cgaggtggcgtaccatgaaaagtacccaaccatatatcatctgaggaagaagcttgtagacagtactg
ataaggctgacttgcggttgatctatctcgcgctggcgcatatgatcaaatttcggggacacttcctc
atcgagggggacctgaacccagacaacagcgatgtcgacaaactctttatccaactggttcagactta
caatcagcttttcgaagagaacccgatcaacgcatccggagttgacgccaaagcaatcctgagcgcta
ggctgtccaaatcccggcggctcgaaaacctcatcgcacagctccctggggagaagaagaacggcctg
tttggtaatcttatcgccctgtcactcgggctgacccccaactttaaatctaacttcgacctggccga
agatgccaagcttcaactgagcaaagacacctacgatgatgatctcgacaatctgctggcccagatcg
gcgaccagtacgcagacctttttttggcggcaaagaacctgtcagacgccattctgctgagtgatatt
ctgcgagtgaacacggagatcaccaaagctccgctgagcgctagtatgatcaagcgctatgatgagca
ccaccaagacttgactttgctgaaggcccttgtcagacagcaactgcctgagaagtacaaggaaattt
tcttcgatcagtctaaaaatggctacgccggatacattgacggggagcaagccaggaggaattttac
aaatttattaagcccatcttggaaaaaatggacggcaccgaggagctgctggtaaagcttaacagaga
agatctgttgcgcaaacagcgcactttcgacaatggaagcatcccccaccagattcacctgggcgaac
tgcacgctatcctcaggcggcaagaggatttctacccctttttgaaagataacagggaaaagattgag
aaaatcctcacatttcggataccctactatgtaggccccctcgcccggggaaattccagattcgcgtg
gatgactcgcaaatcagaagagaccatcactccctggaacttcgaggaagtcgtggataagggggcct
ctgcccagtccttcatcgaaaggatgactaactttgataaaaatctgcctaacgaaaaggtgcttcct
aaacactctctgctgtacgagtacttcacagtttataacgagctcaccaaggtcaaatacgtcacaga
agggatgagaaagccagcattcctgtctggagagcagaagaaagctatcgtggacctcctcttcaaga
cgaaccggaaagttaccgtgaaacagctcaaagaagactatttcaaaaagattgaatgtttcgactct
gttgaaatcagcggagtggaggatcgcttcaacgcatccctgggaacgtatcacgatctcctgaaaat
cattaaagacaaggacttcctggacaatgaggagaacgaggacattcttgaggacattgtcctcaccc
ttacgttgtttgaagatagggagatgattgaagaacgcttgaaaacttacgctcatctcttcgacgac
aaagtcatgaaacagctcaagaggcgccgatatacaggatgggggcggctgtcaagaaaactgatcaa
tgggatccgagacaagcagagtggaaagacaatcctggattttcttaagtccgatggatttgccaacc
ggaacttcatgcagttgatccatgatgactctctcacctttaaggaggacatccagaaagcacaagtt
tctggccagggggacagtcttcacgagcacatcgctaatcttgcaggtagcccagctatcaaaaaggg
aatactgcagaccgttaaggtcgtggatgaactcgtcaaagtaatgggaaggcataagcccgagaata
tcgttatcgagatggcccgagagaaccaaactacccagaagggacagaagaacagtagggaaaggatg
aagaggattgaagagggtataaaagaactggggtcccaaatccttaaggaacacccagttgaaaacac
ccagcttcagaatgagaagctctacctgtactacctgcagaacggcagggacatgtacgtggatcagg
aactggacatcaatcggctctccgactacgacgtggatgccatcgtgccccagtcttttctcaaagat
gattctattgataataaagtgttgacaagatccgataaaaatagagggaagagtgataacgtcccctc
agaagaagttgtcaagaaaatgaaaaattattggcggcagctgctgaacgccaaactgatcacacaac
ggaagttcgataatctgactaaggctgaacgaggtggcctgtctgagttggataaagccggcttcatc
aaaaggcagcttgttgagacacgccagatcaccaagcacgtggcccaaattctcgattcacgcatgaa
caccaagtacgatgaaaatgacaaactgattcgagaggtgaaagttattactctgaagtctaagctgg
tctcagatttcagaaaggactttcagttttataaggtgagagagatcaacaattaccaccatgcgcat
gatgcctacctgaatgcagtggtaggcactgcacttatcaaaaaatatcccaagcttgaatctgaatt
tgtttacggagactataaagtgtacgatgttaggaaaatgatcgcaaagtctgagcaggaaataggca
aggccaccgctaagtacttcttttacagcaatattatgaattttttcaagaccgagattacactggcc
aatggagagattcggaagcgaccacttatcgaaacaaacggagaaacaggagaaatcgtgtgggacaa
gggtagggatttcgcgacagtccggaaggtcctgtccatgccgcaggtgaacatcgttaaaaagaccg
aagtacagaccggaggcttctccaaggaaagtatcctcccgaaaaggaacagcgacaagctgatcgca
cgcaaaaaagattgggaccccaagaaatacggcggattcgattctcctacagtcgcttacagtgtact
ggttgtggccaaagtggagaaagggaagtctaaaaaactcaaaagcgtcaaggaactgctgggcatca
caatcatggagcgatcaagcttcgaaaaaaaccccatcgactttctcgaggcgaaaggatataaagag
gtcaaaaaagacctcatcattaagcttcccaagtactctctctttgagcttgaaaacggccggaaacg
aatgctcgctagtgcgggcgagctgcagaaaggtaacgagctggcactgccctctaaatacgttaatt
tcttgtatctggccagccactatgaaaagctcaaagggtctcccgaagataatgagcagaagcagctg
ttcgtggaacaacacaaacactaccttgatgagatcatcgagcaaataagcgaattctccaaaagagt
gatcctcgccgacgctaacctcgataaggtgctttctgcttacaataagcacagggataagcccatca
gggagcaggcagaaaacattatccacttgtttactctgaccaacttgggcgcgcctgcagccttcaag
tacttcgacaccaccatagacagaaagcggtacacctctacaaaggaggtcctggacgccacactgat
tcatcagtcaattacggggctctatgaaacaagaatcgacctctctcagctcggtggagacagcaggg
ctgaccccaagaagaagaggaaggtggctagcgatgctaagtcactgactgcctggtcccggacactg
gtgaccttcaaggatgtgtttgtggacttcaccagggaggagtggaagctgctggacactgctcagca
gatcctgtacagaaatgtgatgctggagaactataagaacctggtttccttgggttatcagcttacta
agccagatgtgatcctccggttggagaagggagaagagccctggctggtggagagagaaattcaccaa
gagacccatcctgattcagagactgcatttgaaatcaaatcatcagttccgaaaaagaaacgcaaagt
ttga
Polypeptide sequence of Streptococcus pyogenes dCas9-KRAB protein
SEQ ID NO: 40
MDYKDHDGDYKDHDIDYKDDDDKMAPKKKRKVGRGMDKKYSIGLAIGTNSVGWAVITDEYKVPSKKFK
VLGNTDRHSIKKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAHLFDD
KVMKQLKRRRYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLTNLGAPAAFK
YFDTTIDRKRYTSTKEVLDATLIHQSITGLYETRIDLSQLGGDSRADPKKKRKVASDAKSLTAWSRTL
VTFKDVFVDFTREEWKLLDTAQQILYRNVMLENYKNLVSLGYQLTKPDVILRLEKGEEPWLVEREIHQ
ETHPDSETAFEIKSSVPKKKRKV
Polynucleotide sequence of Staphylococcus aureus dCas9-KRAB protein
SEQ ID NO: 41
atggccccaaagaagaagcggaaggtcggtatccacggagtcccagcagccaagcggaactacatcct
gggcctggccatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaagccagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaagggatccgat
gctaagtcactgactgcctggtcccggacactggtgaccttcaaggatgtgtttgtggacttcaccag
ggaggagtggaagctgctggacactgctcagcagatcctgtacagaaatgtgatgctggagaactata
agaacctggtttccttgggttatcagcttactaagccagatgtgatcctccggttggagaagggagaa
gagccctggctggtggagagagaaattcaccaagagacccatcctgattcagagactgcatttgaaat
caaatcatcagttccgaaaaagaaacgcaaagtt
Polypeptide sequence of Staphylococcus aureus dCas9-KRAB protein
SEQ ID NO: 42
MAPKKKRKVGIHGVPAAKRNYILGLAIGITSVGYGIIDYETRDVIDAGVRLFKEANVENNEGRRSKRG
ARRLKRRRRHRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYAYNADLY
NALNDLNNLVITRDENEKLEYYEKFQIIENVFKQKKKPTLKQIAKEILVNEEDIKGYRVTSTGKPEFT
NLKVYHDIKDITARKEIIENAELLDQIAKILTIYQSSEDIQEELTNLNSELTQEEIEQISNLKGYTGT
HNLSLKAINLILDELWHTNDNQIAIFNRLKLVPKKVDLSQQKEIPTTLVDDFILSPVVKRSFIQSIKV
INAIIKKYGLPNDIIIELAREKNSKDAQKMINEMQKRNRQTNERIEEIIRTTGKENAKYLIEKIKLHD
MQEGKCLYSLEAIPLEDLLNNPFNYEVDHIIPRSVSFDNSFNNKVLVKQEEASKKGNRTPFQYLSSSD
SKISYETFKKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLDKAKKVMENQM
FEEKQAESMPEIETEQEYKEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKFVTVKNLDVIKK
ENYYEVNSKCYEEAKKLKKISNQAEFIASFYNNDLIKINGELYRVIGVNNDLLNRIEVNMIDITYREY
LENMNDKRPPRIIKTIASKTQSIKKYSTDILGNLYEVKSKKHPQIIKKGKRPAATKKAGQAKKKKGSD
AKSLTAWSRTLVTFKDVFVDFTREEWKLLDTAQQILYRNVMLENYKNLVSLGYQLTKPDVILRLEKGE
EPWLVEREIHQETHPDSETAFEIKSSVPKKKRKV
Polynucleotide sequence of Tet1CD
SEQ ID NO: 43
CTGCCCACCTGCAGCTGTCTTGATCGAGTTATACAAAAAGACAAAGGCCCATATTATACACACCTTGG
GGCAGGACCAAGTGTTGCTGCTGTCAGGGAAATCATGGAGAATAGGTATGGTCAAAAAGGAAACGCAA
TAAGGATAGAAATAGTAGTGTACACCGGTAAAGAAGGGAAAAGCTCTCATGGGTGTCCAATTGCTAAG
TGGGTTTTAAGAAGAAGCAGTGATGAAGAAAAAGTTCTTTGTTTGGTCCGGCAGCGTACAGGCCACCA
CTGTCCAACTGCTGTGATGGTGGTGCTCATCATGGTGTGGGATGGCATCCCTCTTCCAATGGCCGACC
GGCTATACACAGAGCTCACAGAGAATCTAAAGTCATACAATGGGCACCCTACCGACAGAAGATGCACC
CTCAATGAAAATCGTACCTGTACATGTCAAGGAATTGATCCAGAGACTTGTGGAGCTTCATTCTCTTT
TGGCTGTTCATGGAGTATGTACTTTAATGGCTGTAAGTTTGGTAGAAGCCCAAGCCCCAGAAGATTTA
GAATTGATCCAAGCTCTCCCTTACATGAAAAAAACCTTGAAGATAACTTACAGAGTTTGGCTACACGA
TTAGCTCCAATTTATAAGCAGTATGCTCCAGTAGCTTACCAAAATCAGGTGGAATATGAAAATGTTGC
CCGAGAATGTCGGCTTGGCAGCAAGGAAGGTCGACCCTTCTCTGGGGTCACTGCTTGCCTGGACTTCT
GTGCTCATCCCCACAGGGACATTCACAACATGAATAATGGAAGCACTGTGGTTTGTACCTTAACTCGA
GAAGATAACCGCTCTTTGGGTGTTATTCCTCAAGATGAGCAGCTCCATGTGCTACCTCTTTATAAGCT
TTCAGACACAGATGAGTTTGGCTCCAAGGAAGGAATGGAAGCCAAGATCAAATCTGGGGCCATCGAGG
TCCTGGCACCCCGCCGCAAAAAAAGAACGTGTTTCACTCAGCCTGTTCCCCGTTCTGGAAAGAAGAGG
GCTGCGATGATGACAGAGGTTCTTGCACATAAGATAAGGGCAGTGGAAAAGAAACCTATTCCCCGAAT
CAAGCGGAAGAATAACTCAACAACAACAAACAACAGTAAGCCTTCGTCACTGCCAACCTTAGGGAGTA
ACACTGAGACCGTGCAACCTGAAGTAAAAAGTGAAACCGAACCCCATTTTATCTTAAAAAGTTCAGAC
AACACTAAAACTTATTCGCTGATGCCATCCGCTCCTCACCCAGTGAAAGAGGCATCTCCAGGCTTCTC
CTGGTCCCCGAAGACTGCTTCAGCCACACCAGCTCCACTGAAGAATGACGCAACAGCCTCATGCGGGT
TTTCAGAAAGAAGCAGCACTCCCCACTGTACGATGCCTTCGGGAAGACTCAGTGGTGCCAATGCTGCA
GCTGCTGATGGCCCTGGCATTTCACAGCTTGGCGAAGTGGCTCCTCTCCCCACCCTGTCTGCTCCTGT
GATGGAGCCCCTCATTAATTCTGAGCCTTCCACTGGTGTGACTGAGCCGCTAACGCCTCATCAGCCAA
ACCACCAGCCCTCCTTCCTCACCTCTCCTCAAGACCTTGCCTCTTCTCCAATGGAAGAAGATGAGCAG
CATTCTGAAGCAGATGAGCCTCCATCAGACGAACCCCTATCTGATGACCCCCTGTCACCTGCTGAGGA
GAAATTGCCCCACATTGATGAGTATTGGTCAGACAGTGAGCACATCTTTTTGGATGCAAATATTGGTG
GGGTGGCCATCGCACCTGCTCACGGCTCGGTTTTGATTGAGTGTGCCCGGCGAGAGCTGCACGCTACC
ACTCCTGTTGAGCACCCCAACCGTAATCATCCAACCCGCCTCTCCCTTGTCTTTTACCAGCACAAAAA
CCTAAATAAGCCCCAACATGGTTTTGAACTAAACAAGATTAAGTTTGAGGCTAAAGAAGCTAAGAATA
AGAAAATGAAGGCCTCAGAGCAAAAAGACCAGGCAGCTAATGAAGGTCCAGAACAGTCCTCTGAAGTA
AATGAATTGAACCAAATTCCTTCTCATAAAGCATTAACATTAACCCATGACAATGTTGTCACCGTGTC
CCCTTATGCTCTCACACACGTTGCGGGGCCCTATAACCATTGGGTC
Polypeptide sequence of Tet1CD
SEQ ID NO: 44
LPTCSCLDRVIQKDKGPYYTHLGAGPSVAAVREIMENRYGQKGNAIRIEIVVYTGKEGKSSHGCPIAK
WVLRRSSDEEKVLCLVRQRTGHHCPTAVMVVLIMVWDGIPLPMADRLYTELTENLKSYNGHPTDRRCT
LNENRTCTCQGIDPETCGASFSFGCSWSMYFNGCKFGRSPSPRRFRIDPSSPLHEKNLEDNLQSLATR
LAPIYKQYAPVAYQNQVEYENVARECRLGSKEGRPFSGVTACLDFCAHPHRDIHNMNNGSTVVCTLTR
EDNRSLGVIPQDEQLHVLPLYKLSDTDEFGSKEGMEAKIKSGAIEVLAPRRKKRTCFTQPVPRSGKKR
AAMMTEVLAHKIRAVEKKPIPRIKRKNNSTTTNNSKPSSLPTLGSNTETVQPEVKSETEPHFILKSSD
NTKTYSLMPSAPHPVKEASPGFSWSPKTASATPAPLKNDATASCGFSERSSTPHCTMPSGRLSGANAA
AADGPGISQLGEVAPLPTLSAPVMEPLINSEPSTGVTEPLTPHQPNHQPSFLTSPQDLASSPMEEDEQ
HSEADEPPSDEPLSDDPLSPAEEKLPHIDEYWSDSEHIFLDANIGGVAIAPAHGSVLIECARRELHAT
TPVEHPNRNHPTRLSLVFYQHKNLNKPQHGFELNKIKFEAKEAKNKKMKASEQKDQAANEGPEQSSEV
NELNQIPSHKALTLTHDNVVTVSPYALTHVAGPYNHWV
Protein sequence for VPH
SEQ ID NO: 45
DALDDFDLDMLGSDALDDFDLDMLGSDALDDFDLDMLGSDALDDFDLDMLGSLPSASVEFEGSGGPSG
QISNQALALAPSSAPVLAQTMVPSSAMVPLAQPPAPAPVLTPGPPQSLSAPVPKSTQAGEGTLSEALL
HLQFDADEDIGALLGNSTDPGVFTDLASVDNSEFQQLLNQGVSMSHSTAEPMLMEYPEAITRLVTGSQ
RPPDPAPTPLGTSGLPNGLSGDEDFSSIADMDFSALLSQISSSGQGGGGSGFSVDTSALLDLFSPSVT
VPDMSLPDLDSSLASIQELLSPQEPPRPPEAENSSPDSGKQLVHYTAQPLFLLDPGSVDTGSNDLPVL
FELGEGSYFSEGDGFAEDPTISLLTGSEPPKAKDPTVS
DNA sequence for VPH
SEQ ID NO: 46
Gatgctttagacgattttgacttagatatgcttggttcagacgcgttagacgacttcgacctagacat
gttaggctcagatgcattggacgacttcgatttagatatgttgggctccgatgccctagatgactttg
atctagatatgctagggtcactacccagcgccagcgtcgagttcgaaggcagcggcgggccttcaggg
cagatcagcaaccaggccctggctctggcccctagctccgctccagtgctggcccagactatggtgcc
ctctagtgctatggtgcctctggcccagccacctgctccagcccctgtgctgaccccaggaccacccc
agtcactgagcgccccagtgcccaagtctacacaggccggcgaggggactctgagtgaagctctgctg
cacctgcagttcgacgctgatgaggacctgggagctctgctggggaacagcaccgatcccggagtgtt
cacagatctggcctccgtggacaactctgagtttcagcagctgctgaatcagggcgtgtccatgtctc
atagtacagccgaaccaatgctgatggagtaccccgaagccattacccggctggtgaccggcagccag
cggccccccgaccccgctccaactcccctgggaaccagcggcctgcctaatgggctgtccggagatga
agacttctcaagcatcgctgatatggactttagtgccctgctgtcacagatttcctctagtgggcagg
gaggaggtggaagcggcttcagcgtggacaccagtgccctgctggacctgttcagcccctcggtgacc
gtgcccgacatgagcctgcctgaccttgacagcagcctggccagtatccaagagctcctgtctcccca
ggagccccccaggcctcccgaggcagagaacagcagcccggattcagggaagcagctggtgcactaca
cagcgcagccgctgttcctgctggaccccggctccgtggacaccgggagcaacgacctgccggtgctg
tttgagctgggagagggctcctacttctccgaaggggacggcttcgccgaggaccccaccatctccct
gctgacaggctcggagcctcccaaagccaaggaccccactgtctcc
Protein sequence for VPR
SEQ ID NO: 47
DALDDFDLDMLGSDALDDFDLDMLGSDALDDFDLDMLGSDALDDFDLDMLGSPKKKRKVGSQYLPDTD
DRHRIEEKRKRTYETFKSIMKKSPFSGPTDPRPPPRRIAVPSRSSASVPKPAPQPYPFTSSLSTINYD
EFPTMVFPSGQISQASALAPAPPQVLPQAPAPAPAPAMVSALAQAPAPVPVLAPGPPQAVAPPAPKPT
QAGEGTLSEALLQLQFDDEDLGALLGNSTDPAVFTDLASVDNSEFQQLLNQGIPVAPHTTEPMLMEYP
EAITRLVTGAQRPPDPAPAPLGAPGLPNGLLSGDEDFSSIADMDFSALLSQISSGSGSGSRDSREGMF
LPKPEAGSAISDVFEGREVCQPKRIRPFHPPGSPWANRPLPASLAPTPTGPVHEPVGSLTPAPVPQPL
DPAPAVTPEASHLLEDPDEETSQAVKALREMADTVIPQKEEAAICGQMDLSHPPPRGHLDELTTTLES
MTEDLNLDSPLTPELNEILDTFLNDECLLHAMHISTGLSIFDTSLF
DNA sequence for VPR
SEQ ID NO: 48
gatgctttagacgattttgacttagatatgcttggttcagacgcgttagacgacttcgacctagacat
gttaggctcagatgcattggacgacttcgatttagatatgttgggctccgatgccctagatgactttg
atctagatatgctaggtagtcccaaaaagaagaggaaagtgggatcccagtatctgcccgacacagat
gatagacaccgaatcgaagagaaacgcaagcgaacgtatgaaaccttcaaatcgatcatgaagaaatc
gcccttctcgggtccgaccgatcccaggcccccaccgagaaggattgcggtcccgtcccgctcgtcgg
ccagcgtgccgaagcctgcgccgcagccctaccccttcacgtcgagcctgagcacaatcaattatgac
gagttcccgacgatggtgttcccctcgggacaaatctcacaagcctcggcgctcgcaccagcgcctcc
ccaagtccttccgcaagcgcctgccccagcgcctgcaccggcaatggtgtccgccctcgcacaggccc
ctgcgcccgtccccgtgctcgcgcctggaccgccccaggcggtcgctccaccggctccgaagccgacg
caggccggagagggaacactctccgaagcacttcttcaactccagtttgatgacgaggatcttggagc
actccttggaaactcgacagaccctgcggtgtttaccgacctcgcgtcagtagataactccgaatttc
agcagcttttgaaccagggtatcccggtcgcgccacatacaacggagcccatgttgatggaatacccc
gaagcaatcacgagacttgtgacgggagcgcagcggcctcccgatcccgcacccgcacctttgggggc
acctggcctccctaacggacttttgagcggcgacgaggatttctcctccatcgccgatatggatttct
cagccttgctgtcacagatttccagcggctctggcagcggcagccgggattccagggaagggatgttt
ttgccgaagcctgaggccggctccgctattagtgacgtgtttgagggccgcgaggtgtgccagccaaa
acgaatccggccatttcatcctccaggaagtccatgggccaaccgcccactccccgccagcctcgcac
caacaccaaccggtccagtacatgagccagtcgggtcactgaccccggcaccagtccctcagccactg
gatccagcgcccgcagtgactcccgaggccagtcacctgttggaggatcccgatgaagagacgagcca
ggctgtcaaagcccttcgggagatggccgatactgtgattccccagaaggaagaggctgcaatctgtg
gccaaatggacctttcccatccgcccccaaggggccatctggatgagctgacaaccacacttgagtcc
atgaccgaggatctgaacctggactcacccctgaccccggaattgaacgagattctggataccttcct
gaacgacgagtgcctcttgcatgccatgcatatcagcacaggactgtccatcttcgacacatctctgt
tt
SEQ ID NO: 49
SV40 NLS (Pro-Lys-Lys-Lys-Arg-Lys-Val)
SEQ ID NO: 50
GS linker (Gly-Gly-Gly-Gly-Ser)ₙ, wherein n is an integer between 0 and 10
SEQ ID NO: 51
Gly-Gly-Gly-Gly-Gly
SEQ ID NO: 52
Gly-Gly-Ala-Gly-Gly
SEQ ID NO: 53
Gly-Gly-Gly-Gly-Ser-Ser-Ser
SEQ ID NO: 54
Gly-Gly-Gly-Gly-Ala-Ala-Ala
JCR143: DNA target sequence of gRNA targeting human dystrophin intron
44 region
SEQ ID NO: 55
acatttcctctctatacaaatg
JCR120: DNA target sequence of gRNA targeting human dystrophin intron
55 region
SEQ ID NO: 56
atatagtaatgaaattattggcac
JCR143: gRNA targeting human dystrophin intron 44 region
SEQ ID NO: 57
acauuuccucucuauacaaaug
JCR120: gRNA targeting human dystrophin intron 55 region
SEQ ID NO: 58
auauaguaaugaaauuauuggcac
AAV ITR (wild-type)
SEQ ID NO: 59
cctgcaggcagctgcgcgctcgctcgctcactgaggccgcccgggcgtcgggcgacctttgg
tcgcccggcctcagtgagcgagcgagcgcgcagagagggagtggccaactccatcactaggg
gttcct
AAV ITR (wild-type)
SEQ ID NO: 60
ggggggggggggggggggttggccactccctctctgcgcgctcgctcgctcactgaggccgg
gcgaccaaaggtcgcccgacgcccgggctttgcccgggcggcctcagtgagcgagcgagcgc
gcagagagggagtggccaactccatcactaggg
AAV ITR (wild-type)
SEQ ID NO: 61
gggccactccctctctgcgcgctcgctcgctcactgaggccgggcgaccaaaggtcgcccga
ccccgggctttgcccgggcggcctcagtgagcgagcgagcgcgcagagagggagtggccaa
ctccatcactaggggttcct
mutant ITR
SEQ ID NO: 62
ctagtccactccctctctgcgcgctcgctcgctcactgaggccgggcgaccaaaggtcgccc
gacgcccgggctttgcccgggcggcctcagtgagcgagcgagegcgcagagagggac
U6 promoter
SEQ ID NO: 63
ggtgtttcgtcctttccacaagatatataaagccaagaaatcgaaatactttcaagttacgg
taagcatatgatagtccattttaaaacataattttaaaactgcaaactacccaagaaattat
tactttctacgtcacgtattttgtactaatatctttgtgtttacagtcaaattaattccaat
tatctctctaacagccttgtatcgtatatgcaaatatgaaggaatcatgggaaataggccct
c
H1 promoter
SEQ ID NO: 64
Gaacgctgacgtcatcaacccgctccaaggaatcgcgggcccagtgtcactaggcgggaaca
cccagcgcgcgtgcgccctggcaggaagatggctgtgagggacaggggagtggcgccctgca
atatttgcatgtcgctatgtgttctgggaaatcaccataaacgtgaaatgtctttggatttg
ggaatcttataagttctgtatgagaccac
EFS promoter
SEQ ID NO: 65
Tcgagtggctccggtgcccgtcagtgggcagagcgcacatcgcccacagtccccgagaagtt
ggggggaggggtcggcaattgaaccggtgcctagagaaggtggcgcggggtaaactgggaaa
gtgatgtcgtgtactggctccgcctttttcccgaggggggggagaaccgtatataagtgca
gtagtcgccgtgaacgttctttttcgcaacgggtttgccgccagaacacaggtgtcgtgacc
gcgg
CK8 promoter
SEQ ID NO: 66
ctagactagcatgctgcccatgtaaggaggcaaggcctggggacacccgagatgcctggtta
taattaacccagacatgtggctgcccccccccccccaacacctgctgcctctaaaaataacc
ctgcatgccatgttcccggcgaagggccagctgtcccccgccagctagactcagcacttagt
ttaggaaccagtgagcaagtcagcccttggggcagcccatacaaggccatggggctgggcaa
gctgcacgcctgggtccggggtgggcacggtgcccgggcaacgagctgaaagctcatctgct
ctcaggggcccctccctggggacagcccctcctggctagtcacaccctgtaggctcctctat
ataacccaggggcacaggggctgccctcattctaccaccacctccacagcacagacagacac
tcaggagccagccag
Spc512 promoter
SEQ ID NO: 67
gagctccaccgcggtggcggccgtccgccttcggcaccatcctcacgacacccaaatatggc
gacgggtgaggaatggtggggagttatttttagagcggtgaggaaggtgggcaggcagcagg
tgttggcgctctaaaaataactcccgggagttatttttagagcggaggaatggtggacaccc
aaatatggcgacggttcctcacccgtcgccatatttgggtgtccgccctcggccggggccgc
attcctgggggccgggcggtgctcccgcccgcctcgataaaaggctccggggccggcggcgg
cccacgagctacccggaggagcgggaggcgccaagctctagaactagtggatcccccgggct
gcaggaattcgatat
MHCK7 promoter
SEQ ID NO: 68
gtttaaacaagcttgcatgtctaagctagacccttcagattaaaaataactgaggtaagggc
ctgggtaggggaggtggtgtgagacgctcctgtctctcctctatctgcccatcggccctttg
gggaggaggaatgtgcccaaggactaaaaaaaggccatggagccagaggggcgagggcaaca
gacctttcatgggcaaaccttggggccctgctgtctagcatgccccactacgggtctaggct
gcccatgtaaggaggcaaggcctggggacacccgagatgcctggttataattaacccagaca
tgtggctgcccccccccccccaacacctgctgcctctaaaaataaccctgtccctggtggat
cccctgcatgcgaagatcttcgaacaaggctgtgggggactgagggcaggctgtaacaggct
tgggggccagggcttatacgtgcctgggactcccaaagtattactgttccatgttcccggcg
aagggccagctgtcccccgccagctagactcagcacttagtttaggaaccagtgagcaagtc
agcccttggggcagcccatacaaggccatggggctgggcaagctgcacgcctgggtccgggg
tgggcacggtgcccgggcaacgagctgaaagctcatctgctctcaggggcccctccctgggg
acagcccctcctggctagtcacaccctgtaggctcctctatataacccaggggcacaggggc
tgccctcattctaccaccacctccacagcacagacagacactcaggagccagccagcggcgc
gccc
polynucleotide encoding SaCas9
SEQ ID NO: 69
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
Mini polyadenylation signal
SEQ ID NO: 70
Tagcaataaaggatcgtttattttcattggaagcgtgtgttggttttttgatcaggcgcg
bGH polyadenylation signal
SEQ ID NO: 71
ctagagctcgctgatcagcctcgactgtgccttctagttgccagccatctgttgtttgcccctccccc
gtgccttccttgaccctggaaggtgccactcccactgtcctttcctaataaaatgaggaaattgcatc
gcattgtctgagtaggtgtcattctattctggggggtggggtggggcaggacagcaagggggaggatt
gggaagagaatagcaggcatgctgggga
polynucleotide encoding SV40 intron
SEQ ID NO: 72
tctagaggatccggtactcgaggaactgaaaaaccagaaagttaactggtaagtttagtctttttgtc
ttttatttcaggtcccggatccggtggtggtgcaaatcaaagaactgctcctcagtggatgttgcctt
tacttctaggcctgtacggaagtgttac
Version 1 of vector 1
SEQ ID NO: 73
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGAGAGGGCCTATTTCCCATGATTCCTTCATATTTGCATATACGATACAAGGCTGTTAGAGAGAT
AATTGGAATTAATTTGACTGTAAACACAAAGATATTAGTACAAAATACGTGACGTAGAAAGTAATAAT
TTCTTGGGTAGTTTGCAGTTTTAAAATTATGTTTTAAAATGGACTATCATATGCTTACCGTAACTTGA
AAGTATTTCGATTTCTTGGCTTTATATATCTTGTGGAAAGGACGAAACACCGcatttgtatagagagg
aaatgtgttttagtactctggaaacagaatctactaaaacaaggcaaaatgccgtgtttatctcgtca
acttgttggcgagatttttCTCGAGTCGAGTGGCTCCGGTGCCCGTCAGTGGGCAGAGCGCACATCGC
CCACAGTCCCCGAGAAGTTGGGGGGAGGGGTCGGCAATTGAACCGGTGCCTAGAGAAGGTGGCGCGGG
GTAAACTGGGAAAGTGATGTCGTGTACTGGCTCCGCCTTTTTCCCGAGGGGGGGGAGAACCGTATAT
AAGTGCAGTAGTCGCCGTGAACGTTCTTTTTCGCAACGGGTTTGCCGCCAGAACACAGGTGTCGTGAC
CGCGGCCATGGCCCCAAAGAAGAAGCGGAAGGTCGGTATCCACGGAGTCCCAGCAGCCAAGCGGAACT
ACATCCTGGGCCTGGACATCGGCATCACCAGCGTGGGCTACGGCATCATCGACTACGAGACACGGGAC
GTGATCGATGCCGGCGTGCGGCTGTTCAAAGAGGCCAACGTGGAAAACAACGAGGGCAGGCGGAGCAA
GAGAGGCGCCAGAAGGCTGAAGCGGCGGAGGCGGCATAGAATCCAGAGAGTGAAGAAGCTGCTGTTCG
ACTACAACCTGCTGACCGACCACAGCGAGCTGAGCGGCATCAACCCCTACGAGGCCAGAGTGAAGGGC
CTGAGCCAGAAGCTGAGCGAGGAAGAGTTCTCTGCCGCCCTGCTGCACCTGGCCAAGAGAAGAGGCGT
GCACAACGTGAACGAGGTGGAAGAGGACACCGGCAACGAGCTGTCCACCAAAGAGCAGATCAGCCGGA
ACAGCAAGGCCCTGGAAGAGAAATACGTGGCCGAACTGCAGCTGGAACGGCTGAAGAAAGACGGCGAA
GTGCGGGGCAGCATCAACAGATTCAAGACCAGCGACTACGTGAAAGAAGCCAAACAGCTGCTGAAGGT
GCAGAAGGCCTACCACCAGCTGGACCAGAGCTTCATCGACACCTACATCGACCTGCTGGAAACCCGGC
GGACCTACTATGAGGGACCTGGCGAGGGCAGCCCCTTCGGCTGGAAGGACATCAAAGAATGGTACGAG
ATGCTGATGGGCCACTGCACCTACTTCCCCGAGGAACTGCGGAGCGTGAAGTACGCCTACAACGCCGA
CCTGTACAACGCCCTGAACGACCTGAACAATCTCGTGATCACCAGGGACGAGAACGAGAAGCTGGAAT
ATTACGAGAAGTTCCAGATCATCGAGAACGTGTTCAAGCAGAAGAAGAAGCCCACCCTGAAGCAGATC
GCCAAAGAAATCCTCGTGAACGAAGAGGATATTAAGGGCTACAGAGTGACCAGCACCGGCAAGCCCGA
GTTCACCAACCTGAAGGTGTACCACGACATCAAGGACATTACCGCCCGGAAAGAGATTATTGAGAACG
CCGAGCTGCTGGATCAGATTGCCAAGATCCTGACCATCTACCAGAGCAGCGAGGACATCCAGGAAGAA
CTGACCAATCTGAACTCCGAGCTGACCCAGGAAGAGATCGAGCAGATCTCTAATCTGAAGGGCTATAC
CGGCACCCACAACCTGAGCCTGAAGGCCATCAACCTGATCCTGGACGAGCTGTGGCACACCAACGACA
ACCAGATCGCTATCTTCAACCGGCTGAAGCTGGTGCCCAAGAAGGTGGACCTGTCCCAGCAGAAAGAG
ATCCCCACCACCCTGGTGGACGACTTCATCCTGAGCCCCGTCGTGAAGAGAAGCTTCATCCAGAGCAT
CAAAGTGATCAACGCCATCATCAAGAAGTACGGCCTGCCCAACGACATCATTATCGAGCTGGCCCGCG
AGAAGAACTCCAAGGACGCCCAGAAAATGATCAACGAGATGCAGAAGCGGAACCGGCAGACCAACGAG
CGGATCGAGGAAATCATCCGGACCACCGGCAAAGAGAACGCCAAGTACCTGATCGAGAAGATCAAGCT
GCACGACATGCAGGAAGGCAAGTGCCTGTACAGCCTGGAAGCCATCCCTCTGGAAGATCTGCTGAACA
ACCCCTTCAACTATGAGGTGGACCACATCATCCCCAGAAGCGTGTCCTTCGACAACAGCTTCAACAAC
AAGGTGCTCGTGAAGCAGGAAGAAAACAGCAAGAAGGGCAACCGGACCCCATTCCAGTACCTGAGCAG
CAGCGACAGCAAGATCAGCTACGAAACCTTCAAGAAGCACATCCTGAATCTGGCCAAGGGCAAGGGCA
GAATCAGCAAGACCAAGAAAGAGTATCTGCTGGAAGAACGGGACATCAACAGGTTCTCCGTGCAGAAA
GACTTCATCAACCGGAACCTGGTGGATACCAGATACGCCACCAGAGGCCTGATGAACCTGCTGCGGAG
CTACTTCAGAGTGAACAACCTGGACGTGAAAGTGAAGTCCATCAATGGCGGCTTCACCAGCTTTCTGC
GGCGGAAGTGGAAGTTTAAGAAAGAGCGGAACAAGGGGTACAAGCACCACGCCGAGGACGCCCTGATC
ATTGCCAACGCCGATTTCATCTTCAAAGAGTGGAAGAAACTGGACAAGGCCAAAAAAGTGATGGAAAA
CCAGATGTTCGAGGAAAAGCAGGCCGAGAGCATGCCCGAGATCGAAACCGAGCAGGAGTACAAAGAGA
TCTTCATCACCCCCCACCAGATCAAGCACATTAAGGACTTCAAGGACTACAAGTACAGCCACCGGGTG
GACAAGAAGCCTAATAGAGAGCTGATTAACGACACCCTGTACTCCACCCGGAAGGACGACAAGGGCAA
CACCCTGATCGTGAACAATCTGAACGGCCTGTACGACAAGGACAATGACAAGCTGAAAAAGCTGATCA
ACAAGAGCCCCGAAAAGCTGCTGATGTACCACCACGACCCCCAGACCTACCAGAAACTGAAGCTGATT
ATGGAACAGTACGGCGACGAGAAGAATCCCCTGTACAAGTACTACGAGGAAACCGGGAACTACCTGAC
CAAGTACTCCAAAAAGGACAACGGCCCCGTGATCAAGAAGATTAAGTATTACGGCAACAAACTGAACG
CCCATCTGGACATCACCGACGACTACCCCAACAGCAGAAACAAGGTCGTGAAGCTGTCCCTGAAGCCC
TACAGATTCGACGTGTACCTGGACAATGGCGTGTACAAGTTCGTGACCGTGAAGAATCTGGATGTGAT
CAAAAAAGAAAACTACTACGAAGTGAATAGCAAGTGCTATGAGGAAGCTAAGAAGCTGAAGAAGATCA
GCAACCAGGCCGAGTTTATCGCCTCCTTCTACAACAACGATCTGATCAAGATCAACGGCGAGCTGTAT
AGAGTGATCGGCGTGAACAACGACCTGCTGAACCGGATCGAAGTGAACATGATCGACATCACCTACCG
CGAGTACCTGGAAAACATGAACGACAAGAGGCCCCCCAGGATCATTAAGACAATCGCCTCCAAGACCC
AGAGCATTAAGAAGTACAGCACAGACATTCTGGGCAACCTGTATGAAGTGAAATCTAAGAAGCACCCT
CAGATCATCAAAAAGGGCAAAAGGCCGGCGGCCACGAAAAAGGCCGGCCAGGCAAAAAAGAAAAAGgg
atcctacccatacgatgttccagattacgcttacccatacgatgttccagattacgcttaccCatacg
atgttccagattacgcttaaGaattctagcaataaaggatcgtttattttcattggaagcgtgtgttg
gttttttgatcaggcgcgGGTACCGAACGCTGACGTCATCAACCCGCTCCAAGGAATCGCGGGCCCAG
TGTCACTAGGCGGGAACACCCAGCGCGCGTGCGCCCTGGCAGGAAGATGGCTGTGAGGGACAGGGGAG
TGGCGCCCTGCAATATTTGCATGTCGCTATGTGTTCTGGGAAATCACCATAAACGTGAAATGTCTTTG
GATTTGGGAATCTTATAAGTTCTGTATGAGACCACATATAGTAATGAAATTATTGGCACGTTTTAGTA
CTCTGGAAACAGAATCTACTAAAACAAGGCAAAATGCCGTGTTTATCTCGTCAACTTGTTGGCGAGAT
TTTTGGTACCaggaacccctagtgatggagttggccactccctctctgcgcgctcgctcgctcactga
ggccgggcgaccaaaggtcgcccgacgcccgggctttgcccgggcggcctcagtgagcgagcgagcgc
gcagctgcctgcaggggcgcctgatgcggtattttctccttacgcatctgtgcggtatttcacaccgc
atacgtcaaagcaaccatagtacgcgccctgtagcggcgcattaagcgcggcgggtgtggtggttacg
cgcagcgtgaccgctacacttgccagcgccctagcqcccgctcctttcgctttcttcccttcctttct
cgccacgttcgccggctttccccgtcaagctctaaatcgggggctccctttagggttccgatttagtg
ctttacggcacctcgaccccaaaaaacttgatttgggtgatggttcactagtgggccatcgccctga
tagacggtttttcgccctttgacgttggagtccacgttctttaatagtggactcttgttccaaactgg
aacaacactcaaccctatctcgggctattcttttgatttataagggattttgccgatttcggcctatt
ggttaaaaaatgagctgatttaacaaaaatttaacgcgaattttaacaaaatattaacgtttacaatt
ttatggtgcactctcagtacaatctgctctgatgccgcatagttaagccagccccgacacccgccaac
acccgctgacgcgccctgacgggcttgtctgctcccggcatccgcttacagacaagctgtgaccgtct
ccgggagctgcatgtgtcagaggttttcaccgtcatcaccgaaacgcgcgagacgaaagggcctcgtg
atacgcctatttttataggttaatgtcatgataataatggtttcttagacgtcaggtggcacttttcg
gggaaatgtgcgcggaacccctatttgtttatttttctaaatacattcaaatatgtatccgctcatga
gacaataaccctgataaatgcttcaataatattgaaaaaggaagagtatgagtattcaacatttccgt
gtcgcccttattcccttttttgcggcattttgccttcctgtttttgctcacccagaaacgctggtgaa
agtaaaagatgctgaagatcagttgggtgcacgagtgggttacatcgaactggatctcaacagcggta
agatccttgagagttttcgccccgaagaacgttttccaatgatgagcacttttaaagttctgctatgt
ggcgcggtattatcccgtattgacgccgggcaagagcaactcggtcgccgcatacactattctcagaa
tgacttggttgagtactcaccagtcacagaaaagcatcttacggatggcatgacagtaagagaattat
gcagtgctgccataaccatgagtgataacactgcggccaacttacttctgacaacgatcggaggaccg
aaggagctaaccgcttttttgcacaacatgggggatcatgtaactcgccttgatcgttgggaaccgga
gctgaatgaagccataccaaacgacgagcgtgacaccacgatgcctgtagcaatggcaacaacgttgc
gcaaactattaactggcgaactacttactctagcttcccggcaacaattaatagactggatggaggcg
gataaagttgcaggaccacttctgcgctcggcccttccggctggctggtttattgctgataaatctgg
agccggtgagcgtggaagccgcggtatcattgcagcactggggccagatggtaagccctcccgtatcg
tagttatctacacgacggggagtcaggcaactatggatgaacgaaatagacagatcgctgagataggt
gcctcactgattaagcattggtaactgtcagaccaagtttactcatatatactttagattgatttaaa
acttcatttttaatttaaaaggatctaggtgaagatcctttttgataatctcatgaccaaaatccctt
aacgtgagttttcgttccactgagcgtcagaccccgtagaaaagatcaaaggatcttcttgagatcct
ttttttctgcgcgtaatctgctgcttgcaaacaaaaaaaccaccgctaccagcggtggtttgtttgcc
ggatcaagagctaccaactctttttccgaaggtaactggcttcagcagagcgcagataccaaatactg
tccttctagtgtagccgtagttaggccaccacttcaagaactctgtagcaccgcctacatacctcgct
ctgctaatcctgttaccagtggctgctgccagtggcgataagtcgtgtcttaccgggttggactcaag
acgatagttaccggataaggcgcagcggtcgggctgaacggggggttcgtgcacacagcccagcttgg
agcgaacgacctacaccgaactgagatacctacagcgtgagctatgagaaagcgccacgcttcccgaa
gggagaaaggcggacaggtatccggtaagcggcagggtcggaacaggagagcgcacgagggagcttcc
agggggaaacgcctggtatctttatagtcctgtcgggtttcgccacctctgacttgagcgtcgatttt
tgtgatgctcgtcaggggggcggagcctatggaaaaacgccagcaacgcggcctttttacggttcctg
gccttttgctggccttttgctcacatgt
Version 2 of vector 1
SEQ ID NO: 74
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGAGAGGGCCTATTTCCCATGATTCCTTCATATTTGCATATACGATACAAGGCTGTTAGAGAGAT
AATTGGAATTAATTTGACTGTAAACACAAAGATATTAGTACAAAATACGTGACGTAGAAAGTAATAAT
TTCTTGGGTAGTTTGCAGTTTTAAAATTATGTTTTAAAATGGACTATCATATGCTTACCGTAACTTGA
AAGTATTTCGATTTCTTGGCTTTATATATCTTGTGGAAAGGACGAAACACCGcatttgtatagagagg
aaatgtgttttagtactctggaaacagaatctactaaaacaaggcaaaatgccgtgtttatctcgtca
acttgttggcgagatttttCTCGAGCTAGACTAGCATGCTGCCCATGTAAGGAGGCAAGGCCTGGGGA
CACCCGAGATGCCTGGTTATAATTAACCCAGACATGTGGCTGCCCCCCCCCCCCCAACACCTGCTGCC
TCTAAAAATAACCCTGCATGCCATGTTCCCGGCGAAGGGCCAGCTGTCCCCCGCCAGCTAGACTCAGC
ACTTAGTTTAGGAACCAGTGAGCAAGTCAGCCCTTGGGGCAGCCCATACAAGGCCATGGGGCTGGGCA
AGCTGCACGCCTGGGTCCGGGGTGGGCACGGTGCCCGGGCAACGAGCTGAAAGCTCATCTGCTCTCAG
GGGCCCCTCCCTGGGGACAGCCCCTCCTGGCTAGTCACACCCTGTAGGCTCCTCTATATAACCCAGGG
GCACAGGGGCTGCCCTCATTCTACCACCACCTCCACAGCACAGACAGACACTCAGGAGCCAGCCAGCa
ccggtgccaccATGGCCCCAAAGAAGAAGCGGAAGGTCGGTATCCACGGAGTCCCAGCAGCCAAGCGG
AACTACATCCTGGGCCTGGACATCGGCATCACCAGCGTGGGCTACGGCATCATCGACTACGAGACACG
GGACGTGATCGATGCCGGCGTGCGGCTGTTCAAAGAGGCCAACGTGGAAAACAACGAGGGCAGGCGGA
GCAAGAGAGGCGCCAGAAGGCTGAAGCGGCGGAGGCGGCATAGAATCCAGAGAGTGAAGAAGCTGCTG
TTCGACTACAACCTGCTGACCGACCACAGCGAGCTGAGCGGCATCAACCCCTACGAGGCCAGAGTGAA
GGGCCTGAGCCAGAAGCTGAGCGAGGAAGAGTTCTCTGCCGCCCTGCTGCACCTGGCCAAGAGAAGAG
GCGTGCACAACGTGAACGAGGTGGAAGAGGACACCGGCAACGAGCTGTCCACCAAAGAGCAGATCAGC
CGGAACAGCAAGGCCCTGGAAGAGAAATACGTGGCCGAACTGCAGCTGGAACGGCTGAAGAAAGACGG
CGAAGTGCGGGGCAGCATCAACAGATTCAAGACCAGCGACTACGTGAAAGAAGCCAAACAGCTGCTGA
AGGTGCAGAAGGCCTACCACCAGCTGGACCAGAGCTTCATCGACACCTACATCGACCTGCTGGAAACC
CGGCGGACCTACTATGAGGGACCTGGCGAGGGCAGCCCCTTCGGCTGGAAGGACATCAAAGAATGGTA
CGAGATGCTGATGGGCCACTGCACCTACTTCCCCGAGGAACTGCGGAGCGTGAAGTACGCCTACAACG
CCGACCTGTACAACGCCCTGAACGACCTGAACAATCTCGTGATCACCAGGGACGAGAACGAGAAGCTG
GAATATTACGAGAAGTTCCAGATCATCGAGAACGTGTTCAAGCAGAAGAAGAAGCCCACCCTGAAGCA
GATCGCCAAAGAAATCCTCGTGAACGAAGAGGATATTAAGGGCTACAGAGTGACCAGCACCGGCAAGC
CCGAGTTCACCAACCTGAAGGTGTACCACGACATCAAGGACATTACCGCCCGGAAAGAGATTATTGAG
AACGCCGAGCTGCTGGATCAGATTGCCAAGATCCTGACCATCTACCAGAGCAGCGAGGACATCCAGGA
AGAACTGACCAATCTGAACTCCGAGCTGACCCAGGAAGAGATCGAGCAGATCTCTAATCTGAAGGGCT
ATACCGGCACCCACAACCTGAGCCTGAAGGCCATCAACCTGATCCTGGACGAGCTGTGGCACACCAAC
GACAACCAGATCGCTATCTTCAACCGGCTGAAGCTGGTGCCCAAGAAGGTGGACCTGTCCCAGCAGAA
AGAGATCCCCACCACCCTGGTGGACGACTTCATCCTGAGCCCCGTCGTGAAGAGAAGCTTCATCCAGA
GCATCAAAGTGATCAACGCCATCATCAAGAAGTACGGCCTGCCCAACGACATCATTATCGAGCTGGCC
CGCGAGAAGAACTCCAAGGACGCCCAGAAAATGATCAACGAGATGCAGAAGCGGAACCGGCAGACCAA
CGAGCGGATCGAGGAAATCATCCGGACCACCGGCAAAGAGAACGCCAAGTACCTGATCGAGAAGATCA
AGCTGCACGACATGCAGGAAGGCAAGTGCCTGTACAGCCTGGAAGCCATCCCTCTGGAAGATCTGCTG
AACAACCCCTTCAACTATGAGGTGGACCACATCATCCCCAGAAGCGTGTCCTTCGACAACAGCTTCAA
CAACAAGGTGCTCGTGAAGCAGGAAGAAAACAGCAAGAAGGGCAACCGGACCCCATTCCAGTACCTGA
GCAGCAGCGACAGCAAGATCAGCTACGAAACCTTCAAGAAGCACATCCTGAATCTGGCCAAGGGCAAG
GGCAGAATCAGCAAGACCAAGAAAGAGTATCTGCTGGAAGAACGGGACATCAACAGGTTCTCCGTGCA
GAAAGACTTCATCAACCGGAACCTGGTGGATACCAGATACGCCACCAGAGGCCTGATGAACCTGCTGC
GGAGCTACTTCAGAGTGAACAACCTGGACGTGAAAGTGAAGTCCATCAATGGCGGCTTCACCAGCTTT
CTGCGGCGGAAGTGGAAGTTTAAGAAAGAGCGGAACAAGGGGTACAAGCACCACGCCGAGGACGCCCT
GATCATTGCCAACGCCGATTTCATCTTCAAAGAGTGGAAGAAACTGGACAAGGCCAAAAAAGTGATGG
AAAACCAGATGTTCGAGGAAAAGCAGGCCGAGAGCATGCCCGAGATCGAAACCGAGCAGGAGTACAAA
GAGATCTTCATCACCCCCCACCAGATCAAGCACATTAAGGACTTCAAGGACTACAAGTACAGCCACCG
GGTGGACAAGAAGCCTAATAGAGAGCTGATTAACGACACCCTGTACTCCACCCGGAAGGACGACAAGG
GCAACACCCTGATCGTGAACAATCTGAACGGCCTGTACGACAAGGACAATGACAAGCTGAAAAAGCTG
ATCAACAAGAGCCCCGAAAAGCTGCTGATGTACCACCACGACCCCCAGACCTACCAGAAACTGAAGCT
GATTATGGAACAGTACGGCGACGAGAAGAATCCCCTGTACAAGTACTACGAGGAAACCGGGAACTACC
TGACCAAGTACTCCAAAAAGGACAACGGCCCCGTGATCAAGAAGATTAAGTATTACGGCAACAAACTG
AACGCCCATCTGGACATCACCGACGACTACCCCAACAGCAGAAACAAGGTCGTGAAGCTGTCCCTGAA
GCCCTACAGATTCGACGTGTACCTGGACAATGGCGTGTACAAGTTCGTGACCGTGAAGAATCTGGATG
TGATCAAAAAAGAAAACTACTACGAAGTGAATAGCAAGTGCTATGAGGAAGCTAAGAAGCTGAAGAAG
ATCAGCAACCAGGCCGAGTTTATCGCCTCCTTCTACAACAACGATCTGATCAAGATCAACGGCGAGCT
GTATAGAGTGATCGGCGTGAACAACGACCTGCTGAACCGGATCGAAGTGAACATGATCGACATCACCT
ACCGCGAGTACCTGGAAAACATGAACGACAAGAGGCCCCCCAGGATCATTAAGACAATCGCCTCCAAG
ACCCAGAGCATTAAGAAGTACAGCACAGACATTCTGGGCAACCTGTATGAAGTGAAATCTAAGAAGCA
CCCTCAGATCATCAAAAAGGGCAAAAGGCCGGCGGCCACGAAAAAGGCCGGCCAGGCAAAAAAGAAAA
AGggatcctacccatacgatgttccagattacgcttacccatacgatgttccagattacgcttaccCa
tacgatgttccagattacgcttaaGaattctagcaataaaggatcgtttattttcattggaagcgtgt
gttggttttttgatcaggcgcgGGTACCGAACGCTGACGTCATCAACCCGCTCCAAGGAATCGCGGGC
CCAGTGTCACTAGGCGGGAACACCCAGCGCGCGTGCGCCCTGGCAGGAAGATGGCTGTGAGGGACAGG
GGAGTGGCGCCCTGCAATATTTGCATGTCGCTATGTGTTCTGGGAAATCACCATAAACGTGAAATGTC
TTTGGATTTGGGAATCITATAAGTTCTGTATGAGACCACATATAGTAATGAAATTATTGGCACGTTTT
AGTACTCTGGAAACAGAATCTACTAAAACAAGGCAAAATGCCGTGTTTATCTCGTCAACTTGTTGGCG
AGATTTTTGGTACCaggaacccctagtgatggagttggccactccctctctgcgcgctcgctcgctca
ctgaggccgggcgaccaaaggtcgcccgacgcccgggctttgcccgggcggcctcagtgagcgagcga
gcgcgcagctgcctgcaggggcgcctgatgcggtattttctccttacgcatctgtgcggtatttcaca
ccgcatacgtcaaagcaaccatagtacgcgccctgtagcggcgcattaagcgcggcgggtgtggtggt
tacgcgcagcgtgaccgctacacttgccagcgccctagcgcccgctcctttcgctttcttcccttcct
ttctcgccacgttcgccggctttccccgtcaagctctaaatcgggggctccctttagggttccgattt
agtgctttacggcacctcgaccccaaaaaacttgatttgggtgatggttcacgtagtgggccatcgcc
ctgatagacggtttttcgccctttgacgttggagtccacgttctttaatagtggactcttgttccaaa
ctggaacaacactcaaccctatctcgggctattcttttgatttataagggattttgccgatttcggcc
tattggttaaaaaatgagctgatttaacaaaaatttaacgcgaattttaacaaaatattaacgtttac
aattttatggtgcactctcagtacaatctgctctgatgccgcatagttaagccagccccgacacccgc
caacacccgctgacgcgccctgacgggcttgtctgctcccggcatccgcttacagacaagctgtgacc
gtctccgggagctgcatgtgtcagaggttttcaccgtcatcaccgaaacgcgcgagacgaaagggcct
cgtgatacgcctatttttataggttaatgtcatgataataatggtttcttagacgtcaggtggcactt
ttcggggaaatgtgcgcggaacccctatttgtttatttttctaaatacattcaaatatgtatccgctc
atgagacaataaccctgataaatgcttcaataatattgaaaaaggaagagtatgagtattcaacattt
ccgtgtcgcccttattcccttttttgcggcattttgccttcctgtttttgctcacccagaaacgctgg
tgaaagtaaaagatgctgaagatcagttgggtgcacgagtgggttacatcgaactggatctcaacagc
ggtaagatccttgagagttttcgccccgaagaacgttttccaatgatgagcacttttaaagttctgct
atgtggcgcggtattatcccgtattgacgccgggcaagagcaactcggtcgccgcatacactattctc
agaatgacttggttgagtactcaccagtcacagaaaagcatcttacggatggcatgacagtaagagaa
ttatgcagtgctgccataaccatgagtgataacactgcggccaacttacttctgacaacgatcggagg
accgaaggagctaaccgcttttttgcacaacatgggggatcatgtaactcgccttgatcgttgggaac
cggagctgaatgaagccataccaaacgacgagcgtgacaccacgatgcctgtagcaatggcaacaacg
ttgcgcaaactattaactggcgaactacttactctagcttcccggcaacaattaatagactggatgga
ggcggataaagttgcaggaccacttctgcgctcggcccttccggctggctggtttattgctgataaat
ctggagccggtgagcgtggaagccgcggtatcattgcagcactggggccagatggtaagccctcccgt
atcgtagttatctacacgacggggagtcaggcaactatggatgaacgaaatagacagatcgctgagat
aggtgcctcactgattaagcattggtaactgtcagaccaagtttactcatatatactttagattgatt
taaaacttcatttttaatttaaaaggatctaggtgaagatcctttttgataatctcatgaccaaaatc
ccttaacgtgagttttcgttccactgagcgtcagaccccgtagaaaagatcaaaggatcttcttgaga
tcctttttttctgcgcgtaatctgctgcttgcaaacaaaaaaaccaccgctaccagcggtggtttgtt
tgccggatcaagagctaccaactctttttccgaaggtaactggcttcagcagagcgcagataccaaat
actgtccttctagtgtagccgtagttaggccaccacttcaagaactctgtagcaccgcctacatacct
cgctctgctaatcctgttaccagtggctgctgccagtggcgataagtcgtgtcttaccgggttggact
caagacgatagttaccggataaggcgcagcggtcgggctgaacggggggttcgtgcacacagcccagc
ttggagcgaacgacctacaccgaactgagatacctacagcgtgagctatgagaaagcgccacgcttcc
cgaagggagaaaggcggacaggtatccggtaagcggcagggtcggaacaggagagcgcacgagggagc
ttccagggggaaacgcctggtatctttatagtcctgtcgggtttcgccacctctgacttgagcgtcga
tttttgtgatgctcgtcaggggggcggagcctatggaaaaacgccagcaacgcggcctttttacggtt
cctggccttttgctggccttttgctcacatgt
Version 1 of vector 2
SEQ ID NO: 75
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGAaaaaatctcgccaacaagttgacgagataaacacggcattttgccttgttttagtagattct
gtttccagagtactaaaacacatttcctctctatacaaatgCGGTGTTTCGTCCTTTCCACAAGATAT
ATAAAGCCAAGAAATCGAAATACTTTCAAGTTACGGTAAGCATATGATAGTCCATTTTAAAACATAAT
TTTAAAACTGCAAACTACCCAAGAAATTATTACTTTCTACGTCACGTATTTTGTACTAATATCTTTGT
GTTTACAGTCAAATTAATTCCAATTATCTCTCTAACAGCCTTGTATCGTATATGCAAATATGAAGGAA
TCATGGGAAATAGGCCCTCCTCGAGTCGAGTGGCTCCGGTGCCCGTCAGTGGGCAGAGCGCACATCGC
CCACAGTCCCCGAGAAGTTGGGGGGAGGGGTCGGCAATTGAACCGGTGCCTAGAGAAGGTGGCGCGGG
GTAAACTGGGAAAGTGATGTCGTGTACTGGCTCCGCCTTTTTCCCGAGGGTGGGGGAGAACCGTATAT
AAGTGCAGTAGTCGCCGTGAACGTTCTTTTTCGCAACGGGTTTGCCGCCAGAACACAGGTGTCGTGAC
CGCGGCCATGGCCCCAAAGAAGAAGCGGAAGGTCGGTATCCACGGAGTCCCAGCAGCCAAGCGGAACT
ACATCCTGGGCCTGGACATCGGCATCACCAGCGTGGGCTACGGCATCATCGACTACGAGACACGGGAC
GTGATCGATGCCGGCGTGCGGCTGTTCAAAGAGGCCAACGTGGAAAACAACGAGGGCAGGCGGAGCAA
GAGAGGCGCCAGAAGGCTGAAGCGGCGGAGGCGGCATAGAATCCAGAGAGTGAAGAAGCTGCTGTTCG
ACTACAACCTGCTGACCGACCACAGCGAGCTGAGCGGCATCAACCCCTACGAGGCCAGAGTGAAGGGC
CTGAGCCAGAAGCTGAGCGAGGAAGAGTTCTCTGCCGCCCTGCTGCACCTGGCCAAGAGAAGAGGCGT
GCACAACGTGAACGAGGTGGAAGAGGACACCGGCAACGAGCTGTCCACCAAAGAGCAGATCAGCCGGA
ACAGCAAGGCCCTGGAAGAGAAATACGTGGCCGAACTGCAGCTGGAACGGCTGAAGAAAGACGGCGAA
GTGCGGGGCAGCATCAACAGATTCAAGACCAGCGACTACGTGAAAGAAGCCAAACAGCTGCTGAAGGT
GCAGAAGGCCTACCACCAGCIGGACCAGAGCTTCATCGACACCTACATCGACCTGCTGGAAACCCGGC
GGACCTACTATGAGGGACCTGGCGAGGGCAGCCCCTTCGGCTGGAAGGACATCAAAGAATGGTACGAG
ATGCTGATGGGCCACTGCACCTACTTCCCCGAGGAACTGCGGAGCGTGAAGTACGCCTACAACGCCGA
CCTGTACAACGCCCTGAACGACCTGAACAATCTCGTGATCACCAGGGACGAGAACGAGAAGCTGGAAT
ATTACGAGAAGTTCCAGATCATCGAGAACGTGTTCAAGCAGAAGAAGAAGCCCACCCTGAAGCAGATC
GCCAAAGAAATCCTCGTGAACGAAGAGGATATTAAGGGCTACAGAGTGACCAGCACCGGCAAGCCCGA
GTTCACCAACCTGAAGGTGTACCACGACATCAAGGACATTACCGCCCGGAAAGAGATTATTGAGAACG
CCGAGCTGCTGGATCAGATTGCCAAGATCCTGACCATCTACCAGAGCAGCGAGGACATCCAGGAAGAA
CTGACCAATCTGAACTCCGAGCTGACCCAGGAAGAGATCGAGCAGATCTCTAATCTGAAGGGCTATAC
CGGCACCCACAACCTGAGCCTGAAGGCCATCAACCTGATCCTGGACGAGCTGTGGCACACCAACGACA
ACCAGATCGCTATCTTCAACCGGCTGAAGCTGGTGCCCAAGAAGGTGGACCTGTCCCAGCAGAAAGAG
ATCCCCACCACCCTGGTGGACGACTTCATCCTGAGCCCCGTCGTGAAGAGAAGCTTCATCCAGAGCAT
CAAAGTGATCAACGCCATCATCAAGAAGTACGGCCTGCCCAACGACATCATTATCGAGCTGGCCCGCG
AGAAGAACTCCAAGGACGCCCAGAAAATGATCAACGAGATGCAGAAGCGGAACCGGCAGACCAACGAG
CGGATCGAGGAAATCATCCGGACCACCGGCAAAGAGAACGCCAAGTACCTGATCGAGAAGATCAAGCT
GCACGACATGCAGGAAGGCAAGTGCCTGTACAGCCTGGAAGCCATCCCTCTGGAAGATCTGCTGAACA
ACCCCTTCAACTATGAGGTGGACCACATCATCCCCAGAAGCGTGTCCTTCGACAACAGCTTCAACAAC
AAGGTGCTCGTGAAGCAGGAAGAAAACAGCAAGAAGGGCAACCGGACCCCATTCCAGTACCTGAGCAG
CAGCGACAGCAAGATCAGCTACGAAACCTTCAAGAAGCACATCCTGAATCTGGCCAAGGGCAAGGGCA
GAATCAGCAAGACCAAGAAAGAGTATCTGCTGGAAGAACGGGACATCAACAGGTTCTCCGTGCAGAAA
GACTTCATCAACCGGAACCTGGTGGATACCAGATACGCCACCAGAGGCCTGATGAACCTGCTGCGGAG
CTACTTCAGAGTGAACAACCTGGACGTGAAAGTGAAGTCCATCAATGGCGGCTTCACCAGCTTTCTGC
GGCGGAAGTGGAAGTTTAAGAAAGAGCGGAACAAGGGGTACAAGCACCACGCCGAGGACGCCCTGATC
ATTGCCAACGCCGATTTCATCTTCAAAGAGTGGAAGAAACTGGACAAGGCCAAAAAAGTGATGGAAAA
CCAGATGTTCGAGGAAAAGCAGGCCGAGAGCATGCCCGAGATCGAAACCGAGCAGGAGTACAAAGAGA
TCTTCATCACCCCCCACCAGATCAAGCACATTAAGGACTTCAAGGACTACAAGTACAGCCACCGGGTG
GACAAGAAGCCTAATAGAGAGCTGATTAACGACACCCTGTACTCCACCCGGAAGGACGACAAGGGCAA
CACCCTGATCGTGAACAATCTGAACGGCCTGTACGACAAGGACAATGACAAGCTGAAAAAGCTGATCA
ACAAGAGCCCCGAAAAGCTGCTGATGTACCACCACGACCCCCAGACCTACCAGAAACTGAAGCTGATT
ATGGAACAGTACGGCGACGAGAAGAATCCCCTGTACAAGTACTACGAGGAAACCGGGAACTACCTGAC
CAAGTACTCCAAAAAGGACAACGGCCCCGTGATCAAGAAGATTAAGTATTACGGCAACAAACTGAACG
CCCATCTGGACATCACCGACGACTACCCCAACAGCAGAAACAAGGTCGTGAAGCTGTCCCTGAAGCCC
TACAGATTCGACGTGTACCTGGACAATGGCGTGTACAAGTTCGTGACCGTGAAGAATCTGGATGTGAT
CAAAAAAGAAAACTACTACGAAGTGAATAGCAAGTGCTATGAGGAAGCTAAGAAGCTGAAGAAGATCA
GCAACCAGGCCGAGTTTATCGCCTCCTTCTACAACAACGATCTGATCAAGATCAACGGCGAGCTGTAT
AGAGTGATCGGCGTGAACAACGACCTGCTGAACCGGATCGAAGTGAACATGATCGACATCACCTACCG
CGAGTACCTGGAAAACATGAACGACAAGAGGCCCCCCAGGATCATTAAGACAATCGCCTCCAAGACCC
AGAGCATTAAGAAGTACAGCACAGACATTCTGGGCAACCTGTATGAAGTGAAATCTAAGAAGCACCCT
CAGATCATCAAAAAGGGCAAAAGGCCGGCGGCCACGAAAAAGGCCGGCCAGGCAAAAAAGAAAAAGgg
atcctacccatacgatgttccagattacgcttacccatacgatgttccagattacgcttaccCatacg
atgttccagattacgcttaaGaattctagcaataaaggatcgtttattttcattggaagcgtgtgttg
gttttttgatcaggcgcgGGTACCGAACGCTGACGTCATCAACCCGCTCCAAGGAATCGCGGGCCCAG
TGTCACTAGGCGGGAACACCCAGCGCGCGTGCGCCCTGGCAGGAAGATGGCTGTGAGGGACAGGGGAG
TGGCGCCCTGCAATATTTGCATGTCGCTATGTGTTCTGGGAAATCACCATAAACGTGAAATGTCTTTG
GATTTGGGAATCTTATAAGTTCTGTATGAGACCACATATAGTAATGAAATTATTGGCACGTTTTAGTA
CTCTGGAAACAGAATCTACTAAAACAAGGCAAAATGCCGTGTTTATCTCGTCAACTTGTTGGCGAGAT
TTTTGGTACCaggaacccctagtgatggagttggccactccctctctgcgcgctcgctcgctcactga
ggccgggcgaccaaaggtcgcccgacgcccgggctttgcccgggcggcctcagtgagcgagcgagcgc
gcagctgcctgcaggggcgcctgatgcggtattttctccttacgcatctgtgcggtatttcacaccgc
atacgtcaaagcaaccatagtacgcgccctgtagcggcgcattaagcgcggcgggtgtggtggttacg
cgcagcgtgaccgctacacttgccagcgccctagcgcccgctcctttcgctttcttcccttcctttct
cgccacgttcgccggctttccccgtcaagctctaaatcgggggctccctttagggttccgatttagtg
ctttacggcacctcgaccccaaaaaacttgatttgggtgatggttcacgtagtgggccatcgccctga
tagacggtttttcgccctttgacgttggagtccacgttctttaatagtggactcttgttccaaactgg
aacaacactcaaccctatctcgggctattcttttgatttataagggattttgccgatttcggcctatt
ggttaaaaaatgagctgatttaacaaaaatttaacgcgaattttaacaaaatattaacgtttacaatt
ttatggtgcactctcagtacaatctgctctgatgccgcatagttaagccagccccgacacccgccaac
acccgctgacgcgccctgacgggcttgtctgctcccggcatccgcttacagacaagctgtgaccgtct
ccgggagctgcatgtgtcagaggttttcaccgtcatcaccgaaacgcgcgagacgaaagggcctcgtg
atacgcctatttttataggttaatgtcatgataataatggtttcttagacgtcaggtggcacttttcg
gggaaatgtgcgcggaacccctatttgtttatttttctaaatacattcaaatatgtatccgctcatga
gacaataaccctgataaatgcttcaataatattgaaaaaggaagagtatgagtattcaacatttccgt
gtcgcccttattcccttttttgcggcattttgccttcctgtttttgctcacccagaaacgctggtgaa
agtaaaagatgctgaagatcagttgggtgcacgagtgggttacatcgaactggatctcaacagcggta
agatccttgagagttttcgccccgaagaacgttttccaatgatgagcacttttaaagttctgctatgt
ggcgcggtattatcccgtattgacgccgggcaagagcaactcggtcgccgcatacactattctcagaa
tgacttggttgagtactcaccagtcacagaaaagcatcttacggatggcatgacagtaagagaattat
gcagtgctgccataaccatgagtgataacactgcggccaacttacttctgacaacgatcggaggaccg
aaggagctaaccgcttttttgcacaacatgggggatcatgtaactcgccttgatcgttgggaaccgga
gctgaatgaagccataccaaacgacgagcgtgacaccacgatgcctgtagcaatggcaacaacgttgc
gcaaactattaactggcgaactacttactctagcttcccggcaacaattaatagactggatggaggcg
gataaagttgcaggaccacttctgcgctcggcccttccggctggctggtttattgctgataaatctgg
agccggtgagcgtggaagccgcggtatcattgcagcactggggccagatggtaagccctcccgtatcg
tagttatctacacgacggggagtcaggcaactatggatgaacgaaatagacagatcgctgagataggt
gcctcactgattaagcattggtaactgtcagaccaagtttactcatatatactttagattgatttaaa
acttcatttttaatttaaaaggatctaggtgaagatcctttttgataatctcatgaccaaaatccctt
aacgtgagttttcgttccactgagcgtcagaccccgtagaaaagatcaaaggatcttcttgagatcct
ttttttctgcgcgtaatctgctgcttgcaaacaaaaaaaccaccgctaccagcggtggtttgtttgcc
ggatcaagagctaccaactctttttccgaaggtaactggcttcagcagagcgcagataccaaatactg
tccttctagtgtagccgtagttaggccaccacttcaagaactctgtagcaccgcctacatacctcgct
ctgctaatcctgttaccagtggctgctgccagtggcgataagtcgtgtcttaccgggttggactcaag
acgatagttaccggataaggcgcagcggtcgggctgaacggggggttcgtgcacacagcccagcttgg
agcgaacgacctacaccgaactgagatacctacagcgtgagctatgagaaagcgccacgcttcccgaa
gggagaaaggcggacaggtatccggtaagcggcagggtcggaacaggagagcgcacgagggagcttcc
agggggaaacgcctggtatctttatagtcctgtcgggtttcgccacctctgacttgagcgtcgatttt
tgtgatgctcgtcaggggggcggagcctatggaaaaacgccagcaacgcggcctttttacggttcctg
gccttttgctggccttttgctcacatgt
Version 2 of vector 2
SEQ ID NO: 76
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGAaaaaatctcgccaacaagttgacgagataaacacggcattttgccttgttttagtagattct
gtttccagagtactaaaacacatttcctctctatacaaatgCGGTGTTTCGTCCTTTCCACAAGATAT
ATAAAGCCAAGAAATCGAAATACTTTCAAGTTACGGTAAGCATATGATAGTCCATTTTAAAACATAAT
TTTAAAACTGCAAACTACCCAAGAAATTATTACTTICTACGTCACGTATTTTGTACTAATATCTTTGT
GTTTACAGTCAAATTAATTCCAATTATCTCTCTAACAGCCTTGTATCGTATATGCAAATATGAAGGAA
TCATGGGAAATAGGCCCTCCTCGAGCTAGACTAGCATGCTGCCCATGTAAGGAGGCAAGGCCTGGGGA
CACCCGAGATGCCTGGTTATAATTAACCCAGACATGTGGCTGCCCCCCCCCCCCCAACACCTGCTGCC
TCTAAAAATAACCCTGCATGCCATGTTCCCGGCGAAGGGCCAGCTGTCCCCCGCCAGCTAGACTCAGC
ACTTAGTTTAGGAACCAGTGAGCAAGTCAGCCCTTGGGGCAGCCCATACAAGGCCATGGGGCTGGGCA
AGCTGCACGCCTGGGTCCGGGGTGGGCACGGTGCCCGGGCAACGAGCTGAAAGCTCATCTGCTCTCAG
GGGCCCCTCCCTGGGGACAGCCCCTCCTGGCTAGTCACACCCTGTAGGCTCCTCTATATAACCCAGGG
GCACAGGGGCTGCCCTCATTCTACCACCACCTCCACAGCACAGACAGACACTCAGGAGCCAGCCAGCa
ccggtgccaccATGGCCCCAAAGAAGAAGCGGAAGGTCGGTATCCACGGAGTCCCAGCAGCCAAGCGG
AACTACATCCTGGGCCTGGACATCGGCATCACCAGCGTGGGCTACGGCATCATCGACTACGAGACACG
GGACGTGATCGATGCCGGCGTGCGGCTGTTCAAAGAGGCCAACGTGGAAAACAACGAGGGCAGGCGGA
GCAAGAGAGGCGCCAGAAGGCTGAAGCGGCGGAGGCGGCATAGAATCCAGAGAGTGAAGAAGCTGCTG
TTCGACTACAACCTGCTGACCGACCACAGCGAGCTGAGCGGCATCAACCCCTACGAGGCCAGAGTGAA
GGGCCTGAGCCAGAAGCTGAGCGAGGAAGAGTTCTCTGCCGCCCTGCTGCACCTGGCCAAGAGAAGAG
GCGTGCACAACGTGAACGAGGTGGAAGAGGACACCGGCAACGAGCTGTCCACCAAAGAGCAGATCAGC
CGGAACAGCAAGGCCCTGGAAGAGAAATACGTGGCCGAACTGCAGCTGGAACGGCTGAAGAAAGACGG
CGAAGTGCGGGGCAGCATCAACAGATTCAAGACCAGCGACTACGTGAAAGAAGCCAAACAGCTGCTGA
AGGTGCAGAAGGCCTACCACCAGCTGGACCAGAGCTTCATCGACACCTACATCGACCTGCTGGAAACC
CGGCGGACCTACTATGAGGGACCTGGCGAGGGCAGCCCCTTCGGCTGGAAGGACATCAAAGAATGGTA
CGAGATGCTGATGGGCCACTGCACCTACTTCCCCGAGGAACTGCGGAGCGTGAAGTACGCCTACAACG
CCGACCTGTACAACGCCCTGAACGACCTGAACAATCTCGTGATCACCAGGGACGAGAACGAGAAGCTG
GAATATTACGAGAAGTTCCAGATCATCGAGAACGTGTTCAAGCAGAAGAAGAAGCCCACCCTGAAGCA
GATCGCCAAAGAAATCCTCGTGAACGAAGAGGATATTAAGGGCTACAGAGTGACCAGCACCGGCAAGC
CCGAGTTCACCAACCTGAAGGTGTACCACGACATCAAGGACATTACCGCCCGGAAAGAGATTATTGAG
AACGCCGAGCTGCTGGATCAGATTGCCAAGATCCTGACCATCTACCAGAGCAGCGAGGACATCCAGGA
AGAACTGACCAATCTGAACTCCGAGCTGACCCAGGAAGAGATCGAGCAGATCTCTAATCTGAAGGGCT
ATACCGGCACCCACAACCTGAGCCTGAAGGCCATCAACCTGATCCTGGACGAGCTGTGGCACACCAAC
GACAACCAGATCGCTATCTTCAACCGGCTGAAGCTGGTGCCCAAGAAGGTGGACCTGTCCCAGCAGAA
AGAGATCCCCACCACCCTGGTGGACGACTTCATCCTGAGCCCCGTCGTGAAGAGAAGCTTCATCCAGA
GCATCAAAGTGATCAACGCCATCATCAAGAAGTACGGCCTGCCCAACGACATCATTATCGAGCTGGCC
CGCGAGAAGAACTCCAAGGACGCCCAGAAAATGATCAACGAGATGCAGAAGCGGAACCGGCAGACCAA
CGAGCGGATCGAGGAAATCATCCGGACCACCGGCAAAGAGAACGCCAAGTACCTGATCGAGAAGATCA
AGCTGCACGACATGCAGGAAGGCAAGTGCCTGTACAGCCTGGAAGCCATCCCTCTGGAAGATCTGCTG
AACAACCCCTTCAACTATGAGGTGGACCACATCATCCCCAGAAGCGTGTCCTTCGACAACAGCTTCAA
CAACAAGGTGCTCGTGAAGCAGGAAGAAAACAGCAAGAAGGGCAACCGGACCCCATTCCAGTACCTGA
GCAGCAGCGACAGCAAGATCAGCTACGAAACCTTCAAGAAGCACATCCTGAATCTGGCCAAGGGCAAG
GGCAGAATCAGCAAGACCAAGAAAGAGTATCTGCTGGAAGAACGGGACATCAACAGGTTCTCCGTGCA
GAAAGACTTCATCAACCGGAACCTGGTGGATACCAGATACGCCACCAGAGGCCTGATGAACCTGCTGC
GGAGCTACTTCAGAGTGAACAACCTGGACGTGAAAGTGAAGTCCATCAATGGCGGCTTCACCAGCTTT
CTGCGGCGGAAGTGGAAGTTTAAGAAAGAGCGGAACAAGGGGTACAAGCACCACGCCGAGGACGCCCT
GATCATTGCCAACGCCGATTTCATCTTCAAAGAGTGGAAGAAACTGGACAAGGCCAAAAAAGTGATGG
AAAACCAGATGTTCGAGGAAAAGCAGGCCGAGAGCATGCCCGAGATCGAAACCGAGCAGGAGTACAAA
GAGATCTTCATCACCCCCCACCAGATCAAGCACATTAAGGACTTCAAGGACTACAAGTACAGCCACCG
GGTGGACAAGAAGCCTAATAGAGAGCTGATTAACGACACCCTGTACTCCACCCGGAAGGACGACAAGG
GCAACACCCTGATCGTGAACAATCTGAACGGCCTGTACGACAAGGACAATGACAAGCTGAAAAAGCTG
ATCAACAAGAGCCCCGAAAAGCTGCTGATGTACCACCACGACCCCCAGACCTACCAGAAACTGAAGCT
GATTATGGAACAGTACGGCGACGAGAAGAATCCCCTGTACAAGTACTACGAGGAAACCGGGAACTACC
TGACCAAGTACTCCAAAAAGGACAACGGCCCCGTGATCAAGAAGATTAAGTATTACGGCAACAAACTG
AACGCCCATCTGGACATCACCGACGACTACCCCAACAGCAGAAACAAGGTCGTGAAGCTGTCCCTGAA
GCCCTACAGATTCGACGTGTACCTGGACAATGGCGTGTACAAGTTCGTGACCGTGAAGAATCTGGATG
TGATCAAAAAAGAAAACTACTACGAAGTGAATAGCAAGTGCTATGAGGAAGCTAAGAAGCTGAAGAAG
ATCAGCAACCAGGCCGAGTTTATCGCCTCCTTCTACAACAACGATCTGATCAAGATCAACGGCGAGCT
GTATAGAGTGATCGGCGTGAACAACGACCTGCTGAACCGGATCGAAGTGAACATGATCGACATCACCT
ACCGCGAGTACCTGGAAAACATGAACGACAAGAGGCCCCCCAGGATCATTAAGACAATCGCCTCCAAG
ACCCAGAGCATTAAGAAGTACAGCACAGACATTCTGGGCAACCTGTATGAAGTGAAATCTAAGAAGCA
CCCTCAGATCATCAAAAAGGGCAAAAGGCCGGCGGCCACGAAAAAGGCCGGCCAGGCAAAAAAGAAAA
AGggatcctacccatacgatgttccagattacgcttacccatacgatgttccagattacgcttaccCa
tacgatgttccagattacgcttaaGaattctagcaataaaggatcgtttattttcattggaagcgtgt
gttggttttttgatcaggcgcgGGTACCGAACGCTGACGTCATCAACCCGCTCCAAGGAATCGCGGGC
CCAGTGTCACTAGGCGGGAACACCCAGCGCGCGTGCGCCCTGGCAGGAAGATGGCTGTGAGGGACAGG
GGAGTGGCGCCCTGCAATATTTGCATGTCGCTATGTGTTCTGGGAAATCACCATAAACGTGAAATGTC
TTTGGATTTGGGAATCTTATAAGTTCTGTATGAGACCACATATAGTAATGAAATTATTGGCACGTTTT
AGTACTCTGGAAACAGAATCTACTAAAACAAGGCAAAATGCCGTGTTTATCTCGTCAACTTGTTGGCG
AGATTTTTGGTACCaggaacccctagtgatggagttggccactccctctctgcgcgctcgctcgctca
ctgaggccgggcgaccaaaggtcgcccgacgcccgggctttgcccgggcggcctcagtgagcgagcga
gcgcgcagctgcctgcaggggcgcctgatgcggtattttctccttacgcatctgtgcggtatttcaca
ccgcatacgtcaaagcaaccatagtacgcgccctgtagcggcgcattaagcgcggcgggtgtggtggt
tacgcgcagcgtgaccgctacacttgccagcgccctagcgcccgctcctttcgctttcttcccttcct
ttctcgccacgttcgccggctttccccgtcaagctctaaatcgggggctccctttagggttccgattt
agtgctttacggcacctcgaccccaaaaaacttgatttgggtgatggttcacgtagtgggccatcgcc
ctgatagacggtttttcgccctttgacgttggagtccacgttctttaatagtggactcttgttccaaa
ctggaacaacactcaaccctatctcgggctattcttttgatttataagggattttgccgatttcggcc
tattggttaaaaaatgagctgatttaacaaaaatttaacgcgaattttaacaaaatattaacgtttac
aattttatggtgcactctcagtacaatctgctctgatgccgcatagttaagccagccccgacacccgc
caacacccgctgacgcgccctgacgggcttgtctgctcccggcatccgcttacagacaagctgtgacc
gtctccgggagctgcatgtgtcagaggttttcaccgtcatcaccgaaacgcgcgagacgaaagggcct
cgtgatacgcctatttttataggttaatgtcatgataataatggtttcttagacgtcaggtggcactt
ttcggggaaatgtgcgcggaacccctatttgtttatttttctaaatacattcaaatatgtatccgctc
atgagacaataaccctgataaatgcttcaataatattgaaaaaggaagagtatgagtattcaacattt
ccgtgtcgcccttattcccttttttgcggcattttgccttcctgtttttgctcacccagaaacgctgg
tgaaagtaaaagatgctgaagatcagttgggtgcacgagtgggttacatcgaactggatctcaacagc
ggtaagatccttgagagttttcgccccgaagaacgttttccaatgatgagcacttttaaagttctgct
atgtggcgcggtattatcccgtattgacgccgggcaagagcaactcggtcgccgcatacactattctc
agaatgacttggttgagtactcaccagtcacagaaaagcatcttacggatggcatgacagtaagagaa
ttatgcagtgctgccataaccatgagtgataacactgcggccaacttacttctgacaacgatcggagg
accgaaggagctaaccgcttttttgcacaacatgggggatcatgtaactcgccttgatcgttgggaac
cggagctgaatgaagccataccaaacgacgagcgtgacaccacgatgcctgtagcaatggcaacaacg
ttgcgcaaactattaactggcgaactacttactctagcttcccggcaacaattaatagactggatgga
ggcggataaagttgcaggaccacttctgcgctcggcccttccggctggctggtttattgctgataaat
ctggagccggtgagcgtggaagccgcggtatcattgcagcactggggccagatggtaagccctcccgt
atcgtagttatctacacgacggggagtcaggcaactatggatgaacgaaatagacagatcgctgagat
aggtgcctcactgattaagcattggtaactgtcagaccaagtttactcatatatactttagattgatt
taaaacttcatttttaatttaaaaggatctaggtgaagatcctttttgataatctcatgaccaaaatc
ccttaacgtgagttttcgttccactgagcgtcagaccccgtagaaaagatcaaaggatcttcttgaga
tcctttttttctgcgcgtaatctgctgcttgcaaacaaaaaaaccaccgctaccagcggtggtttgtt
tgccggatcaagagctaccaactctttttccgaaggtaactggcttcagcagagcgcagataccaaat
actgtccttctagtgtagccgtagttaggccaccacttcaagaactctgtagcaccgcctacatacct
cgctctgctaatcctgttaccagtggctgctgccagtggcgataagtcgtgtcttaccgggttggact
caagacgatagttaccggataaggcgcagcggtcgggctgaacggggggttcgtgcacacagcccagc
ttggagcgaacgacctacaccgaactgagatacctacagcgtgagctatgagaaagcgccacgcttcc
cgaagggagaaaggcggacaggtatccggtaagcggcagggtcggaacaggagagcgcacgagggagc
ttccagggggaaacgcctggtatctttatagtcctgtcgggtttcgccacctctgacttgagcgtcga
tttttgtgatgctcgtcaggggggcggagcctatggaaaaacgccagcaacgcggcctttttacggtt
cctggccttttgctggccttttgctcacatgt
Version 3 of vector 2
SEQ ID NO: 77
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGAaaaaatctcgccaacaagttgacgagataaacacggcattttgccttgttttagtagattct
gtttccagagtactaaaacacatttcctctctatacaaatgCGGTGTTTCGTCCTTTCCACAAGATAT
ATAAAGCCAAGAAATCGAAATACTTTCAAGTTACGGTAAGCATATGATAGTCCATTTTAAAACATAAT
TTTAAAACTGCAAACTACCCAAGAAATTATTACTTTCTACGTCACGTATTTTGTACTAATATCTTTGT
GTTTACAGTCAAATTAATTCCAATTATCTCTCTAACAGCCTTGTATCGTATATGCAAATATGAAGGAA
TCATGGGAAATAGGCCCTCCTCGAGGAGCTCCACCGCGGTGGCGGCCGTCCGCCtTCGGCACCATCCT
CACGACACCCAAATATGGCGACGGGTGAGGAATGGTGGGGAGTTATTTTTAGAGCGGTGAGGAAGGTG
GGCAGGCAGCAGGTGTTGGCGCTCTAAAAATAACTCCCGGGAGTTATTTTTAGAGCGGAGGAATGGTG
GACACCCAAATATGGCGACGGTTCCTCACCCGTCGCCATATTTGGGTGTCCGCCCTCGGCCGGGGCCG
CATTCCTGGGGGCCGGGCGGTGCTCCCGCCCGCCTCGATAAAAGGCTCCGGGGCCGGCGGCGGCCCAC
GAGCTACCCGGAGGAGCGGGAGGCGCCAAGCTCTAGAACTAGTGGATCCCCCGGGCTGCAGGAATTCG
ATATaccggtgccaccATGGCCCCAAAGAAGAAGCGGAAGGTCGGTATCCACGGAGTCCCAGCAGCCA
AGCGGAACTACATCCTGGGCCTGGACATCGGCATCACCAGCGTGGGCTACGGCATCATCGACTACGAG
ACACGGGACGTGATCGATGCCGGCGTGCGGCTGTTCAAAGAGGCCAACGTGGAAAACAACGAGGGCAG
GCGGAGCAAGAGAGGCGCCAGAAGGCTGAAGCGGCGGAGGCGGCATAGAATCCAGAGAGTGAAGAAGC
TGCTGTTCGACTACAACCTGCTGACCGACCACAGCGAGCTGAGCGGCATCAACCCCTACGAGGCCAGA
GTGAAGGGCCTGAGCCAGAAGCTGAGCGAGGAAGAGTTCTCTGCCGCCCTGCTGCACCTGGCCAAGAG
AAGAGGCGTGCACAACGTGAACGAGGTGGAAGAGGACACCGGCAACGAGCTGTCCACCAAAGAGCAGA
TCAGCCGGAACAGCAAGGCCCTGGAAGAGAAATACGTGGCCGAACTGCAGCTGGAACGGCTGAAGAAA
GACGGCGAAGTGCGGGGCAGCATCAACAGATTCAAGACCAGCGACTACGTGAAAGAAGCCAAACAGCT
GCTGAAGGTGCAGAAGGCCTACCACCAGCTGGACCAGAGCTTCATCGACACCTACATCGACCTGCTGG
AAACCCGGCGGACCTACTATGAGGGACCTGGCGAGGGCAGCCCCTTCGGCTGGAAGGACATCAAAGAA
TGGTACGAGATGCTGATGGGCCACTGCACCTACTTCCCCGAGGAACTGCGGAGCGTGAAGTACGCCTA
CAACGCCGACCTGTACAACGCCCTGAACGACCTGAACAATCTCGTGATCACCAGGGACGAGAACGAGA
AGCTGGAATATTACGAGAAGTTCCAGATCATCGAGAACGTGTTCAAGCAGAAGAAGAAGCCCACCCTG
AAGCAGATCGCCAAAGAAATCCTCGTGAACGAAGAGGATATTAAGGGCTACAGAGTGACCAGCACCGG
CAAGCCCGAGTTCACCAACCTGAAGGTGTACCACGACATCAAGGACATTACCGCCCGGAAAGAGATTA
TTGAGAACGCCGAGCTGCTGGATCAGATTGCCAAGATCCTGACCATCTACCAGAGCAGCGAGGACATC
CAGGAAGAACTGACCAATCTGAACTCCGAGCTGACCCAGGAAGAGATCGAGCAGATCTCTAATCTGAA
GGGCTATACCGGCACCCACAACCTGAGCCTGAAGGCCATCAACCTGATCCTGGACGAGCTGTGGCACA
CCAACGACAACCAGATCGCTATCTTCAACCGGCTGAAGCTGGTGCCCAAGAAGGTGGACCTGTCCCAG
CAGAAAGAGATCCCCACCACCCTGGTGGACGACTTCATCCTGAGCCCCGTCGTGAAGAGAAGCTTCAT
CCAGAGCATCAAAGTGATCAACGCCATCATCAAGAAGTACGGCCTGCCCAACGACATCATTATCGAGC
TGGCCCGCGAGAAGAACTCCAAGGACGCCCAGAAAATGATCAACGAGATGCAGAAGCGGAACCGGCAG
ACCAACGAGCGGATCGAGGAAATCATCCGGACCACCGGCAAAGAGAACGCCAAGTACCTGATCGAGAA
GATCAAGCTGCACGACATGCAGGAAGGCAAGTGCCTGTACAGCCTGGAAGCCATCCCTCTGGAAGATC
TGCTGAACAACCCCTTCAACTATGAGGTGGACCACATCATCCCCAGAAGCGTGTCCTTCGACAACAGC
TTCAACAACAAGGTGCTCGTGAAGCAGGAAGAAAACAGCAAGAAGGGCAACCGGACCCCATTCCAGTA
CCTGAGCAGCAGCGACAGCAAGATCAGCTACGAAACCTTCAAGAAGCACATCCTGAATCTGGCCAAGG
GCAAGGGCAGAATCAGCAAGACCAAGAAAGAGTATCTGCTGGAAGAACGGGACATCAACAGGTTCTCC
GTGCAGAAAGACTTCATCAACCGGAACCTGGTGGATACCAGATACGCCACCAGAGGCCTGATGAACCT
GCTGCGGAGCTACTTCAGAGTGAACAACCTGGACGTGAAAGTGAAGTCCATCAATGGCGGCTTCACCA
GCTTTCTGCGGCGGAAGTGGAAGTTTAAGAAAGAGCGGAACAAGGGGTACAAGCACCACGCCGAGGAC
GCCCTGATCATTGCCAACGCCGATTTCATCTTCAAAGAGTGGAAGAAACTGGACAAGGCCAAAAAAGT
GATGGAAAACCAGATGTTCGAGGAAAAGCAGGCCGAGAGCATGCCCGAGATCGAAACCGAGCAGGAGT
ACAAAGAGATCTTCATCACCCCCCACCAGATCAAGCACATTAAGGACTTCAAGGACTACAAGTACAGC
CACCGGGTGGACAAGAAGCCTAATAGAGAGCTGATTAACGACACCCTGTACTCCACCCGGAAGGACGA
CAAGGGCAACACCCTGATCGTGAACAATCTGAACGGCCTGTACGACAAGGACAATGACAAGCTGAAAA
AGCTGATCAACAAGAGCCCCGAAAAGCTGCTGATGTACCACCACGACCCCCAGACCTACCAGAAACTG
AAGCTGATTATGGAACAGTACGGCGACGAGAAGAATCCCCTGTACAAGTACTACGAGGAAACCGGGAA
CTACCTGACCAAGTACTCCAAAAAGGACAACGGCCCCGTGATCAAGAAGATTAAGTATTACGGCAACA
AACTGAACGCCCATCTGGACATCACCGACGACTACCCCAACAGCAGAAACAAGGTCGTGAAGCTGTCC
CTGAAGCCCTACAGATTCGACGTGTACCTGGACAATGGCGTGTACAAGTTCGTGACCGTGAAGAATCT
GGATGTGATCAAAAAAGAAAACTACTACGAAGTGAATAGCAAGTGCTATGAGGAAGCTAAGAAGCTGA
AGAAGATCAGCAACCAGGCCGAGTTTATCGCCTCCTTCTACAACAACGATCTGATCAAGATCAACGGC
GAGCTGTATAGAGTGATCGGCGTGAACAACGACCTGCTGAACCGGATCGAAGTGAACATGATCGACAT
CACCTACCGCGAGTACCTGGAAAACATGAACGACAAGAGGCCCCCCAGGATCATTAAGACAATCGCCT
CCAAGACCCAGAGCATTAAGAAGTACAGCACAGACATTCTGGGCAACCTGTATGAAGTGAAATCTAAG
AAGCACCCTCAGATCATCAAAAAGGGCAAAAGGCCGGCGGCCACGAAAAAGGCCGGCCAGGCAAAAAA
GAAAAAGggatcctacccatacgatgttccagattacgcttacccatacgatgttccagattacgctt
accCatacgatgttccagattacgcttaaGaattctagcaataaaggatcgtttattttcattggaag
cgtgtgttggttttttgatcaggcgcgGGTACCGAACGCTGACGTCATCAACCCGCTCCAAGGAATCG
CGGGCCCAGTGTCACTAGGCGGGAACACCCAGCGCGCGTGCGCCCTGGCAGGAAGATGGCTGTGAGGG
ACAGGGGAGTGGCGCCCTGCAATATTTGCATGTCGCTATGTGTTCTGGGAAATCACCATAAACGTGAA
ATGTCTTTGGATTTGGGAATCTTATAAGTTCTGTATGAGACCACATATAGTAATGAAATTATTGGCAC
GTTTTAGTACTCTGGAAACAGAATCTACTAAAACAAGGCAAAATGCCGTGTTTATCTCGTCAACTTGT
TGGCGAGATTTTTGGTACCaggaacccctagtgatggagttggccactccctctctgcgcgctcgctc
gctcactgaggccgggcgaccaaaggtcgcccgacgcccgggctttgcccgggcggcctcagtgagcg
agcgagcgcgcagctgcctgcaggggcgcctgatgcggtattttctccttacgcatctgtgcggtatt
tcacaccgcatacgtcaaagcaaccatagtacgcgccctgtagcggcgcattaagcgcggcgggtgtg
gtggttacgcgcagcgtgaccgctacacttgccagcgccctagcgcccgctcctttcgctttcttccc
ttcctttctcgccacgttcgccggctttccccgtcaagctctaaatcgggggctccctttagggttcc
gatttagtgctttacggcacctcgaccccaaaaaacttgatttgggtgatggttcacgtagtgggcca
tcgccctgatagacggtttttcgccctttgacgttggagtccacgttctttaatagtggactcttgtt
ccaaactggaacaacactcaaccctatctcgggctattcttttgatttataagggattttgccgattt
cggcctattggttaaaaaatgagctgatttaacaaaaatttaacgcgaattttaacaaaatattaacg
tttacaattttatggtgcactctcagtacaatctgctctgatgccgcatagttaagccagccccgaca
cccgccaacacccgctgacgcgccctgacgggcttgtctgctcccggcatccgcttacagacaagctg
tgaccgtctccgggagctgcatgtgtcagaggttttcaccgtcatcaccgaaacgcgcgagacgaaag
ggcctcgtgatacgcctatttttataggttaatgtcatgataataatggtttcttagacgtcaggtgg
cacttttcggggaaatgtgcgcggaacccctatttgtttatttttctaaatacattcaaatatgtatc
cgctcatgagacaataaccctgataaatgcttcaataatattgaaaaaggaagagtatgagtattcaa
catttccgtgtcgcccttattcccttttttgcggcattttgccttcctgtttttgctcacccagaaac
gctggtgaaagtaaaagatgctgaagatcagttgggtgcacgagtgggttacatcgaactggatctca
acagcggtaagatccttgagagttttcgccccgaagaacgttttccaatgatgagcacttttaaagtt
ctgctatgtggcgcggtattatcccgtattgacgccgggcaagagcaactcggtcgccgcatacacta
ttctcagaatgacttggttgagtactcaccagtcacagaaaagcatcttacggatggcatgacagtaa
gagaattatgcagtgctgccataaccatgagtgataacactgcggccaacttacttctgacaacgatc
ggaggaccgaaggagctaaccgcttttttgcacaacatgggggatcatgtaactcgccttgatcgttg
ggaaccggagctgaatgaagccataccaaacgacgagcgtgacaccacgatgcctgtagcaatggcaa
caacgttgcgcaaactattaactggcgaactacttactctagcttcccggcaacaattaatagactgg
atggaggcggataaagttgcaggaccacttctgcgctcggcccttccggctggctggtttattgctga
taaatctggagccggtgagcgtggaagccgcggtatcattgcagcactggggccagatggtaagccct
cccgtatcgtagttatctacacgacggggagtcaggcaactatggatgaacgaaatagacagatcgct
gagataggtgcctcactgattaagcattggtaactgtcagaccaagtttactcatatatactttagat
tgatttaaaacttcatttttaatttaaaaggatctaggtgaagatcctttttgataatctcatgacca
aaatcccttaacgtgagttttcgttccactgagcgtcagaccccgtagaaaagatcaaaggatcttct
tgagatcctttttttctgcgcgtaatctgctgcttgcaaacaaaaaaaccaccgctaccagcggtggt
ttgtttgccggatcaagagctaccaactctttttccgaaggtaactggcttcagcagagcgcagatac
caaatactgtccttctagtgtagccgtagttaggccaccacttcaagaactctgtagcaccgcctaca
tacctcgctctgctaatcctgttaccagtggctgctgccagtggcgataagtcgtgtcttaccgggtt
ggactcaagacgatagttaccggataaggcgcagcggtcgggctgaacggggggttcgtgcacacagc
ccagcttggagcgaacgacctacaccgaactgagatacctacagcgtgagctatgagaaagcgccacg
cttcccgaagggagaaaggcggacaggtatccggtaagcggcagggtcggaacaggagagcgcacgag
ggagcttccagggggaaacgcctggtatctttatagtcctgtcgggtttcgccacctctgacttgagc
gtcgatttttgtgatgctcgtcaggggggcggagcctatggaaaaacgccagcaacgcggccttttta
cggttcctggccttttgctggccttttgctcacatgt
Version 4 of vector 2
SEQ ID NO: 78
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGAaaaaatctcgccaacaagttgacgagataaacacggcattttgccttgttttagtagattct
gtttccagagtactaaaacacatttcctctctatacaaatgCGGTGTTTCGTCCTTTCCACAAGATAT
ATAAAGCCAAGAAATCGAAATACTTTCAAGTTACGGTAAGCATATGATAGTCCATTTTAAAACATAAT
TTTAAAACTGCAAACTACCCAAGAAATTATTACTTTCTACGTCACGTATTTTGTACTAATATCTTTGT
GTTTACAGTCAAATTAATTCCAATTATCTCTCTAACAGCCTTGTATCGTATATGCAAATATGAAGGAA
TCATGGGAAATAGGCCCTCctcgagGtttaaacaagcttgcatgtctaagctagacccttcagattaa
aaataactgaggtaagggcctgggtaggggaggtggtgtgagacgctcctgtctctcctctatctgcc
catcggccctttggggaggaggaatgtgcccaaggactaaaaaaaggccatggagccagaggggcgag
ggcaacagacctttcatgggcaaaccttggggccctgctgtctagcatgccccactacgggtctaggc
tgcccatgtaaggaggcaaggcctggggacacccgagatgcctggttataattaacccagacatgtgg
ctgcccccccccccccaacacctgctgcctctaaaaataaccctgtccctggtggatcccctgcatgc
gaagatcttcgaacaaggctgtgggggactgagggcaggctgtaacaggcttgggggccagggcttat
acgtgcctgggactcccaaagtattactgttccatgttcccggcgaagggccagctgtcccccgccag
ctagactcagcacttagtttaggaaccagtgagcaagtcagcccttggggcagcccatacaaggccat
ggggctgggcaagctgcacgcctgggtccggggtgggcacggtgcccgggcaacgagctgaaagctca
tctgctctcaggggcccctccctggggacagcccctcctggctagtcacaccctgtaggctcctctat
ataacccaggggcacaggggctgccctcattctaccaccacctccacagcacagacagacactcagga
gccagccagcggcgcgcccaccggtgccaccATGGCCCCAAAGAAGAAGCGGAAGGTCGGTATCCACG
GAGTCCCAGCAGCCAAGCGGAACTACATCCTGGGCCTGGACATCGGCATCACCAGCGTGGGCTACGGC
ATCATCGACTACGAGACACGGGACGTGATCGATGCCGGCGTGCGGCTGTTCAAAGAGGCCAACGTGGA
AAACAACGAGGGCAGGCGGAGCAAGAGAGGCGCCAGAAGGCTGAAGCGGCGGAGGCGGCATAGAATCC
AGAGAGTGAAGAAGCTGCTGTTCGACTACAACCTGCTGACCGACCACAGCGAGCTGAGCGGCATCAAC
CCCTACGAGGCCAGAGTGAAGGGCCTGAGCCAGAAGCTGAGCGAGGAAGAGTTCTCTGCCGCCCTGCT
GCACCTGGCCAAGAGAAGAGGCGTGCACAACGTGAACGAGGTGGAAGAGGACACCGGCAACGAGCTGT
CCACCAAAGAGCAGATCAGCCGGAACAGCAAGGCCCTGGAAGAGAAATACGTGGCCGAACTGCAGCTG
GAACGGCTGAAGAAAGACGGCGAAGTGCGGGGCAGCATCAACAGATTCAAGACCAGCGACTACGTGAA
AGAAGCCAAACAGCTGCTGAAGGTGCAGAAGGCCTACCACCAGCTGGACCAGAGCTTCATCGACACCT
ACATCGACCTGCTGGAAACCCGGCGGACCTACTATGAGGGACCTGGCGAGGGCAGCCCCTTCGGCTGG
AAGGACATCAAAGAATGGTACGAGATGCTGATGGGCCACTGCACCTACTTCCCCGAGGAACTGCGGAG
CGTGAAGTACGCCTACAACGCCGACCTGTACAACGCCCTGAACGACCTGAACAATCTCGTGATCACCA
GGGACGAGAACGAGAAGCTGGAATATTACGAGAAGTTCCAGATCATCGAGAACGTGTTCAAGCAGAAG
AAGAAGCCCACCCTGAAGCAGATCGCCAAAGAAATCCTCGTGAACGAAGAGGATATTAAGGGCTACAG
AGTGACCAGCACCGGCAAGCCCGAGTTCACCAACCTGAAGGTGTACCACGACATCAAGGACATTACCG
CCCGGAAAGAGATTATTGAGAACGCCGAGCTGCTGGATCAGATTGCCAAGATCCTGACCATCTACCAG
AGCAGCGAGGACATCCAGGAAGAACTGACCAATCTGAACTCCGAGCTGACCCAGGAAGAGATCGAGCA
GATCTCTAATCTGAAGGGCTATACCGGCACCCACAACCTGAGCCTGAAGGCCATCAACCTGATCCTGG
ACGAGCTGTGGCACACCAACGACAACCAGATCGCTATCTTCAACCGGCTGAAGCTGGTGCCCAAGAAG
GTGGACCTGTCCCAGCAGAAAGAGATCCCCACCACCCTGGTGGACGACTTCATCCTGAGCCCCGTCGT
GAAGAGAAGCTTCATCCAGAGCATCAAAGTGATCAACGCCATCATCAAGAAGTACGGCCTGCCCAACG
ACATCATTATCGAGCTGGCCCGCGAGAAGAACTCCAAGGACGCCCAGAAAATGATCAACGAGATGCAG
AAGCGGAACCGGCAGACCAACGAGCGGATCGAGGAAATCATCCGGACCACCGGCAAAGAGAACGCCAA
GTACCTGATCGAGAAGATCAAGCTGCACGACATGCAGGAAGGCAAGTGCCTGTACAGCCTGGAAGCCA
TCCCTCTGGAAGATCTGCTGAACAACCCCTTCAACTATGAGGTGGACCACATCATCCCCAGAAGCGTG
TCCTTCGACAACAGCTTCAACAACAAGGTGCTCGTGAAGCAGGAAGAAAACAGCAAGAAGGGCAACCG
GACCCCATTCCAGTACCTGAGCAGCAGCGACAGCAAGATCAGCTACGAAACCTTCAAGAAGCACATCC
TGAATCTGGCCAAGGGCAAGGGCAGAATCAGCAAGACCAAGAAAGAGTATCTGCTGGAAGAACGGGAC
ATCAACAGGTTCTCCGTGCAGAAAGACTTCATCAACCGGAACCTGGTGGATACCAGATACGCCACCAG
AGGCCTGATGAACCTGCTGCGGAGCTACTTCAGAGTGAACAACCTGGACGTGAAAGTGAAGTCCATCA
ATGGCGGCTTCACCAGCTTTCTGCGGCGGAAGTGGAAGTTTAAGAAAGAGCGGAACAAGGGGTACAAG
CACCACGCCGAGGACGCCCTGATCATTGCCAACGCCGATTTCATCTTCAAAGAGTGGAAGAAACTGGA
CAAGGCCAAAAAAGTGATGGAAAACCAGATGTTCGAGGAAAAGCAGGCCGAGAGCATGCCCGAGATCG
AAACCGAGCAGGAGTACAAAGAGATCTTCATCACCCCCCACCAGATCAAGCACATTAAGGACTTCAAG
GACTACAAGTACAGCCACCGGGTGGACAAGAAGCCTAATAGAGAGCTGATTAACGACACCCTGTACTC
CACCCGGAAGGACGACAAGGGCAACACCCTGATCGTGAACAATCTGAACGGCCTGTACGACAAGGACA
ATGACAAGCTGAAAAAGCTGATCAACAAGAGCCCCGAAAAGCTGCTGATGTACCACCACGACCCCCAG
ACCTACCAGAAACTGAAGCTGATTATGGAACAGTACGGCGACGAGAAGAATCCCCTGTACAAGTACTA
CGAGGAAACCGGGAACTACCTGACCAAGTACTCCAAAAAGGACAACGGCCCCGTGATCAAGAAGATTA
AGTATTACGGCAACAAACTGAACGCCCATCTGGACATCACCGACGACTACCCCAACAGCAGAAACAAG
GTCGTGAAGCTGTCCCTGAAGCCCTACAGATTCGACGTGTACCTGGACAATGGCGTGTACAAGTTCGT
GACCGTGAAGAATCTGGATGTGATCAAAAAAGAAAACTACTACGAAGTGAATAGCAAGTGCTATGAGG
AAGCTAAGAAGCTGAAGAAGATCAGCAACCAGGCCGAGTTTATCGCCTCCTTCTACAACAACGATCTG
ATCAAGATCAACGGCGAGCTGTATAGAGTGATCGGCGTGAACAACGACCTGCTGAACCGGATCGAAGT
GAACATGATCGACATCACCTACCGCGAGTACCTGGAAAACATGAACGACAAGAGGCCCCCCAGGATCA
TTAAGACAATCGCCTCCAAGACCCAGAGCATTAAGAAGTACAGCACAGACATTCTGGGCAACCTGTAT
GAAGTGAAATCTAAGAAGCACCCTCAGATCATCAAAAAGGGCAAAAGGCCGGCGGCCACGAAAAAGGC
CGGCCAGGCAAAAAAGAAAAAGggatcctacccatacgatgttccagattacgcttacccatacgatg
ttccagattacgcttaccCatacgatgttccagattacgcttaaGaattctagcaataaaggatcgtt
tattttcattggaagcgtgtgttggttttttgatcaggcgcgGGTACCGAACGCTGACGTCATCAACC
CGCTCCAAGGAATCGCGGGCCCAGTGTCACTAGGCGGGAACACCCAGCGCGCGTGCGCCCTGGCAGGA
AGATGGCTGTGAGGGACAGGGGAGTGGCGCCCTGCAATATTTGCATGTCGCTATGTGTTCTGGGAAAT
CACCATAAACGTGAAATGTCTTTGGATTTGGGAATCTTATAAGTTCTGTATGAGACCACATATAGTAA
TGAAATTATTGGCACGTTTTAGTACTCTGGAAACAGAATCTACTAAAACAAGGCAAAATGCCGTGTTT
ATCTCGTCAACTTGTTGGCGAGATTTTTGGTACCaggaacccctagtgatggagttggccactccctc
tctgcgcgctcgctcgctcactgaggccgggcgaccaaaggtcgcccgacgcccgggctttgcccggg
cggcctcagtgagcgagcgagcgcgcagctgcctgcaggggcgcctgatgcggtattttctccttacg
catctgtgcggtatttcacaccgcatacgtcaaagcaaccatagtacgcgccctgtagcggcgcatta
agcgcggcgggtgtggtggttacgcgcagcgtgaccgctacacttgccagcgccctagcgcccgctcc
tttcgctttcttcccttcctttctcgccacgttcgccggctttccccgtcaagctctaaatcgggggc
tccctttagggttccgatttagtgctttacggcacctcgaccccaaaaaacttgatttgggtgatggt
tcacgtagtgggccatcgccctgatagacggtttttcgccctttgacgttggagtccacgttctttaa
tagtggactcttgttccaaactggaacaacactcaaccctatctcgggctattcttttgatttataag
ggattttgccgatttcggcctattggttaaaaaatgagctgatttaacaaaaatttaacgcgaatttt
aacaaaatattaacgtttacaattttatggtgcactctcagtacaatctgctctgatgccgcatagtt
aagccagccccgacacccgccaacacccgctgacgcgccctgacgggcttgtctgctcccggcatccg
cttacagacaagctgtgaccgtctccgggagctgcatgtgtcagaggttttcaccgtcatcaccgaaa
cgcgcgagacgaaagggcctcgtgatacgcctatttttataggttaatgtcatgataataatggtttc
ttagacgtcaggtggcacttttcggggaaatgtgcgcggaacccctatttgtttatttttctaaatac
attcaaatatgtatccgctcatgagacaataaccctgataaatgcttcaataatattgaaaaaggaag
agtatgagtattcaacatttccgtgtcgcccttattcccttttttgcggcattttgccttcctgtttt
tgctcacccagaaacgctggtgaaagtaaaagatgctgaagatcagttgggtgcacgagtgggttaca
tcgaactggatctcaacagcggtaagatccttgagagttttcgccccgaagaacgttttccaatgatg
agcacttttaaagttctgctatgtggcgcggtattatcccgtattgacgccgggcaagagcaactcgg
tcgccgcatacactattctcagaatgacttggttgagtactcaccagtcacagaaaagcatcttacgg
atggcatgacagtaagagaattatgcagtgctgccataaccatgagtgataacactgcggccaactta
cttctgacaacgatcggaggaccgaaggagctaaccgcttttttgcacaacatgggggatcatgtaac
tcgccttgatcgttgggaaccggagctgaatgaagccataccaaacgacgagcgtgacaccacgatgc
ctgtagcaatggcaacaacgttgcgcaaactattaactggcgaactacttactctagcttcccggcaa
caattaatagactggatggaggcggataaagttgcaggaccacttctgcgctcggcccttccggctgg
ctggtttattgctgataaatctggagccggtgagcgtggaagccgcggtatcattgcagcactggggc
cagatggtaagccctcccgtatcgtagttatctacacgacggggagtcaggcaactatggatgaacga
aatagacagatcgctgagataggtgcctcactgattaagcattggtaactgtcagaccaagtttactc
atatatactttagattgatttaaaacttcatttttaatttaaaaggatctaggtgaagatcctttttg
ataatctcatgaccaaaatcccttaacgtgagttttcgttccactgagcgtcagaccccgtagaaaag
atcaaaggatcttcttgagatcctttttttctgcgcgtaatctgctgcttgcaaacaaaaaaaccacc
gctaccagcggtggtttgtttgccggatcaagagctaccaactctttttccgaaggtaactggcttca
gcagagcgcagataccaaatactgtccttctagtgtagccgtagttaggccaccacttcaagaactct
gtagcaccgcctacatacctcgctctgctaatcctgttaccagtggctgctgccagtggcgataagtc
gtgtcttaccgggttggactcaagacgatagttaccggataaggcgcagcggtcgggctgaacggggg
gttcgtgcacacagcccagcttggagcgaacgacctacaccgaactgagatacctacagcgtgagcta
tgagaaagcgccacgcttcccgaagggagaaaggcggacaggtatccggtaagcggcagggtcggaac
aggagagcgcacgagggagcttccagggggaaacgcctggtatctttatagtcctgtcgggtttcgcc
acctctgacttgagcgtcgatttttgtgatgctcgtcaggggggcggagcctatggaaaaacgccagc
aacgcggcctttttacggttcctggccttttgctggccttttgctcacatgt
Version 1 of vector 3
SEQ ID NO: 79
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGAaaaaatctcgccaacaagttgacgagataaacacggcattttgccttgttttagtagattct
gtttccagagtactaaaacacatttcctctctatacaaatgCGGTGTTTCGTCCTTTCCACAAGATAT
ATAAAGCCAAGAAATCGAAATACTTTCAAGTTACGGTAAGCATATGATAGTCCATTTTAAAACATAAT
TTTAAAACTGCAAACTACCCAAGAAATTATTACTTTCTACGTCACGTATTTTGTACTAATATCTTTGT
GTTTACAGTCAAATTAATTCCAATTATCTCTCTAACAGCCTTGTATCGTATATGCAAATATGAAGGAA
TCATGGGAAATAGGCCCTCCTCGAGTCGAGTGGCTCCGGTGCCCGTCAGTGGGCAGAGCGCACATCGC
CCACAGTCCCCGAGAAGTTGGGGGGAGGGGTCGGCAATTGAACCGGTGCCTAGAGAAGGTGGCGCGGG
GTAAACTGGGAAAGTGATGTCGTGTACTGGCTCCGCCTTTTTCCCGAGGGTGGGGGAGAACCGTATAT
AAGTGCAGTAGTCGCCGTGAACGTTCTTTTTCGCAACGGGTTTGCCGCCAGAACACAGGTGTCGTGAC
CGCGGCCATGGCCCCAAAGAAGAAGCGGAAGGTCGGTATCCACGGAGTCCCAGCAGCCAAGCGGAACT
ACATCCTGGGCCTGGACATCGGCATCACCAGCGTGGGCTACGGCATCATCGACTACGAGACACGGGAC
GTGATCGATGCCGGCGTGCGGCTGTTCAAAGAGGCCAACGTGGAAAACAACGAGGGCAGGCGGAGCAA
GAGAGGCGCCAGAAGGCTGAAGCGGCGGAGGCGGCATAGAATCCAGAGAGTGAAGAAGCTGCTGTTCG
ACTACAACCTGCTGACCGACCACAGCGAGCTGAGCGGCATCAACCCCTACGAGGCCAGAGTGAAGGGC
CTGAGCCAGAAGCTGAGCGAGGAAGAGTTCTCTGCCGCCCTGCTGCACCTGGCCAAGAGAAGAGGCGT
GCACAACGTGAACGAGGTGGAAGAGGACACCGGCAACGAGCTGTCCACCAAAGAGCAGATCAGCCGGA
ACAGCAAGGCCCTGGAAGAGAAATACGTGGCCGAACTGCAGCTGGAACGGCTGAAGAAAGACGGCGAA
GTGCGGGGCAGCATCAACAGATTCAAGACCAGCGACTACGTGAAAGAAGCCAAACAGCTGCTGAAGGT
GCAGAAGGCCTACCACCAGCTGGACCAGAGCTTCATCGACACCTACATCGACCTGCTGGAAACCCGGC
GGACCTACTATGAGGGACCTGGCGAGGGCAGCCCCTTCGGCTGGAAGGACATCAAAGAATGGTACGAG
ATGCTGATGGGCCACTGCACCTACTTCCCCGAGGAACTGCGGAGCGTGAAGTACGCCTACAACGCCGA
CCTGTACAACGCCCTGAACGACCTGAACAATCTCGTGATCACCAGGGACGAGAACGAGAAGCTGGAAT
ATTACGAGAAGTTCCAGATCATCGAGAACGTGTTCAAGCAGAAGAAGAAGCCCACCCTGAAGCAGATC
GCCAAAGAAATCCTCGTGAACGAAGAGGATATTAAGGGCTACAGAGTGACCAGCACCGGCAAGCCCGA
GTTCACCAACCTGAAGGTGTACCACGACATCAAGGACATTACCGCCCGGAAAGAGATTATTGAGAACG
CCGAGCTGCTGGATCAGATTGCCAAGATCCTGACCATCTACCAGAGCAGCGAGGACATCCAGGAAGAA
CTGACCAATCTGAACTCCGAGCTGACCCAGGAAGAGATCGAGCAGATCTCTAATCTGAAGGGCTATAC
CGGCACCCACAACCTGAGCCTGAAGGCCATCAACCTGATCCTGGACGAGCTGTGGCACACCAACGACA
ACCAGATCGCTATCTTCAACCGGCTGAAGCTGGTGCCCAAGAAGGTGGACCTGTCCCAGCAGAAAGAG
ATCCCCACCACCCTGGTGGACGACTTCATCCTGAGCCCCGTCGTGAAGAGAAGCTTCATCCAGAGCAT
CAAAGTGATCAACGCCATCATCAAGAAGTACGGCCTGCCCAACGACATCATTATCGAGCTGGCCCGCG
AGAAGAACTCCAAGGACGCCCAGAAAATGATCAACGAGATGCAGAAGCGGAACCGGCAGACCAACGAG
CGGATCGAGGAAATCATCCGGACCACCGGCAAAGAGAACGCCAAGTACCTGATCGAGAAGATCAAGCT
GCACGACATGCAGGAAGGCAAGTGCCTGTACAGCCTGGAAGCCATCCCTCTGGAAGATCTGCTGAACA
ACCCCTTCAACTATGAGGTGGACCACATCATCCCCAGAAGCGTGTCCTTCGACAACAGCTTCAACAAC
AAGGTGCTCGTGAAGCAGGAAGAAAACAGCAAGAAGGGCAACCGGACCCCATTCCAGTACCTGAGCAG
CAGCGACAGCAAGATCAGCTACGAAACCTTCAAGAAGCACATCCTGAATCTGGCCAAGGGCAAGGGCA
GAATCAGCAAGACCAAGAAAGAGTATCTGCTGGAAGAACGGGACATCAACAGGTTCTCCGTGCAGAAA
GACTTCATCAACCGGAACCTGGTGGATACCAGATACGCCACCAGAGGCCTGATGAACCTGCTGCGGAG
CTACTTCAGAGTGAACAACCTGGACGTGAAAGTGAAGTCCATCAATGGCGGCTTCACCAGCTTTCTGC
GGCGGAAGTGGAAGTTTAAGAAAGAGCGGAACAAGGGGTACAAGCACCACGCCGAGGACGCCCTGATC
ATTGCCAACGCCGATTTCATCTTCAAAGAGTGGAAGAAACTGGACAAGGCCAAAAAAGTGATGGAAAA
CCAGATGTTCGAGGAAAAGCAGGCCGAGAGCATGCCCGAGATCGAAACCGAGCAGGAGTACAAAGAGA
TCTTCATCACCCCCCACCAGATCAAGCACATTAAGGACTTCAAGGACTACAAGTACAGCCACCGGGTG
GACAAGAAGCCTAATAGAGAGCTGATTAACGACACCCTGTACTCCACCCGGAAGGACGACAAGGGCAA
CACCCTGATCGTGAACAATCTGAACGGCCTGTACGACAAGGACAATGACAAGCTGAAAAAGCTGATCA
ACAAGAGCCCCGAAAAGCTGCTGATGTACCACCACGACCCCCAGACCTACCAGAAACTGAAGCTGATT
ATGGAACAGTACGGCGACGAGAAGAATCCCCTGTACAAGTACTACGAGGAAACCGGGAACTACCTGAC
CAAGTACTCCAAAAAGGACAACGGCCCCGTGATCAAGAAGATTAAGTATTACGGCAACAAACTGAACG
CCCATCTGGACATCACCGACGACTACCCCAACAGCAGAAACAAGGTCGTGAAGCTGTCCCTGAAGCCC
TACAGATTCGACGTGTACCTGGACAATGGCGTGTACAAGTTCGTGACCGTGAAGAATCTGGATGTGAT
CAAAAAAGAAAACTACTACGAAGTGAATAGCAAGTGCTATGAGGAAGCTAAGAAGCTGAAGAAGATCA
GCAACCAGGCCGAGTTTATCGCCTCCTTCTACAACAACGATCTGATCAAGATCAACGGCGAGCTGTAT
AGAGTGATCGGCGTGAACAACGACCTGCTGAACCGGATCGAAGTGAACATGATCGACATCACCTACCG
CGAGTACCTGGAAAACATGAACGACAAGAGGCCCCCCAGGATCATTAAGACAATCGCCTCCAAGACCC
AGAGCATTAAGAAGTACAGCACAGACATTCTGGGCAACCTGTATGAAGTGAAATCTAAGAAGCACCCT
CAGATCATCAAAAAGGGCAAAAGGCCGGCGGCCACGAAAAAGGCCGGCCAGGCAAAAAAGAAAAAGgg
atcctacccatacgatgttccagattacgcttacccatacgatgttccagattacgcttaccCatacg
atgttccagattacgcttaaGAATTCctagagctcgctgatcagcctcgactgtgccttctagttgcc
agccatctgttgtttgcccctcccccgtgccttccttgaccctggaaggtgccactcccactgtcctt
tcctaataaaatgaggaaattgcatcgcattgtctgagtaggtgtcattctattctggggggtggggt
ggggcaggacagcaagggggaggattgggaagagaatagcaggcatgctggggaGGTACCGAACGCTG
ACGTCATCAACCCGCTCCAAGGAATCGCGGGCCCAGTGTCACTAGGCGGGAACACCCAGCGCGCGTGC
GCCCTGGCAGGAAGATGGCTGTGAGGGACAGGGGAGTGGCGCCCTGCAATATTTGCATGTCGCTATGT
GTTCTGGGAAATCACCATAAACGTGAAATGTCTTTGGATTTGGGAATCTTATAAGTTCTGTATGAGAC
CACATATAGTAATGAAATTATTGGCACGTTTTAGTACTCTGGAAACAGAATCTACTAAAACAAGGCAA
AATGCCGTGTTTATCTCGTCAACTTGTTGGCGAGATTTTTGGTACCaggaacccctagtgatggagtt
ggccactccctctctgcgcgctcgctcgctcactgaggccgggcgaccaaaggtcgcccgacgcccgg
gctttgcccgggcggcctcagtgagcgagcgagcgcgcagctgcctgcaggggcgcctgatgcggtat
tttctccttacgcatctgtgcggtatttcacaccgcatacgtcaaagcaaccatagtacgcgccctgt
agcggcgcattaagcgcggcgggtgtggtggttacgcgcagcgtgaccgctacacttgccagcgccct
agcgcccgctcctttcgctttcttcccttcctttctcgccacgttcgccggctttccccgtcaagctc
taaatcgggggctccctttagggttccgatttagtgctttacggcacctcgaccccaaaaaacttgat
ttgggtgatggttcacgtagtgggccatcgccctgatagacggtttttcgccctttgacgttggagtc
cacgttctttaatagtggactcttgttccaaactggaacaacactcaaccctatctcgggctattctt
ttgatttataagggattttgccgatttcggcctattggttaaaaaatgagctgatttaacaaaaattt
aacgcgaattttaacaaaatattaacgtttacaattttatggtgcactctcagtacaatctgctctga
tgccgcatagttaagccagccccgacacccgccaacacccgctgacgcgccctgacgggcttgtctgc
tcccggcatccgcttacagacaagctgtgaccgtctccgggagctgcatgtgtcagaggttttcaccg
tcatcaccgaaacgcgcgagacgaaagggcctcgtgatacgcctatttttataggttaatgtcatgat
aataatggtttcttagacgtcaggtggcacttttcggggaaatgtgcgcggaacccctatttgtttat
ttttctaaatacattcaaatatgtatccgctcatgagacaataaccctgataaatgcttcaataatat
tgaaaaaggaagagtatgagtattcaacatttccgtgtcgcccttattcccttttttgcggcattttg
ccttcctgtttttgctcacccagaaacgctggtgaaagtaaaagatgctgaagatcagttgggtgcac
gagtgggttacatcgaactggatctcaacagcggtaagatccttgagagttttcgccccgaagaacgt
tttccaatgatgagcacttttaaagttctgctatgtggcgcggtattatcccgtattgacgccgggca
agagcaactcggtcgccgcatacactattctcagaatgacttggttgagtactcaccagtcacagaaa
agcatcttacggatggcatgacagtaagagaattatgcagtgctgccataaccatgagtgataacact
gcggccaacttacttctgacaacgatcggaggaccgaaggagctaaccgcttttttgcacaacatggg
ggatcatgtaactcgccttgatcgttgggaaccggagctgaatgaagccataccaaacgacgagcgtg
acaccacgatgcctgtagcaatggcaacaacgttgcgcaaactattaactggcgaactacttactcta
gcttcccggcaacaattaatagactggatggaggcggataaagttgcaggaccacttctgcgctcggc
ccttccggctggctggtttattgctgataaatctggagccggtgagcgtggaagccgcggtatcattg
cagcactggggccagatggtaagccctcccgtatcgtagttatctacacgacggggagtcaggcaact
atggatgaacgaaatagacagatcgctgagataggtgcctcactgattaagcattggtaactgtcaga
ccaagtttactcatatatactttagattgatttaaaacttcatttttaatttaaaaggatctaggtga
agatcctttttgataatctcatgaccaaaatcccttaacgtgagttttcgttccactgagcgtcagac
cccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaagg
taactggcttcagcagagcgcagataccaaatactgtccttctagtgtagccgtagttaggccaccac
ttcaagaactctgtagcaccgcctacatacctcgctctgctaatcctgttaccagtggctgctgccag
tggcgataagtcgtgtcttaccgggttggactcaagacgatagttaccggataaggcgcagcggtcgg
gctgaacggggggttcgtgcacacagcccagcttggagcgaacgacctacaccgaactgagataccta
cagcgtgagctatgagaaagcgccacgcttcccgaagggagaaaggcggacaggtatccggtaagcgg
cagggtcggaacaggagagcgcacgagggagcttccagggggaaacgcctggtatctttatagtcctg
tcgggtttcgccacctctgacttgagcgtcgatttttgtgatgctcgtcaggggggcggagcctatgg
aaaaacgccagcaacgcggcctttttacggttcctggccttttgctggccttttgctcacatgt
Version 2 of vector 3
SEQ ID NO: 80
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGAaaaaatctcgccaacaagttgacgagataaacacggcattttgccttgttttagtagattct
gtttccagagtactaaaacacatttcctctctatacaaatgCGGTGTTTCGTCCTTTCCACAAGATAT
ATAAAGCCAAGAAATCGAAATACTTTCAAGTTACGGTAAGCATATGATAGTCCATTTTAAAACATAAT
TTTAAAACTGCAAACTACCCAAGAAATTATTACTTTCTACGTCACGTATTTTGTACTAATATCTTTGT
GTTTACAGTCAAATTAATTCCAATTATCTCTCTAACAGCCTTGTATCGTATATGCAAATATGAAGGAA
TCATGGGAAATAGGCCCTCCTCGAGCTAGACTAGCATGCTGCCCATGTAAGGAGGCAAGGCCTGGGGA
CACCCGAGATGCCTGGTTATAATTAACCCAGACATGTGGCTGCCCCCCCCCCCCCAACACCTGCTGCC
TCTAAAAATAACCCTGCATGCCATGTTCCCGGCGAAGGGCCAGCTGTCCCCCGCCAGCTAGACTCAGC
ACTTAGTTTAGGAACCAGTGAGCAAGTCAGCCCTTGGGGCAGCCCATACAAGGCCATGGGGCTGGGCA
AGCTGCACGCCTGGGTCCGGGGTGGGCACGGTGCCCGGGCAACGAGCTGAAAGCTCATCTGCTCTCAG
GGGCCCCTCCCTGGGGACAGCCCCTCCTGGCTAGTCACACCCTGTAGGCTCCTCTATATAACCCAGGG
GCACAGGGGCTGCCCTCATTCTACCACCACCTCCACAGCACAGACAGACACTCAGGAGCCAGCCAGCa
ccggtgccaccATGGCCCCAAAGAAGAAGCGGAAGGTCGGTATCCACGGAGTCCCAGCAGCCAAGCGG
AACTACATCCTGGGCCTGGACATCGGCATCACCAGCGTGGGCTACGGCATCATCGACTACGAGACACG
GGACGTGATCGATGCCGGCGTGCGGCTGTTCAAAGAGGCCAACGTGGAAAACAACGAGGGCAGGCGGA
GCAAGAGAGGCGCCAGAAGGCTGAAGCGGCGGAGGCGGCATAGAATCCAGAGAGTGAAGAAGCTGCTG
TTCGACTACAACCTGCTGACCGACCACAGCGAGCTGAGCGGCATCAACCCCTACGAGGCCAGAGTGAA
GGGCCTGAGCCAGAAGCTGAGCGAGGAAGAGTTCTCTGCCGCCCTGCTGCACCTGGCCAAGAGAAGAG
GCGTGCACAACGTGAACGAGGTGGAAGAGGACACCGGCAACGAGCTGTCCACCAAAGAGCAGATCAGC
CGGAACAGCAAGGCCCTGGAAGAGAAATACGTGGCCGAACTGCAGCTGGAACGGCTGAAGAAAGACGG
CGAAGTGCGGGGCAGCATCAACAGATTCAAGACCAGCGACTACGTGAAAGAAGCCAAACAGCTGCTGA
AGGTGCAGAAGGCCTACCACCAGCTGGACCAGAGCTTCATCGACACCTACATCGACCTGCTGGAAACC
CGGCGGACCTACTATGAGGGACCTGGCGAGGGCAGCCCCTTCGGCTGGAAGGACATCAAAGAATGGTA
CGAGATGCTGATGGGCCACTGCACCTACTTCCCCGAGGAACTGCGGAGCGTGAAGTACGCCTACAACG
CCGACCTGTACAACGCCCTGAACGACCTGAACAATCTCGTGATCACCAGGGACGAGAACGAGAAGCTG
GAATATTACGAGAAGTTCCAGATCATCGAGAACGTGTTCAAGCAGAAGAAGAAGCCCACCCTGAAGCA
GATCGCCAAAGAAATCCTCGTGAACGAAGAGGATATTAAGGGCTACAGAGTGACCAGCACCGGCAAGC
CCGAGTTCACCAACCTGAAGGTGTACCACGACATCAAGGACATTACCGCCCGGAAAGAGATTATTGAG
AACGCCGAGCTGCTGGATCAGATTGCCAAGATCCTGACCATCTACCAGAGCAGCGAGGACATCCAGGA
AGAACTGACCAATCTGAACTCCGAGCTGACCCAGGAAGAGATCGAGCAGATCTCTAATCTGAAGGGCT
ATACCGGCACCCACAACCTGAGCCTGAAGGCCATCAACCTGATCCTGGACGAGCTGTGGCACACCAAC
GACAACCAGATCGCTATCTTCAACCGGCTGAAGCTGGTGCCCAAGAAGGTGGACCTGTCCCAGCAGAA
AGAGATCCCCACCACCCTGGTGGACGACTTCATCCTGAGCCCCGTCGTGAAGAGAAGCTTCATCCAGA
GCATCAAAGTGATCAACGCCATCATCAAGAAGTACGGCCTGCCCAACGACATCATTATCGAGCTGGCC
CGCGAGAAGAACTCCAAGGACGCCCAGAAAATGATCAACGAGATGCAGAAGCGGAACCGGCAGACCAA
CGAGCGGATCGAGGAAATCATCCGGACCACCGGCAAAGAGAACGCCAAGTACCTGATCGAGAAGATCA
AGCTGCACGACATGCAGGAAGGCAAGTGCCTGTACAGCCTGGAAGCCATCCCTCTGGAAGATCTGCTG
AACAACCCCTTCAACTATGAGGTGGACCACATCATCCCCAGAAGCGTGTCCTTCGACAACAGCTTCAA
CAACAAGGTGCTCGTGAAGCAGGAAGAAAACAGCAAGAAGGGCAACCGGACCCCATTCCAGTACCTGA
GCAGCAGCGACAGCAAGATCAGCTACGAAACCTTCAAGAAGCACATCCTGAATCTGGCCAAGGGCAAG
GGCAGAATCAGCAAGACCAAGAAAGAGTATCTGCTGGAAGAACGGGACATCAACAGGTTCTCCGTGCA
GAAAGACTTCATCAACCGGAACCTGGTGGATACCAGATACGCCACCAGAGGCCTGATGAACCTGCTGC
GGAGCTACTTCAGAGTGAACAACCTGGACGTGAAAGTGAAGTCCATCAATGGCGGCTTCACCAGCTTT
CTGCGGCGGAAGTGGAAGTTTAAGAAAGAGCGGAACAAGGGGTACAAGCACCACGCCGAGGACGCCCT
GATCATTGCCAACGCCGATTTCATCTTCAAAGAGTGGAAGAAACTGGACAAGGCCAAAAAAGTGATGG
AAAACCAGATGTTCGAGGAAAAGCAGGCCGAGAGCATGCCCGAGATCGAAACCGAGCAGGAGTACAAA
GAGATCTTCATCACCCCCCACCAGATCAAGCACATTAAGGACTTCAAGGACTACAAGTACAGCCACCG
GGTGGACAAGAAGCCTAATAGAGAGCTGATTAACGACACCCTGTACTCCACCCGGAAGGACGACAAGG
GCAACACCCTGATCGTGAACAATCTGAACGGCCTGTACGACAAGGACAATGACAAGCTGAAAAAGCTG
ATCAACAAGAGCCCCGAAAAGCTGCTGATGTACCACCACGACCCCCAGACCTACCAGAAACTGAAGCT
GATTATGGAACAGTACGGCGACGAGAAGAATCCCCTGTACAAGTACTACGAGGAAACCGGGAACTACC
TGACCAAGTACTCCAAAAAGGACAACGGCCCCGTGATCAAGAAGATTAAGTATTACGGCAACAAACTG
AACGCCCATCTGGACATCACCGACGACTACCCCAACAGCAGAAACAAGGTCGTGAAGCTGTCCCTGAA
GCCCTACAGATTCGACGTGTACCTGGACAATGGCGTGTACAAGTTCGTGACCGTGAAGAATCTGGATG
TGATCAAAAAAGAAAACTACTACGAAGTGAATAGCAAGTGCTATGAGGAAGCTAAGAAGCTGAAGAAG
ATCAGCAACCAGGCCGAGTTTATCGCCTCCTTCTACAACAACGATCTGATCAAGATCAACGGCGAGCT
GTATAGAGTGATCGGCGTGAACAACGACCTGCTGAACCGGATCGAAGTGAACATGATCGACATCACCT
ACCGCGAGTACCTGGAAAACATGAACGACAAGAGGCCCCCCAGGATCATTAAGACAATCGCCTCCAAG
ACCCAGAGCATTAAGAAGTACAGCACAGACATTCTGGGCAACCTGTATGAAGTGAAATCTAAGAAGCA
CCCTCAGATCATCAAAAAGGGCAAAAGGCCGGCGGCCACGAAAAAGGCCGGCCAGGCAAAAAAGAAAA
AGggatcctacccatacgatgttccagattacgcttacccatacgatgttccagattacgcttaccCa
tacgatgttccagattacgcttaaGAATTCctagagctcgctgatcagcctcgactgtgccttctagt
tgccagccatctgttgtttgcccctcccccgtgccttccttgaccctggaaggtgccactcccactgt
cctttcctaataaaatgaggaaattgcatcgcattgtctgagtaggtgtcattctattctggggggtg
gggtggggcaggacagcaagggggaggattgggaagagaatagcaggcatgctggggaGGTACCGAAC
GCTGACGTCATCAACCCGCTCCAAGGAATCGCGGGCCCAGTGTCACTAGGCGGGAACACCCAGCGCGC
GTGCGCCCTGGCAGGAAGATGGCTGTGAGGGACAGGGGAGTGGCGCCCTGCAATATTTGCATGTCGCT
ATGTGTTCTGGGAAATCACCATAAACGTGAAATGTCTTTGGATTTGGGAATCTTATAAGTTCTGTATG
AGACCACATATAGTAATGAAATTATTGGCACGTTTTAGTACTCTGGAAACAGAATCTACTAAAACAAG
GCAAAATGCCGTGTTTATCTCGTCAACTTGTTGGCGAGATTTTTGGTACCaggaacccctagtgatgg
agttggccactccctctctgcgcgctcgctcgctcactgaggccgggcgaccaaaggtcgcccgacgc
ccgggctttgcccgggcggcctcagtgagcgagcgagcgcgcagctgcctgcaggggcgcctgatgcg
gtattttctccttacgcatctgtgcggtatttcacaccgcatacgtcaaagcaaccatagtacgcgcc
ctgtagcggcgcattaagcgcggcgggtgtggtggttacgcgcagcgtgaccgctacacttgccagcg
ccctagcgcccgctcctttcgctttcttcccttcctttctcgccacgttcgccggctttccccgtcaa
gctctaaatcgggggctccctttagggttccgatttagtgctttacggcacctcgaccccaaaaaact
tgatttgggtgatggttcacgtagtgggccatcgccctgatagacggtttttcgccctttgacgttgg
agtccacgttctttaatagtggactcttgttccaaactggaacaacactcaaccctatctcgggctat
tcttttgatttataagggattttgccgatttcggcctattggttaaaaaatgagctgatttaacaaaa
atttaacgcgaattttaacaaaatattaacgtttacaattttatggtgcactctcagtacaatctgct
ctgatgccgcatagttaagccagccccgacacccgccaacacccgctgacgcgccctgacgggcttgt
ctgctcccggcatccgcttacagacaagctgtgaccgtctccgggagctgcatgtgtcagaggttttc
accgtcatcaccgaaacgcgcgagacgaaagggcctcgtgatacgcctatttttataggttaatgtca
tgataataatggtttcttagacgtcaggtggcacttttcggggaaatgtgcgcggaacccctatttgt
ttatttttctaaatacattcaaatatgtatccgctcatgagacaataaccctgataaatgcttcaata
atattgaaaaaggaagagtatgagtattcaacatttccgtgtcgcccttattcccttttttgcggcat
tttgccttcctgtttttgctcacccagaaacgctggtgaaagtaaaagatgctgaagatcagttgggt
gcacgagtgggttacatcgaactggatctcaacagcggtaagatccttgagagttttcgccccgaaga
acgttttccaatgatgagcacttttaaagttctgctatgtggcgcggtattatcccgtattgacgccg
ggcaagagcaactcggtcgccgcatacactattctcagaatgacttggttgagtactcaccagtcaca
gaaaagcatcttacggatggcatgacagtaagagaattatgcagtgctgccataaccatgagtgataa
cactgcggccaacttacttctgacaacgatcggaggaccgaaggagctaaccgcttttttgcacaaca
tgggggatcatgtaactcgccttgatcgttgggaaccggagctgaatgaagccataccaaacgacgag
cgtgacaccacgatgcctgtagcaatggcaacaacgttgcgcaaactattaactggcgaactacttac
tctagcttcccggcaacaattaatagactggatggaggcggataaagttgcaggaccacttctgcgct
cggcccttccggctggctggtttattgctgataaatctggagccggtgagcgtggaagccgcggtatc
attgcagcactggggccagatggtaagccctcccgtatcgtagttatctacacgacggggagtcaggc
aactatggatgaacgaaatagacagatcgctgagataggtgcctcactgattaagcattggtaactgt
cagaccaagtttactcatatatactttagattgatttaaaacttcatttttaatttaaaaggatctag
gtgaagatcctttttgataatctcatgaccaaaatcccttaacgtgagttttcgttccactgagcgtc
agaccccgtagaaaagatcaaaggatcttcttgagatcctttttttctgcgcgtaatctgctgcttgc
aaacaaaaaaaccaccgctaccagcggtggtttgtttgccggatcaagagctaccaactctttttccg
aaggtaactggcttcagcagagcgcagataccaaatactgtccttctagtgtagccgtagttaggcca
ccacttcaagaactctgtagcaccgcctacatacctcgctctgctaatcctgttaccagtggctgctg
ccagtggcgataagtcgtgtcttaccgggttggactcaagacgatagttaccggataaggcgcagcgg
tcgggctgaacggggggttcgtgcacacagcccagcttggagcgaacgacctacaccgaactgagata
cctacagcgtgagctatgagaaagcgccacgcttcccgaagggagaaaggcggacaggtatccggtaa
gcggcagggtcggaacaggagagcgcacgagggagcttccagggggaaacgcctggtatctttatagt
cctgtcgggtttcgccacctctgacttgagcgtcgatttttgtgatgctcgtcaggggggcggagcct
atggaaaaacgccagcaacgcggcctttttacggttcctggccttttgctggccttttgctcacatgt
Version 3 of vector 3
SEQ ID NO: 81
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGAaaaaatctcgccaacaagttgacgagataaacacggcattttgccttgttttagtagattct
gtttccagagtactaaaacacatttcctctctatacaaatgCGGTGTTTCGTCCTTTCCACAAGATAT
ATAAAGCCAAGAAATCGAAATACTTTCAAGTTACGGTAAGCATATGATAGTCCATTTTAAAACATAAT
TTTAAAACTGCAAACTACCCAAGAAATTATTACTTTCTACGTCACGTATTTTGTACTAATATCTTTGT
GTTTACAGTCAAATTAATTCCAATTATCTCTCTAACAGCCTTGTATCGTATATGCAAATATGAAGGAA
TCATGGGAAATAGGCCCTCCTCGAGGAGCTCCACCGCGGTGGCGGCCGTCCGCCtTCGGCACCATCCT
CACGACACCCAAATATGGCGACGGGTGAGGAATGGTGGGGAGTTATTTTTAGAGCGGTGAGGAAGGTG
GGCAGGCAGCAGGTGTTGGCGCTCTAAAAATAACTCCCGGGAGTTATTTTTAGAGCGGAGGAATGGTG
GACACCCAAATATGGCGACGGTTCCTCACCCGTCGCCATATTTGGGTGTCCGCCCTCGGCCGGGGCCG
CATTCCTGGGGGCCGGGCGGTGCTCCCGCCCGCCTCGATAAAAGGCTCCGGGGCCGGGGGGGGCCCAC
GAGCTACCCGGAGGAGCGGGAGGCGCCAAGCTCTAGAACTAGTGGATCCCCCGGGCTGCAGGAATTCG
ATATaccggtgccaccATGGCCCCAAAGAAGAAGCGGAAGGTCGGTATCCACGGAGTCCCAGCAGCCA
AGCGGAACTACATCCTGGGCCTGGACATCGGCATCACCAGCGTGGGCTACGGCATCATCGACTACGAG
ACACGGGACGTGATCGATGCCGGCGTGCGGCTGTTCAAAGAGGCCAACGTGGAAAACAACGAGGGCAG
GCGGAGCAAGAGAGGCGCCAGAAGGCTGAAGCGGCGGAGGCGGCATAGAATCCAGAGAGTGAAGAAGC
TGCTGTTCGACTACAACCTGCTGACCGACCACAGCGAGCTGAGCGGCATCAACCCCTACGAGGCCAGA
GTGAAGGGCCTGAGCCAGAAGCTGAGCGAGGAAGAGTTCTCTGCCGCCCTGCTGCACCTGGCCAAGAG
AAGAGGCGTGCACAACGTGAACGAGGTGGAAGAGGACACCGGCAACGAGCTGTCCACCAAAGAGCAGA
TCAGCCGGAACAGCAAGGCCCTGGAAGAGAAATACGTGGCCGAACTGCAGCTGGAACGGCTGAAGAAA
GACGGCGAAGTGCGGGGCAGCATCAACAGATTCAAGACCAGCGACTACGTGAAAGAAGCCAAACAGCT
GCTGAAGGTGCAGAAGGCCTACCACCAGCTGGACCAGAGCTTCATCGACACCTACATCGACCTGCTGG
AAACCCGGCGGACCTACTATGAGGGACCTGGCGAGGGCAGCCCCTTCGGCTGGAAGGACATCAAAGAA
TGGTACGAGATGCTGATGGGCCACTGCACCTACTTCCCCGAGGAACTGCGGAGCGTGAAGTACGCCTA
CAACGCCGACCTGTACAACGCCCTGAACGACCTGAACAATCTCGTGATCACCAGGGACGAGAACGAGA
AGCTGGAATATTACGAGAAGTTCCAGATCATCGAGAACGTGTTCAAGCAGAAGAAGAAGCCCACCCTG
AAGCAGATCGCCAAAGAAATCCTCGTGAACGAAGAGGATATTAAGGGCTACAGAGTGACCAGCACCGG
CAAGCCCGAGTTCACCAACCTGAAGGTGTACCACGACATCAAGGACATTACCGCCCGGAAAGAGATTA
TTGAGAACGCCGAGCTGCTGGATCAGATTGCCAAGATCCTGACCATCTACCAGAGCAGCGAGGACATC
CAGGAAGAACTGACCAATCTGAACTCCGAGCTGACCCAGGAAGAGATCGAGCAGATCTCTAATCTGAA
GGGCTATACCGGCACCCACAACCTGAGCCTGAAGGCCATCAACCTGATCCTGGACGAGCTGTGGCACA
CCAACGACAACCAGATCGCTATCTTCAACCGGCTGAAGCTGGTGCCCAAGAAGGTGGACCTGTCCCAG
CAGAAAGAGATCCCCACCACCCTGGTGGACGACTTCATCCTGAGCCCCGTCGTGAAGAGAAGCTTCAT
CCAGAGCATCAAAGTGATCAACGCCATCATCAAGAAGTACGGCCTGCCCAACGACATCATTATCGAGC
TGGCCCGCGAGAAGAACTCCAAGGACGCCCAGAAAATGATCAACGAGATGCAGAAGCGGAACCGGCAG
ACCAACGAGCGGATCGAGGAAATCATCCGGACCACCGGCAAAGAGAACGCCAAGTACCTGATCGAGAA
GATCAAGCTGCACGACATGCAGGAAGGCAAGTGCCTGTACAGCCTGGAAGCCATCCCTCTGGAAGATC
TGCTGAACAACCCCTTCAACTATGAGGTGGACCACATCATCCCCAGAAGCGTGTCCTTCGACAACAGC
TTCAACAACAAGGTGCTCGTGAAGCAGGAAGAAAACAGCAAGAAGGGCAACCGGACCCCATTCCAGTA
CCTGAGCAGCAGCGACAGCAAGATCAGCTACGAAACCTTCAAGAAGCACATCCTGAATCTGGCCAAGG
GCAAGGGCAGAATCAGCAAGACCAAGAAAGAGTATCTGCTGGAAGAACGGGACATCAACAGGTTCTCC
GTGCAGAAAGACTTCATCAACCGGAACCTGGTGGATACCAGATACGCCACCAGAGGCCTGATGAACCT
GCTGCGGAGCTACTTCAGAGTGAACAACCTGGACGTGAAAGTGAAGTCCATCAATGGCGGCTTCACCA
GCTTTCTGCGGCGGAAGTGGAAGTTTAAGAAAGAGCGGAACAAGGGGTACAAGCACCACGCCGAGGAC
GCCCTGATCATTGCCAACGCCGATTTCATCTTCAAAGAGTGGAAGAAACTGGACAAGGCCAAAAAAGT
GATGGAAAACCAGATGTTCGAGGAAAAGCAGGCCGAGAGCATGCCCGAGATCGAAACCGAGCAGGAGT
ACAAAGAGATCTTCATCACCCCCCACCAGATCAAGCACATTAAGGACTTCAAGGACTACAAGTACAGC
CACCGGGTGGACAAGAAGCCTAATAGAGAGCTGATTAACGACACCCTGTACTCCACCCGGAAGGACGA
CAAGGGCAACACCCTGATCGTGAACAATCTGAACGGCCTGTACGACAAGGACAATGACAAGCTGAAAA
AGCTGATCAACAAGAGCCCCGAAAAGCTGCTGATGTACCACCACGACCCCCAGACCTACCAGAAACTG
AAGCTGATTATGGAACAGTACGGCGACGAGAAGAATCCCCTGTACAAGTACTACGAGGAAACCGGGAA
CTACCTGACCAAGTACTCCAAAAAGGACAACGGCCCCGTGATCAAGAAGATTAAGTATTACGGCAACA
AACTGAACGCCCATCTGGACATCACCGACGACTACCCCAACAGCAGAAACAAGGTCGTGAAGCTGTCC
CTGAAGCCCTACAGATTCGACGTGTACCTGGACAATGGCGTGTACAAGTTCGTGACCGTGAAGAATCT
GGATGTGATCAAAAAAGAAAACTACTACGAAGTGAATAGCAAGTGCTATGAGGAAGCTAAGAAGCTGA
AGAAGATCAGCAACCAGGCCGAGTTTATCGCCTCCTTCTACAACAACGATCTGATCAAGATCAACGGC
GAGCTGTATAGAGTGATCGGCGTGAACAACGACCTGCTGAACCGGATCGAAGTGAACATGATCGACAT
CACCTACCGCGAGTACCTGGAAAACATGAACGACAAGAGGCCCCCCAGGATCATTAAGACAATCGCCT
CCAAGACCCAGAGCATTAAGAAGTACAGCACAGACATTCTGGGCAACCTGTATGAAGTGAAATCTAAG
AAGCACCCTCAGATCATCAAAAAGGGCAAAAGGCCGGCGGCCACGAAAAAGGCCGGCCAGGCAAAAAA
GAAAAAGggatcctacccatacgatgttccagattacgcttacccatacgatgttccagattacgctt
accCatacgatgttccagattacgcttaaGAATTCctagagctcgctgatcagcctcgactgtgcctt
ctagttgccagccatctgttgtttgcccctcccccgtgccttccttgaccctggaaggtgccactccc
actgtcctttcctaataaaatgaggaaattgcatcgcattgtctgagtaggtgtcattctattctggg
gggtggggtggggcaggacagcaagggggaggattgggaagagaatagcaggcatgctggggaGGTAC
CGAACGCTGACGTCATCAACCCGCTCCAAGGAATCGCGGGCCCAGTGTCACTAGGCGGGAACACCCAG
CGCGCGTGCGCCCTGGCAGGAAGATGGCTGTGAGGGACAGGGGAGTGGCGCCCTGCAATATTTGCATG
TCGCTATGTGTTCTGGGAAATCACCATAAACGTGAAATGTCTTTGGATTTGGGAATCTTATAAGTTCT
GTATGAGACCACATATAGTAATGAAATTATTGGCACGTTTTAGTACTCTGGAAACAGAATCTACTAAA
ACAAGGCAAAATGCCGTGTTTATCTCGTCAACTTGTTGGCGAGATTTTTGGTACCaggaacccctagt
gatggagttggccactccctctctgcgcgctcgctcgctcactgaggccgggcgaccaaaggtcgccc
gacgcccgggctttgcccgggcggcctcagtgagcgagcgagcgcgcagctgcctgcaggggcgcctg
atgcggtattttctccttacgcatctgtgcggtatttcacaccgcatacgtcaaagcaaccatagtac
gcgccctgtagcggcgcattaagcgcggcgggtgtggtggttacgcgcagcgtgaccgctacacttgc
cagcgccctagcgcccgctcctttcgctttcttcccttcctttctcgccacgttcgccggctttcccc
gtcaagctctaaatcgggggctccctttagggttccgatttagtgctttacggcacctcgaccccaaa
aaacttgatttgggtgatggttcacgtagtgggccatcgccctgatagacggtttttcgccctttgac
gttggagtccacgttctttaatagtggactcttgttccaaactggaacaacactcaaccctatctcgg
gctattcttttgatttataagggattttgccgatttcggcctattggttaaaaaatgagctgatttaa
caaaaatttaacgcgaattttaacaaaatattaacgtttacaattttatggtgcactctcagtacaat
ctgctctgatgccgcatagttaagccagccccgacacccgccaacacccgctgacgcgccctgacggg
cttgtctgctcccggcatccgcttacagacaagctgtgaccgtctccgggagctgcatgtgtcagagg
ttttcaccgtcatcaccgaaacgcgcgagacgaaagggcctcgtgatacgcctatttttataggttaa
tgtcatgataataatggtttcttagacgtcaggtggcacttttcggggaaatgtgcgcggaaccccta
tttgtttatttttctaaatacattcaaatatgtatccgctcatgagacaataaccctgataaatgctt
caataatattgaaaaaggaagagtatgagtattcaacatttccgtgtcgcccttattcccttttttgc
ggcattttgccttcctgtttttgctcacccagaaacgctggtgaaagtaaaagatgctgaagatcagt
tgggtgcacgagtgggttacatcgaactggatctcaacagcggtaagatccttgagagttttcgcccc
gaagaacgttttccaatgatgagcacttttaaagttctgctatgtggcgcggtattatcccgtattga
cgccgggcaagagcaactcggtcgccgcatacactattctcagaatgacttggttgagtactcaccag
tcacagaaaagcatcttacggatggcatgacagtaagagaattatgcagtgctgccataaccatgagt
gataacactgcggccaacttacttctgacaacgatcggaggaccgaaggagctaaccgcttttttgca
caacatgggggatcatgtaactcgccttgatcgttgggaaccggagctgaatgaagccataccaaacg
acgagcgtgacaccacgatgcctgtagcaatggcaacaacgttgcgcaaactattaactggcgaacta
cttactctagcttcccggcaacaattaatagactggatggaggcggataaagttgcaggaccacttct
gcgctcggcccttccggctggctggtttattgctgataaatctggagccggtgagcgtggaagccgcg
gtatcattgcagcactggggccagatggtaagccctcccgtatcgtagttatctacacgacggggagt
caggcaactatggatgaacgaaatagacagatcgctgagataggtgcctcactgattaagcattggta
actgtcagaccaagtttactcatatatactttagattgatttaaaacttcatttttaatttaaaagga
tctaggtgaagatcctttttgataatctcatgaccaaaatcccttaacgtgagttttcgttccactga
gcgtcagaccccgtagaaaagatcaaaggatcttcttgagatcctttttttctgcgcgtaatctgctg
cttgcaaacaaaaaaaccaccgctaccagcggtggtttgtttgccggatcaagagctaccaactcttt
ttccgaaggtaactggcttcagcagagcgcagataccaaatactgtccttctagtgtagccgtagtta
ggccaccacttcaagaactctgtagcaccgcctacatacctcgctctgctaatcctgttaccagtggc
tgctgccagtggcgataagtcgtgtcttaccgggttggactcaagacgatagttaccggataaggcgc
agcggtcgggctgaacggggggttcgtgcacacagcccagcttggagcgaacgacctacaccgaactg
agatacctacagcgtgagctatgagaaagcgccacgcttcccgaagggagaaaggcggacaggtatcc
ggtaagcggcagggtcggaacaggagagcgcacgagggagcttccagggggaaacgcctggtatcttt
atagtcctgtcgggtttcgccacctctgacttgagcgtcgatttttgtgatgctcgtcaggggggcgg
agcctatggaaaaacgccagcaacgcggcctttttacggttcctggccttttgctggccttttgctca
catgt
Version 4 of vector 3
SEQ ID NO: 82
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGAaaaaatctcgccaacaagttgacgagataaacacggcattttgccttgttttagtagattct
gtttccagagtactaaaacacatttcctctctatacaaatgCGGTGTTTCGTCCTTTCCACAAGATAT
ATAAAGCCAAGAAATCGAAATACTTTCAAGTTACGGTAAGCATATGATAGTCCATTTTAAAACATAAT
TTTAAAACTGCAAACTACCCAAGAAATTATTACTTTCTACGTCACGTATTTTGTACTAATATCTTTGT
GTTTACAGTCAAATTAATTCCAATTATCTCTCTAACAGCCTTGTATCGTATATGCAAATATGAAGGAA
TCATGGGAAATAGGCCCTCCTCGAGGtttaaacaagcttgcatgtctaagctagacccttcagattaa
aaataactgaggtaagggcctgggtaggggaggtggtgtgagacgctcctgtctctcctctatctgcc
catcggccctttggggaggaggaatgtgcccaaggactaaaaaaaggccatggagccagaggggcgag
ggcaacagacctttcatgggcaaaccttggggccctgctgtctagcatgccccactacgggtctaggc
tgcccatgtaaggaggcaaggcctggggacacccgagatgcctggttataattaacccagacatgtgg
ctgcccccccccccccaacacctgctgcctctaaaaataaccctgtccctggtggatcccctgcatgc
gaagatcttcgaacaaggctgtgggggactgagggcaggctgtaacaggcttgggggccagggcttat
acgtgcctgggactcccaaagtattactgttccatgttcccggcgaagggccagctgtcccccgccag
ctagactcagcacttagtttaggaaccagtgagcaagtcagcccttggggcagcccatacaaggccat
ggggctgggcaagctgcacgcctgggtccggggtgggcacggtgcccgggcaacgagctgaaagctca
tctgctctcaggggcccctccctggggacagcccctcctggctagtcacaccctgtaggctcctctat
ataacccaggggcacaggggctgccctcattctaccaccacctccacagcacagacagacactcagga
gccagccagcggcgcgcccaccggtgccaccATGGCCCCAAAGAAGAAGCGGAAGGTCGGTATCCACG
GAGTCCCAGCAGCCAAGCGGAACTACATCCTGGGCCTGGACATCGGCATCACCAGCGTGGGCTACGGC
ATCATCGACTACGAGACACGGGACGTGATCGATGCCGGCGTGCGGCTGTTCAAAGAGGCCAACGTGGA
AAACAACGAGGGCAGGCGGAGCAAGAGAGGCGCCAGAAGGCTGAAGCGGCGGAGGCGGCATAGAATCC
AGAGAGTGAAGAAGCTGCTGTTCGACTACAACCTGCTGACCGACCACAGCGAGCTGAGCGGCATCAAC
CCCTACGAGGCCAGAGTGAAGGGCCTGAGCCAGAAGCTGAGCGAGGAAGAGTTCTCTGCCGCCCTGCT
GCACCTGGCCAAGAGAAGAGGCGTGCACAACGTGAACGAGGTGGAAGAGGACACCGGCAACGAGCTGT
CCACCAAAGAGCAGATCAGCCGGAACAGCAAGGCCCTGGAAGAGAAATACGTGGCCGAACTGCAGCTG
GAACGGCTGAAGAAAGACGGCGAAGTGCGGGGCAGCATCAACAGATTCAAGACCAGCGACTACGTGAA
AGAAGCCAAACAGCTGCTGAAGGTGCAGAAGGCCTACCACCAGCTGGACCAGAGCTTCATCGACACCT
ACATCGACCTGCTGGAAACCCGGCGGACCTACTATGAGGGACCTGGCGAGGGCAGCCCCTTCGGCTGG
AAGGACATCAAAGAATGGTACGAGATGCTGATGGGCCACTGCACCTACTTCCCCGAGGAACTGCGGAG
CGTGAAGTACGCCTACAACGCCGACCTGTACAACGCCCTGAACGACCTGAACAATCTCGTGATCACCA
GGGACGAGAACGAGAAGCTGGAATATTACGAGAAGTTCCAGATCATCGAGAACGTGTTCAAGCAGAAG
AAGAAGCCCACCCTGAAGCAGATCGCCAAAGAAATCCTCGTGAACGAAGAGGATATTAAGGGCTACAG
AGTGACCAGCACCGGCAAGCCCGAGTTCACCAACCTGAAGGTGTACCACGACATCAAGGACATTACCG
CCCGGAAAGAGATTATTGAGAACGCCGAGCTGCTGGATCAGATTGCCAAGATCCTGACCATCTACCAG
AGCAGCGAGGACATCCAGGAAGAACTGACCAATCTGAACTCCGAGCTGACCCAGGAAGAGATCGAGCA
GATCTCTAATCTGAAGGGCTATACCGGCACCCACAACCTGAGCCTGAAGGCCATCAACCTGATCCTGG
ACGAGCTGTGGCACACCAACGACAACCAGATCGCTATCTTCAACCGGCTGAAGCTGGTGCCCAAGAAG
GTGGACCTGTCCCAGCAGAAAGAGATCCCCACCACCCTGGTGGACGACTTCATCCTGAGCCCCGTCGT
GAAGAGAAGCTTCATCCAGAGCATCAAAGTGATCAACGCCATCATCAAGAAGTACGGCCTGCCCAACG
ACATCATTATCGAGCTGGCCCGCGAGAAGAACTCCAAGGACGCCCAGAAAATGATCAACGAGATGCAG
AAGCGGAACCGGCAGACCAACGAGCGGATCGAGGAAATCATCCGGACCACCGGCAAAGAGAACGCCAA
GTACCTGATCGAGAAGATCAAGCTGCACGACATGCAGGAAGGCAAGTGCCTGTACAGCCTGGAAGCCA
TCCCTCTGGAAGATCTGCTGAACAACCCCTTCAACTATGAGGTGGACCACATCATCCCCAGAAGCGTG
TCCTTCGACAACAGCTTCAACAACAAGGTGCTCGTGAAGCAGGAAGAAAACAGCAAGAAGGGCAACCG
GACCCCATTCCAGTACCTGAGCAGCAGCGACAGCAAGATCAGCTACGAAACCTTCAAGAAGCACATCC
TGAATCTGGCCAAGGGCAAGGGCAGAATCAGCAAGACCAAGAAAGAGTATCTGCTGGAAGAACGGGAC
ATCAACAGGTTCTCCGTGCAGAAAGACTTCATCAACCGGAACCTGGTGGATACCAGATACGCCACCAG
AGGCCTGATGAACCTGCTGCGGAGCTACTTCAGAGTGAACAACCTGGACGTGAAAGTGAAGTCCATCA
ATGGCGGCTTCACCAGCTTTCTGCGGCGGAAGTGGAAGTTTAAGAAAGAGCGGAACAAGGGGTACAAG
CACCACGCCGAGGACGCCCTGATCATTGCCAACGCCGATTTCATCTTCAAAGAGTGGAAGAAACTGGA
CAAGGCCAAAAAAGTGATGGAAAACCAGATGTTCGAGGAAAAGCAGGCCGAGAGCATGCCCGAGATCG
AAACCGAGCAGGAGTACAAAGAGATCTTCATCACCCCCCACCAGATCAAGCACATTAAGGACTTCAAG
GACTACAAGTACAGCCACCGGGTGGACAAGAAGCCTAATAGAGAGCTGATTAACGACACCCTGTACTC
CACCCGGAAGGACGACAAGGGCAACACCCTGATCGTGAACAATCTGAACGGCCTGTACGACAAGGACA
ATGACAAGCTGAAAAAGCTGATCAACAAGAGCCCCGAAAAGCTGCTGATGTACCACCACGACCCCCAG
ACCTACCAGAAACTGAAGCTGATTATGGAACAGTACGGCGACGAGAAGAATCCCCTGTACAAGTACTA
CGAGGAAACCGGGAACTACCTGACCAAGTACTCCAAAAAGGACAACGGCCCCGTGATCAAGAAGATTA
AGTATTACGGCAACAAACTGAACGCCCATCTGGACATCACCGACGACTACCCCAACAGCAGAAACAAG
GTCGTGAAGCTGTCCCTGAAGCCCTACAGATTCGACGTGTACCTGGACAATGGCGTGTACAAGTTCGT
GACCGTGAAGAATCTGGATGTGATCAAAAAAGAAAACTACTACGAAGTGAATAGCAAGTGCTATGAGG
AAGCTAAGAAGCTGAAGAAGATCAGCAACCAGGCCGAGTTTATCGCCTCCTTCTACAACAACGATCTG
ATCAAGATCAACGGCGAGCTGTATAGAGTGATCGGCGTGAACAACGACCTGCTGAACCGGATCGAAGT
GAACATGATCGACATCACCTACCGCGAGTACCTGGAAAACATGAACGACAAGAGGCCCCCCAGGATCA
TTAAGACAATCGCCTCCAAGACCCAGAGCATTAAGAAGTACAGCACAGACATTCTGGGCAACCTGTAT
GAAGTGAAATCTAAGAAGCACCCTCAGATCATCAAAAAGGGCAAAAGGCCGGCGGCCACGAAAAAGGC
CGGCCAGGCAAAAAAGAAAAAGggatcctacccatacgatgttccagattacgcttacccatacgatg
ttccagattacgcttaccCatacgatgttccagattacgcttaaGAATTCctagagctcgctgatcag
cctcgactgtgccttctagttgccagccatctgttgtttgcccctcccccgtgccttccttgaccctg
gaaggtgccactcccactgtcctttcctaataaaatgaggaaattgcatcgcattgtctgagtaggtg
tcattctattctggggggtggggtggggcaggacagcaagggggaggattgggaagagaatagcaggc
atgctggggaGGTACCGAACGCTGACGTCATCAACCCGCTCCAAGGAATCGCGGGCCCAGTGTCACTA
GGCGGGAACACCCAGCGCGCGTGCGCCCTGGCAGGAAGATGGCTGTGAGGGACAGGGGAGTGGCGCCC
TGCAATATTTGCATGTCGCTATGTGTTCTGGGAAATCACCATAAACGTGAAATGTCTTTGGATTTGGG
AATCTTATAAGTTCTGTATGAGACCACATATAGTAATGAAATTATTGGCACGTTTTAGTACTCTGGAA
ACAGAATCTACTAAAACAAGGCAAAATGCCGTGTTTATCTCGTCAACTTGTTGGCGAGATTTTTGGTA
CCaggaacccctagtgatggagttggccactccctctctgcgcgctcgctcgctcactgaggccgggc
gaccaaaggtcgcccgacgcccgggctttgcccgggcggcctcagtgagcgagcgagcgcgcagctgc
ctgcaggggcgcctgatgcggtattttctccttacgcatctgtgcggtatttcacaccgcatacgtca
aagcaaccatagtacgcgccctgtagcggcgcattaagcgcggcgggtgtggtggttacgcgcagcgt
gaccgctacacttgccagcgccctagcgcccgctcctttcgctttcttcccttcctttctcgccacgt
tcgccggctttccccgtcaagctctaaatcgggggctccctttagggttccgatttagtgctttacgg
cacctcgaccccaaaaaacttgatttgggtgatggttcacgtagtgggccatcgccctgatagacggt
ttttcgccctttgacgttggagtccacgttctttaatagtggactcttgttccaaactggaacaacac
tcaaccctatctcgggctattcttttgatttataagggattttgccgatttcggcctattggttaaaa
aatgagctgatttaacaaaaatttaacgcgaattttaacaaaatattaacgtttacaattttatggtg
cactctcagtacaatctgctctgatgccgcatagttaagccagccccgacacccgccaacacccgctg
acgcgccctgacgggcttgtctgctcccggcatccgcttacagacaagctgtgaccgtctccgggagc
tgcatgtgtcagaggttttcaccgtcatcaccgaaacgcgcgagacgaaagggcctcgtgatacgcct
atttttataggttaatgtcatgataataatggtttcttagacgtcaggtggcacttttcggggaaatg
tgcgcggaacccctatttgtttatttttctaaatacattcaaatatgtatccgctcatgagacaataa
ccctgataaatgcttcaataatattgaaaaaggaagagtatgagtattcaacatttccgtgtcgccct
tattcccttttttgcggcattttgccttcctgtttttgctcacccagaaacgctggtgaaagtaaaag
atgctgaagatcagttgggtgcacgagtgggttacatcgaactggatctcaacagcggtaagatcctt
gagagttttcgccccgaagaacgttttccaatgatgagcacttttaaagttctgctatgtggcgcggt
attatcccgtattgacgccgggcaagagcaactcggtcgccgcatacactattctcagaatgacttgg
ttgagtactcaccagtcacagaaaagcatcttacggatggcatgacagtaagagaattatgcagtgct
gccataaccatgagtgataacactgcggccaacttacttctgacaacgatcggaggaccgaaggagct
aaccgcttttttgcacaacatgggggatcatgtaactcgccttgatcgttgggaaccggagctgaatg
aagccataccaaacgacgagcgtgacaccacgatgcctgtagcaatggcaacaacgttgcgcaaacta
ttaactggcgaactacttactctagcttcccggcaacaattaatagactggatggaggcggataaagt
tgcaggaccacttctgcgctcggcccttccggctggctggtttattgctgataaatctggagccggtg
agcgtggaagccgcggtatcattgcagcactggggccagatggtaagccctcccgtatcgtagttatc
tacacgacggggagtcaggcaactatggatgaacgaaatagacagatcgctgagataggtgcctcact
gattaagcattggtaactgtcagaccaagtttactcatatatactttagattgatttaaaacttcatt
tttaatttaaaaggatctaggtgaagatcctttttgataatctcatgaccaaaatcccttaacgtgag
ttttcgttccactgagcgtcagaccccgtagaaaagatcaaaggatcttcttgagatcctttttttct
gcgcgtaatctgctgcttgcaaacaaaaaaaccaccgctaccagcggtggtttgtttgccggatcaag
agctaccaactctttttccgaaggtaactggcttcagcagagcgcagataccaaatactgtccttcta
gtgtagccgtagttaggccaccacttcaagaactctgtagcaccgcctacatacctcgctctgctaat
cctattaccagtggctgctgccagtggcgataagtcgtgtcttaccgggttggactcaagacgatagt
taccggataaggcgcagcggtcgggctgaacggggggttcgtgcacacagcccagcttggagcgaacg
acctacaccgaactgagatacctacagcgtgagctatgagaaagcgccacgcttcccgaagggagaaa
ggcggacaggtatccggtaagcggcagggtcggaacaggagagcgcacgagggagcttccagggggaa
acgcctggtatctttatagtcctgtcgggtttcgccacctctgacttgagcgtcgatttttgtgatgc
tcgtcaggggggcggagcctatggaaaaacgccagcaacgcggcctttttacggttcctggccttttg
ctggccttttgctcacatgt
Version 1 of vector 5
SEQ ID NO: 83
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGACTCGAGTCGAGTGGCTCCGGTGCCCGTCAGTGGGCAGAGCGCACATCGCCCACAGTCCCCGA
GAAGTTGGGGGGAGGGGTCGGCAATTGAACCGGTGCCTAGAGAAGGTGGCGCGGGGTAAACTGGGAAA
GTGATGTCGTGTACTGGCTCCGCCTTTTTCCCGAGGGTGGGGGAGAACCGTATATAAGTGCAGTAGTC
GCCGTGAACGTTCTTTTTCGCAACGGGTTTGCCGCCAGAACACAGGTGTCGTGACCGCGGCCATGGtc
tagaggatccggtactcgaggaactgaaaaaccagaaagttaactggtaagtttagtctttttgtctt
ttatttcaggtcccggatccggtggtggtgcaaatcaaagaactgctcctcagtggatgttgccttta
cttctaggcctgtacggaagtgttacgccaCCATGGCCCCAAAGAAGAAGCGGAAGGTCGGTATCCAC
GGAGTCCCAGCAGCCAAGCGGAACTACATCCTGGGCCTGGACATCGGCATCACCAGCGTGGGCTACGG
CATCATCGACTACGAGACACGGGACGTGATCGATGCCGGCGTGCGGCTGTTCAAAGAGGCCAACGTGG
AAAACAACGAGGGCAGGCGGAGCAAGAGAGGCGCCAGAAGGCTGAAGCGGCGGAGGCGGCATAGAATC
CAGAGAGTGAAGAAGCTGCTGTTCGACTACAACCTGCTGACCGACCACAGCGAGCTGAGCGGCATCAA
CCCCTACGAGGCCAGAGTGAAGGGCCTGAGCCAGAAGCTGAGCGAGGAAGAGTTCTCTGCCGCCCTGC
TGCACCTGGCCAAGAGAAGAGGCGTGCACAACGTGAACGAGGTGGAAGAGGACACCGGCAACGAGCTG
TCCACCAAAGAGCAGATCAGCCGGAACAGCAAGGCCCTGGAAGAGAAATACGTGGCCGAACTGCAGCT
GGAACGGCTGAAGAAAGACGGCGAAGTGCGGGGCAGCATCAACAGATTCAAGACCAGCGACTACGTGA
AAGAAGCCAAACAGCTGCTGAAGGTGCAGAAGGCCTACCACCAGCTGGACCAGAGCTTCATCGACACC
TACATCGACCTGCTGGAAACCCGGCGGACCTACTATGAGGGACCTGGCGAGGGCAGCCCCTTCGGCTG
GAAGGACATCAAAGAATGGTACGAGATGCTGATGGGCCACTGCACCTACTTCCCCGAGGAACTGCGGA
GCGTGAAGTACGCCTACAACGCCGACCTGTACAACGCCCTGAACGACCTGAACAATCTCGTGATCACC
AGGGACGAGAACGAGAAGCTGGAATATTACGAGAAGTTCCAGATCATCGAGAACGTGTTCAAGCAGAA
GAAGAAGCCCACCCTGAAGCAGATCGCCAAAGAAATCCTCGTGAACGAAGAGGATATTAAGGGCTACA
GAGTGACCAGCACCGGCAAGCCCGAGTTCACCAACCTGAAGGTGTACCACGACATCAAGGACATTACC
GCCCGGAAAGAGATTATTGAGAACGCCGAGCTGCTGGATCAGATTGCCAAGATCCTGACCATCTACCA
GAGCAGCGAGGACATCCAGGAAGAACTGACCAATCTGAACTCCGAGCTGACCCAGGAAGAGATCGAGC
AGATCTCTAATCTGAAGGGCTATACCGGCACCCACAACCTGAGCCTGAAGGCCATCAACCTGATCCTG
GACGAGCTGTGGCACACCAACGACAACCAGATCGCTATCTTCAACCGGCTGAAGCTGGTGCCCAAGAA
GGTGGACCTGTCCCAGCAGAAAGAGATCCCCACCACCCTGGTGGACGACTTCATCCTGAGCCCCGTCG
TGAAGAGAAGCTTCATCCAGAGCATCAAAGTGATCAACGCCATCATCAAGAAGTACGGCCTGCCCAAC
GACATCATTATCGAGCTGGCCCGCGAGAAGAACTCCAAGGACGCCCAGAAAATGATCAACGAGATGCA
GAAGCGGAACCGGCAGACCAACGAGCGGATCGAGGAAATCATCCGGACCACCGGCAAAGAGAACGCCA
AGTACCTGATCGAGAAGATCAAGCTGCACGACATGCAGGAAGGCAAGTGCCTGTACAGCCTGGAAGCC
ATCCCTCTGGAAGATCTGCTGAACAACCCCTTCAACTATGAGGTGGACCACATCATCCCCAGAAGCGT
GTCCTTCGACAACAGCTTCAACAACAAGGTGCTCGTGAAGCAGGAAGAAAACAGCAAGAAGGGCAACC
GGACCCCATTCCAGTACCTGAGCAGCAGCGACAGCAAGATCAGCTACGAAACCTTCAAGAAGCACATC
CTGAATCTGGCCAAGGGCAAGGGCAGAATCAGCAAGACCAAGAAAGAGTATCTGCTGGAAGAACGGGA
CATCAACAGGTTCTCCGTGCAGAAAGACTTCATCAACCGGAACCTGGTGGATACCAGATACGCCACCA
GAGGCCTGATGAACCTGCTGCGGAGCTACTTCAGAGTGAACAACCTGGACGTGAAAGTGAAGTCCATC
AATGGCGGCTTCACCAGCTTTCTGCGGCGGAAGTGGAAGTTTAAGAAAGAGCGGAACAAGGGGTACAA
GCACCACGCCGAGGACGCCCTGATCATTGCCAACGCCGATTTCATCTTCAAAGAGTGGAAGAAACTGG
ACAAGGCCAAAAAAGTGATGGAAAACCAGATGTTCGAGGAAAAGCAGGCCGAGAGCATGCCCGAGATC
GAAACCGAGCAGGAGTACAAAGAGATCTTCATCACCCCCCACCAGATCAAGCACATTAAGGACTTCAA
GGACTACAAGTACAGCCACCGGGTGGACAAGAAGCCTAATAGAGAGCTGATTAACGACACCCTGTACT
CCACCCGGAAGGACGACAAGGGCAACACCCTGATCGTGAACAATCTGAACGGCCTGTACGACAAGGAC
AATGACAAGCTGAAAAAGCTGATCAACAAGAGCCCCGAAAAGCTGCTGATGTACCACCACGACCCCCA
GACCTACCAGAAACTGAAGCTGATTATGGAACAGTACGGCGACGAGAAGAATCCCCTGTACAAGTACT
ACGAGGAAACCGGGAACTACCTGACCAAGTACTCCAAAAAGGACAACGGCCCCGTGATCAAGAAGATT
AAGTATTACGGCAACAAACTGAACGCCCATCTGGACATCACCGACGACTACCCCAACAGCAGAAACAA
GGTCGTGAAGCTGTCCCTGAAGCCCTACAGATTCGACGTGTACCTGGACAATGGCGTGTACAAGTTCG
TGACCGTGAAGAATCTGGATGTGATCAAAAAAGAAAACTACTACGAAGTGAATAGCAAGTGCTATGAG
GAAGCTAAGAAGCTGAAGAAGATCAGCAACCAGGCCGAGTTTATCGCCTCCTTCTACAACAACGATCT
GATCAAGATCAACGGCGAGCTGTATAGAGTGATCGGCGTGAACAACGACCTGCTGAACCGGATCGAAG
TGAACATGATCGACATCACCTACCGCGAGTACCTGGAAAACATGAACGACAAGAGGCCCCCCAGGATC
ATTAAGACAATCGCCTCCAAGACCCAGAGCATTAAGAAGTACAGCACAGACATTCTGGGCAACCTGTA
TGAAGTGAAATCTAAGAAGCACCCTCAGATCATCAAAAAGGGCAAAAGGCCGGCGGCCACGAAAAAGG
CCGGCCAGGCAAAAAAGAAAAAGggatccGAATTCtagcaataaaggatcgtttattttcattggaag
cgtgtgttggttttttgatcaggcgcgGGTACCAAAAATCTCGCCAACAAGTTGACGAGATAAACACG
GCATTTTGCCTTGTTTTAGTAGATTCTGTTTCCAGAGTACTAAAACacatttcctctctatacaaatg
CGGTGTTTCGTCCTTTCCACAAGATATATAAAGCCAAGAAATCGAAATACTTTCAAGTTACGGTAAGC
ATATGATAGTCCATTTTAAAACATAATTTTAAAACTGCAAACTACCCAAGAAATTATTACTTTCTACG
TCACGTATTTTGTACTAATATCTTTGTGTTTACAGTCAAATTAATTCCAATTATCTCTCTAACAGCCT
TGTATCGTATATGCAAATATGAAGGAATCATGGGAAATAGGCCCTCCTCGACTAGTAGAAAAATCTCG
CCAACAAGTTGACGAGATAAACACGGCATTTTGCCTTGTTTTAGTAGATTCTGTTTCCAGAGTACTAA
AACGTGCCAATAATTTCATTACTATATCGGTGTTTCGTCCTTTCCACAAGATATATAAAGCCAAGAAA
TCGAAATACTTTCAAGTTACGGTAAGCATATGATAGTCCATTTTAAAACATAATTTTAAAACTGCAAA
CTACCCAAGAAATTATTACTTTCTACGTCACGTATTTTGTACTAATATCTTTGTGTTTACAGTCAAAT
TAATTCCAATTATCTCTCTAACAGCCTTGTATCGTATATGCAAATATGAAGGAATCATGGGAAATAGG
CCCTCGGTACCaggaacccctagtgatggagttggccactccctctctgcgcgctcgctcgctcactg
aggccgggcgaccaaaggtcgcccgacgcccgggctttgcccgggcggcctcagtgagcgagcgagcg
cgcagctgcctgcaggggcgcctgatgcggtattttctccttacgcatctgtgcggtatttcacaccg
catacgtcaaagcaaccatagtacgcgccctgtagcggcgcattaagcgcggcgggtgtggtggttac
gcgcagcgtgaccgctacacttgccagcgccctagcgcccgctcctttcgctttcttcccttcctttc
tcgccacgttcgccggctttccccgtcaagctctaaatcgggggctccctttagggttccgatttagt
gctttacggcacctcgaccccaaaaaacttgatttgggtgatggttcacgtagtgggccatcgccctg
atagacggtttttcgccctttgacgttggagtccacgttctttaatagtggactcttgttccaaactg
gaacaacactcaaccctatctcgggctattcttttgatttataagggattttgccgatttcggcctat
tggttaaaaaatgagctgatttaacaaaaatttaacgcgaattttaacaaaatattaacgtttacaat
tttatggtgcactctcagtacaatctgctctgatgccgcatagttaagccagccccgacacccgccaa
cacccgctgacgcgccctgacgggcttgtctgctcccggcatccgcttacagacaagctgtgaccgtc
tccgggagctgcatgtgtcagaggttttcaccgtcatcaccgaaacgcgcgagacgaaagggcctcgt
gatacgcctatttttataggttaatgtcatgataataatggtttcttagacgtcaggtggcacttttc
ggggaaatgtgcgcggaacccctatttgtttatttttctaaatacattcaaatatgtatccgctcatg
agacaataaccctgataaatgcttcaataatattgaaaaaggaagagtatgagtattcaacatttccg
tgtcgcccttattcccttttttgcggcattttgccttcctgtttttgctcacccagaaacgctggtga
aagtaaaagatgctgaagatcagttgggtgcacgagtgggttacatcgaactggatctcaacagcggt
aagatccttgagagttttcgccccgaagaacgttttccaatgatgagcacttttaaagttctgctatg
tggcgcggtattatcccgtattgacgccgggcaagagcaactcggtcgccgcatacactattctcaga
atgacttggttgagtactcaccagtcacagaaaagcatcttacggatggcatgacagtaagagaatta
tgcagtgctgccataaccatgagtgataacactgcggccaacttacttctgacaacgatcggaggacc
gaaggagctaaccgcttttttgcacaacatgggggatcatgtaactcgccttgatcgttgggaaccgg
agctgaatgaagccataccaaacgacgagcgtgacaccacgatgcctgtagcaatggcaacaacgttg
cgcaaactattaactggcgaactacttactctagcttcccggcaacaattaatagactggatggaggc
ggataaagttgcaggaccacttctgcgctcggcccttccggctggctggtttattgctgataaatctg
gagccggtgagcgtggaagccgcggtatcattgcagcactggggccagatggtaagccctcccgtatc
gtagttatctacacgacggggagtcaggcaactatggatgaacgaaatagacagatcgctgagatagg
tgcctcactgattaagcattggtaactgtcagaccaagtttactcatatatactttagattgatttaa
aacttcatttttaatttaaaaggatctaggtgaagatcctttttgataatctcatgaccaaaatccct
taacgtgagttttcgttccactgagcgtcagaccccgtagaaaagatcaaaggatcttcttgagatcc
tttttttctgcgcgtaatctgctgcttgcaaacaaaaaaaccaccgctaccagcggtggtttgtttgc
cggatcaagagctaccaactctttttccgaaggtaactggcttcagcagagcgcagataccaaatact
gtccttctagtgtagccgtagttaggccaccacttcaagaactctgtagcaccgcctacatacctcgc
tctgctaatcctgttaccagtggctgctgccagtggcgataagtcgtgtcttaccgggttggactcaa
gacgatagttaccggataaggcgcagcggtcgggctgaacggggggttcgtgcacacagcccagcttg
gagcgaacgacctacaccgaactgagatacctacagcgtgagctatgagaaagcgccacgcttcccga
agggagaaaggcggacaggtatccggtaagcggcagggtcggaacaggagagcgcacgagggagcttc
cagggggaaacgcctggtatctttatagtcctgtcgggtttcgccacctctgacttgagcgtcgattt
ttgtgatgctcgtcaggggggcggagcctatggaaaaacgccagcaacgcggcctttttacggttcct
ggccttttgctggccttttgctcacatgt
Version 2 of vector 5
SEQ ID NO: 84
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGACTCGAGCTAGACTAGCATGCTGCCCATGTAAGGAGGCAAGGCCTGGGGACACCCGAGATGCC
TGGTTATAATTAACCCAGACATGTGGCTGCCCCCCCCCCCCCAACACCTGCTGCCTCTAAAAATAACC
CTGCATGCCATGTTCCCGGCGAAGGGCCAGCTGTCCCCCGCCAGCTAGACTCAGCACTTAGTTTAGGA
ACCAGTGAGCAAGTCAGCCCTTGGGGCAGCCCATACAAGGCCATGGGGCTGGGCAAGCTGCACGCCTG
GGTCCGGGGTGGGCACGGTGCCCGGGCAACGAGCTGAAAGCTCATCTGCTCTCAGGGGCCCCTCCCTG
GGGACAGCCCCTCCTGGCTAGTCACACCCTGTAGGCTCCTCTATATAACCCAGGGGCACAGGGGCTGC
CCTCATTCTACCACCACCTCCACAGCACAGACAGACACTCAGGAGCCAGCCAGCCATGGtctagagga
tccggtactcgaggaactgaaaaaccagaaagttaactggtaagtttagtctttttgtcttttatttc
aggtcccggatccggtggtggtgcaaatcaaagaactgctcctcagtggatgttgcctttacttctag
gcctgtacggaagtgttacgccaCCATGGCCCCAAAGAAGAAGCGGAAGGTCGGTATCCACGGAGTCC
CAGCAGCCAAGCGGAACTACATCCTGGGCCTGGACATCGGCATCACCAGCGTGGGCTACGGCATCATC
GACTACGAGACACGGGACGTGATCGATGCCGGCGTGCGGCTGTTCAAAGAGGCCAACGTGGAAAACAA
CGAGGGCAGGCGGAGCAAGAGAGGCGCCAGAAGGCTGAAGCGGCGGAGGCGGCATAGAATCCAGAGAG
TGAAGAAGCTGCTGTTCGACTACAACCTGCTGACCGACCACAGCGAGCTGAGCGGCATCAACCCCTAC
GAGGCCAGAGTGAAGGGCCTGAGCCAGAAGCTGAGCGAGGAAGAGTTCTCTGCCGCCCTGCTGCACCT
GGCCAAGAGAAGAGGCGTGCACAACGTGAACGAGGTGGAAGAGGACACCGGCAACGAGCTGTCCACCA
AAGAGCAGATCAGCCGGAACAGCAAGGCCCTGGAAGAGAAATACGTGGCCGAACTGCAGCTGGAACGG
CTGAAGAAAGACGGCGAAGTGCGGGGCAGCATCAACAGATTCAAGACCAGCGACTACGTGAAAGAAGC
CAAACAGCTGCTGAAGGTGCAGAAGGCCTACCACCAGCTGGACCAGAGCTTCATCGACACCTACATCG
ACCTGCTGGAAACCCGGCGGACCTACTATGAGGGACCTGGCGAGGGCAGCCCCTTCGGCTGGAAGGAC
ATCAAAGAATGGTACGAGATGCTGATGGGCCACTGCACCTACTTCCCCGAGGAACTGCGGAGCGTGAA
GTACGCCTACAACGCCGACCTGTACAACGCCCTGAACGACCTGAACAATCTCGTGATCACCAGGGACG
AGAACGAGAAGCTGGAATATTACGAGAAGTTCCAGATCATCGAGAACGTGTTCAAGCAGAAGAAGAAG
CCCACCCTGAAGCAGATCGCCAAAGAAATCCTCGTGAACGAAGAGGATATTAAGGGCTACAGAGTGAC
CAGCACCGGCAAGCCCGAGTTCACCAACCTGAAGGTGTACCACGACATCAAGGACATTACCGCCCGGA
AAGAGATTATTGAGAACGCCGAGCTGCTGGATCAGATTGCCAAGATCCTGACCATCTACCAGAGCAGC
GAGGACATCCAGGAAGAACTGACCAATCTGAACTCCGAGCTGACCCAGGAAGAGATCGAGCAGATCTC
TAATCTGAAGGGCTATACCGGCACCCACAACCTGAGCCTGAAGGCCATCAACCTGATCCTGGACGAGC
TGTGGCACACCAACGACAACCAGATCGCTATCTTCAACCGGCTGAAGCTGGTGCCCAAGAAGGTGGAC
CTGTCCCAGCAGAAAGAGATCCCCACCACCCTGGTGGACGACTTCATCCTGAGCCCCGTCGTGAAGAG
AAGCTTCATCCAGAGCATCAAAGTGATCAACGCCATCATCAAGAAGTACGGCCTGCCCAACGACATCA
TTATCGAGCTGGCCCGCGAGAAGAACTCCAAGGACGCCCAGAAAATGATCAACGAGATGCAGAAGCGG
AACCGGCAGACCAACGAGCGGATCGAGGAAATCATCCGGACCACCGGCAAAGAGAACGCCAAGTACCT
GATCGAGAAGATCAAGCTGCACGACATGCAGGAAGGCAAGTGCCTGTACAGCCTGGAAGCCATCCCTC
TGGAAGATCTGCTGAACAACCCCTTCAACTATGAGGTGGACCACATCATCCCCAGAAGCGTGTCCTTC
GACAACAGCTTCAACAACAAGGTGCTCGTGAAGCAGGAAGAAAACAGCAAGAAGGGCAACCGGACCCC
ATTCCAGTACCTGAGCAGCAGCGACAGCAAGATCAGCTACGAAACCTTCAAGAAGCACATCCTGAATC
TGGCCAAGGGCAAGGGCAGAATCAGCAAGACCAAGAAAGAGTATCTGCTGGAAGAACGGGACATCAAC
AGGTTCTCCGTGCAGAAAGACTTCATCAACCGGAACCTGGTGGATACCAGATACGCCACCAGAGGCCT
GATGAACCTGCTGCGGAGCTACTTCAGAGTGAACAACCTGGACGTGAAAGTGAAGTCCATCAATGGCG
GCTTCACCAGCTTTCTGCGGCGGAAGTGGAAGTTTAAGAAAGAGCGGAACAAGGGGTACAAGCACCAC
GCCGAGGACGCCCTGATCATTGCCAACGCCGATTTCATCTTCAAAGAGTGGAAGAAACTGGACAAGGC
CAAAAAAGTGATGGAAAACCAGATGTTCGAGGAAAAGCAGGCCGAGAGCATGCCCGAGATCGAAACCG
AGCAGGAGTACAAAGAGATCTTCATCACCCCCCACCAGATCAAGCACATTAAGGACTTCAAGGACTAC
AAGTACAGCCACCGGGTGGACAAGAAGCCTAATAGAGAGCTGATTAACGACACCCTGTACTCCACCCG
GAAGGACGACAAGGGCAACACCCTGATCGTGAACAATCTGAACGGCCTGTACGACAAGGACAATGACA
AGCTGAAAAAGCTGATCAACAAGAGCCCCGAAAAGCTGCTGATGTACCACCACGACCCCCAGACCTAC
CAGAAACTGAAGCTGATTATGGAACAGTACGGCGACGAGAAGAATCCCCTGTACAAGTACTACGAGGA
AACCGGGAACTACCTGACCAAGTACTCCAAAAAGGACAACGGCCCCGTGATCAAGAAGATTAAGTATT
ACGGCAACAAACTGAACGCCCATCTGGACATCACCGACGACTACCCCAACAGCAGAAACAAGGTCGTG
AAGCTGTCCCTGAAGCCCTACAGATTCGACGTGTACCTGGACAATGGCGTGTACAAGTTCGTGACCGT
GAAGAATCTGGATGTGATCAAAAAAGAAAACTACTACGAAGTGAATAGCAAGTGCTATGAGGAAGCTA
AGAAGCTGAAGAAGATCAGCAACCAGGCCGAGTTTATCGCCTCCTTCTACAACAACGATCTGATCAAG
ATCAACGGCGAGCTGTATAGAGTGATCGGCGTGAACAACGACCTGCTGAACCGGATCGAAGTGAACAT
GATCGACATCACCTACCGCGAGTACCTGGAAAACATGAACGACAAGAGGCCCCCCAGGATCATTAAGA
CAATCGCCTCCAAGACCCAGAGCATTAAGAAGTACAGCACAGACATTCTGGGCAACCTGTATGAAGTG
AAATCTAAGAAGCACCCTCAGATCATCAAAAAGGGCAAAAGGCCGGCGGCCACGAAAAAGGCCGGCCA
GGCAAAAAAGAAAAAGggatccGAATTCtagcaataaaggatcgtttattttcattggaagcgtgtgt
tggttttttgatcaggcgcgGGTACCAAAAATCTCGCCAACAAGTTGACGAGATAAACACGGCATTTT
GCCTTGTTTTAGTAGATTCTGTTTCCAGAGTACTAAAACacatttcctctctatacaaatgCGGTGTT
TCGTCCTTTCCACAAGATATATAAAGCCAAGAAATCGAAATACTTTCAAGTTACGGTAAGCATATGAT
AGTCCATTTTAAAACATAATTTTAAAACTGCAAACTACCCAAGAAATTATTACTTTCTACGTCACGTA
TTTTGTACTAATATCTTTGTGTTTACAGTCAAATTAATTCCAATTATCTCTCTAACAGCCTTGTATCG
TATATGCAAATATGAAGGAATCATGGGAAATAGGCCCTCCTCGACTAGTAGAAAAATCTCGCCAACAA
GTTGACGAGATAAACACGGCATTTTGCCTTGTTTTAGTAGATTCTGTTTCCAGAGTACTAAAACGTGC
CAATAATTTCATTACTATATCGGTGTTTCGTCCTTTCCACAAGATATATAAAGCCAAGAAATCGAAAT
ACTTTCAAGTTACGGTAAGCATATGATAGTCCATTTTAAAACATAATTTTAAAACTGCAAACTACCCA
AGAAATTATTACTTTCTACGTCACGTATTTTGTACTAATATCTTTGTGTTTACAGTCAAATTAATTCC
AATTATCTCTCTAACAGCCTTGTATCGTATATGCAAATATGAAGGAATCATGGGAAATAGGCCCTCGG
TACCaggaacccctagtgatggagttggccactccctctctgcgcgctcgctcgctcactgaggccgg
gcgaccaaaggtcgcccgacgcccgggctttgcccgggcggcctcagtgagcgagcgagcgcgcagct
gcctgcaggggcgcctgatgcggtattttctccttacgcatctgtgcggtatttcacaccgcatacgt
caaagcaaccatagtacgcgccctgtagcggcgcattaagcgcggcgggtgtggtggttacgcgcagc
gtgaccgctacacttgccagcgccctagcgcccgctcctttcgctttcttcccttcctttctcgccac
gttcgccggctttccccgtcaagctctaaatcgggggctccctttagggttccgatttagtgctttac
ggcacctcgaccccaaaaaacttgatttgggtgatggttcacgtagtgggccatcgccctgatagacg
gtttttcgccctttgacgttggagtccacgttctttaatagtggactcttgttccaaactggaacaac
actcaaccctatctcgggctattcttttgatttataagggattttgccgatttcggcctattggttaa
aaaatgagctgatttaacaaaaatttaacgcgaattttaacaaaatattaacgtttacaattttatgg
tgcactctcagtacaatctgctctgatgccgcatagttaagccagccccgacacccgccaacacccgc
tgacgcgccctgacgggcttgtctgctcccggcatccgcttacagacaagctgtgaccgtctccggga
gctgcatgtgtcagaggttttcaccgtcatcaccgaaacgcgcgagacgaaagggcctcgtgatacgc
ctatttttataggttaatgtcatgataataatggtttcttagacgtcaggtggcacttttcggggaaa
tgtgcgcggaacccctatttgtttatttttctaaatacattcaaatatgtatccgctcatgagacaat
aaccctgataaatgcttcaataatattgaaaaaggaagagtatgagtattcaacatttccgtgtcgcc
cttattcccttttttgcggcattttgccttcctgtttttgctcacccagaaacgctggtgaaagtaaa
agatgctgaagatcagttgggtgcacgagtgggttacatcgaactggatctcaacagcggtaagatcc
ttgagagttttcgccccgaagaacgttttccaatgatgagcacttttaaagttctgctatgtggcgcg
gtattatcccgtattgacgccgggcaagagcaactcggtcgccgcatacactattctcagaatgactt
ggttgagtactcaccagtcacagaaaagcatcttacggatggcatgacagtaagagaattatgcagtg
ctgccataaccatgagtgataacactgcggccaacttacttctgacaacgatcggaggaccgaaggag
ctaaccgcttttttgcacaacatgggggatcatgtaactcgccttgatcgttgggaaccggagctgaa
tgaagccataccaaacgacgagcgtgacaccacgatgcctgtagcaatggcaacaacgttgcgcaaac
tattaactggcgaactacttactctagcttcccggcaacaattaatagactggatggaggcggataaa
gttgcaggaccacttctgcgctcggcccttccggctggctggtttattgctgataaatctggagccgg
tgagcgtggaagccgcggtatcattgcagcactggggccagatggtaagccctcccgtatcgtagtta
tctacacgacggggagtcaggcaactatggatgaacgaaatagacagatcgctgagataggtgcctca
ctgattaagcattggtaactgtcagaccaagtttactcatatatactttagattgatttaaaacttca
tttttaatttaaaaggatctaggtgaagatcctttttgataatctcatgaccaaaatcccttaacgtg
agttttcgttccactgagcgtcagaccccgtagaaaagatcaaaggatcttcttgagatccttttttt
ctgcgcgtaatctgctgcttgcaaacaaaaaaaccaccgctaccagcggtggtttgtttgccggatca
agagctaccaactctttttccgaaggtaactggcttcagcagagcgcagataccaaatactgtccttc
tagtgtagccgtagttaggccaccacttcaagaactctgtagcaccgcctacatacctcgctctgcta
atcctgttaccagtggctgctgccagtggcgataagtcgtgtcttaccgggttggactcaagacgata
gttaccggataaggcgcagcggtcgggctgaacggggggttcgtgcacacagcccagcttggagcgaa
cgacctacaccgaactgagatacctacagcgtgagctatgagaaagcgccacgcttcccgaagggaga
aaggcggacaggtatccggtaagcggcagggtcggaacaggagagcgcacgagggagcttccaggggg
aaacgcctggtatctttatagtcctgtcgggtttcgccacctctgacttgagcgtcgatttttgtgat
gctcgtcaggggggcggagcctatggaaaaacgccagcaacgcggcctttttacggttcctggccttt
tgctggccttttgctcacatgt
Version 3 of vector 5
SEQ ID NO: 85
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGACTCGAGGAGCTCCACCGCGGTGGCGGCCGTCCGCCtTCGGCACCATCCTCACGACACCCAAA
TATGGCGACGGGTGAGGAATGGTGGGGAGTTATTTTTAGAGCGGTGAGGAAGGTGGGCAGGCAGCAGG
TGTTGGCGCTCTAAAAATAACTCCCGGGAGTTATTTTTAGAGCGGAGGAATGGTGGACACCCAAATAT
GGCGACGGTTCCTCACCCGTCGCCATATTTGGGTGTCCGCCCTCGGCCGGGGCCGCATTCCTGGGGGC
CGGGCGGTGCTCCCGCCCGCCTCGATAAAAGGCTCCGGGGCCGGCGGCGGCCCACGAGCTACCCGGAG
GAGCGGGAGGCGCCAAGCTCTAGAACTAGTGGATCCCCCGGGCTGCAGGAATTCGATATCCATGGtct
agaggatccggtactcgaggaactgaaaaaccagaaagttaactggtaagtttagtctttttgtcttt
tatttcaggtcccggatccggtggtggtgcaaatcaaagaactgctcctcagtggatgttgcctttac
ttctaggcctgtacggaagtgttacgccaCCATGGCCCCAAAGAAGAAGCGGAAGGTCGGTATCCACG
GAGTCCCAGCAGCCAAGCGGAACTACATCCTGGGCCTGGACATCGGCATCACCAGCGTGGGCTACGGC
ATCATCGACTACGAGACACGGGACGTGATCGATGCCGGCGTGCGGCTGTTCAAAGAGGCCAACGTGGA
AAACAACGAGGGCAGGCGGAGCAAGAGAGGCGCCAGAAGGCTGAAGCGGCGGAGGCGGCATAGAATCC
AGAGAGTGAAGAAGCTGCTGTTCGACTACAACCTGCTGACCGACCACAGCGAGCTGAGCGGCATCAAC
CCCTACGAGGCCAGAGTGAAGGGCCTGAGCCAGAAGCTGAGCGAGGAAGAGTTCTCTGCCGCCCTGCT
GCACCTGGCCAAGAGAAGAGGCGTGCACAACGTGAACGAGGTGGAAGAGGACACCGGCAACGAGCTGT
CCACCAAAGAGCAGATCAGCCGGAACAGCAAGGCCCTGGAAGAGAAATACGTGGCCGAACTGCAGCTG
GAACGGCTGAAGAAAGACGGCGAAGTGCGGGGCAGCATCAACAGATTCAAGACCAGCGACTACGTGAA
AGAAGCCAAACAGCTGCTGAAGGTGCAGAAGGCCTACCACCAGCTGGACCAGAGCTTCATCGACACCT
ACATCGACCTGCTGGAAACCCGGCGGACCTACTATGAGGGACCTGGCGAGGGCAGCCCCTTCGGCTGG
AAGGACATCAAAGAATGGTACGAGATGCTGATGGGCCACTGCACCTACTTCCCCGAGGAACTGCGGAG
CGTGAAGTACGCCTACAACGCCGACCTGTACAACGCCCTGAACGACCTGAACAATCTCGTGATCACCA
GGGACGAGAACGAGAAGCTGGAATATTACGAGAAGTTCCAGATCATCGAGAACGTGTTCAAGCAGAAG
AAGAAGCCCACCCTGAAGCAGATCGCCAAAGAAATCCTCGTGAACGAAGAGGATATTAAGGGCTACAG
AGTGACCAGCACCGGCAAGCCCGAGTTCACCAACCTGAAGGTGTACCACGACATCAAGGACATTACCG
CCCGGAAAGAGATTATTGAGAACGCCGAGCTGCTGGATCAGATTGCCAAGATCCTGACCATCTACCAG
AGCAGCGAGGACATCCAGGAAGAACTGACCAATCTGAACTCCGAGCTGACCCAGGAAGAGATCGAGCA
GATCTCTAATCTGAAGGGCTATACCGGCACCCACAACCTGAGCCTGAAGGCCATCAACCTGATCCTGG
ACGAGCTGTGGCACACCAACGACAACCAGATCGCTATCTTCAACCGGCTGAAGCTGGTGCCCAAGAAG
GTGGACCTGTCCCAGCAGAAAGAGATCCCCACCACCCTGGTGGACGACTTCATCCTGAGCCCCGTCGT
GAAGAGAAGCTTCATCCAGAGCATCAAAGTGATCAACGCCATCATCAAGAAGTACGGCCTGCCCAACG
ACATCATTATCGAGCTGGCCCGCGAGAAGAACTCCAAGGACGCCCAGAAAATGATCAACGAGATGCAG
AAGCGGAACCGGCAGACCAACGAGCGGATCGAGGAAATCATCCGGACCACCGGCAAAGAGAACGCCAA
GTACCTGATCGAGAAGATCAAGCTGCACGACATGCAGGAAGGCAAGTGCCTGTACAGCCTGGAAGCCA
TCCCTCTGGAAGATCTGCTGAACAACCCCTTCAACTATGAGGTGGACCACATCATCCCCAGAAGCGTG
TCCTTCGACAACAGCTTCAACAACAAGGTGCTCGTGAAGCAGGAAGAAAACAGCAAGAAGGGCAACCG
GACCCCATTCCAGTACCTGAGCAGCAGCGACAGCAAGATCAGCTACGAAACCTTCAAGAAGCACATCC
TGAATCTGGCCAAGGGCAAGGGCAGAATCAGCAAGACCAAGAAAGAGTATCTGCTGGAAGAACGGGAC
ATCAACAGGTTCTCCGTGCAGAAAGACTTCATCAACCGGAACCTGGTGGATACCAGATACGCCACCAG
AGGCCTGATGAACCTGCTGCGGAGCTACTTCAGAGTGAACAACCTGGACGTGAAAGTGAAGTCCATCA
ATGGCGGCTTCACCAGCTTTCTGCGGCGGAAGTGGAAGTTTAAGAAAGAGCGGAACAAGGGGTACAAG
CACCACGCCGAGGACGCCCTGATCATTGCCAACGCCGATTTCATCTTCAAAGAGTGGAAGAAACTGGA
CAAGGCCAAAAAAGTGATGGAAAACCAGATGTTCGAGGAAAAGCAGGCCGAGAGCATGCCCGAGATCG
AAACCGAGCAGGAGTACAAAGAGATCTTCATCACCCCCCACCAGATCAAGCACATTAAGGACTTCAAG
GACTACAAGTACAGCCACCGGGTGGACAAGAAGCCTAATAGAGAGCTGATTAACGACACCCTGTACTC
CACCCGGAAGGACGACAAGGGCAACACCCTGATCGTGAACAATCTGAACGGCCTGTACGACAAGGACA
ATGACAAGCTGAAAAAGCTGATCAACAAGAGCCCCGAAAAGCTGCTGATGTACCACCACGACCCCCAG
ACCTACCAGAAACTGAAGCTGATTATGGAACAGTACGGCGACGAGAAGAATCCCCTGTACAAGTACTA
CGAGGAAACCGGGAACTACCTGACCAAGTACTCCAAAAAGGACAACGGCCCCGTGATCAAGAAGATTA
AGTATTACGGCAACAAACTGAACGCCCATCTGGACATCACCGACGACTACCCCAACAGCAGAAACAAG
GTCGTGAAGCTGTCCCTGAAGCCCTACAGATTCGACGTGTACCTGGACAATGGCGTGTACAAGTTCGT
GACCGTGAAGAATCTGGATGTGATCAAAAAAGAAAACTACTACGAAGTGAATAGCAAGTGCTATGAGG
AAGCTAAGAAGCTGAAGAAGATCAGCAACCAGGCCGAGTTTATCGCCTCCTTCTACAACAACGATCTG
ATCAAGATCAACGGCGAGCTGTATAGAGTGATCGGCGTGAACAACGACCTGCTGAACCGGATCGAAGT
GAACATGATCGACATCACCTACCGCGAGTACCTGGAAAACATGAACGACAAGAGGCCCCCCAGGATCA
TTAAGACAATCGCCTCCAAGACCCAGAGCATTAAGAAGTACAGCACAGACATTCTGGGCAACCTGTAT
GAAGTGAAATCTAAGAAGCACCCTCAGATCATCAAAAAGGGCAAAAGGCCGGCGGCCACGAAAAAGGC
CGGCCAGGCAAAAAAGAAAAAGggatccGAATTCtagcaataaaggatcgtttattttcattggaagc
gtgtgttggttttttgatcaggcgcgGGTACCAAAAATCTCGCCAACAAGTTGACGAGATAAACACGG
CATTTTGCCTTGTTTTAGTAGATTCTGTTTCCAGAGTACTAAAACacatttcctctctatacaaatgC
GGTGTTTCGTCCTTTCCACAAGATATATAAAGCCAAGAAATCGAAATACTTTCAAGTTACGGTAAGCA
TATGATAGTCCATTTTAAAACATAATTTTAAAACTGCAAACTACCCAAGAAATTATTACTTTCTACGT
CACGTATTTTGTACTAATATCTTTGTGTTTACAGTCAAATTAATTCCAATTATCTCTCTAACAGCCTT
GTATCGTATATGCAAATATGAAGGAATCATGGGAAATAGGCCCTCCTCGACTAGTAGAAAAATCTCGC
CAACAAGTTGACGAGATAAACACGGCATTTTGCCTTGTTTTAGTAGATTCTGTTTCCAGAGTACTAAA
ACGTGCCAATAATTTCATTACTATATCGGTGTTTCGTCCTTTCCACAAGATATATAAAGCCAAGAAAT
CGAAATACTTTCAAGTTACGGTAAGCATATGATAGTCCATTTTAAAACATAATTTTAAAACTGCAAAC
TACCCAAGAAATTATTACTTTCTACGTCACGTATTTTGTACTAATATCTTTGTGTTTACAGTCAAATT
AATTCCAATTATCTCTCTAACAGCCTTGTATCGTATATGCAAATATGAAGGAATCATGGGAAATAGGC
CCTCGGTACCaggaacccctagtgatggagttggccactccctctctgcgcgctcgctcgctcactga
ggccgggcgaccaaaggtcgcccgacgcccgggctttgcccgggcggcctcagtgagcgagcgagcgc
gcagctgcctgcaggggcgcctgatgcggtattttctccttacgcatctgtgcggtatttcacaccgc
atacgtcaaagcaaccatagtacgcgccctgtagcggcgcattaagcgcggcgggtgtggtggttacg
cgcagcgtgaccgctacacttgccagcgccctagcgcccgctcctttcgctttcttcccttcctttct
cgccacgttcgccggctttccccgtcaagctctaaatcgggggctccctttagggttccgatttagtg
ctttacggcacctcgaccccaaaaaacttgatttgggtgatggttcacgtagtgggccatcgccctga
tagacggtttttcgccctttgacgttggagtccacgttctttaatagtggactcttgttccaaactgg
aacaacactcaaccctatctcgggctattcttttgatttataagggattttgccgatttcggcctatt
ggttaaaaaatgagctgatttaacaaaaatttaacgcgaattttaacaaaatattaacgtttacaatt
ttatggtgcactctcagtacaatctgctctgatgccgcatagttaagccagccccgacacccgccaac
acccgctgacgcgccctgacgggcttgtctgctcccggcatccgcttacagacaagctgtgaccgtct
ccgggagctgcatgtgtcagaggttttcaccgtcatcaccgaaacgcgcgagacgaaagggcctcgtg
atacgcctatttttataggttaatgtcatgataataatggtttcttagacgtcaggtggcacttttcg
gggaaatgtgcgcggaacccctatttgtttatttttctaaatacattcaaatatgtatccgctcatga
gacaataaccctgataaatgcttcaataatattgaaaaaggaagagtatgagtattcaacatttccgt
gtcgcccttattcccttttttgcggcattttgccttcctgtttttgctcacccagaaacgctggtgaa
agtaaaagatgctgaagatcagttgggtgcacgagtgggttacatcgaactggatctcaacagcggta
agatccttgagagttttcgccccgaagaacgttttccaatgatgagcacttttaaagttctgctatgt
ggcgcggtattatcccgtattgacgccgggcaagagcaactcggtcgccgcatacactattctcagaa
tgacttggttgagtactcaccagtcacagaaaagcatcttacggatggcatgacagtaagagaattat
gcagtgctgccataaccatgagtgataacactgcggccaacttacttctgacaacgatcggaggaccg
aaggagctaaccgcttttttgcacaacatgggggatcatgtaactcgccttgatcgttgggaaccgga
gctgaatgaagccataccaaacgacgagcgtgacaccacgatgcctgtagcaatggcaacaacgttgc
gcaaactattaactggcgaactacttactctagcttcccggcaacaattaatagactggatggaggcg
gataaagttgcaggaccacttctgcgctcggcccttccggctggctggtttattgctgataaatctgg
agccggtgagcgtggaagccgcggtatcattgcagcactggggccagatggtaagccctcccgtatcg
tagttatctacacgacggggagtcaggcaactatggatgaacgaaatagacagatcgctgagataggt
gcctcactgattaagcattggtaactgtcagaccaagtttactcatatatactttagattgatttaaa
acttcatttttaatttaaaaggatctaggtgaagatcctttttgataatctcatgaccaaaatccctt
aacgtgagttttcgttccactgagcgtcagaccccgtagaaaagatcaaaggatcttcttgagatcct
ttttttctgcgcgtaatctgctgcttgcaaacaaaaaaaccaccgctaccagcggtggtttgtttgcc
ggatcaagagctaccaactctttttccgaaggtaactggcttcagcagagcgcagataccaaatactg
tccttctagtgtagccgtagttaggccaccacttcaagaactctgtagcaccgcctacatacctcgct
ctgctaatcctgttaccagtggctgctgccagtggcgataagtcgtgtcttaccgggttggactcaag
acgatagttaccggataaggcgcagcggtcgggctgaacggggggttcgtgcacacagcccagcttgg
agcgaacgacctacaccgaactgagatacctacagcgtgagctatgagaaagcgccacgcttcccgaa
gggagaaaggcggacaggtatccggtaagcggcagggtcggaacaggagagcgcacgagggagcttcc
agggggaaacgcctggtatctttatagtcctgtcgggtttcgccacctctgacttgagcgtcgatttt
tgtgatgctcgtcaggggggcggagcctatggaaaaacgccagcaacgcggcctttttacggttcctg
gccttttgctggccttttgctcacatgt
Version 4 of vector 5
SEQ ID NO: 86
cctgcaggcagctgcgcgctcgctcgctcactgaggccgcccgggcgtcgggcgacctttggtcgccc
ggcctcagtgagcgagcgagcgcgcagagagggagtggccaactccatcactaggggttcctgcggcc
TCTAGACTCGAGagcttgcatgtctaagctagacccttcagattaaaaataactgaggtaagggcctg
ggtaggggaggtggtgtgagacgctcctgtctctcctctatctgcccatcggccctttggggaggagg
aatgtgcccaaggactaaaaaaaggccatggagccagaggggcgagggcaacagacctttcatgggca
aaccttggggccctgctgtctagcatgccccactacgggtctaggctgcccatgtaaggaggcaaggc
ctggggacacccgagatgcctggttataattaacccagacatgtggctgcccccccccccccaacacc
tgctgcctctaaaaataaccctgtccctggtggatcccctgcatgcgaagatcttcgaacaaggctgt
gggggactgagggcaggctgtaacaggcttgggggccagggcttatacgtgcctgggactcccaaagt
attactgttccatgttcccggcgaagggccagctgtcccccgccagctagactcagcacttagtttag
gaaccagtgagcaagtcagcccttggggcagcccatacaaggccatggggctgggcaagctgcacgcc
tgggtccggggtgggcacggtgcccgggcaacgagctgaaagctcatctgctctcaggggcccctccc
tggggacagcccctcctggctagtcacaccctgtaggctcctctatataacccaggggcacaggggct
gccctcattctaccaccacctccacagcacagacagacactcaggagccagccagcCCATGGtctaga
ggatccggtactcgaggaactgaaaaaccagaaagttaactggtaagtttagtctttttgtcttttat
ttcaggtcccggatccggtggtggtgcaaatcaaagaactgctcctcagtggatgttgcctttacttc
taggcctgtacggaagtgttacgccaCCATGGCCCCAAAGAAGAAGCGGAAGGTCGGTATCCACGGAG
TCCCAGCAGCCAAGCGGAACTACATCCTGGGCCTGGACATCGGCATCACCAGCGTGGGCTACGGCATC
ATCGACTACGAGACACGGGACGTGATCGATGCCGGCGTGCGGCTGTTCAAAGAGGCCAACGTGGAAAA
CAACGAGGGCAGGCGGAGCAAGAGAGGCGCCAGAAGGCTGAAGCGGCGGAGGCGGCATAGAATCCAGA
GAGTGAAGAAGCTGCTGTTCGACTACAACCTGCTGACCGACCACAGCGAGCTGAGCGGCATCAACCCC
TACGAGGCCAGAGTGAAGGGCCTGAGCCAGAAGCTGAGCGAGGAAGAGTTCTCTGCCGCCCTGCTGCA
CCTGGCCAAGAGAAGAGGCGTGCACAACGTGAACGAGGTGGAAGAGGACACCGGCAACGAGCTGTCCA
CCAAAGAGCAGATCAGCCGGAACAGCAAGGCCCTGGAAGAGAAATACGTGGCCGAACTGCAGCTGGAA
CGGCTGAAGAAAGACGGCGAAGTGCGGGGCAGCATCAACAGATTCAAGACCAGCGACTACGTGAAAGA
AGCCAAACAGCTGCTGAAGGTGCAGAAGGCCTACCACCAGCTGGACCAGAGCTTCATCGACACCTACA
TCGACCTGCTGGAAACCCGGCGGACCTACTATGAGGGACCTGGCGAGGGCAGCCCCTTCGGCTGGAAG
GACATCAAAGAATGGTACGAGATGCTGATGGGCCACTGCACCTACTTCCCCGAGGAACTGCGGAGCGT
GAAGTACGCCTACAACGCCGACCTGTACAACGCCCTGAACGACCTGAACAATCTCGTGATCACCAGGG
ACGAGAACGAGAAGCTGGAATATTACGAGAAGTTCCAGATCATCGAGAACGTGTTCAAGCAGAAGAAG
AAGCCCACCCTGAAGCAGATCGCCAAAGAAATCCTCGTGAACGAAGAGGATATTAAGGGCTACAGAGT
GACCAGCACCGGCAAGCCCGAGTTCACCAACCTGAAGGTGTACCACGACATCAAGGACATTACCGCCC
GGAAAGAGATTATTGAGAACGCCGAGCTGCTGGATCAGATTGCCAAGATCCTGACCATCTACCAGAGC
AGCGAGGACATCCAGGAAGAACTGACCAATCTGAACTCCGAGCTGACCCAGGAAGAGATCGAGCAGAT
CTCTAATCTGAAGGGCTATACCGGCACCCACAACCTGAGCCTGAAGGCCATCAACCTGATCCTGGACG
AGCTGTGGCACACCAACGACAACCAGATCGCTATCTTCAACCGGCTGAAGCTGGTGCCCAAGAAGGTG
GACCTGTCCCAGCAGAAAGAGATCCCCACCACCCTGGTGGACGACTTCATCCTGAGCCCCGTCGTGAA
GAGAAGCTTCATCCAGAGCATCAAAGTGATCAACGCCATCATCAAGAAGTACGGCCTGCCCAACGACA
TCATTATCGAGCTGGCCCGCGAGAAGAACTCCAAGGACGCCCAGAAAATGATCAACGAGATGCAGAAG
CGGAACCGGCAGACCAACGAGCGGATCGAGGAAATCATCCGGACCACCGGCAAAGAGAACGCCAAGTA
CCTGATCGAGAAGATCAAGCTGCACGACATGCAGGAAGGCAAGTGCCTGTACAGCCTGGAAGCCATCC
CTCTGGAAGATCTGCTGAACAACCCCTTCAACTATGAGGTGGACCACATCATCCCCAGAAGCGTGTCC
TTCGACAACAGCTTCAACAACAAGGTGCTCGTGAAGCAGGAAGAAAACAGCAAGAAGGGCAACCGGAC
CCCATTCCAGTACCTGAGCAGCAGCGACAGCAAGATCAGCTACGAAACCTTCAAGAAGCACATCCTGA
ATCTGGCCAAGGGCAAGGGCAGAATCAGCAAGACCAAGAAAGAGTATCTGCTGGAAGAACGGGACATC
AACAGGTTCTCCGTGCAGAAAGACTTCATCAACCGGAACCTGGTGGATACCAGATACGCCACCAGAGG
CCTGATGAACCTGCTGCGGAGCTACTTCAGAGTGAACAACCTGGACGTGAAAGTGAAGTCCATCAATG
GCGGCTTCACCAGCTTTCTGCGGCGGAAGTGGAAGTTTAAGAAAGAGCGGAACAAGGGGTACAAGCAC
CACGCCGAGGACGCCCTGATCATTGCCAACGCCGATTTCATCTTCAAAGAGTGGAAGAAACTGGACAA
GGCCAAAAAAGTGATGGAAAACCAGATGTTCGAGGAAAAGCAGGCCGAGAGCATGCCCGAGATCGAAA
CCGAGCAGGAGTACAAAGAGATCTTCATCACCCCCCACCAGATCAAGCACATTAAGGACTTCAAGGAC
TACAAGTACAGCCACCGGGTGGACAAGAAGCCTAATAGAGAGCTGATTAACGACACCCTGTACTCCAC
CCGGAAGGACGACAAGGGCAACACCCTGATCGTGAACAATCTGAACGGCCTGTACGACAAGGACAATG
ACAAGCTGAAAAAGCTGATCAACAAGAGCCCCGAAAAGCTGCTGATGTACCACCACGACCCCCAGACC
TACCAGAAACTGAAGCTGATTATGGAACAGTACGGCGACGAGAAGAATCCCCTGTACAAGTACTACGA
GGAAACCGGGAACTACCTGACCAAGTACTCCAAAAAGGACAACGGCCCCGTGATCAAGAAGATTAAGT
ATTACGGCAACAAACTGAACGCCCATCTGGACATCACCGACGACTACCCCAACAGCAGAAACAAGGTC
GTGAAGCTGTCCCTGAAGCCCTACAGATTCGACGTGTACCTGGACAATGGCGTGTACAAGTTCGTGAC
CGTGAAGAATCTGGATGTGATCAAAAAAGAAAACTACTACGAAGTGAATAGCAAGTGCTATGAGGAAG
CTAAGAAGCTGAAGAAGATCAGCAACCAGGCCGAGTTTATCGCCTCCTTCTACAACAACGATCTGATC
AAGATCAACGGCGAGCTGTATAGAGTGATCGGCGTGAACAACGACCTGCTGAACCGGATCGAAGTGAA
CATGATCGACATCACCTACCGCGAGTACCTGGAAAACATGAACGACAAGAGGCCCCCCAGGATCATTA
AGACAATCGCCTCCAAGACCCAGAGCATTAAGAAGTACAGCACAGACATTCTGGGCAACCTGTATGAA
GTGAAATCTAAGAAGCACCCTCAGATCATCAAAAAGGGCAAAAGGCCGGCGGCCACGAAAAAGGCCGG
CCAGGCAAAAAAGAAAAAGggatccGAATTCtagcaataaaggatcgtttattttcattggaagcgtg
tgttggttttttgatcaggcgcgGGTACCAAAAATCTCGCCAACAAGTTGACGAGATAAACACGGCAT
TTTGCCTTGTTTTAGTAGATTCTGTTTCCAGAGTACTAAAACacatttcctctctatacaaatgCGGT
GTTTCGTCCTTTCCACAAGATATATAAAGCCAAGAAATCGAAATACTTTCAAGTTACGGTAAGCATAT
GATAGTCCATTTTAAAACATAATTTTAAAACTGCAAACTACCCAAGAAATTATTACTTTCTACGTCAC
GTATTTTGTACTAATATCTTTGTGTTTACAGTCAAATTAATTCCAATTATCTCTCTAACAGCCTTGTA
TCGTATATGCAAATATGAAGGAATCATGGGAAATAGGCCCTCCTCGACTAGTAGAAAAATCTCGCCAA
CAAGTTGACGAGATAAACACGGCATTTTGCCTTGTTTTAGTAGATTCTGTTTCCAGAGTACTAAAACG
TGCCAATAATTTCATTACTATATCGGTGTTTCGTCCTTTCCACAAGATATATAAAGCCAAGAAATCGA
AATACTTTCAAGTTACGGTAAGCATATGATAGTCCATTTTAAAACATAATTTTAAAACTGCAAACTAC
CCAAGAAATTATTACTTTCTACGTCACGTATTTTGTACTAATATCTTTGTGTTTACAGTCAAATTAAT
TCCAATTATCTCTCTAACAGCCTTGTATCGTATATGCAAATATGAAGGAATCATGGGAAATAGGCCCT
CGGTACCaggaacccctagtgatggagttggccactccctctctgcgcgctcgctcgctcactgaggc
cgggcgaccaaaggtcgcccgacgcccgggctttgcccgggcggcctcagtgagcgagcgagcgcgca
gctgcctgcaggggcgcctgatgcggtattttctccttacgcatctgtgcggtatttcacaccgcata
cgtcaaagcaaccatagtacgcgccctgtagcggcgcattaagcgcggcgggtgtggtggttacgcgc
agcgtgaccgctacacttgccagcgccctagcgcccgctcctttcgctttcttcccttcctttctcgc
cacgttcgccggctttccccgtcaagctctaaatcgggggctccctttagggttccgatttagtgctt
tacggcacctcgaccccaaaaaacttgatttgggtgatggttcacgtagtgggccatcgccctgatag
acggtttttcgccctttgacgttggagtccacgttctttaatagtggactcttgttccaaactggaac
aacactcaaccctatctcgggctattcttttgatttataagggattttgccgatttcggcctattggt
taaaaaatgagctgatttaacaaaaatttaacgcgaattttaacaaaatattaacgtttacaatttta
tggtgcactctcagtacaatctgctctgatgccgcatagttaagccagccccgacacccgccaacacc
cgctgacgcgccctgacgggcttgtctgctcccggcatccgcttacagacaagctgtgaccgtctccg
ggagctgcatgtgtcagaggttttcaccgtcatcaccgaaacgcgcgagacgaaagggcctcgtgata
cgcctatttttataggttaatgtcatgataataatggtttcttagacgtcaggtggcacttttcgggg
aaatgtgcgcggaacccctatttgtttatttttctaaatacattcaaatatgtatccgctcatgagac
aataaccctgataaatgcttcaataatattgaaaaaggaagagtatgagtattcaacatttccgtgtc
gcccttattcccttttttgcggcattttgccttcctgtttttgctcacccagaaacgctggtgaaagt
aaaagatgctgaagatcagttgggtgcacgagtgggttacatcgaactggatctcaacagcggtaaga
tccttgagagttttcgccccgaagaacgttttccaatgatgagcacttttaaagttctgctatgtggc
gcggtattatcccgtattgacgccgggcaagagcaactcggtcgccgcatacactattctcagaatga
cttggttgagtactcaccagtcacagaaaagcatcttacggatggcatgacagtaagagaattatgca
gtgctgccataaccatgagtgataacactgcggccaacttacttctgacaacgatcggaggaccgaag
gagctaaccgcttttttgcacaacatgggggatcatgtaactcgccttgatcgttgggaaccggagct
gaatgaagccataccaaacgacgagcgtgacaccacgatgcctgtagcaatggcaacaacgttgcgca
aactattaactggcgaactacttactctagcttcccggcaacaattaatagactggatggaggcggat
aaagttgcaggaccacttctgcgctcggcccttccggctggctggtttattgctgataaatctggagc
cggtgagcgtggaagccgcggtatcattgcagcactggggccagatggtaagccctcccgtatcgtag
ttatctacacgacggggagtcaggcaactatggatgaacgaaatagacagatcgctgagataggtgcc
tcactgattaagcattggtaactgtcagaccaagtttactcatatatactttagattgatttaaaact
tcatttttaatttaaaaggatctaggtgaagatcctttttgataatctcatgaccaaaatcccttaac
gtgagttttcgttccactgagcgtcagaccccgtagaaaagatcaaaggatcttcttgagatcctttt
tttctgcgcgtaatctgctgcttgcaaacaaaaaaaccaccgctaccagcggtggtttgtttgccgga
tcaagagctaccaactctttttccgaaggtaactggcttcagcagagcgcagataccaaatactgtcc
ttctagtgtagccgtagttaggccaccacttcaagaactctgtagcaccgcctacatacctcgctctg
ctaatcctgttaccagtggctgctgccagtggcgataagtcgtgtcttaccgggttggactcaagacg
atagttaccggataaggcgcagcggtcgggctgaacggggggttcgtgcacacagcccagcttggagc
gaacgacctacaccgaactgagatacctacagcgtgagctatgagaaagcgccacgcttcccgaaggg
agaaaggcggacaggtatccggtaagcggcagggtcggaacaggagagcgcacgagggagcttccagg
gggaaacgcctggtatctttatagtcctgtcgggtttcgccacctctgacttgagcgtcgatttttgt
gatgctcgtcaggggggcggagcctatggaaaaacgccagcaacgcggcctttttacggttcctggcc
ttttgctggccttttgctcacatgt
pDO242 (SaCas9 used in all JCR89/91 projects and JCR157/160 projects
for in vitro work; SaCas9 in uppercase)
SEQ ID NO: 87
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac
amino acid sequence of an S. aureus Cas9
SEQ ID NO: 88
KRNYILGLDIGITSVGYGIIDYETRDVIDAGVRLFKEANVENNEGRRSKRGARRLKRRRRHRIQRVKK
LLFDYNLLTDHSELSGINPYEARVKGLSQKLSEEEFSAALLHLAKRRGVHNVNEVEEDTGNELSTKEQ
ISRNSKALEEKYVAELQLERLKKDGEVRGSINRFKTSDYVKEAKQLLKVQKAYHQLDQSFIDTYIDLL
ETRRTYYEGPGEGSPFGWKDIKEWYEMLMGHCTYFPEELRSVKYAYNADLYNALNDLNNLVITRDENE
KLEYYEKFQIIENVFKQKKKPTLKQIAKEILVNEEDIKGYRVTSTGKPEFTNLKVYHDIKDITARKEI
IENAELLDQIAKILTIYQSSEDIQEELTNLNSELTQEEIEQISNLKGYTGTHNLSLKAINLILDELWH
TNDNQIAIFNRLKLVPKKVDLSQQKEIPTTLVDDFILSPVVKRSFIQSIKVINAIIKKYGLPNDIIIE
LAREKNSKDAQKMINEMQKRNRQTNERIEEIIRTTGKENAKYLIEKIKLHDMQEGKCLYSLEAIPLED
LLNNPFNYEVDHIIPRSVSFDNSFNNKVLVKQEENSKKGNRTPFQYLSSSDSKISYETFKKHILNLAK
GKGRISKTKKEYLLEERDINRFSVQKDFINRNLVDTRYATRGLMNLLRSYFRVNNLDVKVKSINGGFT
SFLRRKWKFKKERNKGYKHHAEDALIIANADFIFKEWKKLDKAKKVMENQMFEEKQAESMPEIETEQE
YKEIFITPHQIKHIKDFKDYKYSHRVDKKPNRELINDTLYSTRKDDKGNTLIVNNLNGLYDKDNDKLK
KLINKSPEKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKCYEEAKKL
KKISNQAEFIASFYNNDLIKINGELYRVIGVNNDLLNRIEVNMIDITYREYLENMNDKRPPRIIKTIA
SKTQSIKKYSTDILGNLYEVKSKKHPQIIKKG
SaCas9 guide RNA scaffold, DNA
SEQ ID NO: 89
tctcgccaacaagttgacgagataaacacggcattttgccttgttttagtagattctgtttccagagt
actaaaac
SaCas9 guide RNA scaffold, RNA
SEQ ID NO: 90
ucucgccaacaaguugacgagauaaacacggcauuuugccuuguuuuaguagauucuguuuccagagu
acuaaaac
Expression cassette of ssAAV vector with gRNAs for exons 45-55, with
wild-type ITR.
Key:
AAV ITR = gray bold
Human U6 promoter = italics
Intron 44 guide = bold underlined
Intron 55 quide = bold double underlined
SaCas9 guide scaffold = italics underlined
SEQ ID NO: 91

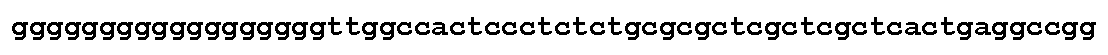

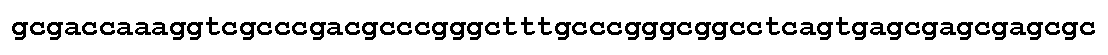

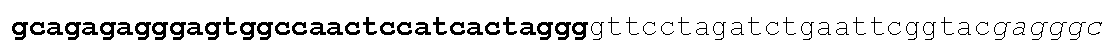

ctatttcccatgattccttcatatttgcatatacgatacaaggctgttagagagataattgg
aattaatttgactgtaaacacaaagatattagtacaaaatacgtgacgtagaaagtaataat
ttcttgggtagtttgcagttttaaaattatgttttaaaatggactatcatatgcttaccgta
acttgaaagtatttcgatttcttggctttatatatcttGTGGAAAGGACGAAACACCg [symbol] [symbol]
gttttagtactctggaaacagaatctactaaaacaaggcaaa
atgccgtgtttatctcgtcaacttgttggcgagatttttTTGCTCCTAGGgagggcctattt
cccatgattccttcatatttgcatatacgatacaaggctgttagagagataattggaattaa
tttgactgtaaacacaaagatattagtacaaaatacgtgacgtagaaagtaataatttcttg
ggtagtttgcagttttaaaattatgttttaaaatggactatcatatgcttaccgtaacttga
aagtatttcgatttcttggctttatatatcttGTGGAAAGGACGAAACACCg [symbol] [symbol]
gttttagtactctggaaacagaatctactaaaacaaggcaaaatgccgtg
tttatctcgtcaacttgttggcgagatttttTctagaggatccggtactcgaggaactgaaa
aaccagaaagttaactggtaagtttagtctttttgtcttttatttcaggtcccggatccggt
ggtggtgcaaatcaaagaactgctcctcagtggatgttgcctttacttctaggcctgtacgg
aagtgttacttctgctctaaaagctgcggaattgtacccgcggcccgggatccaccggtcgc
caccatggtgagcaagggcgaggagctgttcaccggggtggtgcccatcctggtcgagctgg
acggcgacgtaaacggccacaagttcagcgtgtccggcgagggcgagggcgatgccacctac
ggcaagctgaccctgaagttcatctgcaccaccggcaagctgcccgtgccctggcccaccct
cgtgaccaccctgacctacggcgtgcagtgcttcagccgctaccccgaccacatgaagcagc
acgacttcttcaagtccgccatgcccgaaggctacgtccaggagcgcaccatcttcttcaag
gacgacggcaactacaagacccgcgccgaggtgaagttcgagggcgacaccctggtgaaccg
catcgagctgaagggcatcgacttcaaggaggacggcaacatcctggggcacaagctggagt
acaactacaacagccacaacgtctatatcatggccgacaagcagaagaacggcatcaaggtg
aacttcaagatccgccacaacatcgaggacggcagcgtgcagctcgccgaccactaccagca
gaacacccccatcggcgacggccccgtgctgctgcccgacaaccactacctgagcacccagt
ccgccctgagcaaagaccccaacgagaagcgcgatcacatggtcctgctggagttcgtgacc
gccgccgggatcactctcggcatggacgagctgtacaagtaaagcggccgcggggatccaga
catgataagatacattgatgagtttggacaaaccacaactagaatgcagtgaaaaaaatgct
ttatttgtgaaatttgtgatgctattgctttatttgtaaccattataagctgcaataaacaa
gttaacaacaacaattgcattcattttatgtttcaggttcagggggaggtgtgggaggtttt
ttagtcgacctcgagcagtgtggttttgcaagaggaagcaaaaagcctctccacccaggcct
ggaatgtttccacccaagtcgaaggcagtgtggttttgcaagaggaagcaaaaagcctctcc
acccaggcctggaatgtttccacccaatgtcgagcaaccccgcccagcgtcttgtcattggc
gaattcgaacacgcagatgcagtcggggcggcgcggtcccaggtccacttcgcatattaagg
tgacgcgtgtggcctcgaacaccgagcgaccctgcagccaatatgggatcggccattgaaca
agatggattgcacgcaggttctccggccgcttgggtggagaggctattcggctatgactggg
cacaacagacaatcggctgctctgatgccgccgtgttccggctgtcagcgcaggggcgcccg
gttctttttgtcaagaccgacctgtccggtgccctgaatgaactgcaggacgaggcagcgcg
gctatcgtggctggccacgacgggcgttccttgcgcagctgtgctcgacgttgtcactgaag
cgggaagggactggctgctattgggcgaagtgccggggcaggatctcctgtcatctcacctt
gctcctgccgagaaagtatccatcatggctgatgcaatgcggcggctgcatacgcttgatcc
ggctacctgcccattcgaccaccaagcgaaacatcgcatcgagcgagcacgtactcggatgg
aagccggtcttgtcgatcaggatgatctggacgaagagcatcaggggctcgcgccagccgaa
ctgttcgccaggctcaaggcgcgcatgcccgacggcgaggatctcgtcgtgacccatggcga
tgcctgcttgccgaatatcatggtggaaaatggccgcttttctggattcatcgactgtggcc
ggctgggtgtggcggaccgctatcaggacatagcgttggctacccgtgatattgctgaagag
cttggcggcgaatgggctgaccgcttcctcgtgctttacggtatcgccgctcccgattcgca
gcgcatcgccttctatcgccttcttgacgagttcttctgaggggatccgtcgactagagctc
gctgatcagcctcgactgtgccttctagttgccagccatctgttgtttgcccctcccccgtg
ccttccttgaccctggaaggtgccactcccactgtcctttcctaataaaatgaggaaattgc
atcgcattgtctgagtaggtgtcattctattctggggggtggggtggggcaggacagcaagg
gggaggattgggaagacaatagcaggcatgctggggagagatctaggaacccctagtgatgg
agttggccactccctctctgcgcgctcgctcgctcactgaggccgcccgggcaaagcccggg
cgtcgggcgacctttggtcgcccggcctcagtgagcgagcgagcgcgcagagagggagtggc
caacccccccccccccccccctgcagcccagctgcattaatgaatcggccaacgcgcgggga
gaggcggtttgcgtattgggcgctcttccgcttcctcgctcactgactcgctgcgctcggtc
gttcggctgcggcgagcggtatcagctcactcaaaggcggtaatacggttatccacagaatc
aggggataacgcaggaaagaacatgtgagcaaaaggccagcaaaaggccaggaaccgtaaaa
aggccgcgttgctggcgtttttccataggctccgcccccctgacgagcatcacaaaaatcga
cgctcaagtcagaggtggcgaaacccgacaggactataaagataccaggcgtttccccctgg
aagctccctcgtgcgctctcctgttccgaccctgccgcttaccggatacctgtccgcctttc
tcccttcgggaagcgtggcgctttctcaatgctcacgctgtaggtatctcagttcggtgtag
gtcgttcgctccaagctgggctgtgtgcacgaaccccccgttcagcccgaccgctgcgcctt
atccggtaactatcgtcttgagtccaacccggtaagacacgacttatcgccactggcagcag
ccactggtaacaggattagcagagcgaggtatgtaggcggtgctacagagttcttgaagtgg
tggcctaactacggctacactagaaggacagtatttggtatctgcgctctgctgaagccagt
taccttcggaaaaagagttggtagctcttgatccggcaaacaaaccaccgctggtagcggtg
gtttttttgtttgcaagcagcagattacgcgcagaaaaaaaggatctcaagaagatcctttg
atcttttctacggggtctgacgctcagtggaacgaaaactcacgttaagggattttggtcat
gagattatcaaaaaggatcttcacctagatccttttaaattaaaaatgaagttttaaatcaa
tctaaagtatatatgagtaaacttggtctgacagttaccaatgcttaatcagtgaggcacct
atctcagcgatctgtctatttcgttcatccatagttgcctgactccccgtcgtgtagataac
tacgatacgggagggcttaccatctggccccagtgctgcaatgataccgcgagacccacgct
caccggctccagatttatcagcaataaaccagccagccggaagggccgagcgcagaagtggt
cctgcaactttatccgcctccatccagtctattaattgttgccgggaagctagagtaagtag
ttcgccagttaatagtttgcgcaacgttgttgccattgctacaggcatcgtggtgtcacgct
cgtcgtttggtatggcttcattcagctccggttcccaacgatcaaggcgagttacatgatcc
cccatgttgtgcaaaaaagcggttagctccttcggtcctccgatcgttgtcagaagtaagtt
ggccgcagtgttatcactcatggttatggcagcactgcataattctcttactgtcatgccat
ccgtaagatgcttttctgtgactggtgagtactcaaccaagtcattctgagaatagtgtatg
cggcgaccgagttgctcttgcccggcgtcaatacgggataataccgcgccacatagcagaac
tttaaaagtgctcatcattggaaaacgttcttcggggcgaaaactctcaaggatcttaccgc
tgttgagatccagttcgatgtaacccactcgtgcacccaactgatcttcagcatcttttact
ttcaccagcgtttctgggtgagcaaaaacaggaaggcaaaatgccgcaaaaaagggaataag
ggcgacacggaaatgttgaatactcatactcttcctttttcaatattattgaagcatttatc
agggttattgtctcatgagcggatacatatttgaatgtatttagaaaaataaacaaataggg
gttccgcgcacatttccccgaaaagtgccacctgacgtctaagaaaccattattatcatgac
attaacctataaaaataggcgtatcacgaggccctttcgtctcgcgcgtttcggtgatgacg
gtgaaaacctctgacacatgcagctcccggagacggtcacagcttgtctgtaagcggatgcc
gggagcagacaagcccgtcagggcgcgtcagcgggtgttggcgggtgtcggggctggcttaa
ctatgcggcatcagagcagattgtactgagagtgcaccatatgcggtgtgaaataccgcaca
gatgcgtaaggagaaaataccgcatcaggaaattgtaaacgttaatattttgttaaaattcg
cgttaaatttttgttaaatcagctcattttttaaccaataggccgaaatcggcaaaatccct
tataaatcaaaagaatagaccgagatagggttgagtgttgttccagtttggaacaagagtcc
actattaaagaacgtggactccaacgtcaaagggcgaaaaaccgtctatcagggcgatggcc
cactacgtgaaccatcaccctaatcaagttttttggggtcgaggtgccgtaaagcactaaat
cggaaccctaaagggagcccccgatttagagcttgacggggaaagccggcgaacgtggcgag
aaaggaagggaagaaagcgaaaggagcgggcgctagggcgctggcaagtgtagcggtcacgc
tgcgcgtaaccaccacacccgccgcgcttaatgcgccgctacagggcgcgtcgcgccattcg
ccattcaggctacgcaactgttgggaagggcgatcggtgcgggcctcttcgctattacgcca
gctggctgca
Expression cassette of scAAV vector with gRNAs for exons 45-55, with
wild-type and mutant ITR.
Key:
AAV ITR = gray bold
AAV mutant ITR = gray bold italics
Human U6 promoter = italics
Intron 44 quide = bold underlined
Intron 55 guide = bold double underlined
SaCas9 guide scaffold = italics underlined
SEQ ID NO: 92
agcgcccaatacgcaaaccgcctctccccgcgcgttggccgattcattaatgcagctggcac
gacaggtttcccgactggaaagcgggcagtgagcgcaacgcaattaatgtgagttagctcac
tcattaggcaccccaggctttacactttatgcttccggctcgtatgttgtgtggaattgtga
gcggataacaatttcacacaggaaacagctatgaccatgattacgccaagctctcgagatct

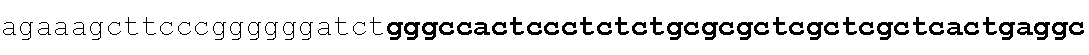

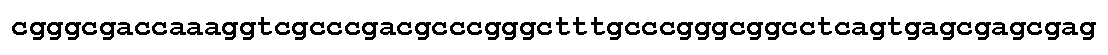

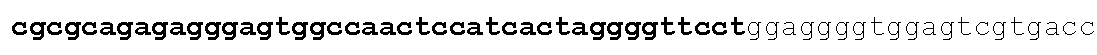

taggcatatgccaagtacgccccctattgacgtcaatgacggtaaatggcccgcctggcatt
atgcccagtacatgaccttatgggactttcctacttggcagtacatctacgtattagtcatc
gctattaccatggtggagggcctatttcccatgattccttcatatttgcatatacgatacaa
ggctgttagagagataattggaattaatttgactgtaaacacaaagatattagtacaaaata
cgtgacgtagaaagtaataatttcttgggtagtttgcagttttaaaattatgttttaaaatg
gactatcatatgcttaccgtaacttgaaagtatttcgatttcttggctttatatatcttGTG
GAAAGGACGAAACACCg [symbol] gttttagtactctggaaacag
aatctactaaaacaaggcaaaatgccgtgtttatctcgtcaacttgttggcgagattttttg
aagagggcctatttcccatgattccttcatatttgcatatacgatacaaggctgttagagag
ataattggaattaatttgactgtaaacacaaagatattagtacaaaatacgtgacgtagaaa
gtaataatttcttgggtagtttgcagttttaaaattatgttttaaaatggactatcatatgc
ttaccgtaacttgaaagtatttcgatttcttggctttatatatcttGTGGAAAGGACGAAAC
ACCg [symbol] gttttagtactctggaaacagaatctactaaaacaa
ggcaaaatgccgtgtttatctcgtcaacttgttggcgagatttttagcggccgctaggcctc
acctgcgatctcgatgctttatttgtgaaatttgtgatgctattgctttatttgtaaccatt
ataagctgcaataaacaagttaacaacaacaattgcattcattttatgtttcaggttcaggg

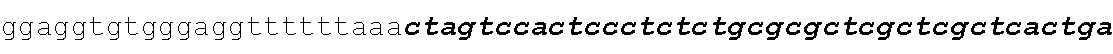

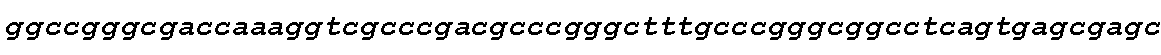

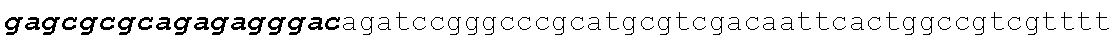

acaacgtcgtgactgggaaaaccctggcgttacccaacttaatcgccttgcagcacatcccc
ctttcgccagctggcgtaatagcgaagaggcccgcaccgatcgcccttcccaacagttgcgc
agcctgaatggcgaatggcgcctgatgcggtattttctccttacgcatctgtgcggtatttc
acaccgcatatggtgcactctcagtacaatctgctctgatgccgcatagttaagccagcccc
gacacccgccaacacccgctgacgcgccctgacgggcttgtctgctcccggcatccgcttac
agacaagctgtgaccgtctccgggagctgcatgtgtcagaggttttcaccgtcatcaccgaa
acgcgcgagacgaaagggcctcgtgatacgcctatttttataggttaatgtcatgataataa
tggtttcttagacgtcaggtggcacttttcggggaaatgtgcgcggaacccctatttgttta
tttttctaaatacattcaaatatgtatccgctcatgagacaataaccctgataaatgcttca
ataatattgaaaaaggaagagtatgagtattcaacatttccgtgtcgcccttattccctttt
ttgcggcattttgccttcctgtttttgctcacccagaaacgctggtgaaagtaaaagatgct
gaagatcagttgggtgcacgagtgggttacatcgaactggatctcaacagcggtaagatcct
tgagagttttcgccccgaagaacgttttccaatgatgagcacttttaaagttctgctatgtg
gcgcggtattatcccgtattgacgccgggcaagagcaactcggtcgccgcatacactattct
cagaatgacttggttgagtactcaccagtcacagaaaagcatcttacggatggcatgacagt
aagagaattatgcagtgctgccataaccatgagtgataacactgcggccaacttacttctga
caacgatcggaggaccgaaggagctaaccgcttttttgcacaacatgggggatcatgtaact
cgccttgatcgttgggaaccggagctgaatgaagccataccaaacgacgagcgtgacaccac
gatgcctgtagcaatggcaacaacgttgcgcaaactattaactggcgaactacttactctag
cttcccggcaacaattaatagactggatggaggcggataaagttgcaggaccacttctgcgc
tcggcccttccggctggctggtttattgctgataaatctggagccggtgagcgtgggtctcg
cggtatcattgcagcactggggccagatggtaagccctcccgtatcgtagttatctacacga
cggggagtcaggcaactatggatgaacgaaatagacagatcgctgagataggtgcctcactg
attaagcattggtaactgtcagaccaagtttactcatatatactttagattgatttaaaact
tcatttttaatttaaaaggatctaggtgaagatcctttttgataatctcatgaccaaaatcc
cttaacgtgagttttcgttccactgagcgtcagaccccgtagaaaagatcaaaggatcttct
tgagatcctttttttctgcgcgtaatctgctgcttgcaaacaaaaaaaccaccgctaccagc
ggtggtttgtttgccggatcaagagctaccaactctttttccgaaggtaactggcttcagca
gagcgcagataccaaatactgttcttctagtgtagccgtagttaggccaccacttcaagaac
tctgtagcaccgcctacatacctcgctctgctaatcctgttaccagtggctgctgccagtgg
cgataagtcgtgtcttaccgggttggactcaagacgatagttaccggataaggcgcagcggt
cgggctgaacggggggttcgtgcacacagcccagcttggagcgaacgacctacaccgaactg
agatacctacagcgtgagctatgagaaagcgccacgcttcccgaagggagaaaggcggacag
gtatccggtaagcggcagggtcggaacaggagagcgcacgagggagcttccagggggaaacg
cctggtatctttatagtcctgtcgggtttcgccacctctgacttgagcgtcgatttttgtga
tgctcgtcaggggggcggagcctatggaaaaacgccagcaacgcggcctttttacggttcct
ggccttttgctggccttttgctcacatgttctttcctgcgttatcccctgattctgtggata
accgtattaccgcctttgagtgagctgataccgctcgccgcagccgaacgaccgagcgcagc
gagtcagtgagcgaggaagcggaag
Expression cassette of ssAAV
SEQ ID NO: 128
ggggggggggggggggggttggccactccctctctgcgcgctcgctcgctcactgaggccgggcgacc
aaaggtcgcccgacgcccgggctttgcccgggcggcctcagtgagcgagcgagcgcgcagagagggag
tggccaactccatcactaggggttcctagatctgaattcggtacgagggcctatttcccatgattcct
tcatatttgcatatacgatacaaggctgttagagagataattggaattaatttgactgtaaacacaaa
gatattagtacaaaatacgtgacgtagaaagtaataatttcttgggtagtttgcagttttaaaattat
gttttaaaatggactatcatatgcttaccgtaacttgaaagtatttcgatttcttggctttatatatc
ttGTGGAAAGGACGAAACACCgATATAGTAATGAAATTATTGGCACgttttagtactctggaaacaga
atctactaaaacaaggcaaaatgccgtgtttatctcgtcaacttgttggcgagatttttTTGCTCCTA
GGgagggcctatttcccatgattccttcatatttgcatatacgatacaaggctgttagagagataatt
ggaattaatttgactgtaaacacaaagatattagtacaaaatacgtgacgtagaaagtaataatttct
tgggtagtttgcagttttaaaattatgttttaaaatggactatcatatgcttaccgtaacttgaaagt
atttcgatttcttggctttatatatcttGTGGAAAGGACGAAACACCgcatttgtatagagaggaaat
gtgttttagtactctggaaacagaatctactaaaacaaggcaaaatgccgtgtttatctcgtcaactt
gttggcgagattttt
Expression cassette of scAAV
SEQ ID NO: 129
gggccactccctctctgcgcgctcgctcgctcactgaggccgggcgaccaaaggtcgcccgacgcccg
ggctttgcccgggcggcctcagtgagcgagcgagcgcgcagagagggagtggccaactccatcactag
gggttcctggaggggtggagtcgtgacctaggcatatgccaagtacgccccctattgacgtcaatgac
ggtaaatggcccgcctggcattatgcccagtacatgaccttatgggactttcctacttggcagtacat
ctacgtattagtcatcgctattaccatggtggagggcctatttcccatgattccttcatatttgcata
tacgatacaaggctgttagagagataattggaattaatttgactgtaaacacaaagatattagtacaa
aatacgtgacgtagaaagtaataatttcttgggtagtttgcagttttaaaattatgttttaaaatgga
ctatcatatgcttaccgtaacttgaaagtatttcgatttcttggctttatatatcttGTGGAAAGGAC
GAAACACCgATATAGTAATGAAATTATTGGCACgttttagtactctggaaacagaatctactaaaaca
aggcaaaatgccgtgtttatctcgtcaacttgttggcgagattttttgaagagggcctatttcccatg
attccttcatatttgcatatacgatacaaggctgttagagagataattggaattaatttgactgtaaa
cacaaagatattagtacaaaatacgtgacgtagaaagtaataatttcttgggtagtttgcagttttaa
aattatgttttaaaatggactatcatatgcttaccgtaacttgaaagtatttcgatttcttggcttta
tatatcttGTGGAAAGGACGAAACACCgcatttgtatagagaggaaatgtgttttagtactctggaaa
cagaatctactaaaacaaggcaaaatgccgtgtttatctcgtcaacttgttggcgagatttttagcgg
ccgctaggcctcacctgcgatctcgatgctttatttgtgaaatttgtgatgctattgctttatttgta
accattataagctgcaataaacaagttaacaacaacaattgcattcattttatgtttcaggttcaggg
ggaggtgtgggaggttttttaaactagtccactccctctctgcgcgctcgctcgctcactgaggccgg
gcgaccaaaggtcgcccgacgcccgggctttgcccgggcggcctcagtgagcgagcgagcgcgcagag
agggac
ssAAV with gRNAs for Exon 45-55
SEQ ID NO: 130
ggggggggggggggggggttggccactccctctctgcgcgctcgctcgctcactgaggccgggcgacc
aaaggtcgcccgacgcccgggctttgcccgggcggcctcagtgagcgagcgagcgcgcagagagggag
tggccaactccatcactaggggttcctagatctgaattcggtacgagggcctatttcccatgattcct
tcatatttgcatatacgatacaaggctgttagagagataattggaattaatttgactgtaaacacaaa
gatattagtacaaaatacgtgacgtagaaagtaataatttcttgggtagtttgcagttttaaaattat
gttttaaaatggactatcatatgcttaccgtaacttgaaagtatttcgatttcttggctttatatatc
ttGTGGAAAGGACGAAACACCgATATAGTAATGAAATTATTGGCACgttttagtactctggaaacaga
atctactaaaacaaggcaaaatgccgtgtttatctcgtcaacttgttggcgagatttttTTGCTCCTA
GGgagggcctatttcccatgattccttcatatttgcatatacgatacaaggctgttagagagataatt
ggaattaatttgactgtaaacacaaagatattagtacaaaatacgtgacgtagaaagtaataatttct
tgggtagtttgcagttttaaaattatgttttaaaatggactatcatatgcttaccgtaacttgaaagt
atttcgatttcttggctttatatatcttGTGGAAAGGACGAAACACCgcatttgtatagagaggaaat
gtgttttagtactctggaaacagaatctactaaaacaaggcaaaatgccgtgtttatctcgtcaactt
gttggcgagatttttTctagaggatccggtactcgaggaactgaaaaaccagaaagttaactggtaag
tttagtctttttgtcttttatttcaggtcccggatccggtggtggtgcaaatcaaagaactgctcctc
agtggatgttgcctttacttctaggcctgtacggaagtgttacttctgctctaaaagctgcggaattg
tacccgcggcccgggatccaccggtcgccaccatggtgagcaagggcgaggagctgttcaccggggtg
gtgcccatcctggtcgagctggacggcgacgtaaacggccacaagttcagcgtgtccggcgagggcga
gggcgatgccacctacggcaagctgaccctgaagttcatctgcaccaccggcaagctgcccgtgccct
ggcccaccctcgtgaccaccctgacctacggcgtgcagtgcttcagccgctaccccgaccacatgaag
cagcacgacttcttcaagtccgccatgcccgaaggctacgtccaggagcgcaccatcttcttcaagga
cgacggcaactacaagacccgcgccgaggtgaagttcgagggcgacaccctggtgaaccgcatcgagc
tgaagggcatcgacttcaaggaggacggcaacatcctggggcacaagctggagtacaactacaacagc
cacaacgtctatatcatggccgacaagcagaagaacggcatcaaggtgaacttcaagatccgccacaa
catcgaggacggcagcgtgcagctcgccgaccactaccagcagaacacccccatcggcgacggccccg
tgctgctgcccgacaaccactacctgagcacccagtccgccctgagcaaagaccccaacgagaagcgc
gatcacatggtcctgctggagttcgtgaccgccgccgggatcactctcggcatggacgagctgtacaa
gtaaagcggccgcggggatccagacatgataagatacattgatgagtttggacaaaccacaactagaa
tgcagtgaaaaaaatgctttatttgtgaaatttgtgatgctattgctttatttgtaaccattataagc
tgcaataaacaagttaacaacaacaattgcattcattttatgtttcaggttcagggggaggtgtggga
ggttttttagtcgacctcgagcagtgtggttttgcaagaggaagcaaaaagcctctccacccaggcct
ggaatgtttccacccaagtcgaaggcagtgtggttttgcaagaggaagcaaaaagcctctccacccag
gcctggaatgtttccacccaatgtcgagcaaccccgcccagcgtcttgtcattggcgaattcgaacac
gcagatgcagtcggggcggcgcggtcccaggtccacttcgcatattaaggtgacgcgtgtggcctcga
acaccgagcgaccctgcagccaatatgggatcggccattgaacaagatggattgcacgcaggttctcc
ggccgcttgggtggagaggctattcggctatgactgggcacaacagacaatcggctgctctgatgccg
ccgtgttccggctgtcagcgcaggggcgcccggttctttttgtcaagaccgacctgtccggtgccctg
aatgaactgcaggacgaggcagcgcggctatcgtggctggccacgacgggcgttccttgcgcagctgt
gctcgacgttgtcactgaagcgggaagggactggctgctattgggcgaagtgccggggcaggatctcc
tgtcatctcaccttgctcctgccgagaaagtatccatcatggctgatgcaatgcggcggctgcatacg
cttgatccggctacctgcccattcgaccaccaagcgaaacatcgcatcgagcgagcacgtactcggat
ggaagccggtcttgtcgatcaggatgatctggacgaagagcatcaggggctcgcgccagccgaactgt
tcgccaggctcaaggcgcgcatgcccgacggcgaggatctcgtcgtgacccatggcgatgcctgcttg
ccgaatatcatggtggaaaatggccgcttttctggattcatcgactgtggccggctgggtgtggcgga
ccgctatcaggacatagcgttggctacccgtgatattgctgaagagcttggcggcgaatgggctgacc
gcttcctcgtgctttacggtatcgccgctcccgattcgcagcgcatcgccttctatcgccttcttgac
gagttcttctgaggggatccgtcgactagagctcgctgatcagcctcgactgtgccttctagttgcca
gccatctgttgtttgcccctcccccgtgccttccttgaccctggaaggtgccactcccactgtccttt
cctaataaaatgaggaaattgcatcgcattgtctgagtaggtgtcattctattctggggggtggggtg
gggcaggacagcaagggggaggattgggaagacaatagcaggcatgctggggagagatctaggaaccc
ctagtgatggagttggccactccctctctgcgcgctcgctcgctcactgaggccgcccgggcaaagcc
cgggcgtcgggcgacctttggtcgcccggcctcagtgagcgagcgagcgcgcagagagggagtggcca
acccccccccccccccccctgcagcccagctgcattaatgaatcggccaacgcgcggggagaggcggt
ttgcgtattgggcgctcttccgcttcctcgctcactgactcgctgcgctcggtcgttcggctgcggcg
agcggtatcagctcactcaaaggcggtaatacggttatccacagaatcaggggataacgcaggaaaga
acatgtgagcaaaaggccagcaaaaggccaggaaccgtaaaaaggccgcgttgctggcgtttttccat
aggctccgcccccctgacgagcatcacaaaaatcgacgctcaagtcagaggtggcgaaacccgacagg
actataaagataccaggcgtttccccctggaagctccctcgtgcgctctcctgttccgaccctgccgc
ttaccggatacctgtccgcctttctcccttcgggaagcgtggcgctttctcaatgctcacgctgtagg
tatctcagttcggtgtaggtcgttcgctccaagctgggctgtgtgcacgaaccccccgttcagcccga
ccgctgcgccttatccggtaactatcgtcttgagtccaacccggtaagacacgacttatcgccactgg
cagcagccactggtaacaggattagcagagcgaggtatgtaggcggtgctacagagttcttgaagtgg
tggcctaactacggctacactagaaggacagtatttggtatctgcgctctgctgaagccagttacctt
cggaaaaagagttggtagctcttgatccggcaaacaaaccaccgctggtagcggtggtttttttgttt
gcaagcagcagattacgcgcagaaaaaaaggatctcaagaagatcctttgatcttttctacggggtct
gacgctcagtggaacgaaaactcacgttaagggattttggtcatgagattatcaaaaaggatcttcac
ctagatccttttaaattaaaaatgaagttttaaatcaatctaaagtatatatgagtaaacttggtctg
acagttaccaatgcttaatcagtgaggcacctatctcagcgatctgtctatttcgttcatccatagtt
gcctgactccccgtcgtgtagataactacgatacgggagggcttaccatctggccccagtgctgcaat
gataccgcgagacccacgctcaccggctccagatttatcagcaataaaccagccagccggaagggccg
agcgcagaagtggtcctgcaactttatccgcctccatccagtctattaattgttgccgggaagctaga
gtaagtagttcgccagttaatagtttgcgcaacgttgttgccattgctacaggcatcgtggtgtcacg
ctcgtcgtttggtatggcttcattcagctccggttcccaacgatcaaggcgagttacatgatccccca
tgttgtgcaaaaaagcggttagctccttcggtcctccgatcgttgtcagaagtaagttggccgcagtg
ttatcactcatggttatggcagcactgcataattctcttactgtcatgccatccgtaagatgcttttc
tgtgactggtgagtactcaaccaagtcattctgagaatagtgtatgcggcgaccgagttgctcttgcc
cggcgtcaatacgggataataccgcgccacatagcagaactttaaaagtgctcatcattggaaaacgt
tcttcggggcgaaaactctcaaggatcttaccgctgttgagatccagttcgatgtaacccactcgtgc
acccaactgatcttcagcatcttttactttcaccagcgtttctgggtgagcaaaaacaggaaggcaaa
atgccgcaaaaaagggaataagggcgacacggaaatgttgaatactcatactcttcctttttcaatat
tattgaagcatttatcagggttattgtctcatgagcggatacatatttgaatgtatttagaaaaataa
acaaataggggttccgcgcacatttccccgaaaagtgccacctgacgtctaagaaaccattattatca
tgacattaacctataaaaataggcgtatcacgaggccctttcgtctcgcgcgtttcggtgatgacggt
gaaaacctctgacacatgcagctcccggagacggtcacagcttgtctgtaagcggatgccgggagcag
acaagcccgtcagggcgcgtcagcgggtgttggcgggtgtcggggctggcttaactatgcggcatcag
agcagattgtactgagagtgcaccatatgcggtgtgaaataccgcacagatgcgtaaggagaaaatac
cgcatcaggaaattgtaaacgttaatattttgttaaaattcgcgttaaatttttgttaaatcagctca
ttttttaaccaataggccgaaatcggcaaaatcccttataaatcaaaagaatagaccgagatagggtt
gagtgttgttccagtttggaacaagagtccactattaaagaacgtggactccaacgtcaaagggcgaa
aaaccgtctatcagggcgatggcccactacgtgaaccatcaccctaatcaagttttttggggtcgagg
tgccgtaaagcactaaatcggaaccctaaagggagcccccgatttagagcttgacggggaaagccggc
gaacgtggcgagaaaggaagggaagaaagcgaaaggagcgggcgctagggcgctggcaagtgtagcgg
tcacgctgcgcgtaaccaccacacccgccgcgcttaatgcgccgctacagggcgcgtcgcgccattcg
ccattcaggctacgcaactgttgggaagggcgatcggtgcgggcctcttcgctattacgccagctggc
tgca
scAAV with gRNAs for Exon 45-55
SEQ ID NO: 131
agcgcccaatacgcaaaccgcctctccccgcgcgttggccgattcattaatgcagctggcacgacagg
tttcccgactggaaagcgggcagtgagcgcaacgcaattaatgtgagttagctcactcattaggcacc
ccaggctttacactttatgcttccggctcgtatgttgtgtggaattgtgagcggataacaatttcaca
caggaaacagctatgaccatgattacgccaagctctcgagatctagaaagcttcccggggggatctgg
gccactccctctctgcgcgctcgctcgctcactgaggccgggcgaccaaaggtcgcccgacgcccggg
ctttgcccgggcggcctcagtgagcgagcgagcgcgcagagagggagtggccaactccatcactaggg
gttcctggaggggtggagtcgtgacctaggcatatgccaagtacgccccctattgacgtcaatgacgg
taaatggcccgcctggcattatgcccagtacatgaccttatgggactttcctacttggcagtacatct
acgtattagtcatcgctattaccatggtggagggcctatttcccatgattccttcatatttgcatata
cgatacaaggctgttagagagataattggaattaatttgactgtaaacacaaagatattagtacaaaa
tacgtgacgtagaaagtaataatttcttgggtagtttgcagttttaaaattatgttttaaaatggact
atcatatgcttaccgtaacttgaaagtatttcgatttcttggctttatatatcttGTGGAAAGGACGA
AACACCgATATAGTAATGAAATTATTGGCACgttttagtactctggaaacagaatctactaaaacaag
gcaaaatgccgtgtttatctcgtcaacttgttggcgagattttttgaagagggcctatttcccatgat
tccttcatatttgcatatacgatacaaggctgttagagagataattggaattaatttgactgtaaaca
caaagatattagtacaaaatacgtgacgtagaaagtaataatttcttgggtagtttgcagttttaaaa
ttatgttttaaaatggactatcatatgcttaccgtaacttgaaagtatttcgatttcttggctttata
tatcttGTGGAAAGGACGAAACACCgcatttgtatagagaggaaatgtgttttagtactctggaaaca
gaatctactaaaacaaggcaaaatgccgtgtttatctcgtcaacttgttggcgagatttttagcggcc
gctaggcctcacctgcgatctcgatgctttatttgtgaaatttgtgatgctattgctttatttgtaac
cattataagctgcaataaacaagttaacaacaacaattgcattcattttatgtttcaggttcaggggg
aggtgtgggaggttttttaaactagtccactccctctctgcgcgctcgctcgctcactgaggccgggc
gaccaaaggtcgcccgacgcccgggctttgcccgggcggcctcagtgagcgagcgagcgcgcagagag
ggacagatccgggcccgcatgcgtcgacaattcactggccgtcgttttacaacgtcgtgactgggaaa
accctggcgttacccaacttaatcgccttgcagcacatccccctttcgccagctggcgtaatagcgaa
gaggcccgcaccgatcgcccttcccaacagttgcgcagcctgaatggcgaatggcgcctgatgcggta
ttttctccttacgcatctgtgcggtatttcacaccgcatatggtgcactctcagtacaatctgctctg
atgccgcatagttaagccagccccgacacccgccaacacccgctgacgcgccctgacgggcttgtctg
ctcccggcatccgcttacagacaagctgtgaccgtctccgggagctgcatgtgtcagaggttttcacc
gtcatcaccgaaacgcgcgagacgaaagggcctcgtgatacgcctatttttataggttaatgtcatga
taataatggtttcttagacgtcaggtggcacttttcggggaaatgtgcgcggaacccctatttgttta
tttttctaaatacattcaaatatgtatccgctcatgagacaataaccctgataaatgcttcaataata
ttgaaaaaggaagagtatgagtattcaacatttccgtgtcgcccttattcccttttttgcggcatttt
gccttcctgtttttgctcacccagaaacgctggtgaaagtaaaagatgctgaagatcagttgggtgca
cgagtgggttacatcgaactggatctcaacagcggtaagatccttgagagttttcgccccgaagaacg
ttttccaatgatgagcacttttaaagttctgctatgtggcgcggtattatcccgtattgacgccgggc
aagagcaactcggtcgccgcatacactattctcagaatgacttggttgagtactcaccagtcacagaa
aagcatcttacggatggcatgacagtaagagaattatgcagtgctgccataaccatgagtgataacac
tgcggccaacttacttctgacaacgatcggaggaccgaaggagctaaccgcttttttgcacaacatgg
gggatcatgtaactcgccttgatcgttgggaaccggagctgaatgaagccataccaaacgacgagcgt
gacaccacgatgcctgtagcaatggcaacaacgttgcgcaaactattaactggcgaactacttactct
agcttcccggcaacaattaatagactggatggaggcggataaagttgcaggaccacttctgcgctcgg
cccttccggctggctggtttattgctgataaatctggagccggtgagcgtgggtctcgcggtatcatt
gcagcactggggccagatggtaagccctcccgtatcgtagttatctacacgacggggagtcaggcaac
tatggatgaacgaaatagacagatcgctgagataggtgcctcactgattaagcattggtaactgtcag
accaagtttactcatatatactttagattgatttaaaacttcatttttaatttaaaaggatctaggtg
aagatcctttttgataatctcatgaccaaaatcccttaacgtgagttttcgttccactgagcgtcaga
ccccgtagaaaagatcaaaggatcttcttgagatcctttttttctgcgcgtaatctgctgcttgcaaa
caaaaaaaccaccgctaccagcggtggtttgtttgccggatcaagagctaccaactctttttccgaag
gtaactggcttcagcagagcgcagataccaaatactgttcttctagtgtagccgtagttaggccacca
cttcaagaactctgtagcaccgcctacatacctcgctctgctaatcctgttaccagtggctgctgcca
gtggcgataagtcgtgtcttaccgggttggactcaagacgatagttaccggataaggcgcagcggtcg
ggctgaacggggggttcgtgcacacagcccagcttggagcgaacgacctacaccgaactgagatacct
acagcgtgagctatgagaaagcgccacgcttcccgaagggagaaaggcggacaggtatccggtaagcg
gcagggtcggaacaggagagcgcacgagggagcttccagggggaaacgcctggtatctttatagtcct
gtcgggtttcgccacctctgacttgagcgtcgatttttgtgatgctcgtcaggggggcggagcctatg
gaaaaacgccagcaacgcggcctttttacggttcctggccttttgctggccttttgctcacatgttct
ttcctgcgttatcccctgattctgtggataaccgtattaccgcctttgagtgagctgataccgctcgc
cgcagccgaacgaccgagcgcagcgagtcagtgagcgaggaagcggaag
First AAV ITR sequence (wild-type ITR sequence)
SEQ ID NO: 132
ggggggggggggggggggttggccactccctctctgcgcgctcgctcgctcactgaggccgggcgacc
aaaggtcgcccgacgcccgggctttgcccgggcggcctcagtgagcgagcgagcgcgcagagagggag
tggccaactccatcactaggg
Human U6 promoter sequence
SEQ ID NO: 133
gagggcctatttcccatgattccttcatatttgcatatacgatacaaggctgttagagagataattgg
aattaatttgactgtaaacacaaagatattagtacaaaatacgtgacgtagaaagtaataatttcttg
ggtagtttgcagttttaaaattatgttttaaaatggactatcatatgcttaccgtaacttgaaagtat
ttcgatttcttggctttatatatcttGTGGAAAGGACGAAACACC
Intron 55 guide sequence (gRNA targeting intron 55)
SEQ ID NO: 134
ATATAGTAATGAAATTATTGGCAC
Intron 44 guide sequence (gRNA targeting intron 44)
SEQ ID NO: 135
CATTTGTATAGAGAGGAAATGT
Intron 55 guide sequence (gRNA targeting intron 55)
SEQ ID NO: 136
AUAUAGUAAUGAAAUUAUUGGCAC
Intron 44 guide sequence (gRNA targeting intron 44)
SEQ ID NO: 137
CAUUUGUAUAGAGAGGAAAUGU
SaCas9 guide RNA scaffold
SEQ ID NO: 138
GTTTTAGTACTCTGGAAACAGAATCTACTAAAACAAGGCAAAATGCCGTGTTTATCTCGTCAACTTGT
TGGCGAGATTTTT
SaCas9 guide RNA scaffold
SEQ ID NO: 139
GUUUUAGUACUCUGGAAACAGAAUCUACUAAAACAAGGCAAAAUGCCGUGUUUAUCUCGUCAACUUGU
UGGCGAGAUUUUU
Second AAV ITR sequence (mutant ITR sequence)
SEQ ID NO: 140
ctagtccactccctctctgcgcgctcgctcgctcactgaggccgggcgaccaaaggtcgcccgacgcc
cgggctttgcccgggggcctcagtgagcgagcgagcgcgcagagagggac

What is claimed is:

1. A CRISPR-Cas vector system comprising one or more vectors, wherein at least one of the one or more vectors comprises a sequence encoding:
(a) first guide RNA (gRNA) targeting an intron or an exon of dystrophin and a second gRNA targeting an intron or an exon of dystrophin; and
(b) a Cas9 protein.

2. The CRISPR-Cas vector system of claim 1, wherein the system comprises a first vector and a second vector, the first vector encoding the first gRNA and the second gRNA and the second vector encoding the Cas9 protein.

3. A CRISPR-Cas dual vector system comprising:
(a) a first vector encoding a first guide RNA (gRNA) targeting an intron or an exon of dystrophin and a second gRNA targeting an intron or an exon of dystrophin; and
(b) a second vector encoding a Cas9 protein.

4. The system of claim 2 or 3, wherein the first vector comprises a first ITR and a second ITR.

5. The system of claim 4, wherein the first ITR is operably linked to and upstream of the polynucleotide sequences encoding the first gRNA and the second gRNA, and wherein the second ITR is operably linked to and downstream of the polynucleotide sequence encoding the first gRNA and the second gRNA.

6. The system of any one of claims 4-5, wherein the first ITR or second ITR is a wild-type ITR, and the other of the first ITR and second ITR is a mutant ITR, and wherein the mutant ITR directs vector genome replication to generate a self-complementary transcript that forms a double-stranded polynucleotide.

7. The system of claim 6, wherein the wild-type ITR comprises a polynucleotide having a sequence selected from SEQ ID NOs: 59-61 or 132.

8. The system of claim 4 or 5, wherein the mutant ITR comprises a polynucleotide having the sequence of SEQ ID NO: 62 or 140.

9. The system of any one of claims 1-8, wherein the first vector comprises a first promoter operably linked to the polynucleotide sequence encoding the first gRNA molecule, and a second promoter operably linked to the polynucleotide sequence encoding the second gRNA molecule.

10. The system of claim 9, wherein the first vector comprises an expression cassette comprising 5′-[wild-type ITR]-[promoter]-[first gRNA]-[promoter]-[second gRNA]-[mutant ITR]-3′, wherein “-” is an optional linker independently comprising a polynucleotide of 0-60 nucleotides.

11. The system of claim 10, wherein the vector genome replicated from the first vector is self-complementary and comprises 5′-[wild-type ITR]-[promoter]-[first gRNA]-[promoter]-[second gRNA]-[mutant ITR]-[second gRNA]-[promoter]-[first gRNA]-[promoter]-[wild-type ITR]-3′ and forms a double-stranded RNA hairpin.

12. The system of any one of claims 9-11, wherein the first promoter and the second promoter comprise the same or different polynucleotide sequence.

13. The system of any one of claims 9-12, wherein the first promoter and the second promoter are each independently selected from a ubiquitous promoter or a tissue-specific promoter.

14. The system of any one of claims 9-13, wherein the first promoter and the second promoter are each independently selected from a human U6 promoter and a H1 promoter.

15. The system of any one of claims 2-14, wherein the second vector comprises a third promoter driving expression of the Cas9 protein, and wherein the third promoter comprises a ubiquitous promoter or a tissue-specific promoter.

16. The system of claim 13, where the ubiquitous promoter comprises a CMV promoter.

17. The system of claim 13 or 15, where the tissue-specific promoter is a muscle-specific promoter comprising a MHCK7 promoter, a CK8 promoter, or a Spc512 promoter.

18. The system of any one of claims 2-17, wherein the first vector further encodes at least one Cas9 gRNA scaffold.

19. The system of any one of claims 1-18, wherein the first gRNA and the second gRNA each comprise a Cas9 gRNA scaffold.

20. The system of claim 18 or 19, wherein the Cas9 gRNA scaffold comprises the polynucleotide sequence of SEQ ID NO: 89 or 18 or 138.

21. The system of any one of claims 1-20, wherein the first or second gRNA targets intron 44 of dystrophin.

22. The system of any one of claims 1-21, wherein the first or second gRNA targets intron 55 of dystrophin.

23. The system of any one of claims 1-22, wherein the first gRNA targets intron 44 of dystrophin and the second gRNA targets intron 55 of dystrophin, or wherein the first gRNA targets intron 55 of dystrophin and the second gRNA targets intron 44 of dystrophin.

24. The system of claim 21 or 23, wherein the first or second gRNA targeting intron 44 of dystrophin targets a polynucleotide comprising the sequence of SEQ ID NO: 55 or 135 or a 5′ truncation thereof.

25. The system of any one of claims 1-22, wherein the first gRNA or the second gRNA targets intron 44 of dystrophin and comprises the polynucleotide sequence of SEQ ID NO: 57 or 137 or a 5′ truncation thereof.

26. The system of claim 22 or 23, wherein the first or second gRNA targeting intron 55 of dystrophin targets a polynucleotide comprising the sequence of SEQ ID NO: 56 or 134 or a 5′ truncation thereof.

27. The system of any one of claims 1-26, wherein the first gRNA or the second gRNA targets intron 55 of dystrophin and comprises the polynucleotide sequence of SEQ ID NO: 58 or 136 or a 5′ truncation thereof.

28. The system of any one of claims 1-27, wherein the Cas9 protein comprises SpCas9, SaCas9, or St1Cas9 protein.

29. The system of any one of claims 1-28, wherein the Cas9 protein comprises a SaCas9 protein comprising the amino acid sequence of SEQ ID NO: 88 or is encoded by a polynucleotide comprising the sequence of SEQ ID NO: 69.

30. The system of any one of claims 2-29, wherein the first vector comprises a polynucleotide having the sequence selected from SEQ ID NOs: 91, 92, 128, 129, 130, or 131.

31. The system of any one of claims 2-30, wherein the first vector and/or the second vector is a viral vector.

32. The system of claim 31, wherein the viral vector is an Adeno-associated virus (AAV) vector.

33. The system of claim 32, wherein the AAV vector is AAV1, AAV2, AAV3, AAV4, AAV5, AAV6, AAV7, AAV8, AAV9, AAV-10, AAV-11, AAV-12, AAV-13, orAAVrh.74.

34. The system of any one of claims 2-33, wherein the first vector is present in a concentration at least 2-fold, at least 3-fold, at least 4-fold, at least 5-fold, at least 6-fold, at least 7-fold, at least 8-fold, at least 9-fold, or at least 10-fold greater than the concentration of the second vector.

35. The CRISPR-Cas vector system of any one of claims 1-34, the system comprising one or more vectors, wherein at least one vector of the one or more vectors comprises a sequence encoding, from the 5′ to 3′ direction:
(a) a first ITR:
(b) a first promoter;
(c) a first gRNA targeting an intron or exon of the dystrophin gene;
(d) a Cas9 gRNA scaffold;
(e) a second promoter;
(f) a second gRNA targeting an intron or exon of dystrophin the gene;
(g) a Cas9 gRNA scaffold; and
(h) a second ITR.

36. The system of claim 35, wherein vector genome replication from the at least one vector results in a genome comprising, from the 5′ to 3′ direction:
(a) a complementary sequence of the second ITR;
(b) a complementary sequence of the second gRNA;
(c) a complementary sequence of the second promoter:
(d) a complementary sequence of the Cas9 gRNA scaffold;
(e) a complementary sequence of the first gRNA;
(f) a complementary sequence of the first promoter;
(h) the first ITR;
(i) the first promoter;
(g) the first gRNA;
(k) the Cas9 gRNA scaffold;
(l) the second promoter;
(m) the second gRNA; and
(n) the second ITR.

37. A cell comprising the system of any one of claims 1-36.

38. A kit comprising the system of any one of claims 1-36.

39. A method of correcting a mutant dystrophin gene in a cell, the method comprising administering to a cell the system of any one of claims 1-36.

40. A method of genome editing a mutant dystrophin gene in a subject, the method comprising administering to the subject the system of any one of claims 1-36 or the cell of claim 37.

41. A method of treating a subject having a mutant dystrophin gene, the method comprising administering to the subject the system of any one of claims 1-36 or the cell of claim 37.

42. The method of any one of claims 40-41, wherein the subject is an adult, an adolescent, or a pre-adolescent.

43. The method of claim 42, wherein the subject is an adult.

44. The method of any one of claims 40-43, wherein the system of any one of claims 1-36 or the cell of claim 37 is administered to the subject intravenously.

45. The method of any one of claims 40-44, wherein the system of any one of claims 1-36 or the cell of claim 37 is administered to the subject systemically.

46. A CRISPR-Cas dual vector system comprising one or more vectors, wherein the one or more vectors comprises a vector that comprises an expression cassette, from the 5′ to 3′ direction, comprising:
(a) a first AAV ITR sequence;
(b) a first promoter sequence;
(c) a guide sequence targeting a first intron of dystrophin gene;
(d) a Cas9 scaffold sequence;
(e) a second promoter sequence;
(f) a guide sequence targeting a second intron of dystrophin gene; and
(g) a second AAV ITR sequence.

47. The system of claim 46, wherein the expression cassette is a single stranded (“ss”) expression cassette or a self-complementary (“sc”) expression cassette.

48. The system of 47, wherein the self-complementary (“sc”) expression cassette, from the 5′ to 3′ direction, comprises:
(a) a complementary sequence of the second AAV ITR sequence;
(b) a complementary sequence of the guide sequence targeting the second intron of dystrophin gene;
(c) a complementary sequence of the second promoter sequence;
(d) a complementary sequence of the Cas9 scaffold sequence;
(e) a complementary sequence of the guide sequence targeting a first intron of dystrophin gene;
(f) a complementary sequence of the first promoter sequence;
(h) a first AAV ITR sequence;
(i) a first promoter sequence;
(g) a guide sequence targeting a first intron of dystrophin gene;
(k) a Cas9 scaffold sequence;
(l) a second promoter sequence;
(m) a guide sequence targeting a second intron of dystrophin gene; and
(n) a second AAV ITR sequence.

49. The system of any one of claims 46-48, wherein the first intron is intron 44 and the second intron is intron 55 of the dystrophin gene, or wherein the first intron is intron 55 and the second intron is intron of 44 of the dystrophin gene.

50. The system of any one of claims 46-49, wherein the dystrophin gene comprises a mutation compared to a wild-type dystrophin gene.

51. The system of claim 46, wherein the guide sequence targeting a first intron of dystrophin gene comprises a nucleotide sequence of SEQ ID NO: 134 or 56 and the guide sequence targeting a second intron of dystrophin gene comprises a nucleotide sequence of SEQ ID NO: 135 or 55, or wherein the guide sequence targeting a first intron of dystrophin gene comprises a nucleotide sequence of SEQ ID NO: 135 or 55 and the guide sequence targeting a second intron of dystrophin gene comprises a nucleotide sequence of SEQ ID NO: 134 or 56.

52. The system of any one of claims 46-51, wherein the promoter is a constitutive promoter or a tissue-specific promoter.

53. The system of any one of claims 46-52, wherein the promoter is a muscle-specific promoter.

54. The system of claim 53, wherein the muscle-specific promoter comprises a human skeletal actin gene element, cardiac actin gene element, myocyte-specific enhancer binding factor mef, muscle creatine kinase (MCK), truncated MCK (tMCK), myosin heavy chain (MHC), MHCK7, C5-12, murine creatine kinase enhancer element, skeletal fast-twitch troponin c gene element, slow-twitch cardiac troponin c gene element, the slow-twitch troponin i gene element, hypoxia-inducible nuclear factor binding element, steroid-inducible element, or glucocorticoid response element (gre).

55. The system of claim 52, wherein the constitutive promoter comprises CMV, human U6 promoter, or H1 promoter.

56. The system of claim 52, wherein the constitutive promoter comprises a sequence of SEQ ID NO: 133 or 63.

57. The system of claim 46, wherein the first AAV ITR sequence comprises a sequence of SEQ ID NO: 132 or 59.

58. The system of claim 46, wherein the second AAV ITR sequence comprises a sequence of SEQ ID NO: 140 or 62.

59. The system of any one of claims 46-58, wherein the expression cassette comprises a sequence of SEQ ID NO: 128.

60. The system of any one of claims 46-59, wherein the expression cassette comprises a sequence of SEQ ID NO: 129.

61. The system of claim 46, wherein the Cas9 scaffold sequence is a spCas9 scaffold sequence or SaCas9 scaffold sequence.

62. The system of claim 61, wherein the Cas9 scaffold sequence is a SaCas9 scaffold sequence.

63. The system of claim 62, wherein the Cas9 scaffold sequence comprises a sequence of SEQ ID NO: 138 or 139 or 89 or 90.

64. The system of claim 46, wherein the one or more vectors encodes a Cas9 protein.

65. The system of claim 64, wherein the Cas9 protein is a SaCas9 or a spCas9 protein.

66. The system of claim 65, wherein the SaCas9 protein comprises an amino acid sequence of SEQ ID NO: 21 or 88 or is encoded by a polynucleotide comprising the sequence of SEQ ID NO: 69.

67. The system of any one of claims 46-66, wherein the one or more vectors are viral vectors.

68. The system of claim 67, wherein the viral vector is an Adeno-associated virus (AAV) vector.

69. The system of claim 68, wherein the AAV vector is AAV1, AAV2, AAV3, AAV4, AAV5, AAV6, AAV7, AAV8, AAV9, AAV-10, AAV-11, AAV-12, AAV-13, or AAVrh.74.

70. The system of any one of claims 46-69, wherein the vector that comprises an expression cassette is present in a concentration at least 2-fold, at least 3-fold, at least 4-fold, at least 5-fold, at least 6-fold, at least 7-fold, or at least 8-fold greater than the concentration of the vector encoding the Cas9 protein.

71. A cell comprising the system of any one of claims 46-70.

72. A kit comprising the system of any one of claims 46-70.

73. A method of correcting a mutant dystrophin gene in a cell, the method comprising administering to a cell the system of any one of claims 46-70.

74. A method of genome editing a mutant dystrophin gene in a subject, the method comprising administering to the subject the system of any one of claims 46-70 or the cell of claim 71.

75. A method of treating a subject having a mutant dystrophin gene, the method comprising administering to the subject the system of any one of claims 46-70 or the cell of claim 71.

76. The method of any one of claims 73-75, wherein the subject is a human.

77. The method of any one of claims 73-76, wherein the system of any one of claims 46-70 or the cell of claim 71 is administered to the subject intravenously.

78. The method of any one of claims 73-78, wherein the system of any one of claims 46-70 or the cell of claim 71 is administered to the subject systemically.

79. A plasmid expressing the expression cassette of claim 46, wherein the plasmid comprises a sequence selected from SEQ ID NOs: 87, 91, 92, 128, 129, 130, or 131.
